(19) United States
(12) Patent Application Publication
(10) Pub. No.: US 2025/0282794 A1
(43) Pub. Date: Sep. 11, 2025

(54) PYRIDONE AND PYRIMIDINONE INHIBITORS OF HEMATOPOIETIC PROGENITOR KINASE 1

(71) Applicant: CURADEV PHARMA LTD, Sandwich (GB)

(72) Inventors: David Cameron PRYDE, Sandwich (GB); Ritesh Kumar SHRIVASTAVA, Noida (IN); Dharmendra B. YADAV, Nodia (IN); Rajib GHOSH, Noida (IN); Sourav BASU, Noida (IN); Monali BANERJEE, Noida (IN); Sandip Kumar MIDDYA, Noida (IN); Sabyasachi DEBNATH, Noida (IN); Arjun SURYA, Noida (IN)

(21) Appl. No.: 19/207,325

(22) Filed: May 13, 2025

Related U.S. Application Data
(63) Continuation of application No. PCT/GB2023/052936, filed on Nov. 10, 2023.

(30) Foreign Application Priority Data
Nov. 15, 2022 (GB) 2216996.5
Nov. 15, 2022 (IN) 202211065483

(51) Int. Cl. C07D 498/04; A61K 31/5383; A61K 31/5386; A61K 31/541; C07D 519/00

(52) U.S. Cl. CPC C07D 498/04; A61K 31/5383; A61K 31/5386; A61K 31/541; C07D 519/00

(57) ABSTRACT

The present invention relates to compounds of formula (I). The compounds may be used to modulate activity of the HPK-1 protein, and thereby treat cancer, viral infection and immune-mediated disorders.

The present invention relates to compounds and methods useful for inhibiting Hematopoietic Progenitor Kinase 1 (HPK-1, MAP4K1). Accordingly, the inhibitors may be for use in the treatment of diseases, such as cancer and viral infections, and so on. The invention extends to the compounds per se, pharmaceutical compositions, methods of making the compounds and methods of inhibiting the HPK-1 protein.

HPK-1 is a member of the Ste20 family of serine/threonine kinases, expressed predominantly in hematopoietic cells. HPK-1 functions as a MAP4K kinase by phosphorylating MAP3K proteins including MEKK1, MLK3 and TAK1 and is thereby involved in modulating various downstream signalling pathways via activation of the JNK/SAPK signalling pathway (Hu et al., Genes Develop., 1996, 10, 2251-2264). This results in a functional modulation of various cellular processes such as cell proliferation, differentiation and stress responses to help maintain cellular hematopoiesis. Other members of the MAP4K family include MAP4K2/GCK, MAP4K3/GLK, MAP4K4/HGK, MAP4K5/KHS and MAP4K6/MINK. Whereas most kinases positively regulate cell function, HPK-1 is a negative regulator of T-cell receptor signalling which has led to it being investigated as a potential immune modulatory target for the treatment of cancer and viral infections.

Immune oncology is playing an increasingly important role as a treatment for cancer through mobilizing immune cells to recognize and ultimately eliminate cancer cells. There have been recent significant advances with monoclonal antibodies targeting T-cell inhibitory checkpoints such as CTLA-4, PD1 and PD-L1 being notable successes in driving durable anti-tumor responses in patients (Lee et al., Molecules, 2019, 24, 1190-1205).

In a productive immune response to tumors, the release of tumor antigens engages antigen receptors on immune cells including dendritic cells and antigen presenting cells to ultimately prime and activate T-cells. These antigen-specific T-cells traffic to the tumor site, infiltrate the tumor and kill the target cancer cells that comprise the tumor (Chen and Mellman, Immunity, 2013, 39, 1-10). However, this delicate balance of processes can be interrupted by mechanisms designed to evade detection by the immune system and bypass normal immune surveillance. Response rates to checkpoint inhibitor treatments are varied depending on the type of cancer being targeted, and appear to be sensitive to suppressive factors in the tumor microenvironment that lead to reduced T-cell effector functions and resistance to treatment (Sharma et al., Cell, 2017, 168, 707-723). Antibody treatments are also limited to targeting only extracellular inhibitory checkpoints/negative regulators, with a different modality required to access intracellular targets, for example with cell-permeable small molecules.

There is therefore a need for alternative means to modulate an immune response and in particular T-cell immune responses to broaden the clinical applicability of immune oncology treatments, for example by using small molecules.

Several small molecule kinase inhibitors that target specific pathways involved in negative regulation of T-cell responses to tumors have been proposed (Adams et al., Nat. Revs. Drug Disc., 2015, 14, 603-622; Weinmann, ChemMedChem, 2016, 11, 450-466; Sasikumar et al., BioDrugs, 2018, 32, 481-497).

HPK-1 has been studied as a possible immune oncology target due to its restricted cellular expression in hematopoietic cells such as T-cells, B-cells, macrophages, dendritic cells, neutrophils and mast cells (Kiefer et al., EMBO J., 1996, 15, 7013-7025). It has been shown to negatively regulate T-cell and dendritic cell function. HPK-1 knockout (KO) mice studies have confirmed its role in regulating T-cell activation, with HPK-1 KO or kinase-dead mice showing enhanced antigen presentation capacity, enhanced ERK1/2 activation and are able to mount augmented anti-tumor responses (Sawasdikisol et al., Immunol. Res., 2012, 54, 262-265; Liu et al., PLOS One, 2019, 14, e0212670; Hernandez et al., Cell Rep., 2018, 25, 80-94; Shui et al., Nat. Immunol., 2007, 8, 84-91). More recently, a potent small molecule inhibitor of HPK-1 has been shown to stimulate cytokine secretion from activated human T-cells and fully reverse the immune suppression driven by prostaglandin E2 and adenosine pathways (Wang et al., PLOS One, 2020, 15, e0243145). Another potent small molecule has also been shown to suppress tumor growth in an MC38 mouse syngeneic tumor model and in a CT26 mouse tumor explant model when combined with an anti-PD1 antibody (You et al., J. Immunother. Cancer, 2021, 9, c001402). These data clearly show that a loss of HPK-1 functional activity through both genetic deletion and pharmacological inhibition promoted T-cell activation and anti-tumor efficacy in pre-clinical models.

The activation of T-cells involves several enzymes, including kinases, to propagate the activation signal. HPK-1 kinase activity is induced by activation of T-cell and B-cell receptors (Liou et al., Immunity, 2000, 12, 399-408; Han et al., Immunity, 2003, 19, 621-632; Sauer et al., J. Biol. Chem., 2001, 276, 45207-45216), transforming growth factor receptors (TGF-PR) (Wang et al., J. Biol. Chem., 1997, 272, 22771-22775), PGE2 receptors EP2 and EP4 (Ikegami et al., J. Immunol., 2001, 166, 4689-4696), LPS receptors (Alzabin et al., J. Immunol., 2009, 182, 6187-6194) and by a caspase-mediated proteolytic cleavage of HPK-1 (Chen et al., Oncogene, 1999, 18, 7370-7377; Arnold et al., J. Biol. Chem., 2001, 276, 14675-14684). The activation, signalling and immunobiology of HPK-1 was reviewed recently by Sawasdikosol et al. (eLife, 2020, 9, e55122). HPK-1 is fully activated by ZAP-70 phosphorylation of Tyr379, along with the autophosphorylation of Thr165 and Ser171 (Sauer et al., Mol. Cell Biol., 2005, 25, 2364-2383). Once catalytically active, HPK-1 acts as a negative regulator of various immune cells including T-cells by phosphorylating SLP-76 at Ser376, disrupting downstream T-cell activating signalosome complexes and eventually leading to proteasome-mediated degradation of SLP-76 (DiBartolo et al., J. Expt. Med., 2007, 204, 681-691; Lasserre et al., J. Cell Biol., 2011, 195, 839-853; Wang et al., J. Biol. Chem., 2012, 287, 34091-34100). The negative regulation of B-cells operates in a very similar manner (Sauer et al., J. Biol. Chem., 2001, 276, 45207-45216; Wang et al., J. Biol. Chem., 2012, 287, 11037-11048).

The structure of the HPK-1 kinase domain has recently been solved in apo form as well as in co-structures with small molecule ligands (Wu et al., Structure, 2019, 27, 125-133; Johnson et al., J. Biol. Chem., 2019, 294, 9029-9036). The HPK-1 protein consists of multiple domains: an N-terminal kinase domain, a C-terminal citron homology domain, and an intrinsically disordered central domain containing four proline-rich (PR) motifs. The PR motifs mediate interaction with SH3 domain-containing proteins, such as Grb2 and Gads and the central domain contains the caspase cleavage site. The reported structures consist of domain-swapped dimers in which the activation segment presents a well-conserved dimer interface.

Several small molecule inhibitors of HPK-1 have been reported, but as an off-target rather than HPK-1 inhibition as a primary pharmacology. Such inhibitors include staurosporine, bosutinib, sunitinib, lestaurtinib, crizotinib, foretinib, dovitinib and KW-2449. All these compounds are potent inhibitors of receptor tyrosine kinases with HPK-1 inhibition being a weaker, off-target activity. Subsequently, several more potent HPK-1 inhibitors have been described in the literature (You et al., J. Immunother. Cancer, 2021, 9, e001402; Yu et al., ACS Med Chem Letts., 2021, 12, 459-466; Degnan et al., ACS Med Chem Letts., 2021, 12, 443-450; Vara et al., ACS Med Chem Letts., 2021, 12, 653-661) with varying degrees of HPK-1 enzyme potency, cell-based potency and kinase selectivity.

Accordingly, there remains a need in the art for improved therapies for treating diseases, such as cancer, which can be refractory to traditional therapeutic approaches. Immunologic strategies show promise for the treatment of cancer, and there is a need to develop improved compositions and methods in this field. In particular, there is a need for potent and selective small molecule inhibitors of HPK-1, as well as methods for treating diseases that can benefit from such inhibition.

Pyridone inhibitors of kinases are known in the literature. For example, Georges et. al. (WO2009024332) have described pyridone amide inhibitors of focal adhesion kinase (FAK), Bryan et. al. (ACS Med. Chem. Letts., 2016, 7, 100-104) have described pyridone inhibitors of the T790M double mutant of the epidermal growth factor receptor kinase and Pierre et. al. (Beilstein J. Org. Chem., 2021, 17, 156-165) have detailed a synthetic method for making pyridone amides. None of these reports indicate any HPK-1 activity data. The first two reports describe specific substituents that were designed to optimise activity at FAK and EGFR respectively, particularly 4-aminophenyl substituents, which are not preferred substituents for HPK-1 activity. The inventors have found that certain substituted pyridone carboxamides are potent and selective inhibitors of HPK-1.

In accordance with a first aspect of the invention, there is provided a compound of formula (I):

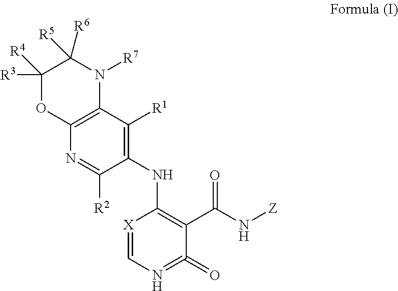

wherein:
X is CH or N;
Z is a phenyl or 5 or 6 membered heteroaryl, wherein the phenyl or heteroaryl is substituted with one or more substituents selected from the group consisting of a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 10 membered heterocyclyl, an optionally substituted 5 to 10 membered heteroaryl, an optionally substituted C₆-10 aryl or an optionally substituted C₃₋₉ cycloalkyl; and/or where adjacent substituents of the phenyl or heteroaryl, together with the atoms to which they are attached, may combine to form an optionally substituted 3 to 6 membered heterocycle or an optionally substituted 5 or 6 membered heteroaryl;
R¹ to R⁷ are independently hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl; and/or R³ and R⁴ and/or R⁵ and R⁶, together with the C atom they are bound to, form a C═O group, an optionally substituted C₃-C₆ cycloalkyl or an optionally substituted 3 to 8 membered heterocyclyl; and/or R³ and R⁵, together with the C atoms they are bound to, form an optionally substituted C₃-C₆ cycloalkyl or an optionally substituted 3 to 8 membered heterocyclyl;
R⁸ and R⁹ are independently hydrogen, an optionally substituted C₁-C₁₂ alkyl, an optionally substituted C₂-C₁₂ alkenyl, an optionally substituted C₂-C₁₂ alkynyl, optionally substituted C₆-12 aryl, optionally substituted C₃-C₆ cycloalkyl, optionally substituted 3 to 8 membered heterocyclyl, and an optionally substituted 5 to 10 membered heteroaryl; and
R¹⁰ and R¹¹ are independently hydrogen, an optionally substituted C₁-C₆ alkyl, an optionally substituted C₂-C₆ alkenyl, an optionally substituted C₂-C₆ alkynyl, optionally substituted C₆-12 aryl, optionally substituted C₃-C₆ cycloalkyl, optionally substituted 3 to 8 membered heterocyclyl, and an optionally substituted 5 to 10 membered heteroaryl; or a pharmaceutically acceptable complex, salt, solvate, tautomeric form or polymorphic form thereof.

In a second aspect of the invention, there is provided a pharmaceutical composition comprising a compound according to the first aspect, or a pharmaceutically acceptable salt, solvate, tautomeric form or polymorphic form thereof, and a pharmaceutically acceptable vehicle.

The invention also provides, in a third aspect, a process for making the composition according to the second aspect, the process comprising contacting a therapeutically effective amount of a compound of the first aspect, or a pharmaceutically acceptable salt, solvate, tautomeric form or polymorphic form thereof, and a pharmaceutically acceptable vehicle.

The inventors have found that compounds of the present invention are useful in therapy or as a medicament.

Hence, in a fourth aspect, there is provided a compound of formula (I), or a pharmaceutically acceptable complex, salt, solvate, tautomeric form or polymorphic form thereof, or a composition of the second aspect, for use in therapy.

The inventors have found that compounds of formula (I) are effective in modulating the activity of HPK-1.

Hence, in a fifth aspect, there is provided a compound of formula (I), or a pharmaceutically acceptable complex, salt, solvate, tautomeric form or polymorphic form thereof, or a composition of the second aspect, for use in modulating activity of the HPK-1 protein.

In a sixth aspect, there is provided use of a compound of formula (I) or a pharmaceutically acceptable complex, salt, solvate, tautomeric form or polymorphic form thereof in the manufacture of a medicament for modulating activity of the HPK-1 protein.

In a seventh aspect there is provided a method of modulating activity of the HPK-1 protein comprising administering a therapeutically effective amount of a compound of formula (I), or a pharmaceutically acceptable complex, salt, solvate, tautomeric form or polymorphic form thereof, or a composition of the second aspect, to a subject in need thereof.

Preferably, the compound of formula (I) is for use in inhibiting, or antagonising, the HPK-1 protein.

It will be appreciated that an ‘inhibitor’, as it relates to a ligand and HPK-1, comprises a molecule, combination of molecules, or a complex, that inhibits, antagonizes, counteracts, downregulates, and/or desensitizes HPK-1. ‘Inhibitor’ encompasses any reagent that inhibits a constitutive activity of HPK-1. A constitutive activity is one that is manifest in the absence of a ligand/HPK-1 interaction. ‘Inhibitor’ also encompasses any reagent that inhibits or prevents a stimulated (or regulated) activity of HPK-1.

Compounds of the present invention, and pharmaceutically relevant compositions thereof, are useful for treating a variety of diseases, disorders and conditions associated with regulation of signalling pathways that involve HPK-1.

By administering a compound of the present invention which inhibits the HPK-1 protein, an enhanced immune response may be manifest in a subject in need thereof. This enhanced immune response may include a T-cell population that shows enhanced priming, enhanced activation, enhanced migration, enhanced proliferation, enhanced survival and enhanced cytolytic activity relative to prior to the administration of the compound or pharmaceutical composition. T-cell activation is characterised by elevated levels of secreted cytokines such as IFNγ and IL-2, or elevated numbers of CD8 T-cells relative to prior to the administration of the compound or pharmaceutical composition. In certain aspects of this embodiment, the T-cell is an antigen-specific CD8 T-cell. In certain aspects of this embodiment, the antigen presenting cells in the subject have enhanced maturation and activation relative to prior to the administration of the compound or pharmaceutical composition. In certain aspects of this embodiment, the antigen presenting cells are dendritic cells.

The compounds of the present invention bind directly to the HPK-1 protein and inhibit its kinase activity. In certain embodiments, compounds of the present invention reduce, inhibit, antagonize or otherwise diminish the HPK-1-mediated phosphorylation of SLP76 and/or Gads.

The compounds of the present invention may or may not be specific inhibitors of HPK-1. A specific HPK-1 inhibitor reduces the biological activity of HPK-1 by an amount that is statistically greater than the inhibitory effect of the inhibitor on any other protein or biological target, for example another serine/threonine kinase or another type of kinase. In certain embodiments, compounds of the present invention specifically inhibit the serine/threonine kinase activity of HPK-1. In some of these embodiments, the IC₅₀ of the HPK-1 inhibitor for HPK-1 is 90%, 80%, 70%, 60%, 50%, 40%, 30%, 20%, 10%, 0.1%, 0.01%, 0.001% or less of the IC₅₀ of the HPK-1 inhibitor for another type of kinase.

Any method known in the art to measure the kinase activity of HPK-1 may be used to determine if HPK-1 has been inhibited, including in vitro enzymatic kinase assays, immunoblots with antibodies specific for phosphorylated targets of HPK-1 such as SLP76, FRET-based detection of specific phosphorylated targets of HPK-1 such as SLP76 or the measurement of a downstream biological effect of HPK-1 kinase activity for example T-cell or B-cell activation.

Compounds of the present invention can be used to treat diseases, disorders and conditions associated with regulation of signalling pathways that involve HPK-1. These diseases, disorders and conditions are pathological states in which HPK-1 activity is necessary for the creation or maintenance of the pathological state. In certain embodiments, the pathological state is cancer.

Compounds of the present invention can be used to treat diseases resulting from T-cell dysfunction, which is characterized by a decreased or absent responsiveness to antigenic stimulation. In a particular embodiment, T-cell dysfunction is associated with increased HPK-1 kinase activity. T-cell dysfunction can result in ineffective control of a pathogen or tumor. Examples of diseases resulting from T-cell dysfunction include unresolved acute infection, chronic infection and cancer.

By inhibiting the HPK-1 protein, it is possible to treat, ameliorate or prevent cancer, viral infection and immune-mediated disorders.

Accordingly, in an eighth aspect there is provided a compound of formula (I), or a pharmaceutically acceptable complex, salt, solvate, tautomeric form or polymorphic form thereof, or a composition of the second aspect, for use in treating, ameliorating or preventing a disease selected from cancer, viral infection and immune-mediated disorders.

Preferably, the treatment, amelioration or prevention comprises inhibiting the HPK-1 protein.

In a ninth aspect, there is provided use of a compound of formula (I) or a pharmaceutically acceptable complex, salt, solvate, tautomeric form or polymorphic form thereof in the manufacture of a medicament for treating, ameliorating or preventing a disease selected from cancer, viral infection and immune-mediated disorders.

In a tenth aspect, there is provided a method of treating, ameliorating or preventing a disease selected from cancer, viral infection and immune-mediated disorder, the method comprising administering, to a subject in need of such treatment, a therapeutically effective amount of a compound of formula (I), or a pharmaceutically acceptable complex, salt, solvate, tautomeric form or polymorphic form thereof, or a composition of the second aspect.

It may be appreciated that the term “preventing” can mean “reducing the likelihood of”.

In an eleventh aspect there is provided a compound of formula (I) for use in modulation of activity of the HPK-1 protein:

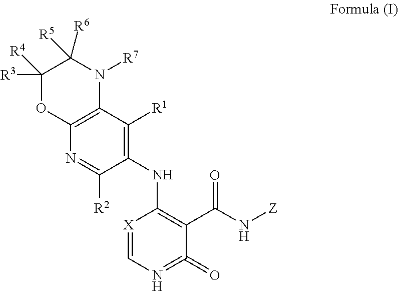

X is CH or N;
Z is a phenyl or 5 or 6 membered heteroaryl, wherein the phenyl or heteroaryl is substituted with one or more substituents selected from the group consisting of a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 10 membered heterocyclyl, an optionally substituted 5 to 10 membered heteroaryl, an optionally substituted C₆-10 aryl or an optionally substituted C₃₋₉ cycloalkyl; and/or where adjacent substituents of the phenyl or heteroaryl, together with the atoms to which they are attached, may combine to form an optionally substituted 3 to 6 membered heterocycle or an optionally substituted 5 or 6 membered heteroaryl;
R¹ to R⁷ are independently hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl; and/or R³ and R⁴ and/or R⁵ and R⁶, together with the C atom they are bound to, form a C═O group, an optionally substituted C₃-C₆ cycloalkyl or an optionally substituted 3 to 8 membered heterocyclyl; and/or R³ and R⁵, together with the C atoms they are bound to, form an optionally substituted C₃-C₆ cycloalkyl or an optionally substituted 3 to 8 membered heterocyclyl;
R⁸ and R⁹ are independently hydrogen, an optionally substituted C₁-C₁₂ alkyl, an optionally substituted C₂-C₁₂ alkenyl, an optionally substituted C₂-C₁₂ alkynyl, optionally substituted C₆-12 aryl, optionally substituted C₃-C₆ cycloalkyl, optionally substituted 3 to 8 membered heterocyclyl, and an optionally substituted 5 to 10 membered heteroaryl; and
R¹⁰ and R¹¹ are independently hydrogen, an optionally substituted C₁-C₆ alkyl, an optionally substituted C₂-C₆ alkenyl, an optionally substituted C₂-C₆ alkynyl, optionally substituted C₆-12 aryl, optionally substituted C₃-C₆ cycloalkyl, optionally substituted 3 to 8 membered heterocyclyl, and an optionally substituted 5 to 10 membered heteroaryl; or a pharmaceutically acceptable complex, salt, solvate, tautomeric form or polymorphic form thereof.

In some embodiments, compounds of the present invention can be used to treat viral infections in a subject of need thereof. In some embodiments, compounds of the present invention can be used as an adjuvant to increase the efficacy of vaccination.

The viral disease may be Hepatitis B, Hepatitis C or HIV.

In one preferred embodiment, the disease is cancer. The cancer may be selected from the group consisting of colorectal cancer, aero-digestive squamous cancer, lung cancer, brain cancer, liver cancer, stomach cancer, sarcoma, leukaemia, lymphoma, multiple myeloma, ovarian cancer, uterine cancer, breast cancer, melanoma, prostate cancer, bladder cancer, glioma, pancreatic carcinoma or renal carcinoma.

In some embodiments, cancers that are treatable using the compounds of formula (I) include but are not limited to solid tumors (e.g. prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer), haematological cancers (e.g. lymphoma, leukemia such as acute lymphoblastic leukemia (ALL), acute myelogenous leukemia (AML), chronic lymphocytic leukemia (CLL), chronic myelogenous leukemia (CML), non-Hodgkin lymphoma, Hodgkin lymphoma or multiple myeloma) and combinations of said cancers.

In some embodiments, HPK-1 inhibitors may be used to treat tumors producing PGE2 (COX-2 overexpressing tumors) and/or adenosine (CD73 and CD39 overexpressing tumors). Overexpression of COX-2 has been detected in several tumors such as colorectal, breast, pancreatic and lung cancers. CD73 is up-regulated in various carcinomas including those of the colon, lung, pancreas and ovary.

In an alternative preferred embodiment, the disease is a viral infection. The viral infection may be a hepatitis B, hepatitis C virus (HCV) infection or a human immunodeficiency virus (HIV) infection.

The following definitions are used in connection with the compounds of the present invention unless the context indicates otherwise.

Throughout the description and the claims of this specification the word “comprise” and other forms of the word, such as “comprising” and “comprises,” means including but not limited to, and is not intended to exclude for example, other additives, components, integers, or steps.

As used in the description and the appended claims, the singular forms “a”, “an”, and “the” include plural referents unless the context clearly dictates otherwise. Thus, for example, reference to “a composition” includes mixtures of two or more such compositions.

“Optional” or “optionally” means that the subsequently described event, operation or circumstances can or cannot occur, and that the description includes instances where the event, operation or circumstance occurs and instances where it does not.

The term “alkyl” as used herein, unless otherwise specified, refers to a saturated straight or branched hydrocarbon. In certain embodiments, the alkyl group is a primary, secondary, or tertiary hydrocarbon. In certain embodiments, the alkyl group includes one to six carbon atoms, i.e. C₁-C₆ alkyl. C₁-C₆ alkyl includes for example methyl, ethyl, n-propyl (1-propyl) and isopropyl (2-propyl, 1-methylethyl), butyl, pentyl, hexyl, isobutyl, sec-butyl, tert-butyl, isopentyl, neopentyl and isohexyl. In alternative embodiments, the alkyl group includes one to three carbon atoms, i.e. C₁-C₃ alkyl. An alkyl group can be unsubstituted or substituted with one or more of halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. Accordingly, it will be appreciated that an optionally substituted C₁-C₆ alkyl may be an optionally substituted C₁-C₆ haloalkyl, i.e. a C₁-C₆ alkyl substituted with at least one halogen, and optionally further substituted with one or more of oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. The optionally substituted C₁-C₆ alkyl may be a polyfluoroalkyl, preferably a C₁-C₃ polyfluoroalkyl, and most preferably CF₃.

R¹⁶ and R¹⁷ are independently hydrogen, an optionally substituted C₁-C₆ alkyl, an optionally substituted C₂-C₆ alkenyl, an optionally substituted C₂-C₆ alkynyl, optionally substituted C₆₋₁₂ aryl, optionally substituted C₃-C₆ cycloalkyl, optionally substituted 3 to 8 membered heterocyclyl or an optionally substituted 5 to 10 membered heteroaryl.

The term “halo” or “halogen” includes fluoro (—F), chloro (—Cl), bromo (—Br) and iodo (—I).

The term “polyfluoroalkyl” may denote a C₁-C₃ alkyl group in which two or more hydrogen atoms are replaced by fluorine atoms. The term may include perfluoroalkyl groups, i.e. a C₁-C₃ alkyl group in which all the hydrogen atoms are replaced by fluorine atoms. Accordingly, the term C₁-C₃ polyfluoroalkyl includes, but is not limited to, difluoromethyl, trifluoromethyl, 2,2,2-trifluoroethyl, pentafluoroethyl, 3,3,3-trifluoropropyl, 2,2,3,3,3-pentafluoropropyl, and 2,2,2-trifluoro-1-(trifluoromethyl)ethyl.

“Alkenyl” refers to an olefinically unsaturated hydrocarbon groups which can be unbranched or branched. In certain embodiments, the alkenyl group has 2 to 6 carbons, i.e. it is a C₂-C₆ alkenyl. C₂-C₆ alkenyl includes for example vinyl, allyl, propenyl, butenyl, pentenyl and hexenyl. In alternative embodiments, the alkenyl group has 2 to 3 carbons, i.e. it is a C₂-C₃ alkenyl. An alkenyl group can be unsubstituted or substituted with one or more of optionally substituted C₂-C₆ alkynyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. R¹⁶ and R¹⁷ may be as defined above.

“Alkynyl” refers to an acetylenically unsaturated hydrocarbon groups which can be unbranched or branched. In certain embodiments, the alkynyl group has 2 to 6 carbons, i.e. it is a C₂-C₆ alkynyl. C₂-C₆ alkynyl includes for example propargyl, propynyl, butynyl, pentynyl and hexynyl. In alternative embodiments, the alkynyl group has 2 to 3 carbons, i.e. it is a C₂-C₃ alkynyl. An alkynyl group can be unsubstituted or substituted with one or more of optionally substituted C₂-C₆ alkenyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. R¹⁶ and R¹⁷ may be as defined above.

“Cycloalkyl” refers to a non-aromatic, saturated or partially saturated, monocyclic, bicyclic or polycyclic hydrocarbon 3 to 6 membered ring system. Representative examples of a C₃-C₆ cycloalkyl include, but are not limited to, cyclopropyl, cyclobutyl, cyclopentyl, cyclohexyl. A cycloalkyl group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. R¹⁶ and R¹⁷ may be as defined above.

“Heteroaryl”, unless specified otherwise, refers to a monocyclic or bicyclic aromatic 5 to 10 membered ring system in which at least one ring atom is a heteroatom. The term includes bicyclic groups where one of the rings is aromatic and the other is not. For groups where one of the rings is aromatic and the other is not, the group will be considered to be a heteroaryl group if either or both rings contain at least one ring atom which is a heteroatom. In some embodiments, the heteroaryl is a monocyclic 5 or 6 membered ring system in which at least one ring atom is a heteroatom. The or each heteroatom may be independently selected from the group consisting of oxygen, sulfur and nitrogen. The heteroaryl may contain 1, 2, 3 or 4 heteroatoms. Examples of 5 to 10 membered heteroaryl groups include furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic 5 to 10 membered heteroaryl groups include those where a phenyl, pyridine, pyrimidine, pyrazine or pyridazine ring is fused to a 5 or 6-membered monocyclic heteroaryl ring. A heteroaryl group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. R¹⁶ and R¹⁷ may be as defined above.

“Heterocycle” or “heterocyclyl”, unless specified otherwise, refers to 3 to 10 membered monocyclic, bicyclic or bridged molecules in which at least one ring atom is a heteroatom. In some embodiments, a heterocycle is a 3 to 6 monocyclic molecule in which at least one ring atom is a heteroatom. The or each heteroatom may be independently selected from the group consisting of oxygen, sulfur and nitrogen. The heterocycle may contain 1, 2, 3 or 4 heteroatoms. A heterocycle may be saturated or partially saturated. Exemplary 3 to 8 membered heterocycle groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, morpholine, piperazine, thiane, thiine, piperazine, azepane, diazepane and oxazine. A heterocycle group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. R¹⁶ and R¹⁷ may be as defined above.

“Aryl” refers to an aromatic 6 to 12 membered hydrocarbon group. The term includes bicyclic groups where one of the rings is aromatic and the other is not. It may be appreciated that in aryl groups all of the ring atoms are carbon. Examples of a C₆-C₁₂ aryl group include, but are not limited to, phenyl, α-naphthyl, β-naphthyl, biphenyl, tetrahydronaphthyl and indanyl. An optionally substituted aryl group may be an optionally substituted phenyl group. An optionally substituted aryl group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. R¹⁶ and R¹⁷ may be as defined above.

The term “bicycle” or “bicyclic” as used herein refers to a molecule that features two rings. Similarly, “tricyclic” as used herein refers to a molecule that features three rings. Similarly, “polycyclic” as used herein refers to a molecule that features three or more rings. In each case, the rings may be optionally substituted phenyl, optionally substituted cycloalkyl, optionally substituted heterocyclyl, optionally substituted heteroaryl or a combination thereof. In one embodiment, the rings are fused across a bond between two atoms. The moiety formed therefrom shares a bond between the rings, and may be referred to as “fused”. In another embodiment, the moiety is formed by the fusion of two rings across a sequence of atoms of the rings to form a bridgehead. Similarly, a “bridge” is an unbranched chain of one or more atoms connecting two bridgeheads in a bicyclic or polycyclic compound. In another embodiment, the molecule is a “spiro” or “spirocyclic” moiety. The spirocyclic group may be a C₃-C₆ cycloalkyl or a mono or bicyclic 3 to 8 membered heterocycle which is bound through a single carbon atom of the spirocyclic moiety to a single carbon atom of a carbocyclic or heterocyclic moiety. In one embodiment, the spirocyclic group is a cycloalkyl and is bound to another cycloalkyl. In another embodiment, the spirocyclic group is a cycloalkyl and is bound to a heterocyclyl. In a further embodiment, the spirocyclic group is a heterocyclyl and is bound to another heterocyclyl. In still another embodiment, the spirocyclic group is a heterocyclyl and is bound to a cycloalkyl.

In some embodiments, R⁸ and R⁹ are independently hydrogen, an optionally substituted C₁-C₃ alkyl, an optionally substituted C₂-C₃ alkenyl, an optionally substituted C₂-C₃ alkynyl, optionally substituted C₆₋₁₂ aryl, optionally substituted C₃-C₆ cycloalkyl, optionally substituted 3 to 8 membered heterocyclyl, and an optionally substituted 5 to 10 membered heteroaryl.

R¹ may be hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹ or NR¹⁰R¹¹. More preferably, R¹ is hydrogen, an optionally substituted C₁-C₃ alkyl, an optionally substituted C₂-C₃ alkenyl, an optionally substituted C₂-C₃ alkynyl, a halogen, CN or OR¹⁰. Even more preferably, R¹ is hydrogen, C₁-C₃ alkyl, CN or OR¹⁰. R⁸ and R⁹ may independently be hydrogen, an optionally substituted C₁-C₃ alkyl, an optionally substituted C₂-C₃ alkenyl or an optionally substituted C₂-C₃ alkynyl. More preferably, R⁸ and R⁹ are independently hydrogen, a C₁-C₃ alkyl, a C₂-C₃ alkenyl or a C₂-C₃ alkynyl. Even more preferably, R⁸ and R⁹ are H or methyl, and most preferably are methyl. R¹⁰ and R¹¹ may independently be hydrogen, an optionally substituted C₁-C₆ alkyl, an optionally substituted C₂-C₆ alkenyl or an optionally substituted C₂-C₆ alkynyl. More preferably, R¹⁰ and R¹¹ are independently hydrogen, an optionally substituted C₁-C₃ alkyl, an optionally substituted C₂-C₃ alkenyl or an optionally substituted C₂-C₃ alkynyl. More preferably, R¹⁰ and R¹¹ are independently hydrogen, a C₁-C₃ alkyl, a C₂-C₃ alkenyl or a C₂-C₃ alkynyl. Even more preferably, R¹⁰ and R¹¹ are H or methyl, and most preferably are methyl. Accordingly, R¹ may be hydrogen, methyl, CN or OCH₃.

R² may be hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹ or NR¹⁰R¹¹. More preferably, R² is hydrogen, an optionally substituted C₁-C₃ alkyl, an optionally substituted C₂-C₃ alkenyl, an optionally substituted C₂-C₃ alkynyl, a halogen, CN or OR¹⁰. Even more preferably, R² is hydrogen, C₁-C₃ alkyl, CN or OR¹⁰. R⁸ to R¹¹ may be as defined above in relation to the definition of R¹. Most preferably, R² is hydrogen.

R³ and R⁴ may independently be hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹°, COOR⁸, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl or R³ and R⁴ together with the C atom they are bound to, may form a C═O group, an optionally substituted C₃-C₆ cycloalkyl or an optionally substituted 3 to 8 membered heterocyclyl. More preferably, R³ and R⁴ are independently hydrogen, an optionally substituted C₁-C₃ alkyl, an optionally substituted C₂-C₃ alkenyl, an optionally substituted C₂-C₃ alkynyl, a halogen, CN, OR¹⁰, an optionally substituted C₃-C₆ cycloalkyl or an optionally substituted 3 to 8 membered heterocyclyl, or R³ and R⁴ together with the C atom they are bound to, may form an optionally substituted C₃-C₆ cycloalkyl or an optionally substituted 3 to 8 membered heterocyclyl. Even more preferably, R³ and R⁴ are independently hydrogen, a C₁-C₃ alkyl or a halogen, or R³ and R⁴ together with the C atom they are bound to, may form a C₃-C₆ cycloalkyl. R⁸ to R¹¹ may be as defined above in relation to the definition of R¹. Most preferably, R³ and R⁴ are independently hydrogen, methyl, ethyl, i-propyl or fluorine, or R³ and R⁴ together with the C atom they are bound to form cyclopropyl.

R⁵ and R⁶ may independently be hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹°, COOR⁸, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl or R⁵ and R⁶ together with the C atom they are bound to, may form a C═O group, an optionally substituted C₃-C₆ cycloalkyl or an optionally substituted 3 to 8 membered heterocyclyl. More preferably, R⁵ and R⁶ are independently hydrogen, an optionally substituted C₁-C₃ alkyl, an optionally substituted C₂-C₃ alkenyl, an optionally substituted C₂-C₃ alkynyl, a halogen, CN, OR¹⁰, an optionally substituted C₃-C₆ cycloalkyl or an optionally substituted 3 to 8 membered heterocyclyl, or R⁵ and R⁶ together with the C atom they are bound to, may form a C═O group. Even more preferably, R⁵ and R⁶ are independently hydrogen, a C₁-C₃ alkyl or a halogen, or R⁵ and R⁶ together with the C atom they are bound to, may form a C═O group. R⁸ to R¹¹ may be as defined above in relation to the definition of R¹. Most preferably, R⁵ and R⁶ are hydrogen, or R⁵ and R⁶ together with the C atom they are bound to form a C═O group.

R⁷ may be hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹ or NR¹⁰R¹¹. More preferably, R⁷ is hydrogen, an optionally substituted C₁-C₃ alkyl, an optionally substituted C₂-C₃ alkenyl, an optionally substituted C₂-C₃ alkynyl, a halogen, CN or OR¹⁰. Even more preferably, R⁷ is hydrogen, C₁-C₃ alkyl, CN or OR¹⁰. R⁸ to R¹¹ may be as defined above in relation to the definition of R¹. Most preferably, R⁷ is hydrogen or methyl.

Accordingly, Y may be

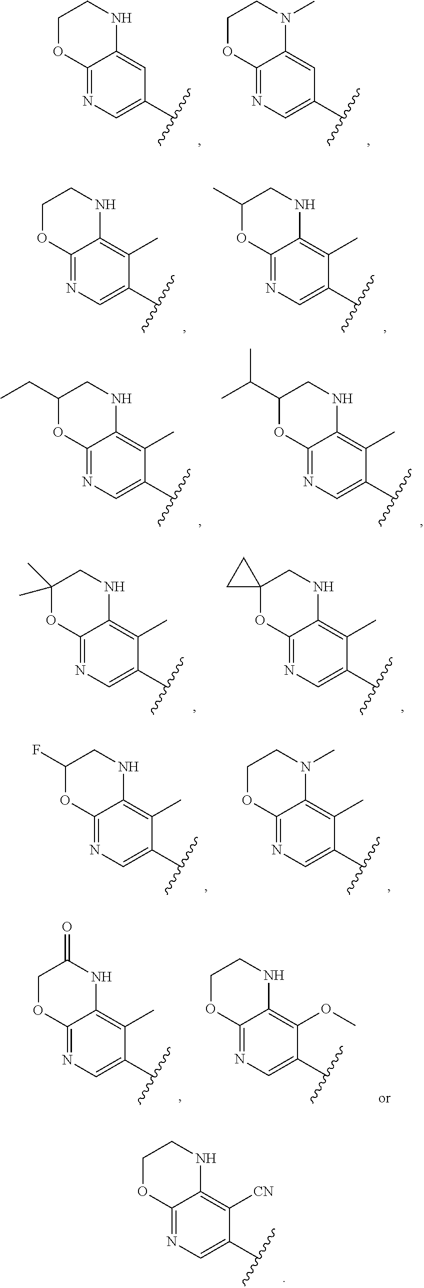

Z may be a substituted phenyl, a substituted pyrazole or a substituted pyridinyl group.

In one preferred arrangement, adjacent substituents of the phenyl or 5 or 6 membered heteroaryl group Z are linked to form, with the C atoms of the phenyl or heteroaryl group they are bound to, a 5- or 6-membered heterocyclic or heteroaromatic group. It will be appreciated that in these embodiments the group Z is an optionally substituted fused group. Furthermore, adjacent substituents of the heterocyclic or heteroaryl group, together with the atoms to which they are attached, may combine to form a further optionally substituted 3 to 6 membered heterocycle or a further optionally substituted 5 or 6 membered heteroaryl. Accordingly, the optionally substituted fused group may be an optionally substituted bicyclic fused group or an optionally substituted tricyclic fused group. The optionally substituted bicyclic or tricyclic fused group may be an optionally substituted 8-to 14-membered heterocyclic or heteroaromatic group. In some embodiments, Z is an the optionally substituted bicyclic fused group, and is an optionally substituted 9- or 10-membered heterocyclic or heteroaromatic group. In alternative embodiments, Z is an optionally substituted tricyclic fused group, and is an optionally substituted 12- to 14-membered heterocyclic or heteroaromatic group. A preferred fused group Z is optionally substituted benzo[d][1,3]dioxole, optionally substituted indoline, optionally substituted 1H-indazole, optionally substituted 1H-benzo[d]imidazole, optionally substituted benzo[d]thiazole, optionally substituted tetrahydroquinoline, optionally substituted tetrahydroisoquinoline, optionally substituted 3,4-dihydro-2H-benzo[b][1,4]oxazinc, optionally substituted 2,3-dihydrobenzo[b][1,4]dioxine, optionally substituted isoquinoline, optionally substituted quinoxaline or optionally substituted 1,2,3,4,4a,5-hexahydrobenzo[b]pyrazino[1,2-d][1,4]oxazinc. The fused group may be unsubstituted or substituted with one or more of optionally substituted C₁-C₆ alkyl, optionally substituted C₂-C₆ alkenyl, optionally substituted C₂-C₆ alkynyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. More preferably, the fused group is unsubstituted or substituted with one or more of optionally substituted C₁-C₃ alkyl, optionally substituted C₂-C₃ alkenyl, optionally substituted C₂-C₃ alkynyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷ or NR¹⁶R¹⁷. Most preferably, the fused group is unsubstituted or substituted with one or more of optionally substituted C₁-C₃ alkyl, oxo, OR¹⁶ or NR¹⁶COR¹⁷. In embodiments where the fused group is substituted with an optionally substituted alkyl, an optionally substituted alkenyl or an optionally substituted alkynyl, the alkyl, alkenyl or alkynyl may be unsubstituted or substituted with one or more of halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷ and NR¹⁶R¹⁷. More preferably, the alkyl, alkenyl or alkynyl is unsubstituted or substituted with OR¹⁶. R¹⁶ and R¹⁷ may be as defined above. Preferably, R¹⁶ and R¹⁷ may independently be H, optionally substituted C₁₋₃ alkyl or optionally substituted C₃₋₆ cycloalkyl, wherein the alkyl or cycloalkyl is unsubstituted or substituted with halogen or OH. Most preferably, R¹⁶ and R¹⁷ are independently H, methyl and optionally substituted cyclopropyl, wherein the cyclopropyl is optionally substituted with a fluorine. The fused group may be unsubstituted or substituted with between one and six substituents. More preferably, the fused group is unsubstituted or substituted with 1, 2 or 3 substituents. Optional substituents include, without limitation, C₁-C₆ alkyl or F. More preferably, optional substituents are CH₃ or F. In a most preferred embodiment, the fused group is unsubstituted or substituted with one or more of, methyl, ethyl,

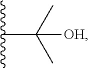 [SMILES: *C(C)(C)O]

### OH, OCH₃, oxo or

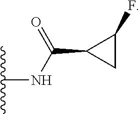 [SMILES: *NC(=O)[C@@H]1C[C@@H]1F]

A preferred group Z has formula:

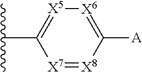

wherein X⁵, X⁶, X⁷ and X⁸ are each independently selected from N and CR²¹ with the proviso that no more than one of X⁵, X⁶, X⁷ and X⁸ is N; R²¹ in each occurrence is independently H or a halogen, preferably F; and A is selected from an optionally substituted C₁-C₆ alkyl, COR⁸, COOR⁸, CONR⁸R⁹, NR⁸COR⁹, NR⁸R⁹, an optionally substituted 3 to 10 membered heterocyclyl and an optionally substituted 5 to 10 membered heteroaryl. R⁸ and R⁹ may independently be hydrogen, an optionally substituted C₁-C₃ alkyl, an optionally substituted C₂-C₃ alkenyl, an optionally substituted C₂-C₃ alkynyl, optionally substituted phenyl, optionally substituted C₃-C₆ cycloalkyl, optionally substituted 3 to 8 membered heterocyclyl, or optionally substituted 5 to 10 membered heteroaryl. More preferably, R⁸ and R⁹ are independently hydrogen, an optionally substituted C₁-C₃ alkyl or optionally substituted 5 or 6 membered heterocyclyl. The alkyl may be unsubstituted or substituted with one or more of halogen, OR¹⁶ or NR¹⁶R¹⁷. R¹⁶ and R¹⁷ may be as defined above. In some embodiments, R¹⁶ and R¹⁷ may independently be H or C₁-C₃ alkyl.

An alternative preferred group Z has formula:

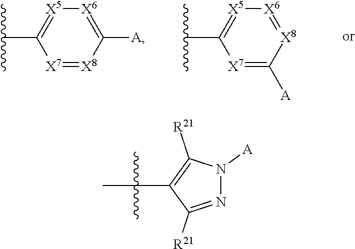

wherein X⁵, X⁶, X⁷ and X⁸ are N or CR²¹ with the proviso that no more than one of X⁵, X⁶, X⁷ and X⁸ is N;

R²¹ in each occurrence is independently H, a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 6 membered heterocyclyl, an optionally substituted 5 or 6 membered heteroaryl, an optionally substituted C₆₋₁₀ aryl or an optionally substituted C₃₋₉ cycloalkyl; and

A is selected from an optionally substituted C₁-C₆ alkyl, OR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 10 membered heterocyclyl and an optionally substituted 5 to 10 membered heteroaryl.

In some embodiments, X⁵, X⁶, X⁷ and X⁸ are all CR²¹.

In alternative embodiments, X⁵, X⁷ and X⁸ are all CR²¹ and X⁶ is N.

In one embodiment, the phenyl or 5 or 6 membered heteroaryl group Z is substituted with an optionally substituted 3 to 10 membered heterocyclyl, an optionally substituted 5 to 10 membered heteroaryl, an optionally substituted C₆₋₁₀ aryl or an optionally substituted C₃₋₉ cycloalkyl. It may be appreciated that the optionally substituted heterocyclyl, optionally substituted heteroaryl, optionally substituted aryl or optionally substituted cycloalkyl may be group A in the above formula. In some embodiments, the phenyl or 5 or 6 membered heteroaryl group Z is substituted with an optionally substituted 3 to 10 membered heterocyclyl or an optionally substituted 5 to 10 membered heteroaryl.

A preferred optionally substituted heteroaryl group which is a substituent on the Z group, which may optionally be A in the above formula, is optionally substituted pyridinyl, optionally substituted pyrazolyl, optionally substituted oxazolyl or optionally substituted 2H-1,2,3-triazolyl. A preferred heteroaryl group which is a substituent on the Z group, which may optionally be A in the above formula, is pyridyl, pyrazolyl or oxazolyl.

A preferred optionally substituted heterocyclic group which is a substituent on the Z group, which may optionally be A in the above formula, is optionally substituted pyrrolidinyl, optionally substituted piperazinyl, optionally substituted piperidinyl, optionally substituted tetrahydropyranyl, optionally substituted morpholinyl, optionally substituted thiomorpholinyl, optionally substituted azepanyl, optionally substituted octahydropyrrolo[1,2-a]pyrazinyl, optionally substituted octahydroimidazo[1,5-a]pyrazinyl, optionally substituted octahydropyrazino[2,1-c][1,4]oxazinyl, optionally substituted octahydro-2H-pyrido[1,2-a]pyrazinyl, optionally substituted 1,5-diazabicyclo[2.2.1]heptanyl, optionally substituted 3,8-diazabicyclo[3.2.1]octanyl or optionally substituted 2,5-diazabicyclo[2.2.2]octanyl.

A preferred heterocyclyl group which is a substituent on the Z group, which may optionally be A in the above formula, is a group of formula (i) or (j):

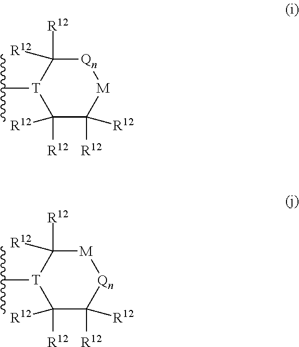

wherein:
T is N and M is NR¹³, CR¹⁴R¹⁵, O, S or SO₂; or T is CR¹⁸ and M is NR¹³, O, S or SO₂;
Q is C(R¹²)₂ and n is 0, 1 or 2;
R¹² in each occurrence is independently H, optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl; and/or two R¹² groups bonded to the same carbon may define an oxo group, two R¹² groups bonded to adjacent carbon atoms may be linked to form a fused group or two R¹² groups bonded to non-adjacent carbon atoms may be linked to form a bicyclic bridged group;
R¹³ is H, optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl;
R¹⁴ and R¹⁵ are each independently selected from H, optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl.

A preferred heterocyclyl group which is a substituent on the Z group, which may optionally be A in the above formula, is a group of formula (i) or (j):

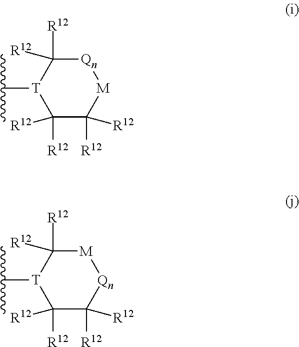

wherein:
T is N and M is NR¹³, CR¹⁴R¹⁵, O, S or SO₂; or T is CR¹⁸ and M is NR¹³, O, S or SO₂; Q is C(R¹²)₂ and n is 1 or 2;
R¹² in each occurrence is independently H, halogen, optionally substituted C₁₋₆ alkyl, optionally substituted C₂₋₆ alkenyl, optionally substituted C₂₋₆ alkynyl, optionally substituted C₃₋₆ cycloalkyl, OR¹⁹; or NR¹⁹R²⁰; and/or two R¹² groups bonded to adjacent carbon atoms may be linked to form a fused group or two R¹² groups bonded to non-adjacent carbon atoms may be linked to form a bicyclic bridged group;
R¹³ is H, optionally substituted C₁-C₆ alkyl, optionally substituted C₂₋₆ alkenyl, optionally substituted C₂₋₆ alkynyl, optionally substituted C₃₋₆ cycloalkyl, optionally substituted phenyl, optionally substituted 5 or 6 membered heteroaryl, COR¹⁹ or CONR¹⁹R²⁰;
R¹⁴ and R¹⁵ are each independently selected from hydrogen, halogen, an optionally substituted C₁-C₆ alkyl, optionally substituted C₂₋₆ alkenyl, optionally substituted C₂₋₆ alkynyl, optionally substituted C₃-C₆ cycloalkyl, OR¹⁹ and NR¹⁹R²⁰, or R¹⁴ and R¹⁵ together with the C atom they are both bonded may be linked to form an optionally substituted 3 to 6 membered heterocyclyl or an optionally substituted C₃₋₆ cycloalkyl;
R¹⁸ is hydrogen or optionally substituted C₁-C₆ alkyl; and
R¹⁹ and R²⁰ are each independently H, optionally substituted C₁₋₆ alkyl, optionally substituted C₂₋₆ alkenyl, optionally substituted C₂₋₆ alkynyl, optionally substituted C₃₋₆ cycloalkyl group, optionally substituted 5 or 6 membered heteroaryl or optionally substituted 3 to 6 membered heterocyclyl.

The heterocyclyl or heteroaryl which is a substituent on the Z group may be unsubstituted or substituted with one or more optional substituents, which may be selected from the group consisting of optionally substituted C₁-C₆ alkyl, optionally substituted C₂-C₆ alkenyl, optionally substituted C₂-C₆ alkynyl, optionally substituted C₁-C₆ alkoxy, optionally substituted C₃₋₆ cycloalkyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷ or NR¹⁶R¹⁷. More preferably, the heterocyclyl or heteroaryl which is a substituent on the Z group may be unsubstituted or substituted with one or more substituents which may be selected from the group consisting of optionally substituted C₁-C₃ alkyl, optionally substituted C₂-C₆ alkenyl, optionally substituted C₂-C₆ alkynyl, optionally substituted C₃₋₆ cycloalkyl, halogen, oxo, COR¹⁶, COOR¹⁶, CONR¹⁶R¹⁷, NR¹⁶COR¹⁷ or NR¹⁶R¹⁷. The alkyl may be substituted with OR¹⁶ and NR¹⁶R¹⁷. R¹⁶ and R¹⁷ may be as defined above. In some embodiments, R¹⁶ and R¹⁷ may each independently be selected from the group consisting of H, C₁-C₃ alkyl or optionally halogenated 5 or 6 membered heterocycle.

The heterocyclyl, heteroaryl, aryl or cycloalkyl which is a substituent on the Z group, and may be group A in the above formula, may be unsubstituted or substituted with one or more optional substituents, which may be selected from the group consisting of optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. It will be appreciated that these substituents may be the groups R¹², R¹³, R¹⁴ and/or R¹⁵. Accordingly, R¹³, R¹⁴ and/or R¹⁵ may be H or a substituent, as defined below. Similarly, R¹² be H or a substituent, as defined below, and/or two R¹² groups bonded to the same carbon may define an oxo group, two R¹² groups bonded to adjacent carbon atoms may be linked to form a fused group or two R¹² groups bonded to non-adjacent carbon atoms may be linked to form a bicyclic bridged group. Most preferably, these substituents may be the groups R¹³ or R¹⁴. More preferably, the heterocyclyl, heteroaryl, aryl or cycloalkyl which is a substituent on the Z group may be unsubstituted or substituted with one or more substituents selected from the group consisting of optionally substituted C₁-C₆ alkyl, oxo, CN, OR¹⁶, SO₂R¹⁶, COR¹⁶, COOR¹⁶, CONR¹⁶R¹⁷, NR¹⁶COR¹⁷, NR¹⁶R¹⁷, optionally substituted C₃₋₆ cycloalkyl, optionally substituted 3 to 6 membered heterocycle or optionally substituted 5 or 6 membered heteroaryl. R¹⁶ and R¹⁷ may be as defined above. Preferably, R¹⁶ and R¹⁷ are independently hydrogen, an optionally substituted C₁-C₆ alkyl, optionally substituted C₃-C₆ cycloalkyl, optionally substituted 3 to 8 membered heterocyclyl or an optionally substituted 5 to 10 membered heteroaryl. More preferably, R¹⁶ and R¹⁷ are independently hydrogen, an optionally substituted C₁-C₆ alkyl, optionally substituted C₃-C₆ cycloalkyl, optionally substituted 4 to 8 membered heterocyclyl or an optionally substituted 5 or 6 membered heteroaryl. When the heterocyclyl, heteroaryl, aryl or cycloalkyl which is a substituent on the Z group is substituted, either directly or indirectly, with an optionally substituted alkyl, an optionally substituted alkenyl or an optionally substituted alkynyl, the alkyl, alkenyl or alkynyl may be unsubstituted or substituted with one or more of halogen, oxo, CN, OR^{16a}, SR^{16a}, SOR^{16a}, SO₂R^{16a}, COR^{16a}, COOR^{16a}, CONR^{16a}R^{17a}, NR^{16a}COR^{17a}, NR^{16a}R^{17a}, optionally substituted C₆₋₁₂ aryl, optionally substituted C₃₋₆ cycloalkyl, optionally substituted 3 to 6 membered heterocycle or optionally substituted 5 to 10 membered heteroaryl. More preferably, the alkyl, alkenyl or alkynyl is unsubstituted or substituted with one or more of fluoro, OR¹⁶a, SO₂R^{16a}, optionally substituted C₃₋₆ cycloalkyl, optionally substituted 4 to 6 membered heterocycle or optionally substituted 5 or 6 membered heteroaryl. R^{16a} and R^{17a} may be the same as R¹⁶ and R¹⁷ above. More preferably, R^{16a} and R^{17a} are independently hydrogen, an optionally substituted C₁-C₃ alkyl, an optionally substituted C₂-C₃ alkenyl or an optionally substituted C₂-C₃ alkynyl. More preferably, R^{16a} and R^{17a} are independently hydrogen and an optionally substituted C₁-C₃ alkyl. When the heterocyclyl, heteroaryl, aryl or cycloalkyl which is a substituent on the Z group is substituted, either directly or indirectly, with an optionally substituted cycloalkyl, an optionally substituted heterocycle or an optionally substituted heteroaryl, the cycloalkyl, heterocycle or heteroaryl may be unsubstituted or substituted with one or more of optionally substituted C₁-C₆ alkyl, optionally substituted C₂-C₆ alkenyl, optionally substituted C₂-C₆ alkynyl, halogen, oxo, CN, OR^{16b}, SR^{16b}, SOR^{16b}, SO₂R^{16b}, COR^{16b}, COOR^{16b}, CONR^{16b}R^{17b}, NR^{16b}COR^{17b} or NR^{16b}R^{17b}. More preferably, the cycloalkyl, heterocycle or heteroaryl is unsubstituted or substituted with one or more of optionally substituted C₁-C₃ alkyl, halogen, oxo, CN, OR^{16b}, COOR¹⁶b, CONR¹⁶bR^{17b} or NR¹⁶bR^{17b}. Most preferably, the cycloalkyl, heterocycle or heteroaryl is unsubstituted or substituted with one or more of C₁-C₃ alkyl optionally substituted with fluoro, OH or OCH₃, fluoro, oxo or CONR¹⁶bR^{17b}. R^{16b} and R^{17b} may be the same as R¹⁶ and R¹⁷ above. More preferably, R^{16b} and R^{17b} are independently hydrogen, an optionally substituted C₁-C₃ alkyl, an optionally substituted C₂-C₃ alkenyl or an optionally substituted C₂-C₃ alkynyl. More preferably, R^{16b} and R^{17b} are CH₃. The heterocyclyl, heteroaryl, aryl or cycloalkyl may be understood to be indirectly substituted with a group if it is has a substituent which comprises that group. In embodiments where an aryl or cycloalkyl is substituent on the Z group, preferably the aryl or cycloalkyl is unsubstituted. In embodiments where a heterocyclyl or heteroaryl is substituent on the Z group, preferably the heterocyclyl or heteroaryl is substituted. Preferably, the heterocyclyl or heteroaryl has 1, 2 or 3 substituents, and most preferably has 1 substituent. Most preferably, the heterocyclyl or heteroaryl which is a substituent on the Z group may be unsubstituted or substituted with one or more substituents which may be selected from the group consisting of methyl, ethyl,

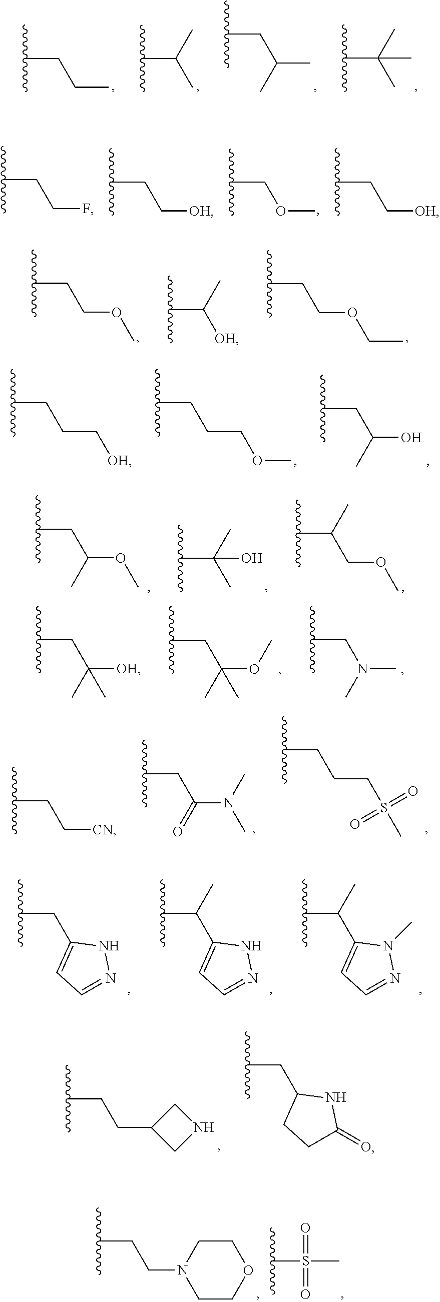

oxo, CN, OH, OCH₃,

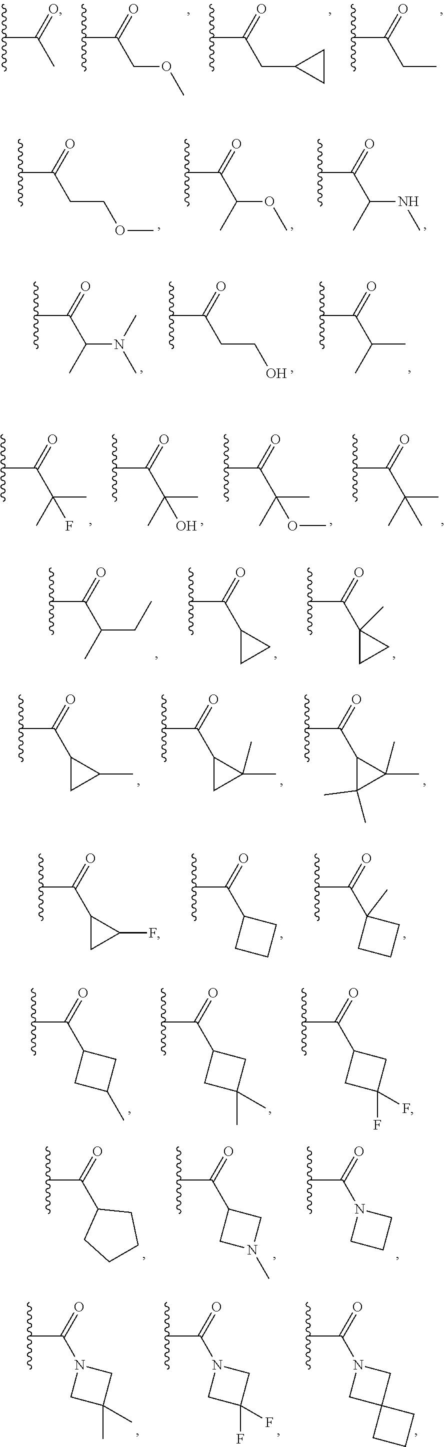

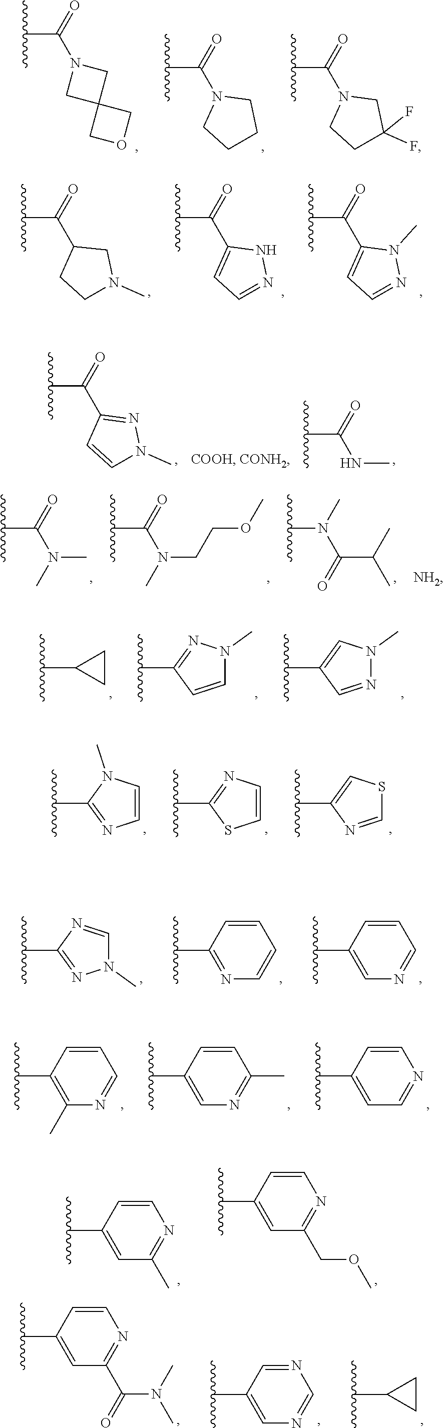

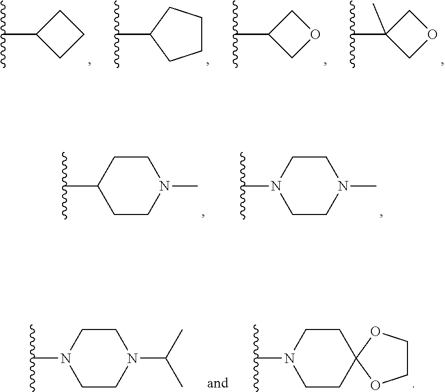

Accordingly. R¹³. R¹⁴ and R¹⁵ may independently be H, methyl, ethyl.

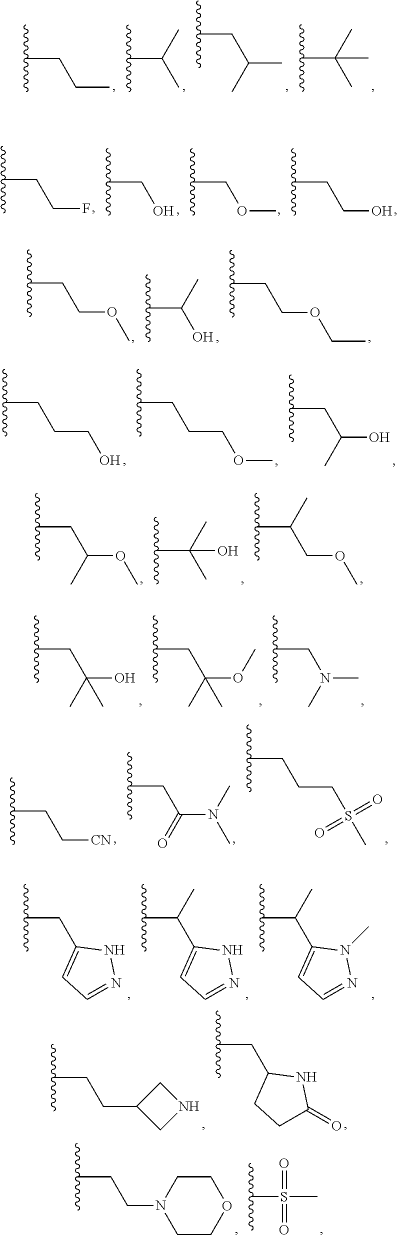

oxo, CN, OH, OCH₃.

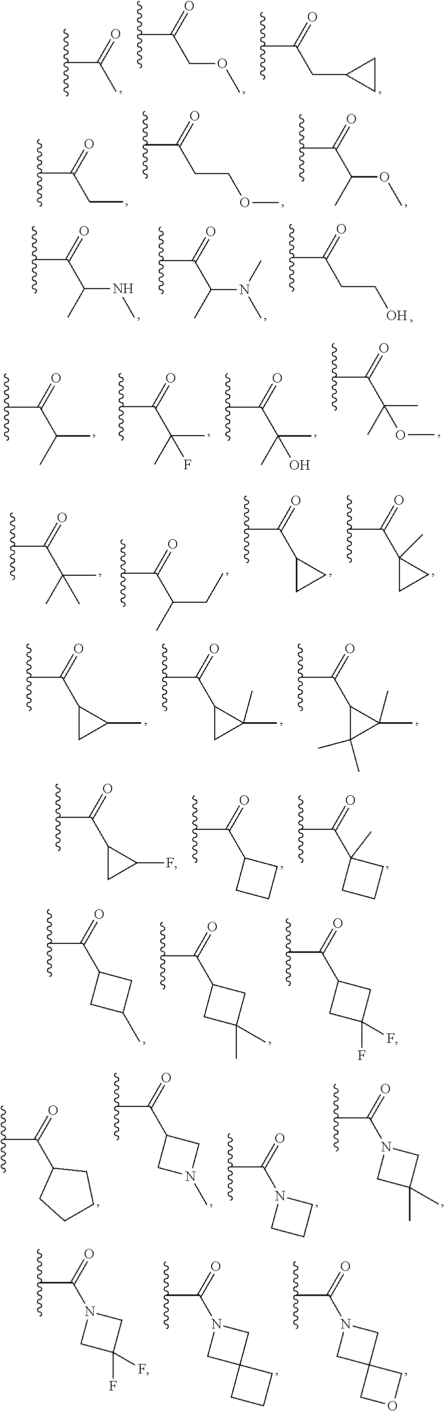

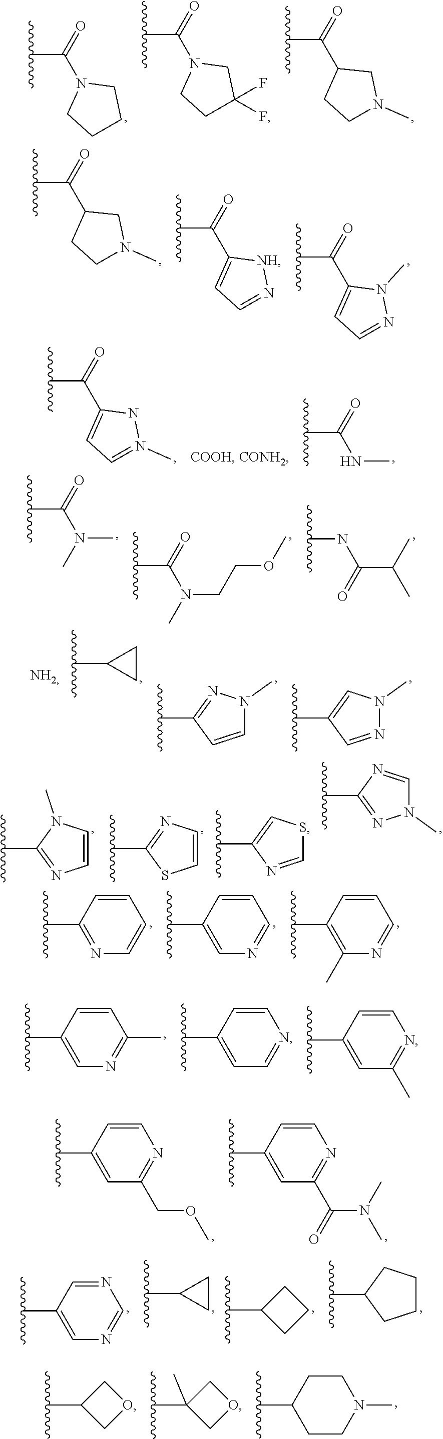

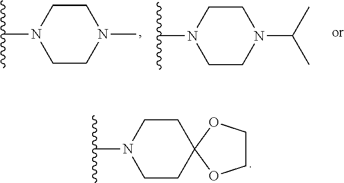

In embodiments where M is CR¹⁴R¹⁵, R¹⁵ may be H and R¹⁴ may be as defined above.

In another embodiment, the phenyl or 5 or 6 membered heteroaryl group Z is not substituted directly with an optionally substituted heterocyclyl, an optionally substituted heteroaryl, an optionally substituted aryl or an optionally substituted cycloalkyl.

Accordingly, in this embodiment, the phenyl or 5 or 6 membered heteroaryl group Z is substituted with one or more substituents selected from the group consisting of a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹ and NR⁸R⁹. It may be appreciated that these substituents may be group A. More preferably, the phenyl or 5 or 6 membered heteroaryl group Z is substituted with one or more substituents selected from the group consisting of a halogen, an optionally substituted C₁-C₆ alkyl, OR⁸, SO₂R⁸, SO₂NR⁸R⁹, COR⁸, COOR⁸, CONR⁸R⁹, NR⁸SO₂R⁹ and NR⁸R⁹. Most preferably, the phenyl or 5 or 6 membered heteroaryl group Z is substituted with one or more substituents selected from the group consisting of an optionally substituted C₁-C₃ alkyl, OR⁸, SO₂R⁸, SO₂NR⁸R⁹, COR⁸, CONR⁸R⁹, NR⁸SO₂R⁹ and NR⁸R⁹. R⁸ and R⁹ may independently be hydrogen, an optionally substituted C₁-C₁₂ alkyl, optionally substituted C₆₋₁₂ aryl, optionally substituted C₃-C₆ cycloalkyl, optionally substituted 3 to 8 membered heterocyclyl, and an optionally substituted 5 to 10 membered heteroaryl. More preferably, R⁸ and R⁹ are independently hydrogen, an optionally substituted C₁-C₁₀ alkyl, optionally substituted phenyl, optionally substituted C₃-C₆ cycloalkyl, optionally substituted 3 to 8 membered heterocyclyl or an optionally substituted 5 to 10 membered heteroaryl. Most preferably, R⁸ and R⁹ may be H, an optionally substituted C₁-C₈alkyl, cyclopropyl, an optionally substituted 5 or 6 membered heterocycle or an optionally substituted phenyl. The alkyl group can be unsubstituted or substituted with one or more of halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. More preferably, the alkyl group is unsubstituted or substituted with one or more of halogen, OR¹⁶, NR¹⁶R¹⁷, optionally substituted phenyl, optionally substituted C₃₋₆ cycloalkyl, optionally substituted 3 to 6 membered heterocycle or optionally substituted 5 or 6 membered heteroaryl. R¹⁶ and R¹⁷ are preferably H or C₁₋₃ alkyl. Most preferably, the alkyl group is unsubstituted or substituted with one or more of F, OH, N(CH₃)₂, N(CH₂CH₃)₂, optionally substituted 5 or 6 membered heterocycle, optionally substituted 5 or 6 membered heteroaryl or optionally substituted phenyl. When R⁸ or R⁹ is an optionally substituted cycloalkyl, optionally substituted heterocycle or optionally substituted heteroaryl or the alkyl group is substituted with an optionally substituted cycloalkyl, optionally substituted heterocycle or optionally substituted heteroaryl, the optionally substituted cycloalkyl, optionally substituted heterocycle or optionally substituted heteroaryl may be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. Most preferably, the optionally substituted cycloalkyl, optionally substituted heterocycle or optionally substituted heteroaryl may be unsubstituted or substituted with one or more of C₁₋₃ alkyl, halogen, oxo, OR¹⁶ and NR¹⁶R¹⁷. Preferably, R¹⁶ and R¹⁷ are H or C₁₋₃ alkyl. Most preferably, the optionally substituted cycloalkyl, optionally substituted heterocycle or optionally substituted heteroaryl is unsubstituted or substituted with one or more of CH₃, OCH₃ and/or oxo. When R⁸ or R⁹ is an optionally substituted aryl or the alkyl group is substituted with an optionally substituted aryl, the optionally substituted aryl may be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl. Most preferably, the optionally substituted aryl is unsubstituted or substituted with one or more of C₁₋₃ alkyl, halogen, OR¹⁶ and NR¹⁶R¹⁷. Preferably, R¹⁶ and R¹⁷ are H or C₁₋₃ alkyl. Most preferably, the optionally substituted aryl is unsubstituted or substituted with one or more of CH₃ and/or OCH₃. The phenyl or 5 or 6 membered heteroaryl group Z may be substituted with one or more substituents selected from the group consisting of CH₃, CH₂CF₃, CF₃,

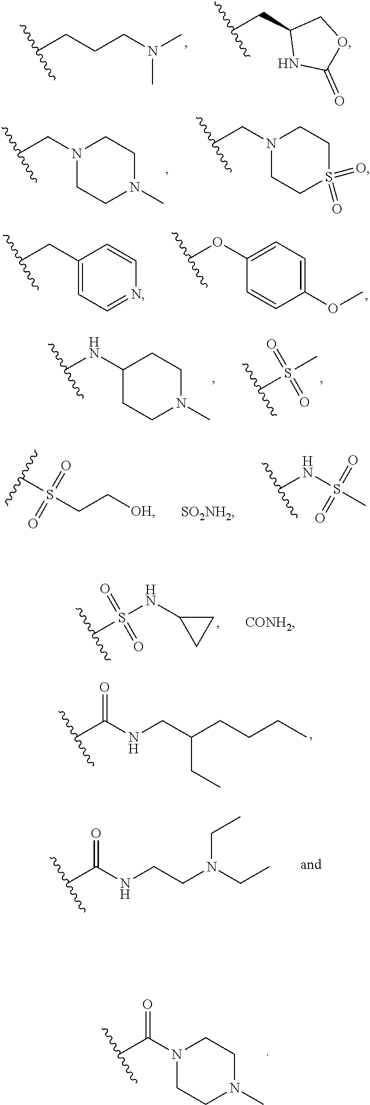

In a preferred embodiment, the heterocyclyl or heteroaryl which is a substituent on the Z group, which may optionally be A in the above formula, is an optionally substituted 5 or 6 membered heterocyclyl or an optionally substituted 5 or 6 membered heteroaryl. In a preferred embodiment, the heterocyclyl or heteroaryl which is a substituent on the Z group, which may optionally be A in the above formula, is an optionally substituted 6 membered heterocyclyl or an optionally substituted 6 membered heteroaryl.

The heterocyclyl or heteroaryl group which is a substituent on the Z group, which may optionally be A in the above formula, can be unsubstituted or substituted with one or more of optionally substituted C₁-C₆ alkyl, optionally substituted C₂-C₆ alkenyl, optionally substituted C₂-C₆ alkynyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ Cycloalkyl, optionally substituted 3 to 6 membered heterocycle or optionally substituted 5 to 10 membered heteroaryl, or pairs of non-adjacent substituents of the 3 to 8 membered heterocyclyl may combine to form a bridging group. More preferably, the optionally substituted heterocyclyl or an optionally substituted heteroaryl which is a substituent on the Z group, which may optionally be A in the above formula, can be unsubstituted or substituted with one or more of optionally substituted C₁-C₃ alkyl, halogen, oxo, COR¹⁶, COOR¹⁶, CONR¹⁶R¹⁷, NR¹⁶COR¹⁷ or NR¹⁶R¹⁷, or pairs of non-adjacent substituents of the 3 to 8 membered heterocyclyl may combine to form a bridging group. Even more preferably, the heterocyclyl or heteroaryl group which is a substituent on the Z group, which may optionally be A in the above formula, can be unsubstituted or substituted with one or more of optionally substituted C₁-C₃ alkyl, fluorine, COR¹⁶ or CONR¹⁶R¹⁷, or pairs of non-adjacent substituents of the 3 to 8 membered heterocyclyl may combine to form a bridging group. The alkyl may be unsubstituted or substituted with one or more of halogen, OR¹⁶ or NR¹⁶R¹⁷. R¹⁶ and R¹⁷ may be as defined above. In some embodiments, R¹⁶ and R¹⁷ may each independently be selected from the group consisting of H, optionally substituted C₁-C₃ alkyl, optionally substituted C₃₋₆ cycloalkyl, optionally substituted 3 to 6 membered heterocycle or optionally substituted 5 to 10 membered heteroaryl. Preferably, R¹⁶ and R¹⁷ are each independently selected from the group consisting of H, methyl and optionally halogenated 5 or 6 membered heterocycle. Preferably, the bridging group is optionally substituted methylene or ethylene.

n may be 1 or 2. In some embodiments, n is 1.

In some embodiments, T is N and M is NR¹³. In some embodiments, T is CR¹⁸ and M is NR¹³.

R¹³ may be H, optionally substituted C₁-C₆ alkyl, C₃-6 cycloalkyl, COR¹⁹ or CONR¹⁹R²⁰. R¹⁹ and R²⁰ may each independently be H, optionally substituted C₁₋₃ alkyl or optionally substituted 5 or 6 membered heterocyclyl. More preferably, R¹⁹ and R²⁰ are each independently H, optionally substituted C₁₋₃ alkyl or optionally halogenated 5 or 6 membered heterocyclyl. The alkyl may be unsubstituted or substituted with one or more of halogen, oxo, CN, OR¹⁶ or NR¹⁶R¹⁷. R¹⁶ and R¹⁷ may be as defined above. Preferably, R¹⁶ and R¹⁷ are H or CH₃. R¹³ may be CH₃, CH₂CH₃,

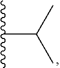 [SMILES: *C(C)C]

CH₂CH₂OH, CH₂CH₂OCH₃,

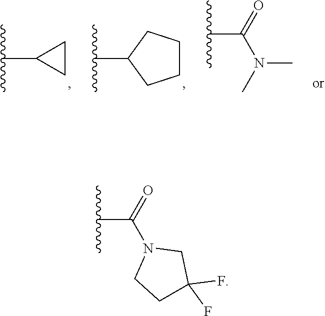

In some embodiments, T is N and M is CR¹⁴R¹⁵

R¹⁴ and R¹⁵ may each independently be selected from hydrogen, halogen, an optionally substituted C₁-C₃ alkyl, optionally substituted C₃-C₆ cycloalkyl, OR¹⁹-and-NR¹⁹R²⁰, or R¹⁴ and R¹⁵ together with the C atom they are both bonded may be linked to form an optionally substituted 3 to 6 membered heterocyclyl. More preferably, R¹⁴ and R¹⁵ are each independently selected from hydrogen, halogen, an optionally substituted C₁-C₃ alkyl and NR¹⁹R²⁰, or R¹⁴ and R¹⁵ together with the C atom they are both bonded may be linked to form an optionally substituted 3 to 6 membered hetercyclyl. R¹⁹ and R²⁰ may each independently be H or optionally substituted C₁₋₃ alkyl. Preferably, R¹⁹ and R²⁰ are each H. R¹⁴ and R¹⁵ may each independently be H, F, NH₂ or R¹⁴ and R¹⁵ together with the C atom they are both bonded may be linked to form a 5 membered hetercyclyl.

In some embodiments, T is N and M is O or SO₂.

Preferably, R¹⁸ is hydrogen.

Preferably, R¹² in each occurrence is independently H, halogen or optionally substituted C₁₋₃ alkyl; and/or two R¹² groups bonded to the same carbon atom define an oxo group; and/or two R¹² groups bonded to non-adjacent carbon atoms are linked to form a bicyclic bridged group. More preferably, R¹² in each occurrence is H or CH₃; and/or two R¹² groups bonded to the same carbon atom define an oxo group; and/or two R¹² groups bonded to non-adjacent carbon atoms are linked to form a bicyclic bridged group.

Exemplary bridged groups have formulae:

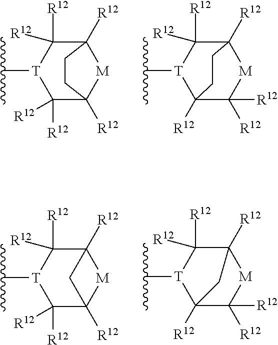

The group A or the heterocyclyl or heteroaryl group may be the only substituent of the phenyl or pyridyl group Z, or one or more further substituents may be present. Where present, the one or more further substituents may be selected from halogen; Ak; —OH; —OAK; —NH₂; —NHAk; NAK₂; optionally substituted heteroaryl; and optionally substituted heterocyclyl, wherein Ak in each occurrence is independently a C₁₋₆ alkyl group or a C₃₋₆ cycloalkyl group. The one or more further substituents are preferably halogen, more preferably F.

The phenyl or 5 or 6 membered heteroaryl group may not comprise any further substituents. Accordingly, each R²¹ may be H.

Alternatively, the phenyl or 5 or 6 membered heteroaryl group may be further optionally substituted with one or more substituents selected from the group consisting of a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 6 membered heterocyclyl or an optionally substituted 5 or 6 membered heteroaryl. It may be appreciated that these substituents may be one or more R²¹ groups in the above formula. Further R²¹ groups in the above formula may be H. Accordingly, each R²¹ may be H or a substituent as defined below. The phenyl or 5 or 6 membered heteroaryl group may be further optionally substituted with one or more substituents selected from the group consisting of a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹ and NR⁸R⁹. R⁸ and R⁹ may independently be hydrogen, an optionally substituted C₁-C₃ alkyl, an optionally substituted C₂-C₃ alkenyl or an optionally substituted C₂-C₃ alkynyl, and preferably R⁸ and R⁹ are independently H or C₁₋₃ alkyl. When the phenyl or 5 or 6 membered heteroaryl group is further substituted with an optionally substituted alkyl, an optionally substituted alkenyl or an optionally substituted alkynyl, the alkyl, alkenyl or alkynyl may be unsubstituted or substituted with one or more of halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷ and NR¹⁶R¹⁷. More preferably, the alkyl, alkenyl or alkynyl is unsubstituted or substituted with fluorine, OR¹⁶ and NR¹⁶R¹⁷. R¹⁶ and R¹⁷ may be as defined above. Preferably, R¹⁶ and R¹⁷ may independently be hydrogen, an optionally substituted C₁-C₃ alkyl, an optionally substituted C₂-C₃ alkenyl or an optionally substituted C₂-C₃ alkynyl, and preferably R¹⁶ and R¹⁷ are independently H or C₁₋₃ alkyl. In some embodiments, the phenyl or 5 or 6 membered heteroaryl group Z is substituted with an optionally substituted 5 to 10 membered heterocyclyl, an optionally substituted 5 to 10 membered heteroaryl, an optionally substituted phenyl or an optionally substituted C₃₋₆ cycloalkyl. In some embodiments, the phenyl or 5 or 6 membered heteroaryl group Z is substituted with an optionally substituted 5 to 10 membered heterocyclyl or an optionally substituted 5 to 10 membered heteroaryl. The phenyl or 5 or 6 membered heteroaryl group may not comprise any further substituents. Alternatively, the phenyl or 5 or 6 membered heteroaryl group may be further optionally substituted with one or more substituents selected from the group consisting of a halogen, an optionally substituted C₁-C₃ alkyl, CN, OR⁸, COOR⁸, CONR⁸R⁹, NR⁸R⁹ or a 5 or 6 membered heteroaryl. The phenyl or 5 or 6 membered heteroaryl group may be further optionally substituted with a halogen. In some embodiments, the phenyl or 5 or 6 membered heteroaryl group Z is substituted with an optionally substituted 5 to 7 membered heterocyclyl, an optionally substituted 5 or 6 membered heteroaryl, phenyl or cyclohexyl. In some embodiments, the phenyl or 5 or 6 membered heteroaryl group Z is substituted with an optionally substituted 5 to 7 membered heterocyclyl or an optionally substituted 5 or 6 membered heteroaryl. The phenyl or 5 or 6 membered heteroaryl group may not comprise any further substituents. Alternatively, the phenyl or 5 or 6 membered heteroaryl group may be further optionally substituted with a halogen. The halogen may be fluorine. In some embodiments, the phenyl or 5 or 6 membered heteroaryl group Z is substituted with an optionally substituted heterocyclyl or an optionally substituted heteroaryl and is further substituted with one or more of F, Cl, Br, CH₃, CF₃, CH₂OH, CH₂CH₂OH, CH₂NH₂, CH₂N(CH₃)₂, CN, OCH₃, OCH₂CH₃, COOH, CON(CH₃)₂, NH₂, NHCH₃, N(CH₃)₂ or

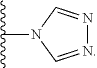 [SMILES: *n1cnnc1]

Accordingly, each R²¹ may independently be H, F, Cl, Br, CH₃, CF₃, CHEOH, CHECHLOH, CH—NH₂, CHN(CH₃)₂. CN, OCH₃, OCH₂CH₃, COOH, CON(CH₃)₂, NH₂, NHCH₃, N(CH₃)₂ or

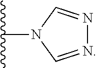 [SMILES: *n1cnnc1]

In some embodiments, the phenyl or 5 or 6 membered heteroaryl group Z is further substituted with a halogen, preferably fluorine.
Z may be

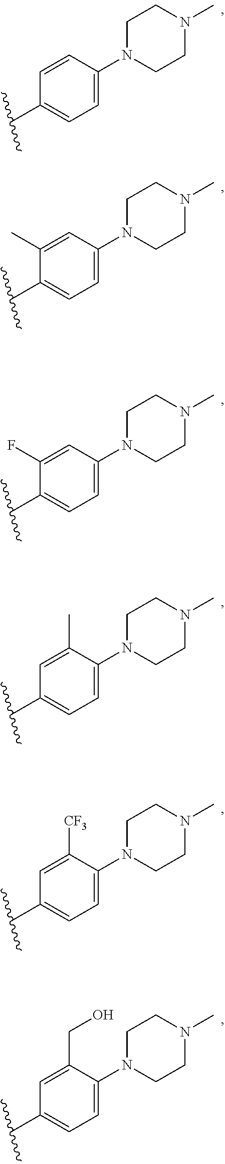 [SMILES: *c1ccc(N2CCN(C)CC2)cc1 ; *c1ccc(N2CCN(C)CC2)cc1C ; *c1ccc(N2CCN(C)CC2)cc1F ; *c1ccc(N2CCN(C)CC2)c(C)c1 ; *c1ccc(N2CCN(C)CC2)c(C(F)(F)F)c1 ; *c1ccc(N2CCN(C)CC2)c(CO)c1]

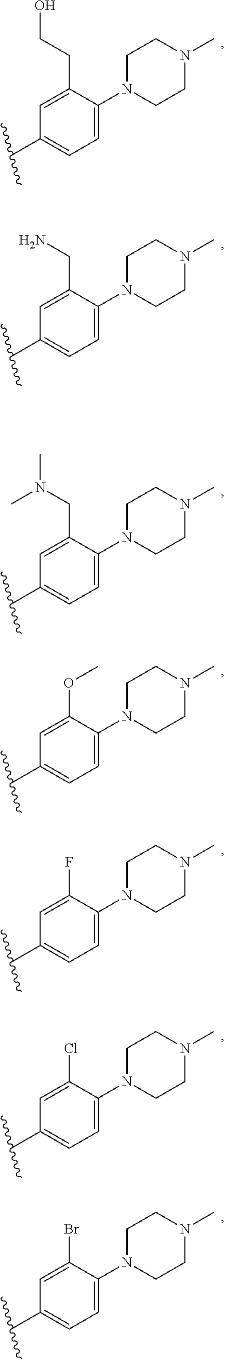

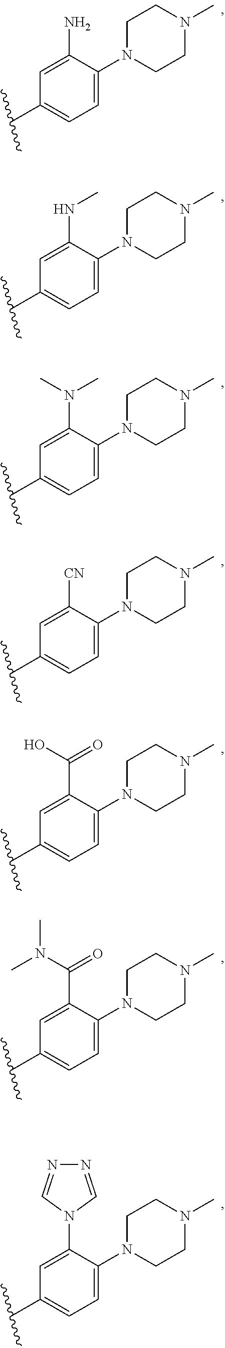

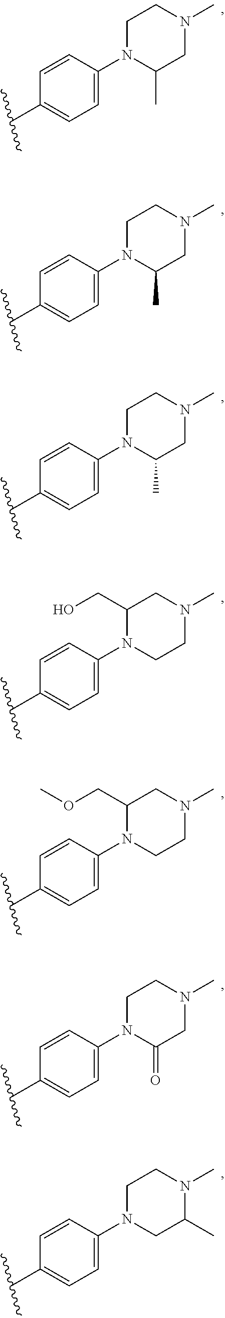

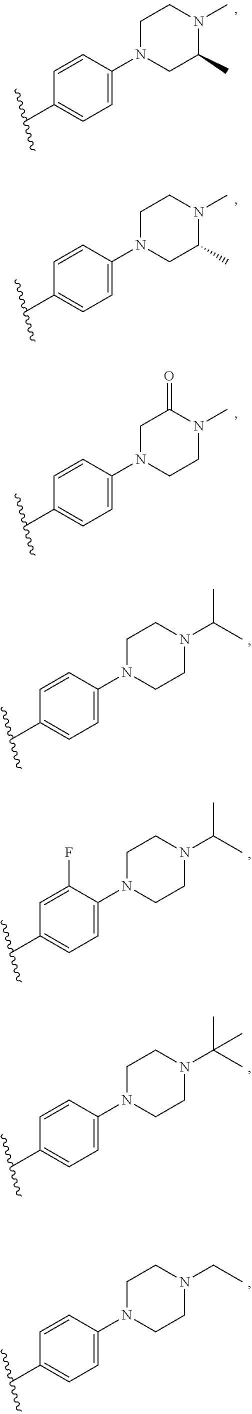

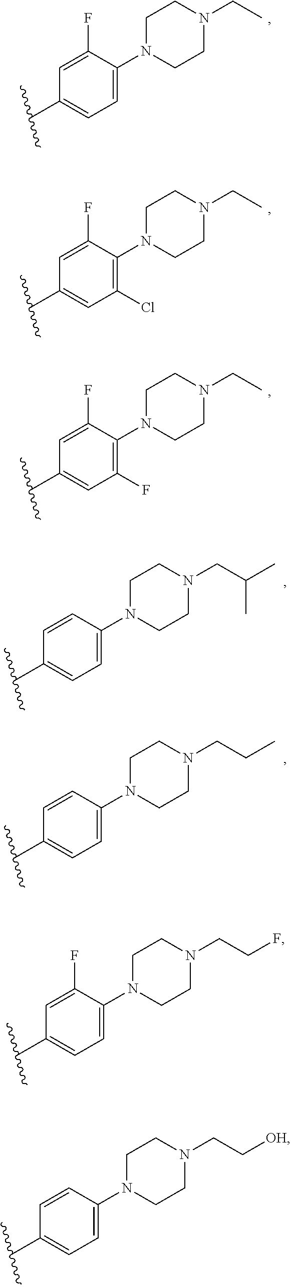

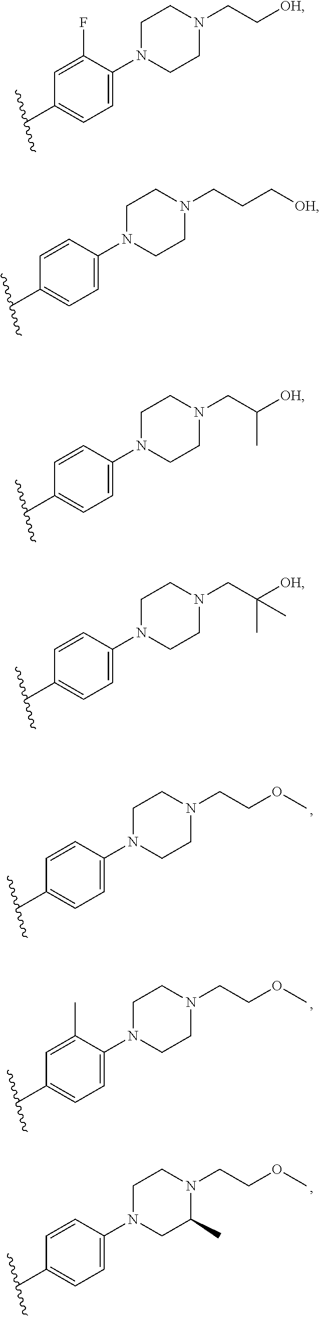

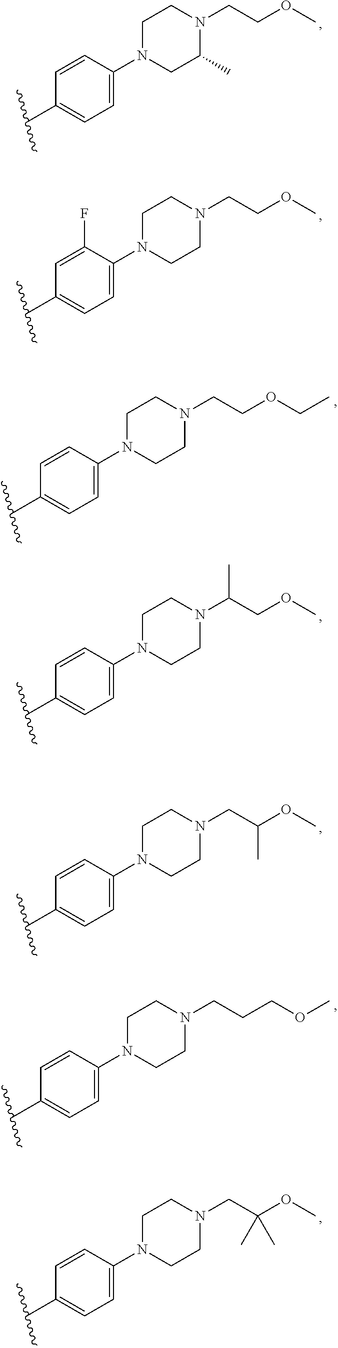

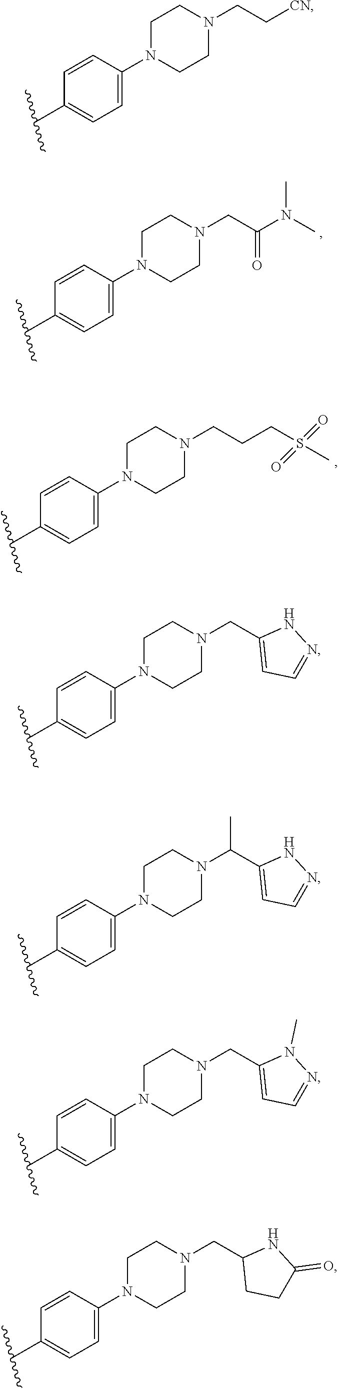

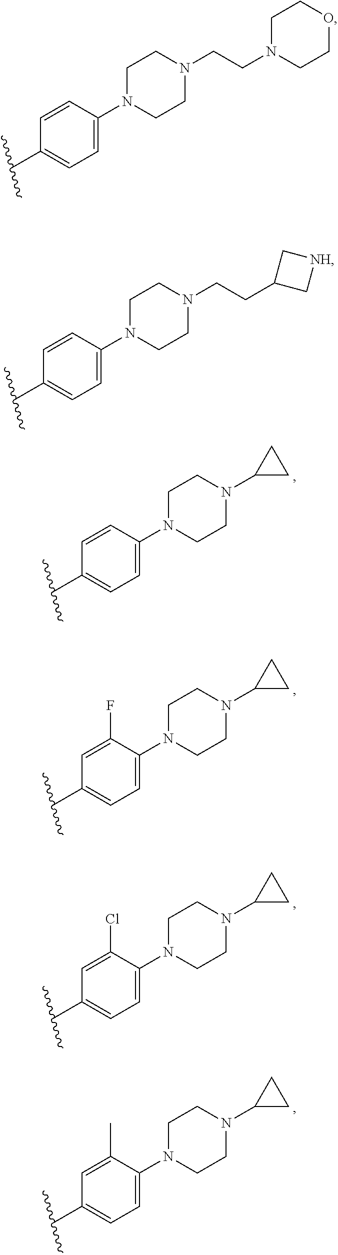 [SMILES: *c1ccc(N2CCN(CCN3CCOCC3)CC2)cc1 ; *c1ccc(N2CCN(CCC3CNC3)CC2)cc1 ; *c1ccc(N2CCN(C3CC3)CC2)cc1 ; *c1ccc(N2CCN(C3CC3)CC2)c(F)c1 ; *c1ccc(N2CCN(C3CC3)CC2)c(Cl)c1 ; *c1ccc(N2CCN(C3CC3)CC2)c(C)c1]

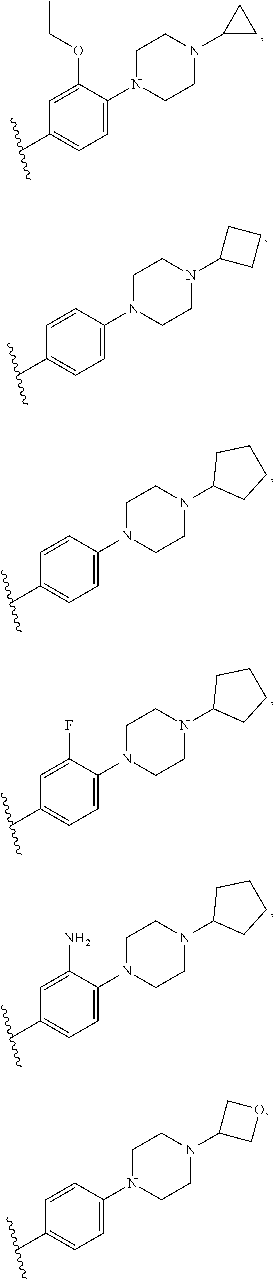 [SMILES: *c1ccc(N2CCN(C3CC3)CC2)c(OCC)c1 ; *c1ccc(N2CCN(C3CCC3)CC2)cc1 ; *c1ccc(N2CCN(C3CCCC3)CC2)cc1 ; *c1ccc(N2CCN(C3CCCC3)CC2)c(F)c1 ; *c1ccc(N2CCN(C3CCCC3)CC2)c(N)c1 ; *c1ccc(N2CCN(C3COC3)CC2)cc1]

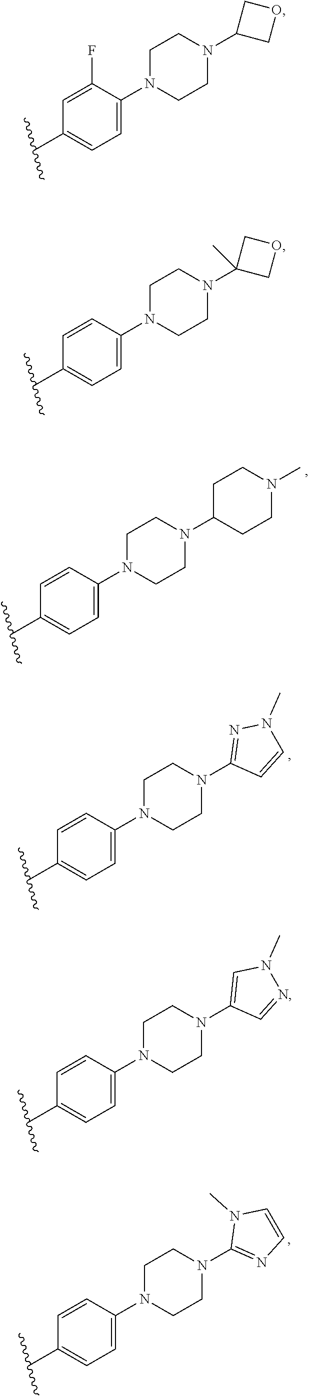 [SMILES: *c1ccc(N2CCN(C3COC3)CC2)c(F)c1 ; *c1ccc(N2CCN(C3(C)COC3)CC2)cc1 ; *c1ccc(N2CCN(C3CCN(C)CC3)CC2)cc1 ; *c1ccc(N2CCN(c3ccn(C)n3)CC2)cc1 ; *c1ccc(N2CCN(c3cnn(C)c3)CC2)cc1 ; *c1ccc(N2CCN(c3nccn3C)CC2)cc1]

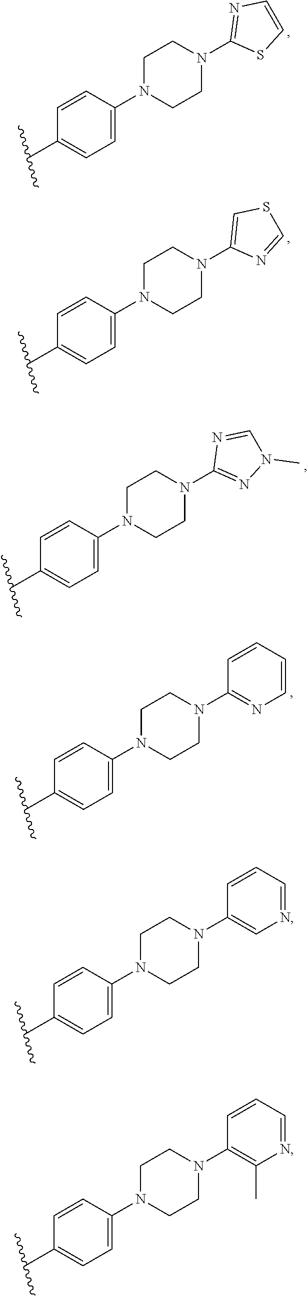 [SMILES: *c1ccc(N2CCN(c3nccs3)CC2)cc1 ; *c1ccc(N2CCN(c3cscn3)CC2)cc1 ; *c1ccc(N2CCN(c3ncn(C)n3)CC2)cc1 ; *c1ccc(N2CCN(c3ccccn3)CC2)cc1 ; *c1ccc(N2CCN(c3cccnc3)CC2)cc1 ; *c1ccc(N2CCN(c3cccnc3C)CC2)cc1]

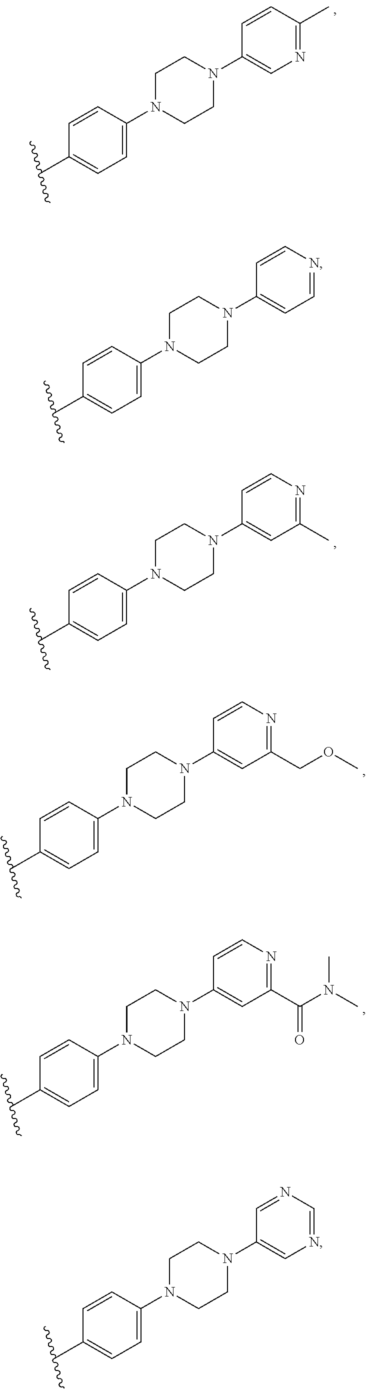 [SMILES: *c1ccc(N2CCN(c3ccc(C)nc3)CC2)cc1 ; *c1ccc(N2CCN(c3ccncc3)CC2)cc1 ; *c1ccc(N2CCN(c3ccnc(C)c3)CC2)cc1 ; *c1ccc(N2CCN(c3ccnc(COC)c3)CC2)cc1 ; *c1ccc(N2CCN(c3ccnc(C(=O)N(C)C)c3)CC2)cc1 ; *c1ccc(N2CCN(c3cncnc3)CC2)cc1]

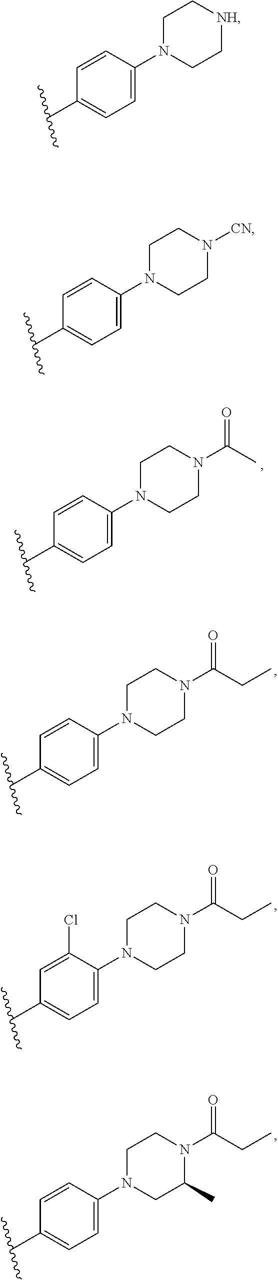 [SMILES: *c1ccc(N2CCNCC2)cc1 ; *c1ccc(N2CCN(C#N)CC2)cc1 ; *c1ccc(N2CCN(C(C)=O)CC2)cc1 ; *c1ccc(N2CCN(C(=O)CC)CC2)cc1 ; *c1ccc(N2CCN(C(=O)CC)CC2)c(Cl)c1 ; *c1ccc(N2CCN(C(=O)CC)[C@@H](C)C2)cc1]

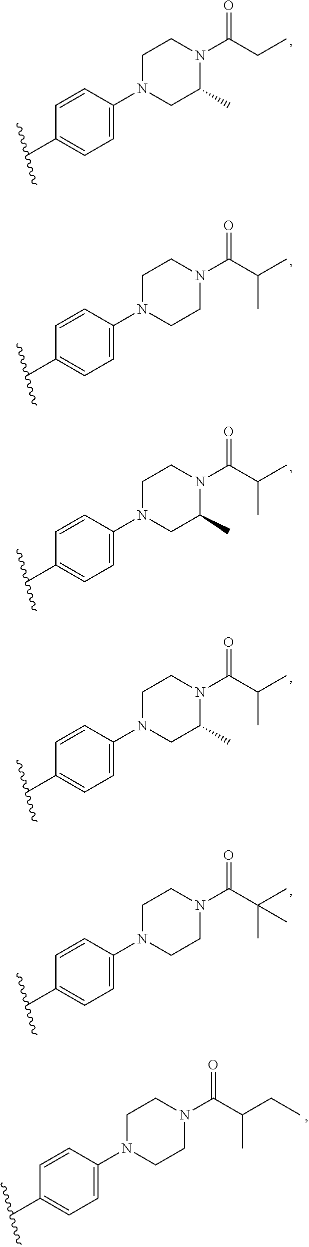 [SMILES: *c1ccc(N2CCN(C(=O)CC)[C@H](C)C2)cc1 ; *c1ccc(N2CCN(C(=O)C(C)C)CC2)cc1 ; *c1ccc(N2CCN(C(=O)C(C)C)[C@@H](C)C2)cc1 ; *c1ccc(N2CCN(C(=O)C(C)C)[C@H](C)C2)cc1 ; *c1ccc(N2CCN(C(=O)C(C)(C)C)CC2)cc1 ; *c1ccc(N2CCN(C(=O)C(C)CC)CC2)cc1]

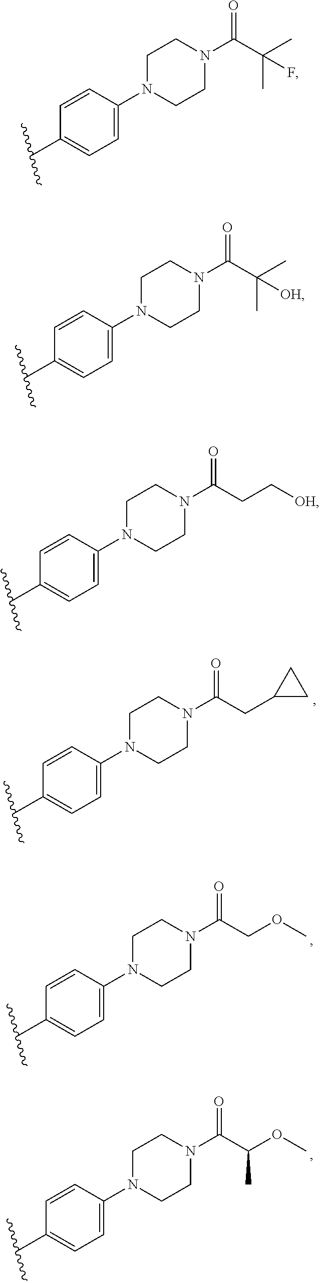 [SMILES: *c1ccc(N2CCN(C(=O)C(C)(C)F)CC2)cc1 ; *c1ccc(N2CCN(C(=O)C(C)(C)O)CC2)cc1 ; *c1ccc(N2CCN(C(=O)CCO)CC2)cc1 ; *c1ccc(N2CCN(C(=O)CC3CC3)CC2)cc1 ; *c1ccc(N2CCN(C(=O)COC)CC2)cc1 ; *c1ccc(N2CCN(C(=O)[C@H](C)OC)CC2)cc1]

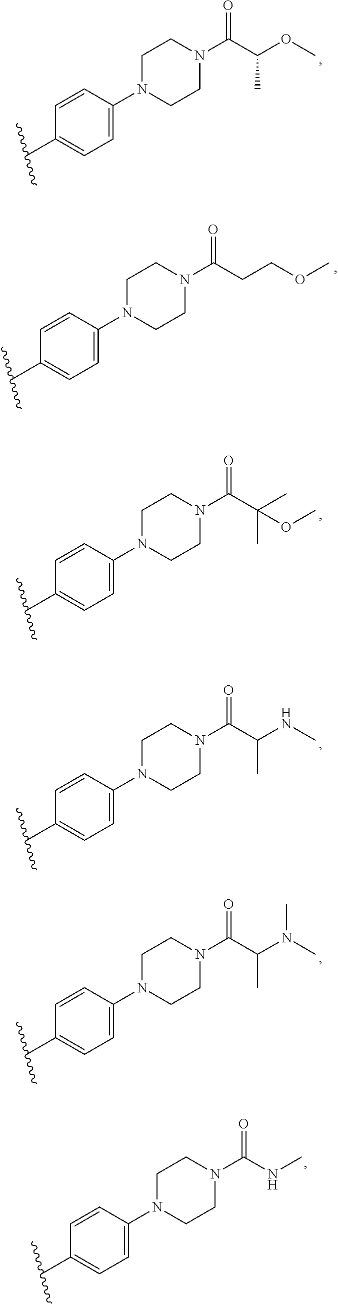 [SMILES: *c1ccc(N2CCN(C(=O)[C@@H](C)OC)CC2)cc1 ; *c1ccc(N2CCN(C(=O)CCOC)CC2)cc1 ; *c1ccc(N2CCN(C(=O)C(C)(C)OC)CC2)cc1 ; *c1ccc(N2CCN(C(=O)C(C)NC)CC2)cc1 ; *c1ccc(N2CCN(C(=O)C(C)N(C)C)CC2)cc1 ; *c1ccc(N2CCN(C(=O)NC)CC2)cc1]

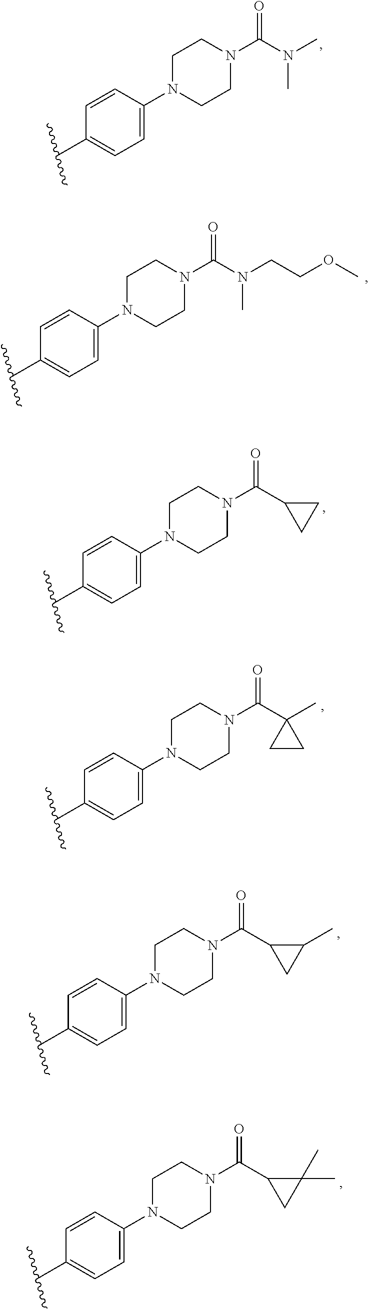 [SMILES: *c1ccc(N2CCN(C(=O)N(C)C)CC2)cc1 ; *c1ccc(N2CCN(C(=O)N(C)CCOC)CC2)cc1 ; *c1ccc(N2CCN(C(=O)C3CC3)CC2)cc1 ; *c1ccc(N2CCN(C(=O)C3(C)CC3)CC2)cc1 ; *c1ccc(N2CCN(C(=O)C3CC3C)CC2)cc1 ; *c1ccc(N2CCN(C(=O)C3CC3(C)C)CC2)cc1]

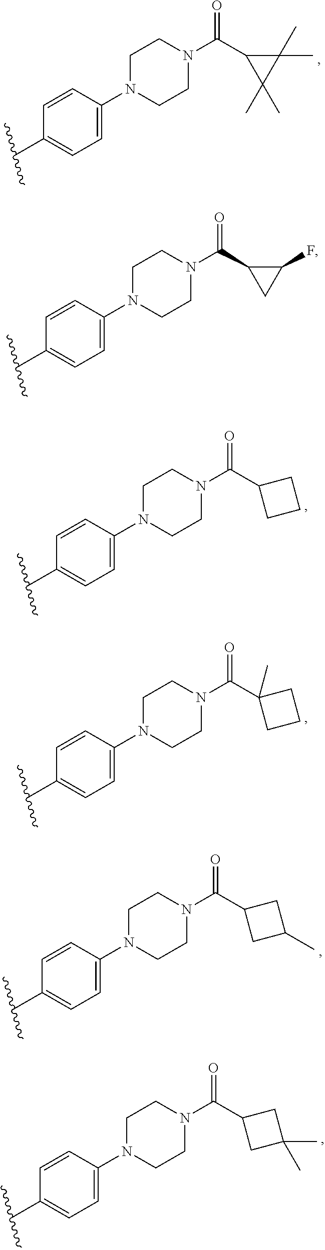 [SMILES: *c1ccc(N2CCN(C(=O)C3C(C)(C)C3(C)C)CC2)cc1 ; *c1ccc(N2CCN(C(=O)[C@@H]3C[C@@H]3F)CC2)cc1 ; *c1ccc(N2CCN(C(=O)C3CCC3)CC2)cc1 ; *c1ccc(N2CCN(C(=O)C3(C)CCC3)CC2)cc1 ; *c1ccc(N2CCN(C(=O)C3CC(C)C3)CC2)cc1 ; *c1ccc(N2CCN(C(=O)C3CC(C)(C)C3)CC2)cc1]

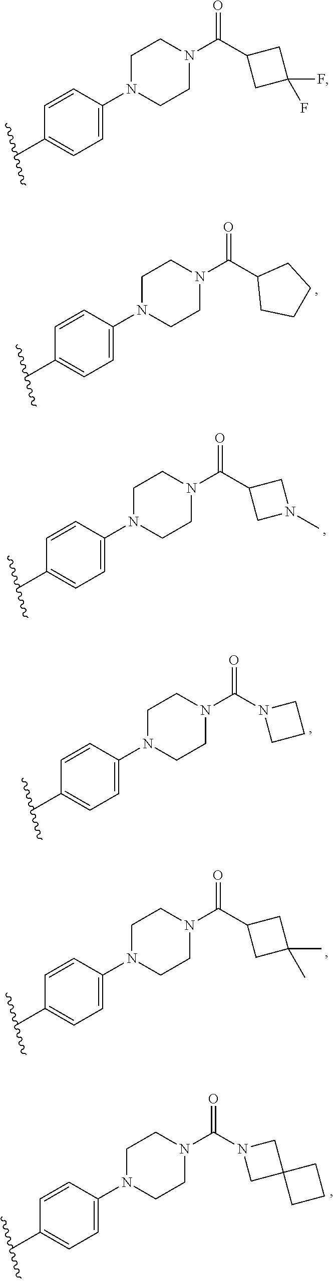 [SMILES: *c1ccc(N2CCN(C(=O)C3CC(F)(F)C3)CC2)cc1 ; *c1ccc(N2CCN(C(=O)C3CCCC3)CC2)cc1 ; *c1ccc(N2CCN(C(=O)C3CN(C)C3)CC2)cc1 ; *c1ccc(N2CCN(C(=O)N3CCC3)CC2)cc1 ; *c1ccc(N2CCN(C(=O)C3CC(C)(C)C3)CC2)cc1 ; *c1ccc(N2CCN(C(=O)N3CC4(CCC4)C3)CC2)cc1]

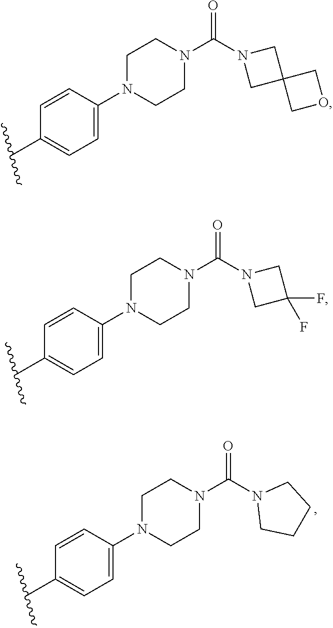 [SMILES: *c1ccc(N2CCN(C(=O)N3CC4(COC4)C3)CC2)cc1 ; *c1ccc(N2CCN(C(=O)N3CC(F)(F)C3)CC2)cc1 ; *c1ccc(N2CCN(C(=O)N3CCCC3)CC2)cc1]

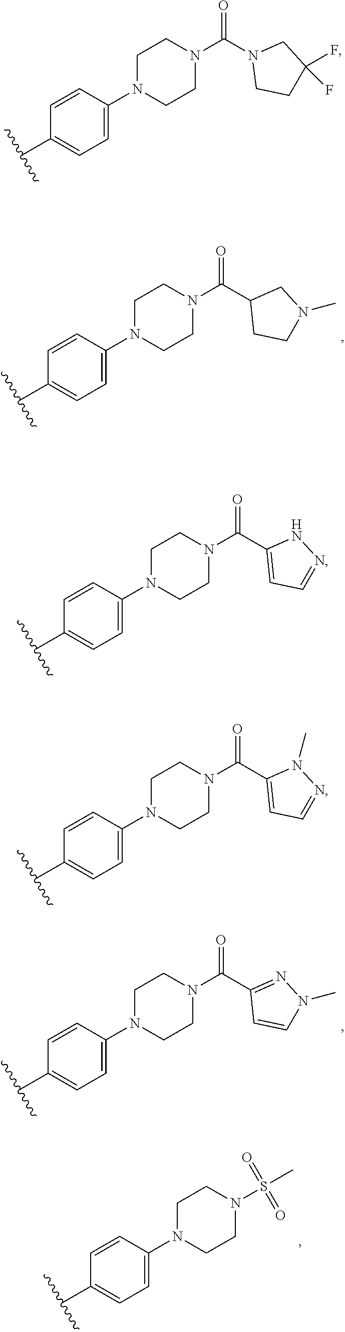 [SMILES: *c1ccc(N2CCN(C(=O)N3CCC(F)(F)C3)CC2)cc1 ; *c1ccc(N2CCN(C(=O)C3CCN(C)C3)CC2)cc1 ; *c1ccc(N2CCN(C(=O)c3ccn[nH]3)CC2)cc1 ; *c1ccc(N2CCN(C(=O)c3ccnn3C)CC2)cc1 ; *c1ccc(N2CCN(C(=O)c3ccn(C)n3)CC2)cc1 ; *c1ccc(N2CCN(S(C)(=O)=O)CC2)cc1]

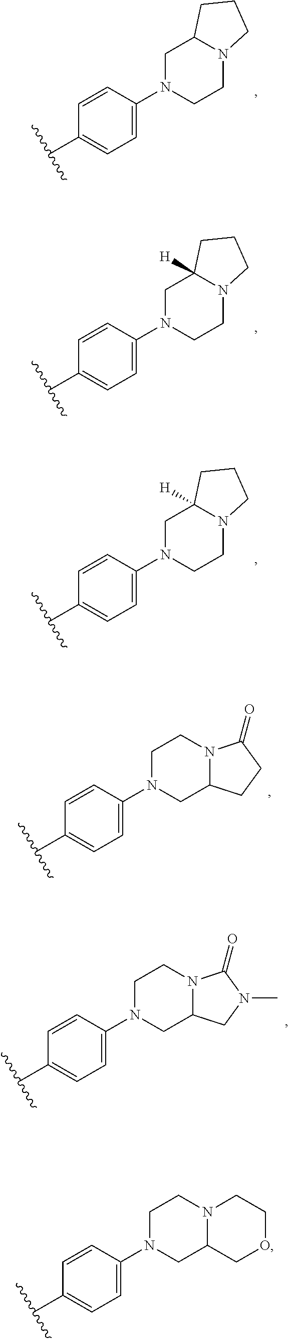 [SMILES: *c1ccc(N2CCN3CCCC3C2)cc1 ; *c1ccc(N2CCN3CCC[C@H]3C2)cc1 ; *c1ccc(N2CCN3CCC[C@@H]3C2)cc1 ; *c1ccc(N2CCN3C(=O)CCC3C2)cc1 ; *c1ccc(N2CCN3C(=O)N(C)CC3C2)cc1 ; *c1ccc(N2CCN3CCOCC3C2)cc1]

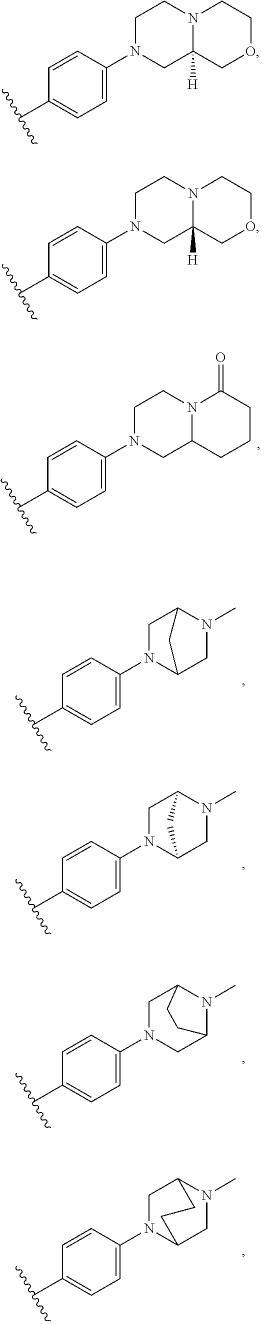

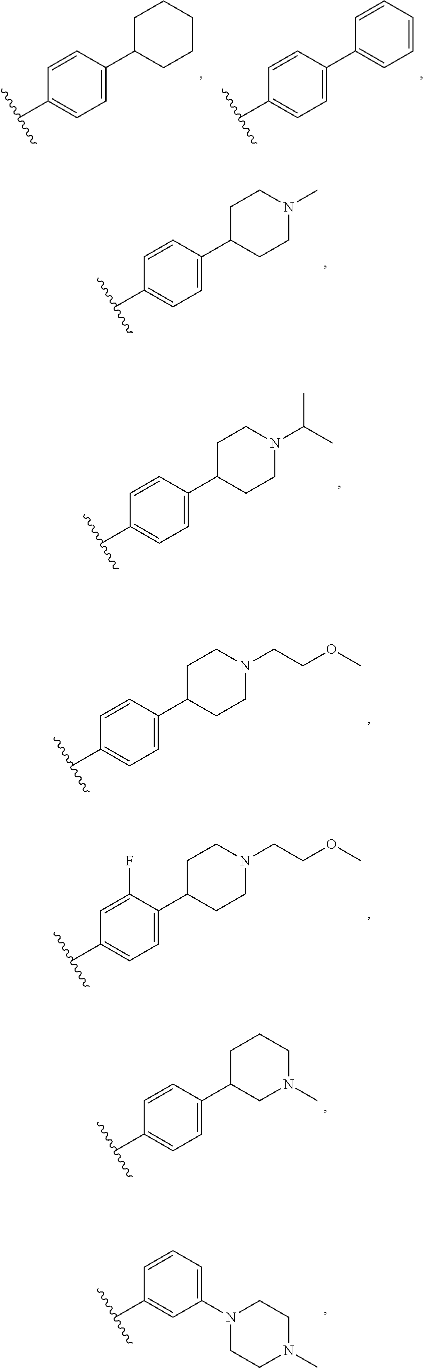

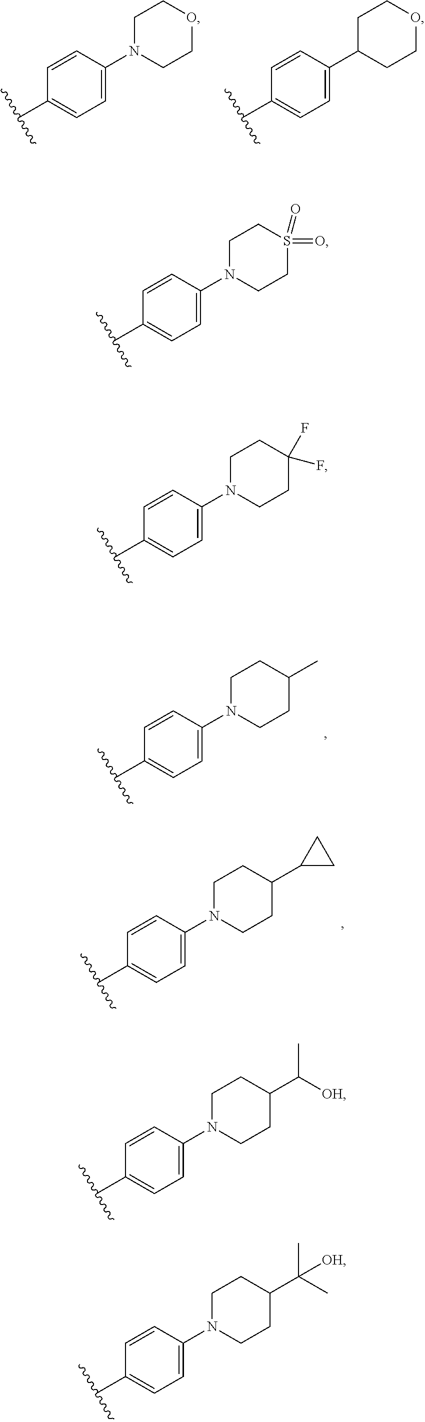

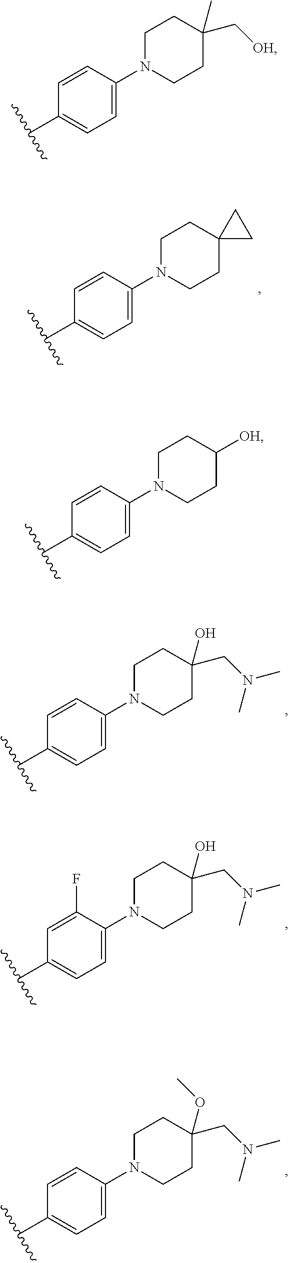 [SMILES: *c1ccc(N2CCC(C)(CO)CC2)cc1 ; *c1ccc(N2CCC3(CC2)CC3)cc1 ; *c1ccc(N2CCC(O)CC2)cc1 ; *c1ccc(N2CCC(O)(CN(C)C)CC2)cc1 ; *c1ccc(N2CCC(O)(CN(C)C)CC2)c(F)c1 ; *c1ccc(N2CCC(CN(C)C)(OC)CC2)cc1]

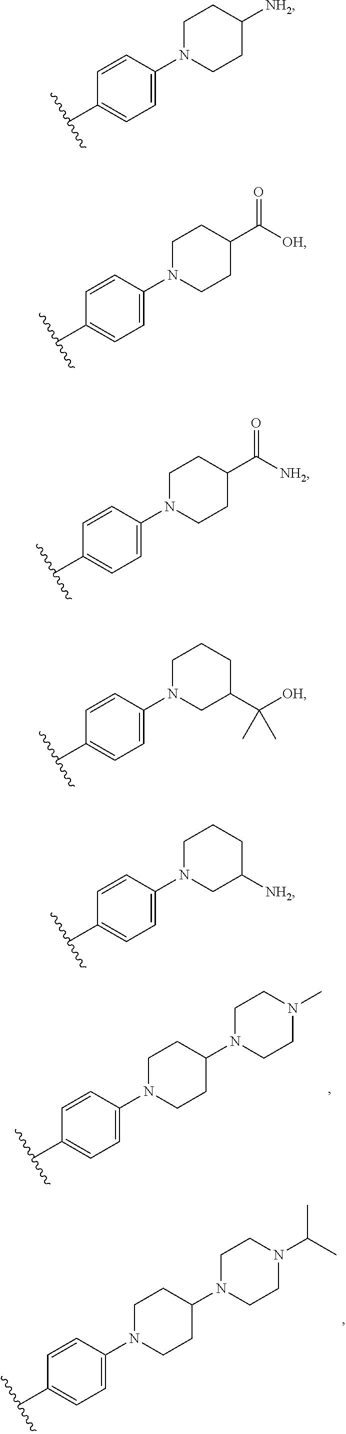

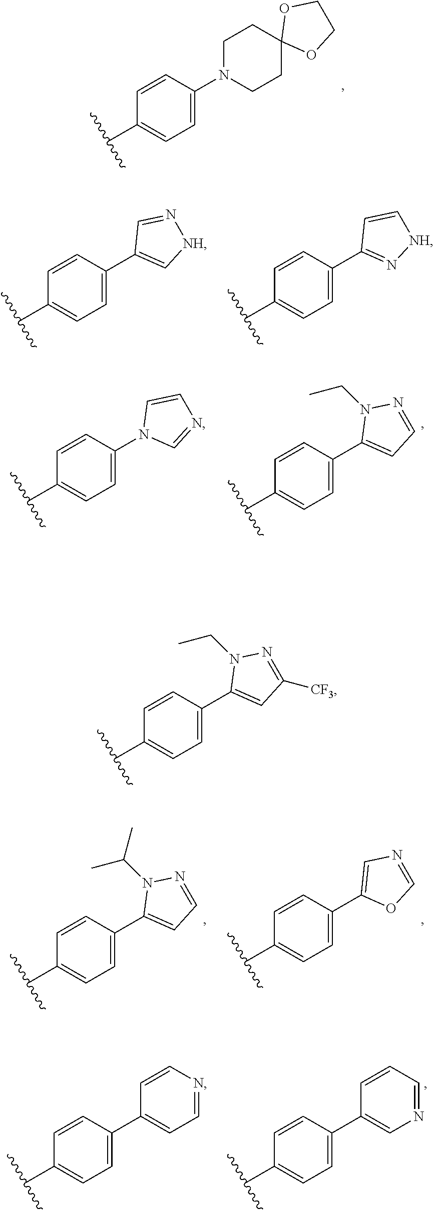

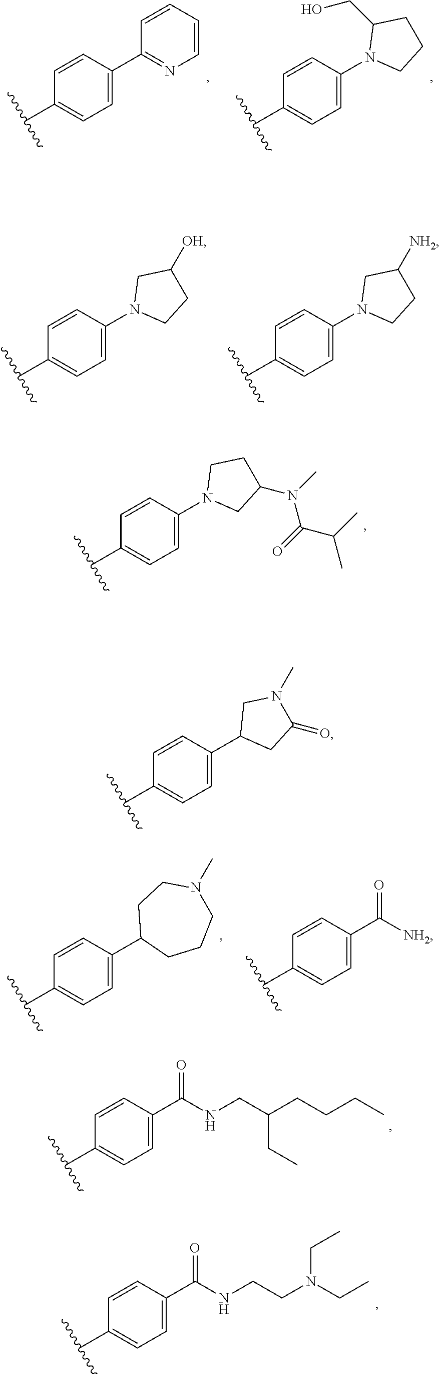

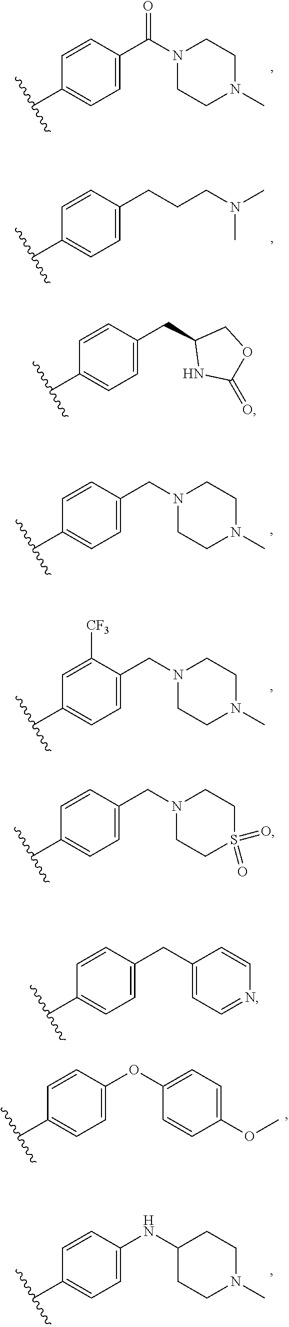

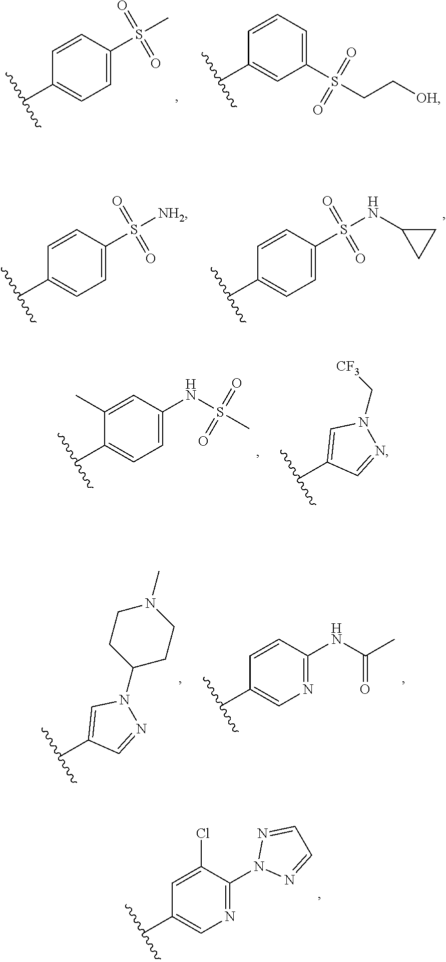

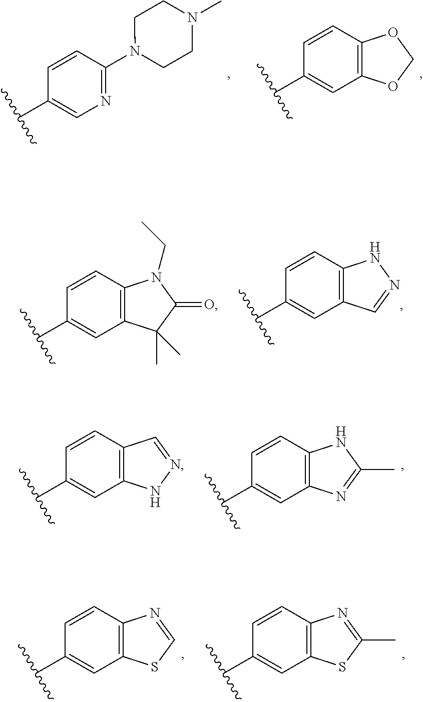

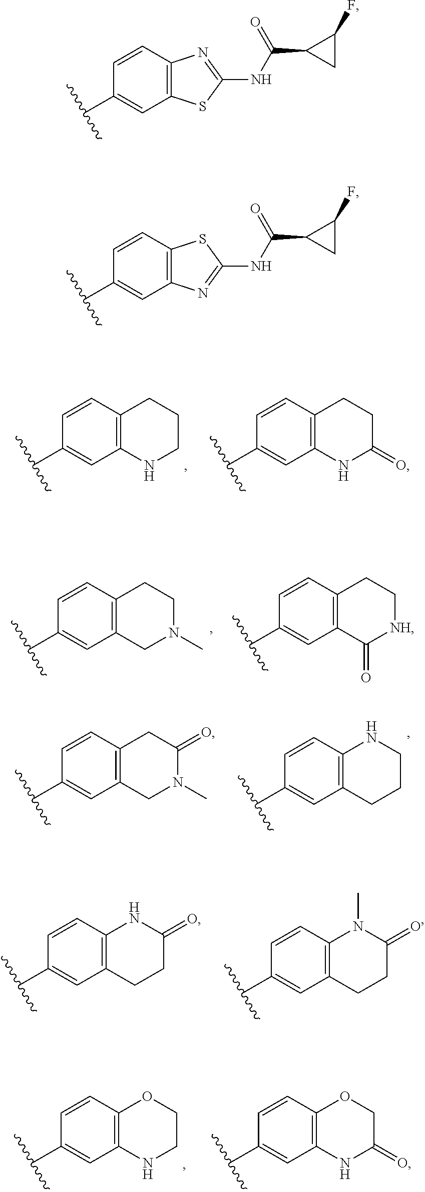

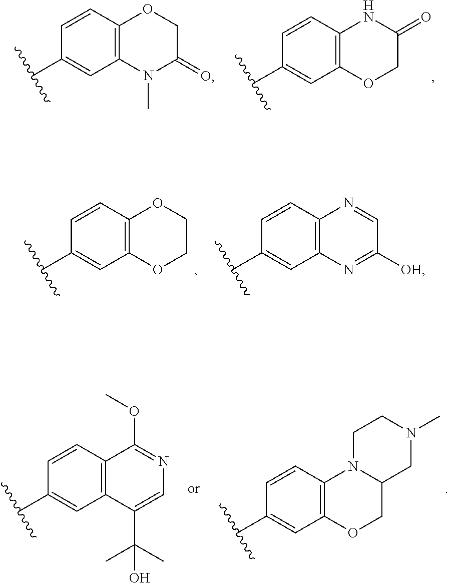 [SMILES: *c1ccc2c(c1)N(C)C(=O)CO2 ; *c1ccc2c(c1)OCC(=O)N2 ; *c1ccc2c(c1)OCCO2 ; *c1ccc2ncc(O)nc2c1 ; *c1ccc2c(OC)ncc(C(C)(C)O)c2c1 ; *c1ccc2c(c1)OCC1CN(C)CCN21]

It will be understood that the above compounds may exist as enantiomers and as diastereoisomeric pairs. These isomers also represent further embodiments of the invention.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Mixtures of stereoisomers may be separated by conventional techniques known to those skilled in the art; see, for example, “Stereochemistry of Organic Compounds” by E. L. Eliel and S. H. Wilen (Wiley, New York, 1994).

It will be appreciated that the compounds described herein or a pharmaceutically acceptable salt, solvate, tautomeric form or polymorphic form thereof may be used in a medicament which may be used in a monotherapy (i.e. use of the compound alone), for inhibiting the HPK-1 protein and/or treating, ameliorating or preventing a disease.

Alternatively, the compounds or a pharmaceutically acceptable salt, solvate, tautomeric form or polymorphic form thereof may be used as an adjunct to, or in combination with, known therapies for inhibiting the HPK-1 protein and/or treating, ameliorating or preventing a disease.

Accordingly, in one aspect, a second therapeutic agent may be administered with a compound of Formula (I). The compound of Formula (I) may be administered before, after, and/or together with the second therapeutic agent. The second therapeutic agent may comprise an antiviral agent, an anti-inflammation agent, conventional chemotherapy, an anti-cancer vaccine and/or hormonal therapy or an antiproliferative compound. Alternatively, or additionally, the second therapeutic agent may comprise a B7 costimulatory molecule, interleukin-2, interferon-γ, GM-CSF, a CTLA-4 antagonist (such as Ipilimumab and tremilimumab), an IDO inhibitor or IDO/TDO inhibitor (such as Epacadostat, CRD1152 and GDC-0919), a PD-1 inhibitor (such as Nivolumab, Pembrolizumab, Pidilizumab), a PD-L1 inhibitor (such as Durvalumab, Avelumab and Atczolizumab), an OX-40 agonist, a LAG3 inhibitor, a TIM-3 inhibitor, an anti-TIGIT monoclonal antibody, a CD40 ligand, a 4-1BB/CD137 agonist, a GITR agonist, ICOS agonists, a KIR inhibitor, CD47 inhibitors, CD73 inhibitors, CSFIR inhibitors, an NKG2A inhibitor, a CD27 agonist, Bacille Calmette-Guerin (BCG), liposomes, alum, Freund's complete or incomplete adjuvant, a TLR agonist (such as Poly I: C, MPL, LPS, bacterial flagellin, imiquimod, resiquimod, loxoribine and a CpG dinucleotide), CAR T-cells and/or detoxified endotoxins. Antiproliferative compounds include, but are not limited to aromatase inhibitors (formestane, anastrozole), antiestrogens (Tamoxifen, raloxifene, fulvestrant), topoisomerase inhibitors (camptothecin, irinotecan, doxorubicin, mitoxantrone, etoposide, epirubicin), microtubule active compounds (paclitaxel docetaxel, vinblastine, vincristine, discodermolide, colchicine, epothilonc), alkylating agents (ifosfamide, cyclophosphamide), histone deacetylase inhibitors (SAHA), antincoplastic metabolite (5-fluorouracil, gemcitabine, 5-azacytidine, methotrexate, pemetrexed), cyclooxygenase inhibitors (celecoxib, rofecoxib, valdecoxib), MMP inhibitors, mTOR inhibitors (sirolimus), platin compounds (cisplatin, oxaliplatin), kinase inhibitors (imatinib, sunitinib, nilotinib, dasatinib, Herceptin, iressa, tarceva, pacritinib, tofacitinib, ruxolitinib, ibrutinib, fostamatinib), anti-angiogenic compounds (thalidomide), gonadorclin agonists (abarelix, goserelin), anti-androgens (bicalutamide), bisphosphonates, antiproliferative antibodies (Herceptin, Erbitux, bevacizumab, rituximab), heparanase inhibitors, inhibitors of Ras oncogenic isoforms, telomerase inhibitors (telomestatin), proteasome inhibitors (bortezomib), HSP-90 inhibitors, temozolomide, kinesin spindle protein inhibitors, MEK inhibitors. Furthermore, the second therapeutic agent may comprise adefovir, tenofovir disoproxil fumarate+emtricitabine (Truvada), tenofovir disoproxil fumarate (Vircad), entecavir, lamivudine, tenofovir alafenamide, telbivudine, clevudine, emtricitabine, peginterferon alpha 2b, multiferon, interferon alpha 1b, interferon alpha 2b, pegylated interferon alpha 2a, interferon alpha n1, ribavirin, interferon beta 1a, bioferon, interferon alpha 2b, 4-ethynyl-2-fluoro-deoxyadenosine, HIV/HBV/HCV vaccines, HBV/HCV DNA polymerase inhibitors, HIV reverse transcriptase inhibitors, HIV/HBV/HCV protease inhibitors, HIV integrase inhibitors, HIV maturation inhibitors, HIV capsid inhibitors, HIV Vif inhibitors, gp41 inhibitors, CXCR4 antagonists, gp120 inhibitors, C5a antagonists, cyclophilin inhibitors, surface antigen inhibitors, viral entry inhibitors, antisense oligonucleotides targeting viral mRNA, CCR2 antagonists, CCR5 antagonists, cytokines, RIG-I stimulators, NOD2 stimulators, PI3K inhibitors and pharmacokinetic enhancers.

Methods for co-administration with an additional therapeutic agent are well known in the art (Hardman et. al. (eds.), Goodman and Gilman's The Pharmacological Basis of Therapeutics, 10th ed., 2001, McGraw-Hill New York, NY; Poole and Peterson (eds.), Pharmacotherapeutics for Advanced Practice: A Practical Approach, 2001, Lippincott, Williams and Wilkins, Philadelphia, PA; Chabner and Longo (eds.), Cancer Chemotherapy and Biotherapy, 2001, Lippincott, Williams and Wilkins, Philadelphia, PA).

In one aspect, the disease is cancer and a chemotherapeutic agent may be administered with a compound of Formula (I). The chemotherapeutic agent may be selected from a group further consisting of a cancer vaccine, a targeted drug, a targeted antibody, an antibody fragment, an antimetabolite, an antincoplastic, an antifolate, a toxin, an alkylating agent, a DNA strand breaking agent, a DNA minor groove binding agent, a pyrimidine analogue, a ribonucleotide reductase inhibitor, a tubulin interactive agent, an anti-hormonal agent, an immunomodulator, an anti-adrenal agent, a cytokine, radiation therapy, a cell therapy, cell depletion therapy such as B-cell depletion therapy and a hormone therapy. Alternatively or additionally, the chemotherapeutic agent may comprise abiraterone, altretamine, anhydrovinblastine, auristatin, bexarotene, bicalutamide, bleomycin, cachectin, cemadotin, chlorambucil, cyclophosphamide, docetaxol, doxetaxel, carboplatin, cysplatin, cytarabinc, dactinomycin, daunorubicin, decitabine, doxorubicin, etoposide, 5-fluorouracil, finasteride, flutamide, hydroxyurea, streptozocin, mitomycin, methotrexate, taxanes, tamoxifen, vinblastine, vincristine and/or vindesine.

A complex of the compound of formula (I) may be understood to be a multi-component complex, wherein the drug and at least one other component are present in stoichiometric or non-stoichiometric amounts. The complex may be other than a salt or solvate. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together-see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The term “pharmaceutically acceptable salt” may be understood to refer to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, adepic, aspartic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) base addition salts formed when an acidic proton present in the parent compound either (a) is replaced by a metal ion, e.g., an alkali metal ion, an alkaline earth ion or an aluminium ion, or alkali metal or alkaline earth metal hydroxides, such as sodium, potassium, calcium, magnesium, aluminium, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts may include, sodium, potassium, calcium, magnesium, ammonium, tetraalkylammonium and the like, and when the compound contains a basic functionality, salts of non-toxic organic or inorganic acids, such as hydrohalides, e.g. hydrochloride, hydrobromide and hydroiodide, carbonate or bicarbonate, sulfate or bisulfate, borate, phosphate, hydrogen phosphate, dihydrogen phosphate, pyroglutamate, saccharate, stearate, sulfamate, nitrate, orotate, oxalate, palmitate, pamoate, acetate, trifluoroacetate, trichloroacetate, propionate, hexanoate, cyclopentylpropionate, glycolate, glutarate, pyruvate, lactate, malonate, succinate, tannate, tartrate, tosylate, sorbate, ascorbate, malate, maleate, fumarate, tartarate, camsylate, citrate, cyclamate, benzoate, isethionate, esylate, formate, 3-(4-hydroxybenzoyl)benzoate, picrate, cinnamate, mandelate, phthalate, laurate, methanesulfonate (mesylate), methylsulphate, naphthylate, 2-napsylate, nicotinate, ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluceptate, gluconate, glucoronate, hexafluorophosphate, hibenzate, benzoate, glutamate, hydroxynaphthoate, salicylate, stearate, cyclohexylsulfamate, quinate, muconate, xinofoate and the like.

Hemisalts of acids and bases may also be formed, for example, hemisulphate salts. The skilled person will appreciate that the aforementioned salts include ones wherein the counterion is optically active, for example D-lactate, or racemic, for example DL-tartrate.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The term “solvate” may be understood to refer to a compound provided herein or a salt thereof, that further includes a stoichiometric or non-stoichiometric amount of solvent bound by non-covalent intermolecular forces. Where the solvent is water, the solvate is a hydrate. Pharmaceutically acceptable solvates in accordance with the invention include those wherein the solvent of crystallization may be isotopically substituted, e.g. D₂O, d6-acetone and d6-DMSO.

A currently accepted classification system for organic hydrates is one that defines isolated site, channel, or metal-ion coordinated hydrates-see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may exist in a continuum of solid states ranging from fully amorphous to fully crystalline, including polymorphs of said crystalline material. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO—Na+, —COO—K+, or —SO3—Na+) or non-ionic (such as —N—N+ (CH3)₃) polar head group. For more information, see Crystals and the Polarizing Microscope by N. H. Hartshorne and A. Stuart, 4th Edition (Edward Arnold, 1970), incorporated herein by reference.

The compound of Formula (I) may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the compounds described herein may be used in a number of ways. Suitable modes of administration include oral, intra-tumoral, parenteral, topical, inhaled/intranasal, rectal/intravaginal, and ocular/aural administration.

Formulations suitable for the aforementioned modes of administration may be formulated to be immediate and/or modified release. Modified release formulations include delayed-, sustained-, pulsed-, controlled-, targeted and programmed release.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays, liquid formulations and buccal/mucoadhesive patches.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25 (2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic) acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated-see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as L-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

In the case of dry powder inhalers and aerosols, the dosage unit is determined by means of a valve which delivers a metered amount. Units in accordance with the invention are typically arranged to administer a metered dose or “puff” containing from 1 μg to 100 mg of the compound of formula (I). The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, microbicide, vaginal ring or enema. Cocoa butter is a traditional suppository base, but various alternatives may be used as appropriate.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compounds of the invention may also be administered directly to a site of interest by injection of a solution or suspension containing the active drug substance. The site of interest may be a tumour and the compound may be administered via intratumoral injection. Typical injection solutions are comprised of propylene glycol, sterile water, ethanol and sodium chloride. Alternative solvents which may be used instead of propylene glycol include glycerol and polyethylene glycol.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

It will be appreciated that the amount of the compound that is required is determined by its biological activity and bioavailability, which in turn depends on the mode of administration, the physiochemical properties of the compound, and whether it is being used as a monotherapy, or in a combined therapy. The frequency of administration will also be influenced by the half-life of the compound within the subject being treated. Optimal dosages to be administered may be determined by those skilled in the art, and will vary with the particular compound in use, the strength of the pharmaceutical composition, the mode of administration, and the advancement of the disease. Additional factors depending on the particular subject being treated will result in a need to adjust dosages, including subject age, weight, gender, diet, and time of administration. In this regard, the amount of compound in compositions of this invention are sufficient to measurably inhibit HPK-1, or a mutant thereof, in a biological sample or in a patient.

Generally, for administration to a human, the total daily dose of the compounds of the invention is typically in the range 100 μg to 10 g, such as 1 mg to 1 g, for example 10 mg to 500 mg. For example, oral administration may require a total daily dose of from 25 mg to 250 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

However, it is appreciated by those skilled in the art that for agents that modulate the immune system, both the dose and the frequency of administration may be different to those of more traditional therapies. In particular, for agents that stimulate the immune system, for example through modulation of HPK-1, they may be administered in small doses, and quite infrequently, for example twice weekly, weekly or monthly. Smaller doses may also be effective when administered topically to a small area of skin.

The compound may be administered before, during or after onset of the disease to be treated.

Known procedures, such as those conventionally employed by the pharmaceutical industry (e.g. in vivo experimentation, clinical trials, etc.), may be used to form specific formulations comprising the compounds according to the invention and precise therapeutic regimes (such as daily doses of the compounds and the frequency of administration). The inventors believe that they are the first to describe a pharmaceutical composition for treating a disease, based on the use of the compounds of the invention.

Hence, in a tenth aspect of the invention, there is provided a pharmaceutical composition comprising a compound according to the first aspect, or a pharmaceutically acceptable salt, solvate, tautomeric form or polymorphic form thereof, and a pharmaceutically acceptable vehicle.

The invention also provides, in an eleventh aspect, a process for making the composition according to the tenth aspect, the process comprising contacting a therapeutically effective amount of a compound of the first aspect, or a pharmaceutically acceptable salt, solvate, tautomeric form or polymorphic form thereof, and a pharmaceutically acceptable vehicle.

A “subject” may be a vertebrate, mammal, or domestic animal. Hence, compounds, compositions and medicaments according to the invention may be used to treat any mammal, for example livestock (e.g. a horse), pets, or may be used in other veterinary applications. Most preferably, however, the subject is a human being.

A “therapeutically effective amount” of compound is any amount which, when administered to a subject, is the amount of drug that is needed to treat the target disease, or produce the desired effect, i.e. modulate the HPK-1 protein.

For example, the therapeutically effective amount of compound used may be from about 0.01 mg to about 800 mg, and preferably from about 0.01 mg to about 500 mg. It is preferred that the amount of compound is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the compound according to the first, second, third and sixth aspects) according to the invention. In tablets, the active compound may be mixed with a vehicle having the necessary compression properties in suitable proportions and compacted in the shape and size desired. The powders and tablets preferably contain up to 99% of the active compound. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The compound according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The compound may be prepared as a sterile solid composition that may be dissolved or suspended at the time of administration using sterile water, saline, or other appropriate sterile injectable medium.

The compound and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compounds used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Also included within the scope of the invention are soft drugs or antedrugs which are compounds of formula (I) which contain metabolically or hydrolytically labile moieties which in vivo are converted into inactive derivatives. The processes by which the active drug substance is converted into an inactive derivative include, but are not limited to, ester hydrolysis, S-oxidation, N-oxidation, dealkylation and metabolic oxidation as described for example in Pearce et al., Drug Metab. Dispos., 2006, 34, 1035-1040 and B. Testa, Prodrug and Soft Drug Design, in Comprehensive Medicinal Chemistry II, Volume 5, Elsevier, Oxford, 2007, pp. 1009-1041 and Bodor, N. Chem. Tech. 1984, 14, 28-38.

It will be known to those skilled in the art that active drug ingredients may be converted into a prodrug, which is a metabolically labile derivative that is converted within the body into the active drug substance. Also included within the scope of the invention are prodrugs which are compounds of formula (I) which contain metabolically or hydrolytically labile moieties which in vivo are converted into the active drug of formula (I). The processes by which the prodrug is converted into the active drug substance include, but are not limited to, ester hydrolysis, phosphate ester hydrolysis, S-oxidation, N-oxidation, dealkylation and metabolic oxidation as described in Beaumont et. al., Curr. Drug Metab., 2003, 4, 461-485 and Huttenen et. al., Pharmacol. Revs., 2011, 63, 750-771. The aforementioned prodrug moieties may therefore encompass functional groups which include carbonates, carbamates, esters, amides, ureas and lactams. Such prodrug derivatives may offer improved solubility, stability or permeability compared to the parent drug substance, or may better allow the drug substance to be administered by an alternative route of administration, for example as an intravenous solution.

The compounds contained herein may also be used as an active ingredient in a proteolysis-targeting chimera (PROTAC). Inspired by the normal use by cells of the ubiquitin-proteasome system (UPS) to maintain intracellular homeostasis, PROTACs use the endogenous ubiquitinylating machinery to recognise and degrade specific proteins that have been tagged by a ligand with affinity for that protein. The PROTAC molecules are bifunctional and consist of three main constituents; a protein targeting moiety (PTM), a linker (L) and a moiety that targets and recruits an E3 ubiquintinylating ligase complex (ULM) to degrade the target protein. It will be appreciated that compounds of the invention may be used as the PTM component.

Accordingly, in accordance with a further aspect, there is provided a PROTAC of formula (II):

PTM-L-ULM (II),

wherein PTM is a protein targeting moiety, and is a compound of formula (I);
L is a linker; and
ULM is an E3 ubiquintinylating ligase complex;
or a pharmaceutically acceptable salt, solvate, tautomeric form or polymorphic form thereof.

Suitable linkers and E3 ubiquintinylating ligase complexes are known in the art.

The compound of formula (I) may be attached to the linker through the Z group. Accordingly, the Z group may be as defined above except with a hydrogen atom removed therefrom to cause the group to be bivalent.

Accordingly, the PROTAC of formula (II) is preferably a PROTAC of formula (IIa):

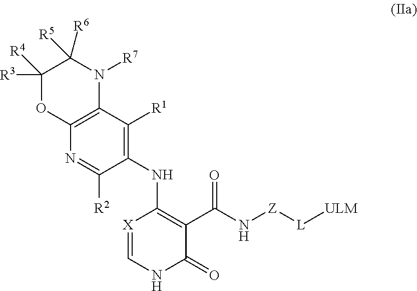

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the invention, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with isotopes such as deuterium, i.e. 2H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

In accordance with a further aspect, there is provided a compound of formula (I):

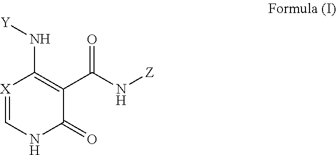

wherein:
Y is selected from formulae (a)-(h):

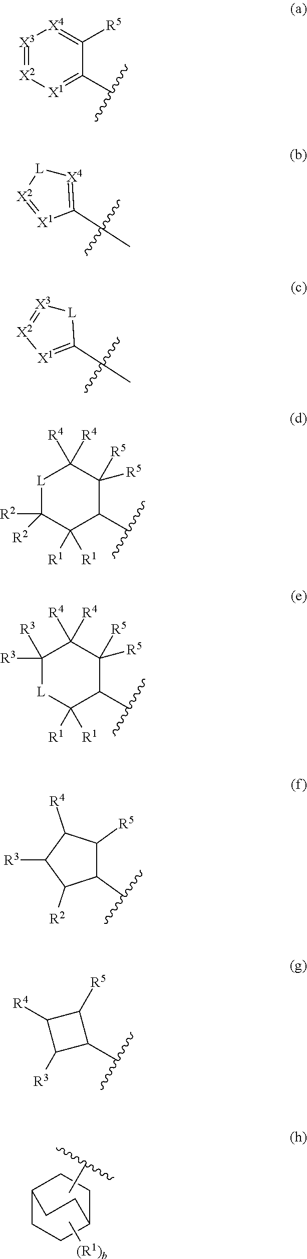

X is N or CH;
X¹ is N or CR¹;
X² is N or CR²;
X³ is N or CR³;
X⁴ is N or CR⁴;
L is O, S, NR⁶ or CR⁶R⁷;
Z is a phenyl or 5 or 6 membered heteroaryl, wherein the phenyl or heteroaryl is substituted with one or more substituents selected from the group consisting of a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 10 membered heterocyclyl, and an optionally substituted 5 to 10 membered heteroaryl and/or where adjacent substituents of the phenyl or heteroaryl, together with the atoms to which they are attached, may combine to form an optionally substituted 3 to 6 membered heterocycle or an optionally substituted 5 or 6 membered heteroaryl;
R¹ to R⁷ are independently hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl; and/or one or more pairs of adjacent R¹ to R⁷ groups, together with the atoms to which they are attached, combine to form an optionally substituted 3 to 6 membered heterocycle, an optionally substituted 5 or 6 membered heteroaryl, an optionally substituted 3 to 6 membered cycloalkyl or an optionally substituted phenyl, and/or a pair of R¹ to R⁵ groups on the same C atom and/or R⁶ and R⁷, together with the C atom they are bound to, form a C═O group;
R⁸ and R⁹ are independently hydrogen, an optionally substituted C₁-C₃ alkyl, an optionally substituted C₂-C₃ alkenyl, an optionally substituted C₂-C₃ alkynyl, optionally substituted C₆₋₁₂ aryl, optionally substituted C₃-C₆ cycloalkyl, optionally substituted 3 to 8 membered heterocyclyl, and an optionally substituted 5 to 10 membered heteroaryl;
R¹⁰ and R¹¹ are independently hydrogen, an optionally substituted C₁-C₆ alkyl, an optionally substituted C₂-C₆ alkenyl or an optionally substituted C₂-C₆ alkynyl, optionally substituted C₆₋₁₂ aryl, optionally substituted C₃-C₆ cycloalkyl, optionally substituted 3 to 8 membered heterocyclyl, and an optionally substituted 5 to 10 membered heteroaryl; and bis 0, 1, 2, 3 or 4;
or a pharmaceutically acceptable complex, salt, solvate, tautomeric form or polymorphic form thereof;
with the proviso that the compound is not:

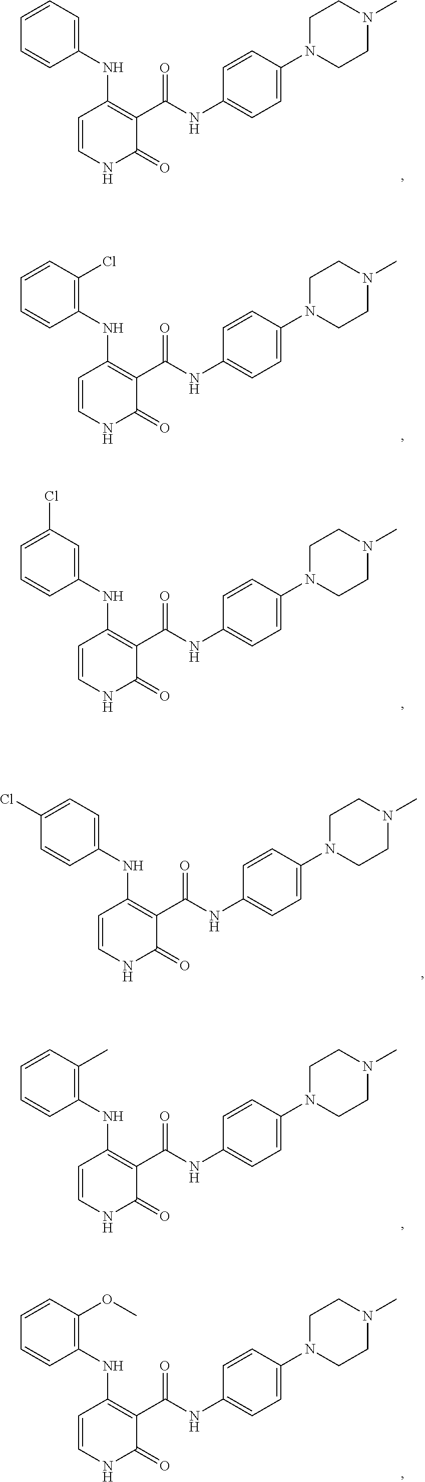 [SMILES: CN1CCN(c2ccc(NC(=O)c3c(Nc4ccccc4)cc[nH]c3=O)cc2)CC1 ; CN1CCN(c2ccc(NC(=O)c3c(Nc4ccccc4Cl)cc[nH]c3=O)cc2)CC1 ; CN1CCN(c2ccc(NC(=O)c3c(Nc4cccc(Cl)c4)cc[nH]c3=O)cc2)CC1 ; CN1CCN(c2ccc(NC(=O)c3c(Nc4ccc(Cl)cc4)cc[nH]c3=O)cc2)CC1 ; Cc1ccccc1Nc1cc[nH]c(=O)c1C(=O)Nc1ccc(N2CCN(C)CC2)cc1 ; COc1ccccc1Nc1cc[nH]c(=O)c1C(=O)Nc1ccc(N2CCN(C)CC2)cc1]

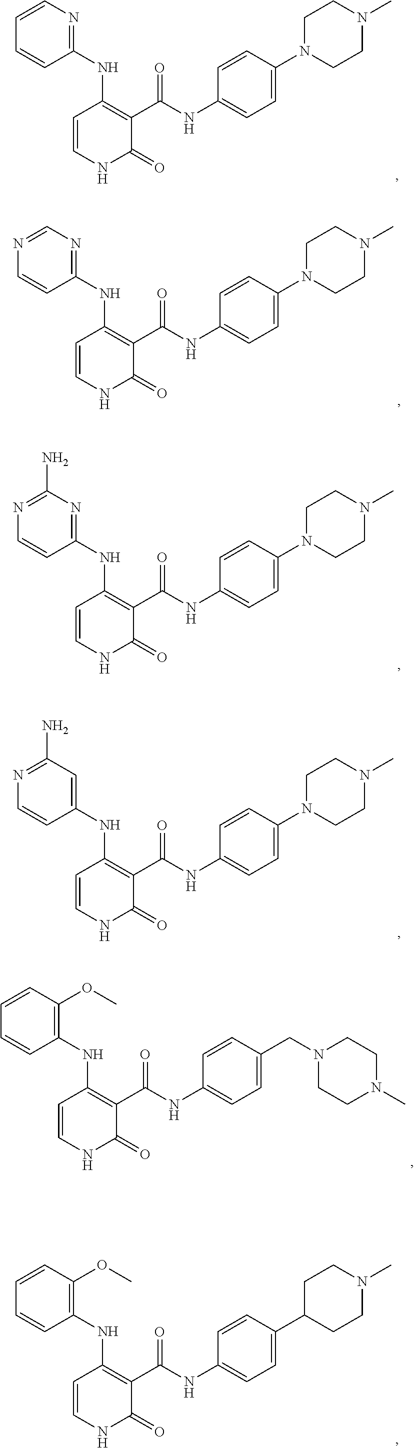 [SMILES: CN1CCN(c2ccc(NC(=O)c3c(Nc4ccccn4)cc[nH]c3=O)cc2)CC1 ; CN1CCN(c2ccc(NC(=O)c3c(Nc4ccncn4)cc[nH]c3=O)cc2)CC1 ; CN1CCN(c2ccc(NC(=O)c3c(Nc4ccnc(N)n4)cc[nH]c3=O)cc2)CC1 ; CN1CCN(c2ccc(NC(=O)c3c(Nc4ccnc(N)c4)cc[nH]c3=O)cc2)CC1 ; COc1ccccc1Nc1cc[nH]c(=O)c1C(=O)Nc1ccc(CN2CCN(C)CC2)cc1 ; COc1ccccc1Nc1cc[nH]c(=O)c1C(=O)Nc1ccc(C2CCN(C)CC2)cc1]

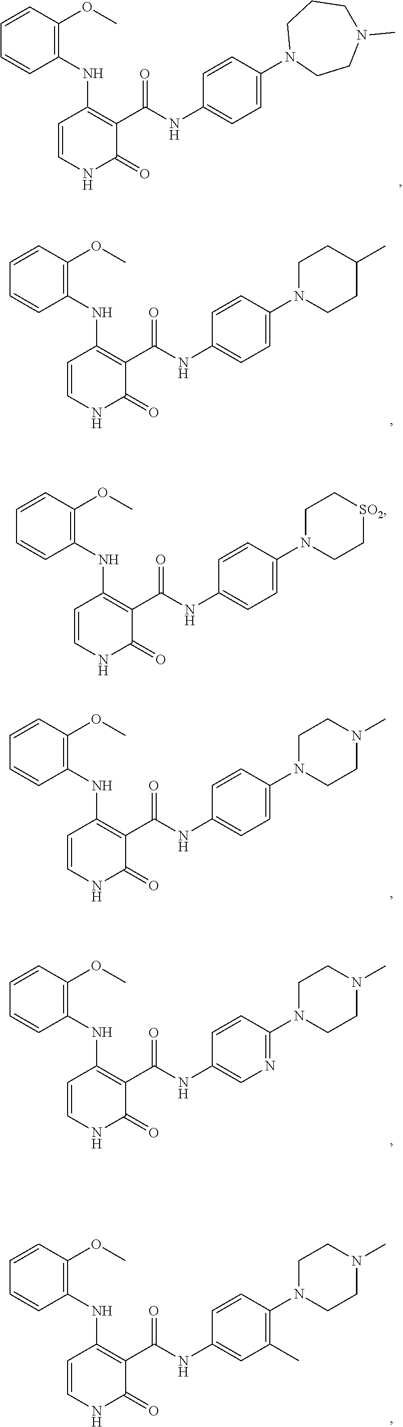 [SMILES: COc1ccccc1Nc1cc[nH]c(=O)c1C(=O)Nc1ccc(N2CCCN(C)CC2)cc1 ; COc1ccccc1Nc1cc[nH]c(=O)c1C(=O)Nc1ccc(N2CCC(C)CC2)cc1 ; COc1ccccc1Nc1cc[nH]c(=O)c1C(=O)Nc1ccc(N2CCS(=O)(=O)CC2)cc1 ; COc1ccccc1Nc1cc[nH]c(=O)c1C(=O)Nc1ccc(N2CCN(C)CC2)cc1 ; COc1ccccc1Nc1cc[nH]c(=O)c1C(=O)Nc1ccc(N2CCN(C)CC2)nc1 ; COc1ccccc1Nc1cc[nH]c(=O)c1C(=O)Nc1ccc(N2CCN(C)CC2)c(C)c1]

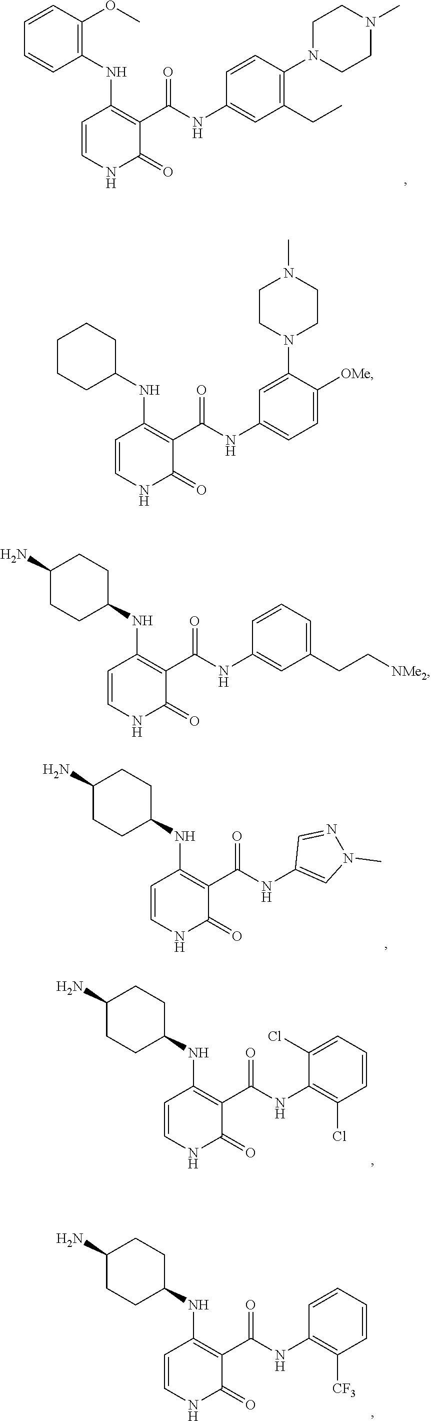 [SMILES: CCc1cc(NC(=O)c2c(Nc3ccccc3OC)cc[nH]c2=O)ccc1N1CCN(C)CC1 ; COc1ccc(NC(=O)c2c(NC3CCCCC3)cc[nH]c2=O)cc1N1CCN(C)CC1 ; CN(C)CCc1cccc(NC(=O)c2c(N[C@H]3CC[C@@H](N)CC3)cc[nH]c2=O)c1 ; Cn1cc(NC(=O)c2c(N[C@H]3CC[C@@H](N)CC3)cc[nH]c2=O)cn1 ; N[C@H]1CC[C@@H](Nc2cc[nH]c(=O)c2C(=O)Nc2c(Cl)cccc2Cl)CC1 ; N[C@H]1CC[C@@H](Nc2cc[nH]c(=O)c2C(=O)Nc2ccccc2C(F)(F)F)CC1]

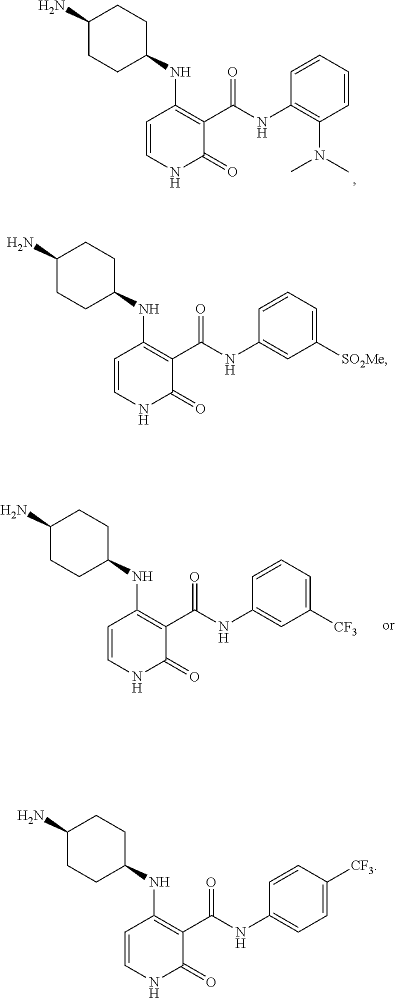 [SMILES: CN(C)c1ccccc1NC(=O)c1c(N[C@H]2CC[C@@H](N)CC2)cc[nH]c1=O ; CS(=O)(=O)c1cccc(NC(=O)c2c(N[C@H]3CC[C@@H](N)CC3)cc[nH]c2=O)c1 ; N[C@H]1CC[C@@H](Nc2cc[nH]c(=O)c2C(=O)Nc2cccc(C(F)(F)F)c2)CC1 ; "or" ; N[C@H]1CC[C@@H](Nc2cc[nH]c(=O)c2C(=O)Nc2ccc(C(F)(F)F)cc2)CC1]

Preferred fused groups Y include the following groups. For simplicity, each of these groups is illustrated without substituents however it will be understood that each ring of these fused groups may independently be unsubstituted or substituted with one or more substituents independently selected from non-H groups of R¹-R⁷ as applicable:

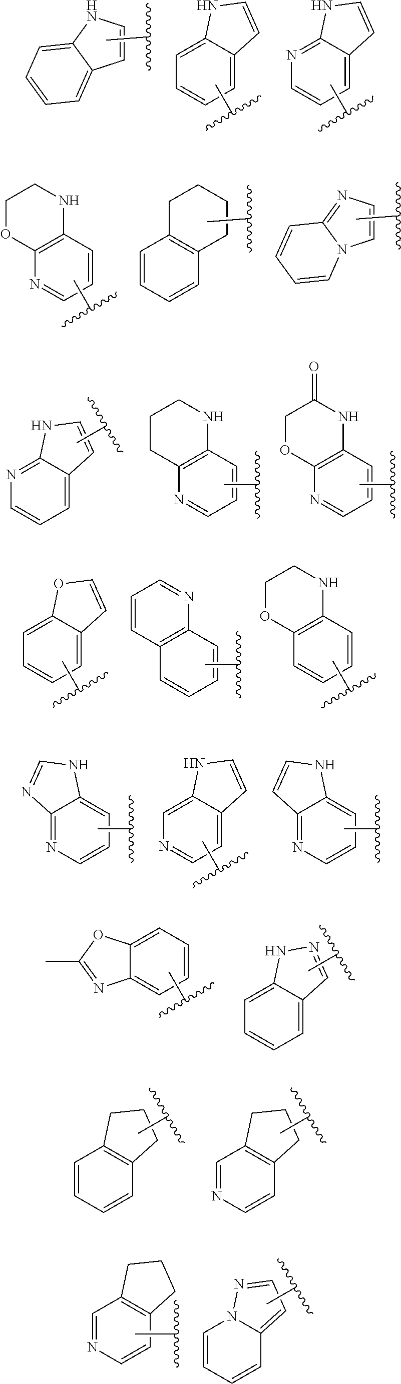

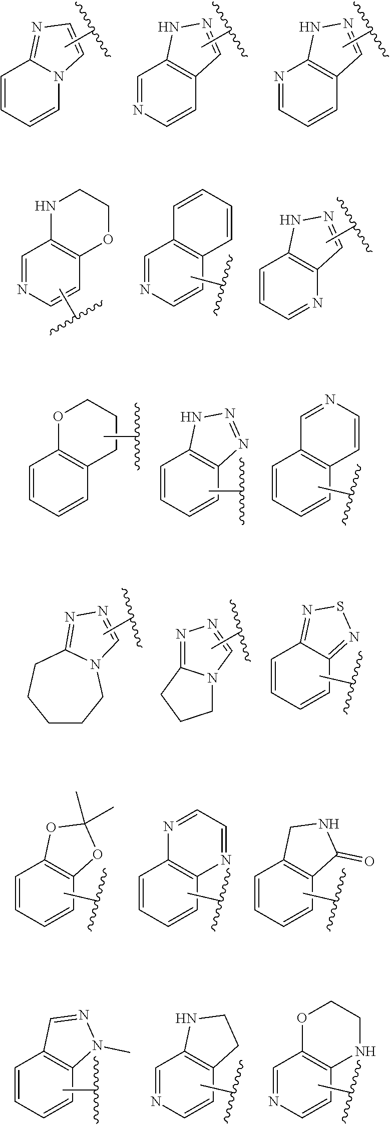

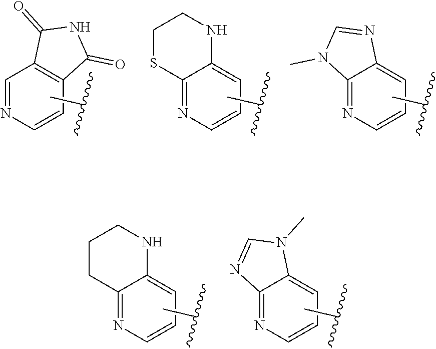

A bicyclic group Y is preferably a group of the following formula which may be unsubstituted or substituted with one or more substituents R¹, preferably OH:

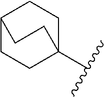 [SMILES: *C12CCC(CC1)CC2]

Preferred substituents of groups of formulae (a)-(h) are selected from: C₁₋₆ alkyl; C₃₋₆ cycloalkyl; —C₁₋₆ alkylene-OH; —OAk; OH, —C(═O)Ak; —NH₂; —NHAk; NAK₂; —CONH₂; CONHAK; CON(Ak)₂; C₁₋₆ perfluoroalkyl, C₁₋₆ perfluoroalkoxy; CN; —NHC(═O) Ak; —NHC(═O)Ar; —NHSO₂Ak; halogen, preferably F or C₁; optionally substituted phenyl; optionally substituted pyridyl; and a heterocyclic group of N and C ring atoms, for example pyrrolidinyl, piperidinyl or piperazinyl which is unsubstituted or substituted with one or more substituents, for example one or more C₁₋₆ alkyl groups, wherein Ak in each occurrence is independently a C₁₋₆ alkyl group or a C₃₋₆ cycloalkyl group; and Ar is an aryl or heteroaryl group which is unsubstituted or substituted with one or more substituents.

Ar is preferably selected from phenyl and a 5- or 6-membered heteroaryl having ring atoms selected from C atoms, N atoms and, optionally, O or S atoms. Ar may be unsubstituted or substituted with one or more substituents, e.g. one or more groups selected from C₁₋₆ alkyl, C₁₋₆ alkoxy, F, Cl, NO₂ and CN.

As explained above, Y is selected from formulae (a)-(h).

R¹ to R⁷ may independently be hydrogen, an optionally substituted C₁-C₆ alkyl, a halogen, CN, OR¹⁰, COR¹⁰, COOR⁸, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl; and/or one or more pairs of adjacent R¹ to R⁷ groups, together with the atoms to which they are attached, may combine to form an optionally substituted 3 to 6 membered heterocycle, an optionally substituted 5 or 6 membered heteroaryl, an optionally substituted 3 to 6 membered cycloalkyl or an optionally substituted phenyl, and/or a pair of R¹ to R⁵ groups on the same C atom and/or R⁶ and R⁷, together with the C atom they are bound to may form a C═O group. Preferably, R¹ to R⁷ are independently hydrogen, an optionally substituted C₁-C₃ alkyl, a halogen, CN, OR¹⁰, COR¹⁰, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl; and/or one or more pairs of adjacent R¹ to R⁷ groups, together with the atoms to which they are attached, may combine to form an optionally substituted 3 to 6 membered heterocycle, an optionally substituted 5 or 6 membered heteroaryl, an optionally substituted 3 to 6 membered cycloalkyl or an optionally substituted phenyl, and/or a pair of R¹ to R⁵ groups on the same C atom and/or R⁶ and R⁷, together with the C atom they are bound to may form a C═O group. R⁸ and R⁹ may independently be hydrogen, an optionally substituted C₁-C₃ alkyl, optionally substituted phenyl, optionally substituted C₃-C₆ cycloalkyl, optionally substituted 5 or 6 membered heterocyclyl, and an optionally substituted 5 or 6 membered heteroaryl. The or each alkyl may be unsubstituted or substituted with one or more of halogen, OH or OCH₃. The or each cycloalkyl, heterocyclyl or heteroaryl may be unsubstituted or substituted with one or more of halogen, C₁₋₃ alkyl, OH and oxo, more preferably the or each cycloalkyl, heterocyclyl or heteroaryl may be unsubstituted or substituted C₁, CH₃ or oxo.

R¹ to R⁷ may independently be hydrogen, methyl, CF₃, CH₂OH, F, Cl, CN, OH, OCH₃, OCF₃, COCH₃, CONH₂, NHCOCH₃,

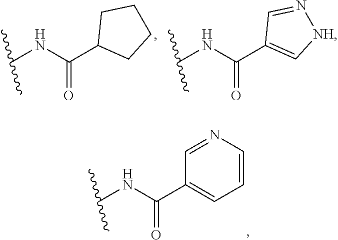

NHSO₂CH₃, NH₂,

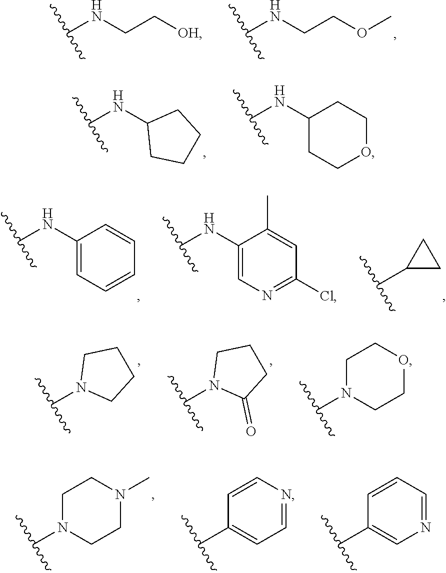

or phenyl and/or one or more pairs of adjacent R¹ to R⁷ groups, together with the atoms to which they are attached, combine to form an optionally substituted 3 to 6 membered heterocycle, an optionally substituted 5 or 6 membered heteroaryl, an optionally substituted 3 to 6 membered cycloalkyl or an optionally substituted phenyl, and/or a pair of R¹ to R⁵ groups on the same C atom and/or R⁶ and R⁷, together with the C atom they are bound to, form a C═O group

In some embodiments, a pair of adjacent R¹ to R⁷ groups, together with the atoms to which they are attached, combine to form an optionally substituted 3 to 6 membered heterocycle, an optionally substituted 5 or 6 membered heteroaryl, an optionally substituted 3 to 6 membered cycloalkyl or an optionally substituted phenyl. More preferably, in some embodiments, a pair of adjacent R¹ to R⁵ groups, together with the atoms to which they are attached, combine to form an optionally substituted 5 or 6 membered heterocycle, an optionally substituted 5 or 6 membered heteroaryl or an optionally substituted phenyl. The heterocycle, heteroaryl, cycloalkyl or phenyl formed by the pair of adjacent R¹ to R⁵ groups may be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷ or NR¹⁶R¹⁷.

R¹⁶ and R¹⁷ may each independently be selected from the group consisting of H, optionally substituted C₁-C₆ alkyl, optionally substituted C₂-C₆ alkenyl, optionally substituted C₂-C₆ alkynyl, optionally substituted C₆₋₁₂ aryl, optionally substituted C₃₋₆ cycloalkyl, optionally substituted 3 to 6 membered heterocycle or optionally substituted 5 to 10 membered heteroaryl. R¹⁶ and R¹⁷ may each independently be selected from the group consisting of H, optionally halogenated C₁-C₆ alkyl, optionally halogenated C₂-C₆ alkenyl or optionally halogenated C₂-C₆ alkynyl. R¹⁶ and R¹⁷ may each independently be H or methyl. In some embodiments, R¹⁶ and R¹⁷ are both H.

More preferably, the heterocycle, heteroaryl, cycloalkyl or phenyl formed by the pair of adjacent R¹ to R⁵ groups may be unsubstituted or substituted with one or more of C₁-C₃ alkyl, C₂-C₃ alkenyl, C₂-C₃ alkynyl, C₁-C₃ alkoxy, halogen, oxo, OH and NH₂. Most preferably, the heterocycle, heteroaryl, cycloalkyl or phenyl formed by the pair of adjacent R¹ to R⁵ groups may be unsubstituted or substituted with one or more of methyl, fluorine and oxo.

In some embodiments, Y has formula (a). In some embodiments, X¹ is CR¹, X² is CR², X³ is CR³ and X⁴ is CR⁴. In some embodiments, X¹ is N, X² is CR², X³ is CR³ and X⁴ is CR⁴. In some embodiments, X¹ is CR¹, X² is N, X³ is CR³ and X⁴ is CR⁴. In some embodiments, X¹ is CR¹, X² is CR², X³ is N and X⁴ is CR⁴. In some embodiments, X¹ is CR¹, X² is CR², X³ is CR³ and X⁴ is N. In some embodiments, X¹ is N, X² is N, X³ is CR³ and X⁴ is CR⁴. In some embodiments, X¹ is CR¹, X² is N, X³ is CR³ and X⁴ is N.

Accordingly, Y may be

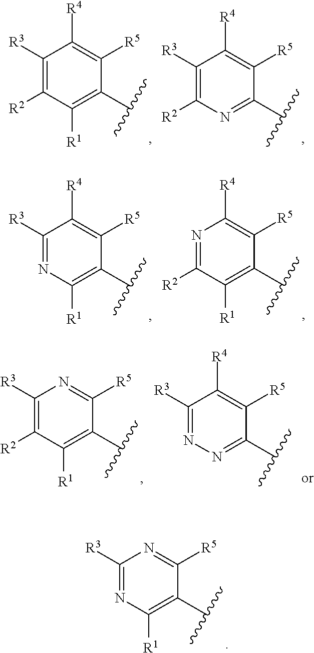

In embodiments where X¹ is CR¹, X² is CR², X³ is CR³ and X⁴ is CR⁴ then preferably one or more pairs of adjacent R¹ to R⁵ groups, together with the atoms to which they are attached, combine to form an optionally substituted 3 to 6 membered heterocycle, an optionally substituted 5 or 6 membered heteroaryl, an optionally substituted 3 to 6 membered cycloalkyl or an optionally substituted phenyl. In embodiments where Y is a bicyclic group, Y may be

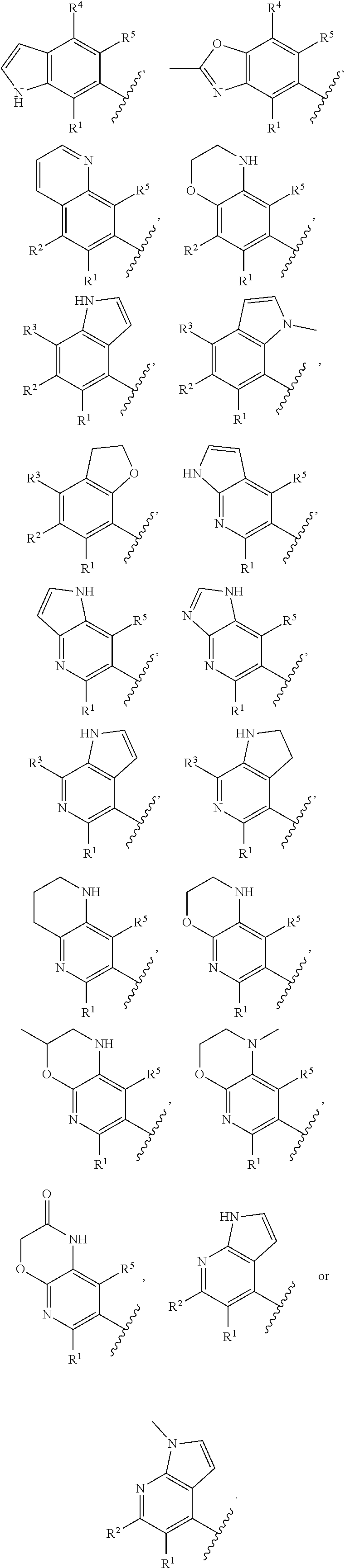

In some embodiments, Y may be

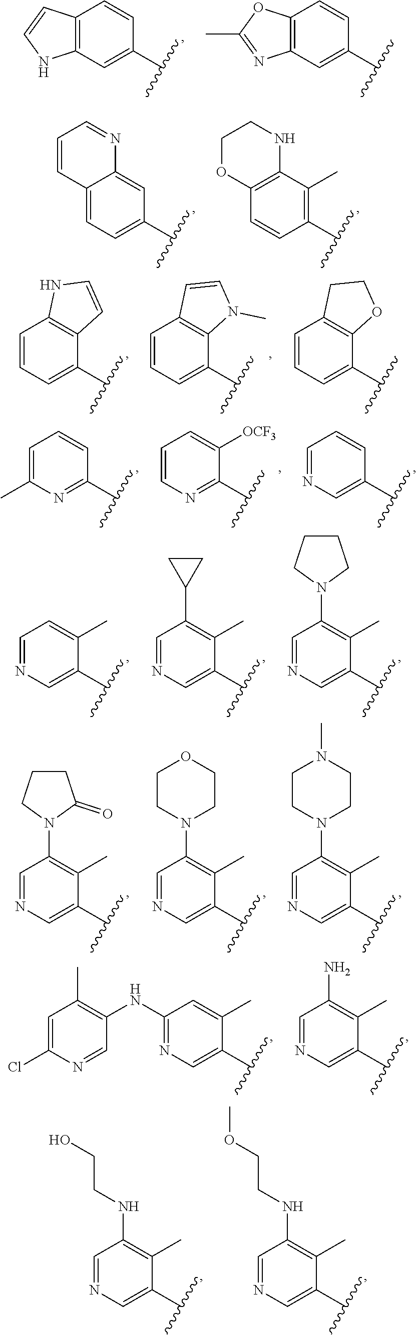

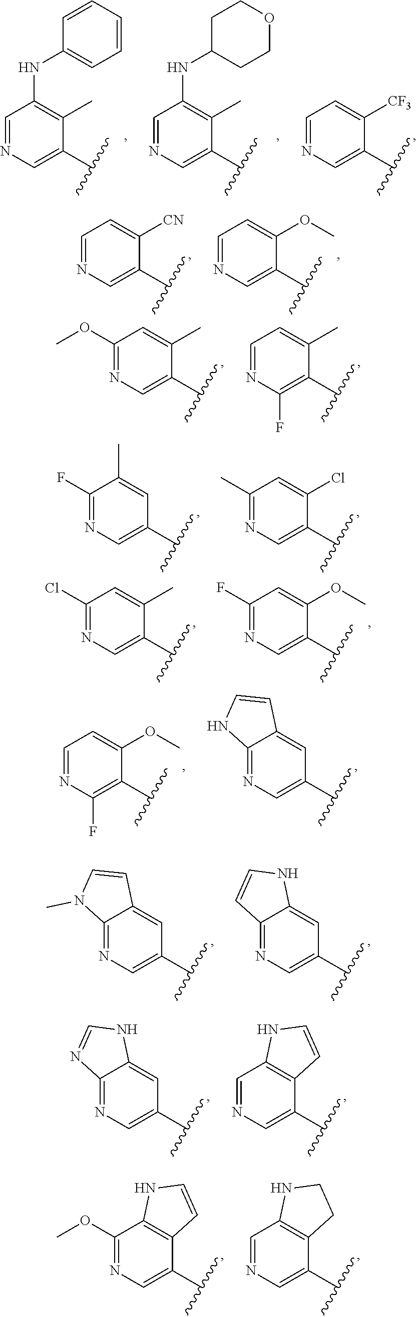

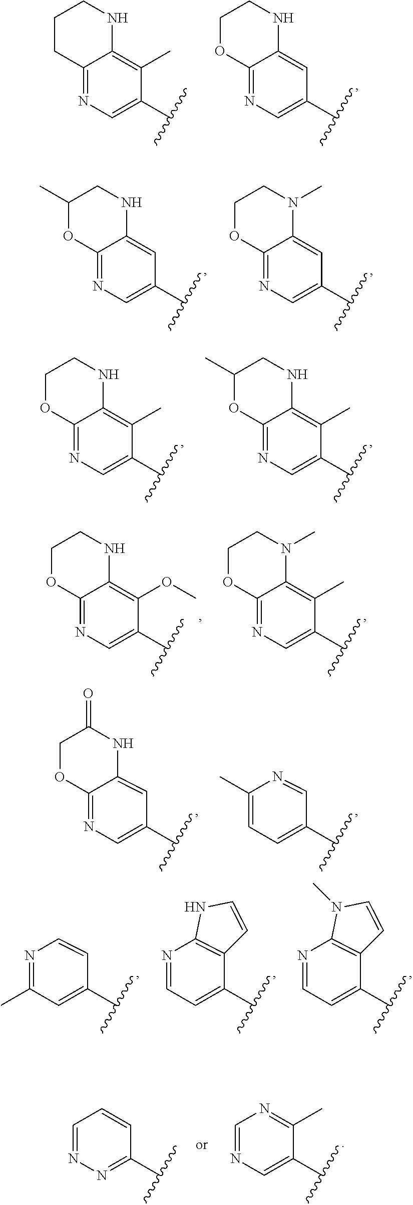

In some embodiments, Y has formula (b). L may be NR⁶. X¹ may be CR¹. X² may be CR². X⁴ may be CR⁴. Accordingly, Y may be

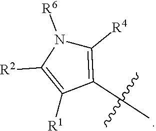

In embodiments where Y is a bicyclic group, Y may be

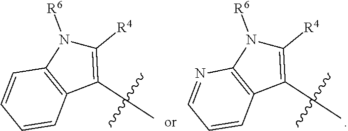

In some embodiments, Y may be

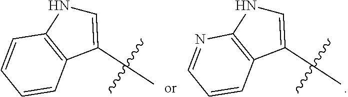 [SMILES: *c1c[nH]c2ccccc12 ; *c1c[nH]c2ncccc12]

In some embodiments, Y has formula (c). L may be NR⁶ or S. X¹ may be CR¹, X² may be CR² and X³ may be CR³. Alternatively, X¹ may be N, X² may be CR² and X³ may be CR³. Alternatively, X¹ may be CR¹, X² may be N and X³ may be CR³. Alternatively, X¹ may be CR¹, X² may be CR² and X³ may be N. Accordingly, Y may be

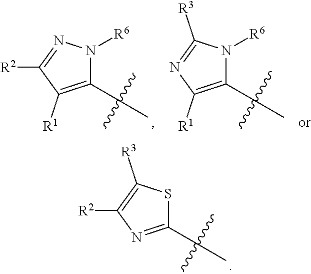

In embodiments where Y is a bicyclic group, Y may be

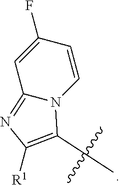

In some embodiments, Y may be

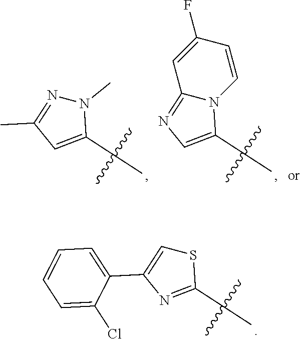

In some embodiments, Y has formula (d) or (e). L may be O, NR⁶ or CR⁶R⁷.

Accordingly, Y may be

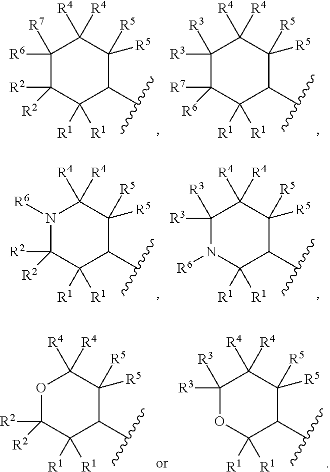

In embodiments where Y is a bicyclic group, Y may be

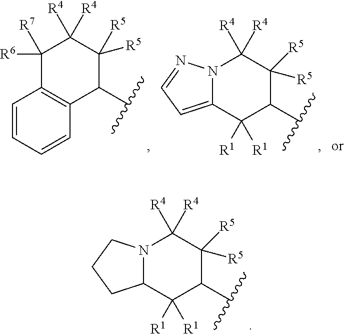

In some embodiments, Y may be

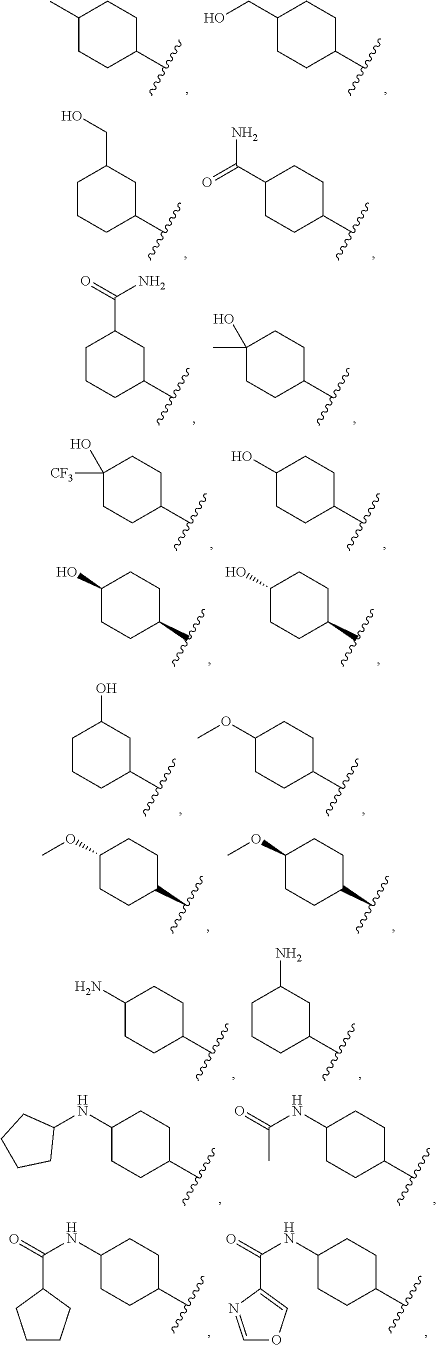

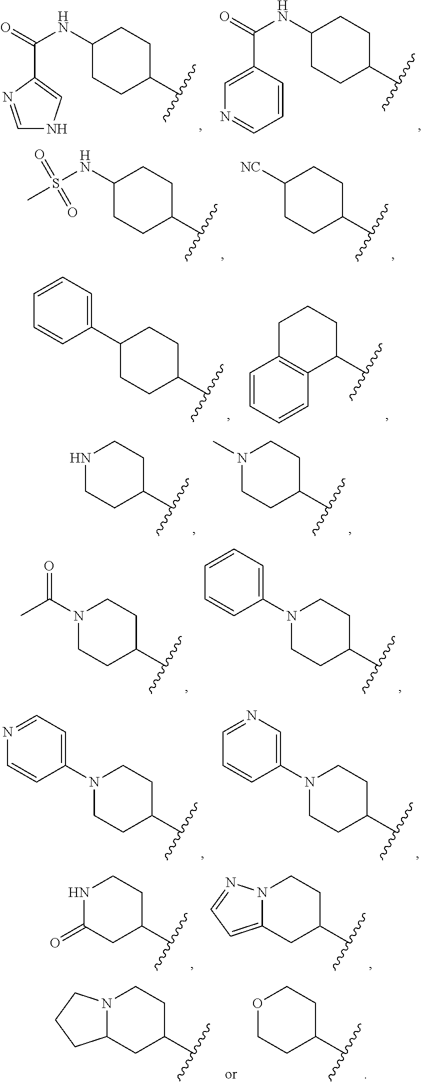

In preferred embodiments, Y is not cyclohexyl or 4-aminocyclohexyl.

In some embodiments, Y has formula (f), (g) or (h). Accordingly, Y may be

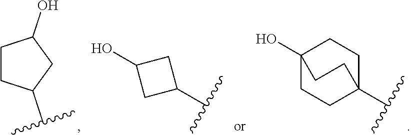 [SMILES: *C1CCC(O)C1 ; *C1CC(O)C1 ; *C12CCC(O)(CC1)CC2]

Z may be a substituted phenyl or a substituted pyridinyl group.

In one preferred arrangement, adjacent substituents of the phenyl or 5 or 6 membered heteroaryl group Z are linked to form, with the C atoms of the phenyl or heteroaryl group they are bound to, a 5- or 6-membered heterocyclic or heteroaromatic group. It will be appreciated that in these embodiments the group Z is an optionally substituted fused group. A preferred fused group Z is optionally substituted tetrahydroquinoline or optionally substituted tetrahydroisoquinoline. Optional substituents include, without limitation, C₁₋₆ alkyl or F. More preferably, optional substituents are CH₃ or F.

In another preferred arrangement, the phenyl or 5 or 6 membered heteroaryl group Z is substituted with an optionally substituted 3 to 10 membered heterocyclyl or an optionally substituted 5 to 10 membered heteroaryl, and the phenyl or phenyl or 5 or 6 membered heteroaryl group may be further optionally substituted with one or more substituents selected from the group consisting of a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹ and NR⁸R⁹ each of which is optionally substituted with one or more substituents. In some embodiments, the phenyl or 5 or 6 membered heteroaryl group Z is substituted with an optionally substituted 5 to 10 membered heterocyclyl or an optionally substituted 5 to 10 membered heteroaryl, and the phenyl or phenyl or 5 or 6 membered heteroaryl group may be further optionally substituted with a halogen. In some embodiments, the phenyl or 5 or 6 membered heteroaryl group Z is substituted with an optionally substituted 5 to 7 membered heterocyclyl or an optionally substituted 5 or 6 membered heteroaryl, and the phenyl or phenyl or 5 or 6 membered heteroaryl group may be further optionally substituted with a halogen. The halogen may be fluorine. The heterocyclyl or heteroaryl which is a substituent on the Z group may be unsubstituted or substituted with one or more optional substituents, which may be selected from the group consisting of optionally substituted C₁-C₆ alkyl, optionally substituted C₂-C₆ alkenyl, optionally substituted C₂-C₆ alkynyl, optionally substituted C₁-C₆ alkoxy, optionally substituted C₃₋₆ cycloalkyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷ or NR¹⁶R¹⁷. More preferably, the heterocyclyl or heteroaryl which is a substituent on the Z group may be unsubstituted or substituted with one or more substituents which may be selected from the group consisting of optionally substituted C₁-C₃ alkyl, optionally substituted C₂-C₆ alkenyl, optionally substituted C₂-C₆ alkynyl, optionally substituted C₃₋₆ cycloalkyl, halogen, oxo, COR¹⁶, COOR¹⁶, CONR¹⁶R¹⁷, NR¹⁶COR¹⁷ or NR¹⁶R¹⁷. The alkyl may be substituted with OR¹⁶ and NR¹⁶R¹⁷. R¹⁶ and R¹⁷ may be as defined above. In some embodiments, R¹⁶ and R¹⁷ may each independently be selected from the group consisting of H, C₁-C₃ alkyl or optionally halogenated 5 or 6 membered heterocycle.

A preferred group Z has formula:

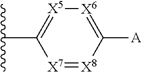

wherein X⁵, X⁶, X⁷ and X⁸ are each independently selected from N and CR¹¹ with the proviso that no more than one of X⁵, X⁶, X⁷ and X⁸ is N; R¹¹ in each occurrence is independently H or a halogen, preferably F; and A is selected from an optionally substituted C₁-C₆ alkyl, COR⁸, COOR⁸, CONR⁸R⁹, NR⁸COR⁹, NR⁸R⁹, an optionally substituted 3 to 10 membered heterocyclyl and an optionally substituted 5 to 10 membered heteroaryl. R⁸ and R⁹ may independently be hydrogen, an optionally substituted C₁-C₃ alkyl, an optionally substituted C₂-C₃ alkenyl, an optionally substituted C₂-C₃ alkynyl, optionally substituted phenyl, optionally substituted C₃-C₆ cycloalkyl, optionally substituted 3 to 8 membered heterocyclyl, or optionally substituted 5 to 10 membered heteroaryl. More preferably, R⁸ and R⁹ are independently hydrogen, an optionally substituted C₁-C₃ alkyl or optionally substituted 5 or 6 membered heterocyclyl. The alkyl may be unsubstituted or substituted with one or more of halogen, OR¹⁶ or NR¹⁶R¹⁷. R¹⁶ and R¹⁷ may be as defined above. In some embodiments, R¹⁶ and R¹⁷ may independently be H or C₁-C₃ alkyl.

In a preferred embodiment, the heterocyclyl or heteroaryl which is a substituent on the Z group, which may optionally be A in the above formula, is an optionally substituted 5 or 6 membered heterocyclyl or an optionally substituted 5 or 6 membered heteroaryl. In a preferred embodiment, the heterocyclyl or heteroaryl which is a substituent on the Z group, which may optionally be A in the above formula, is an optionally substituted 6 membered heterocyclyl or an optionally substituted 6 membered heteroaryl.

The heterocyclyl or heteroaryl group which is a substituent on the Z group, which may optionally be A in the above formula, can be unsubstituted or substituted with one or more of optionally substituted C₁-C₆ alkyl, optionally substituted C₂-C₆ alkenyl, optionally substituted C₂-C₆ alkynyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl, or pairs of non-adjacent substituents of the 3 to 8 membered heterocyclyl may combine to form a bridging group. More preferably, the optionally substituted heterocyclyl or an optionally substituted heteroaryl which is a substituent on the Z group, which may optionally be A in the above formula, can be unsubstituted or substituted with one or more of optionally substituted C₁-C₃ alkyl, halogen, oxo, COR¹⁶, COOR¹⁶, CONR¹⁶R¹⁷, NR¹⁶COR¹⁷ or NR¹⁶R¹⁷, or pairs of non-adjacent substituents of the 3 to 8 membered heterocyclyl may combine to form a bridging group. Even more preferably, the heterocyclyl or heteroaryl group which is a substituent on the Z group, which may optionally be A in the above formula, can be unsubstituted or substituted with one or more of optionally substituted C₁-C₃ alkyl, fluorine, COR¹⁶ or CONR¹⁶R¹⁷, or pairs of non-adjacent substituents of the 3 to 8 membered heterocyclyl may combine to form a bridging group. The alkyl may be unsubstituted or substituted with one or more of halogen, OR¹⁶ or NR¹⁶R¹⁷. R¹⁶ and R¹⁷ may be as defined above. In some embodiments, R¹⁶ and R¹⁷ may each independently be selected from the group consisting of H, optionally substituted C₁-C₃ alkyl, optionally substituted C₃₋₆ cycloalkyl, optionally substituted 3 to 6 membered heterocycle or optionally substituted 5 to 10 membered heteroaryl. Preferably, R¹⁶ and R¹⁷ are each independently selected from the group consisting of H, methyl and optionally halogenated 5 or 6 membered heterocycle. Preferably, the bridging group is optionally substituted methylene or ethylene.

A preferred heteroaryl group which is a substituent on the Z group, which may optionally be A in the above formula, is pyridyl, pyrazolyl or oxazolyl.

A preferred heterocyclyl group which is a substituent on the Z group, which may optionally be A in the above formula, is a group of formula (i) or (j):

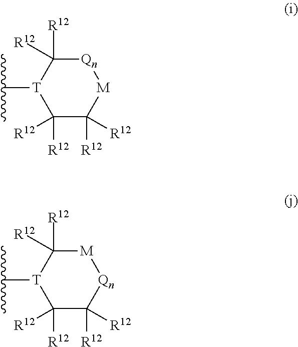

wherein:
T is N and M is NR¹³, CR¹⁴R¹⁵, O, S or SO₂; or T is CR¹⁸ and M is NR¹³, O, S or SO₂;
Q is C(R¹²)₂ and n is 1 or 2;
R¹² in each occurrence is independently H, halogen, optionally substituted C₁₋₆ alkyl, optionally substituted C₂₋₆ alkenyl, optionally substituted C₂₋₆ alkynyl, optionally substituted C₃₋₆ cycloalkyl, OR¹⁹; or NR¹⁹R²⁰; and/or two R¹² groups bonded to adjacent carbon atoms may be linked to form a fused group A or two R¹² groups bonded to non-adjacent carbon atoms may be linked to form a bicyclic bridged group A;
R¹³ is H, optionally substituted C₁-C₆ alkyl, optionally substituted C₂₋₆ alkenyl, optionally substituted C₂₋₆ alkynyl, optionally substituted C₃₋₆ cycloalkyl, optionally substituted phenyl, optionally substituted 5 or 6 membered heteroaryl, COR¹⁹ or CONR¹⁹R²⁰;
R¹⁴ and R¹⁵ are each independently selected from hydrogen, halogen, an optionally substituted C₁-C₆ alkyl, optionally substituted C₂₋₆ alkenyl, optionally substituted C₂₋₆ alkynyl, optionally substituted C₃-C₆ cycloalkyl, OR¹⁹ and NR¹⁹R²⁰, or R¹⁴ and R¹⁵ together with the C atom they are both bonded may be linked to form an optionally substituted 3 to 6 membered hetercyclyl or an optionally substituted C₃₋₆ cycloalkyl;
R¹⁸ is hydrogen or optionally substituted C₁-C₆ alkyl; and
R¹⁹ and R²⁰ are each independently H, optionally substituted C₁₋₆ alkyl, optionally substituted C₂₋₆ alkenyl, optionally substituted C₂₋₆ alkynyl, optionally substituted C₃₋₆ cycloalkyl group, optionally substituted 5 or 6 membered heteroaryl or optionally substituted 3 to 6 membered heterocyclyl.

In some embodiments, n is 1.

In some embodiments, T is N and M is NR¹³. In some embodiments, T is CR¹⁸ and M is NR¹³.

R¹³ may be H, optionally substituted C₁-C₆ alkyl, C₃₋₆ cycloalkyl, COR¹⁹ or CONR¹⁹R²⁰, R¹⁹ and R²⁰ may each independently be H, optionally substituted C₁₋₃ alkyl or optionally substituted 5 or 6 membered heterocyclyl. More preferably, R¹⁹ and R²⁰ are each independently H, optionally substituted C₁₋₃ alkyl or optionally halogenated 5 or 6 membered heterocyclyl. The alkyl may be unsubstituted or substituted with one or more of halogen, oxo, CN, OR¹⁶ or NR¹⁶R¹⁷. R¹⁶ and R¹⁷ may be as defined above. Preferably, R¹⁶ and R¹⁷ are H or CH₃. R¹³ may be CH₃, CH₂CH₃,

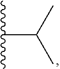 [SMILES: *C(C)C]

CH₂CH₂OH, CH₂CH₂OCH₃,

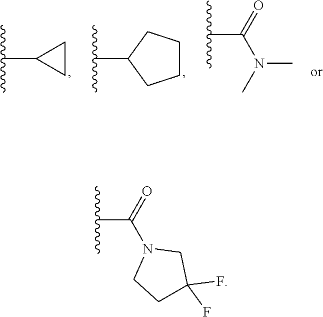

In some embodiments, T is N and M is CR¹⁴R¹⁵
R¹⁴ and R¹⁵ may each independently be selected from hydrogen, halogen, an optionally substituted C₁-C₃ alkyl, optionally substituted C₃-C₆ cycloalkyl, OR¹⁹-and-NR¹⁹R²⁰, or R¹⁴ and R¹⁵ together with the C atom they are both bonded may be linked to form an optionally substituted 3 to 6 membered heterocyclyl. More preferably, R¹⁴ and R¹⁵ are each independently selected from hydrogen, halogen, an optionally substituted C₁-C₃ alkyl and NR¹⁹R²⁰, or R¹⁴ and R¹⁵ together with the C atom they are both bonded may be linked to form an optionally substituted 3 to 6 membered hetercyclyl. R¹⁹ and R²⁰ may each independently be H or optionally substituted C₁₋₃ alkyl. Preferably, R¹⁹ and R²⁰ are each H. R¹⁴ and R¹⁵ may each independently be H, F, NH₂ or R¹⁴ and R¹⁵ together with the C atom they are both bonded may be linked to form a 5 membered hetercyclyl.

In some embodiments, T is N and M or O or SO₂.

Preferably, R¹⁸ is hydrogen.

Preferably, R¹² in each occurrence is independently H, halogen or optionally substituted C₁₋₃ alkyl; and/or two R¹² groups bonded to non-adjacent carbon atoms are linked to form a bicyclic bridged group. More preferably, R¹² in each occurrence is H; and/or two R¹² groups bonded to non-adjacent carbon atoms are linked to form a bicyclic bridged group.

Exemplary bridged groups have formulae:

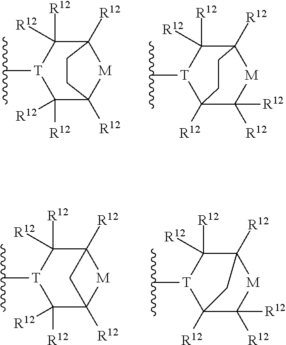

The group A or the heterocyclyl or heteroaryl group may be the only substituent of the phenyl or pyridyl group Z, or one or more further substituents may be present. Where present, the one or more further substituents may be selected from halogen; Ak; —OH;—OAk; —NH₂; —NHAk; NAK₂; optionally substituted heteroaryl; and optionally substituted heterocyclyl, wherein Ak in each occurrence is independently a C₁₋₆ alkyl group or a C₃₋₆ cycloalkyl group. The one or more further substituents are preferably halogen, more preferably F.

In some embodiments, the phenyl or 5 or 6 membered heteroaryl group Z is further substituted with a halogen, preferably fluorine.
Z may be

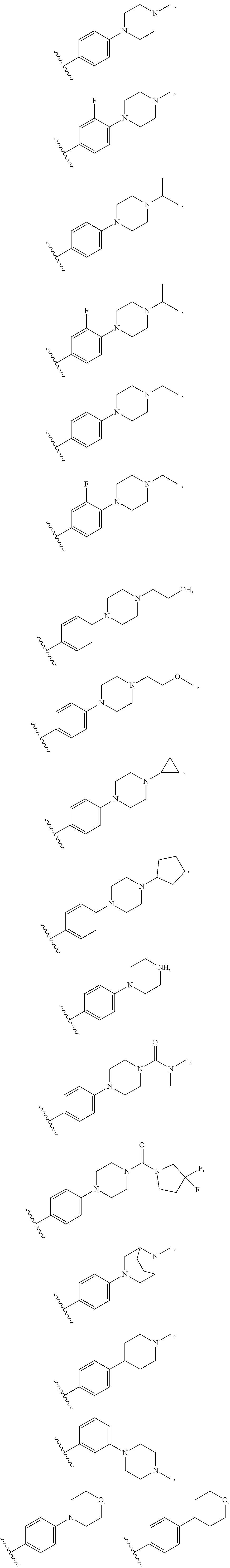

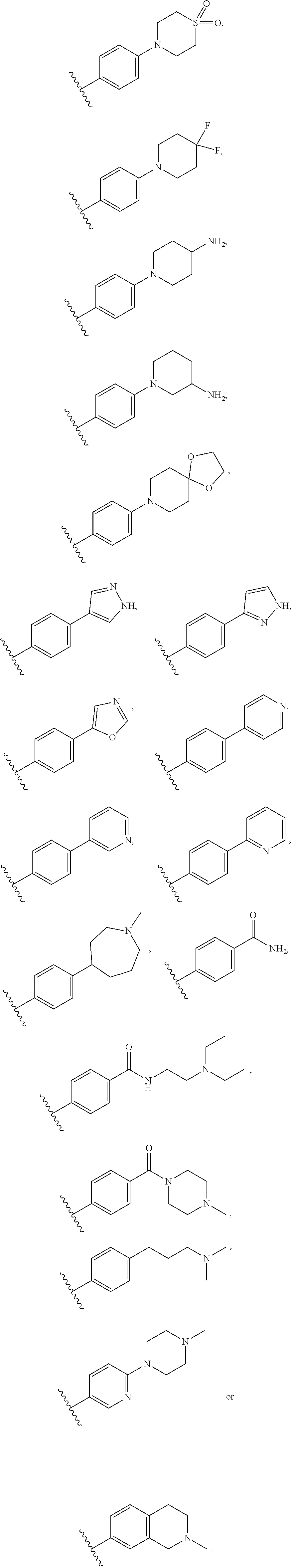

All features described herein (including any accompanying claims, abstract and drawings), and/or all of the steps of any method or process so disclosed, may be combined with any of the above aspects in any combination, except combinations where at least some of such features and/or steps are mutually exclusive.

### General Schemes

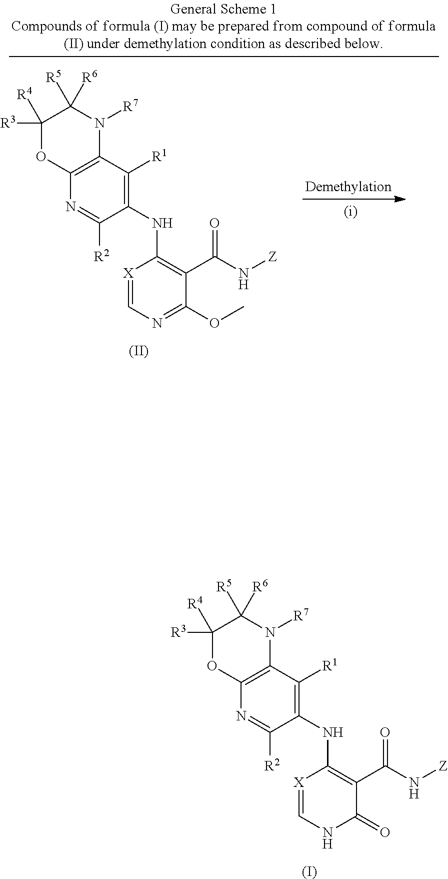

The demethylation reaction is typically performed in an acidic reaction mixture using, for example, aqueous H₂SO₄ or HCl with or without a co-solvent such as dioxane, ethers or alcohols and typically at elevated temperature. Alternatively, a nucleophilic bromine source can also be used as a reagent for the demethylation, for example by using BBr₃, LiBr/pTSA or aqueous HBr with or without a co-solvent such as dioxane, DMF or ethers under heating at 60-100° C. The compounds of formula (II) may be synthesized by those skilled in the art according to the methods described below.

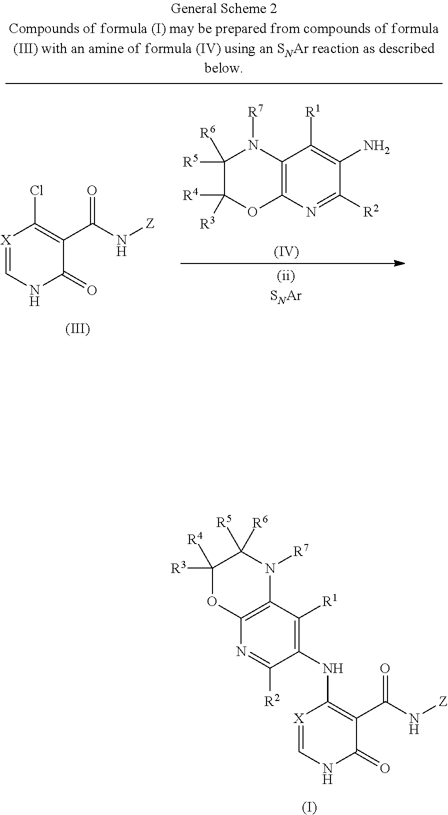

An amine of formula (IV) displaces a halogen atom, for example a chlorine atom from the pyridone intermediate (III). The SNAr reaction may employ any of the reaction conditions known in the art, which typically use an excess of neat amines at elevated temperatures.

Alternatively, and in particular for volatile or precious amines, the amine is dissolved in a suitable solvent such as EtOH, ⁱPrOH, ⁿBuOH or ^{t}BuOH along with a separate base, typically Et₃N, DIPEA, or NMM and the mixtures heated at 80-120° C. for up to 24 h. In cases where either component may be of limited solubility, a phase transfer catalyst such as TBAI in a suitable solvent such as toluene or xylene with a suitable base such as Et₃N, DIPEA, or NMM may also be employed. Amines of formula (IV) are either commercially available or may be synthesized by those skilled in the art.

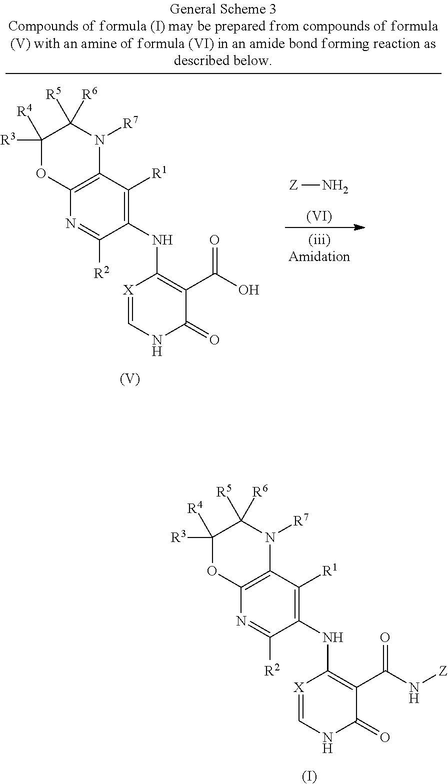

Typical conditions employ activation of the carboxylic acid of the compound of formula (V) using a suitable organic base and a suitable coupling agent. Preferred coupling agents are either EDCI with HOBt, T3P, HATU, HBTU or BOP. Preferred organic bases comprise either DIPEA or TEA in a suitable organic solvent such as DCM, DMF, DMA, THE, MeOH or MeCN. The reaction may be shaken or stirred at room temperature, typically for up to 24 h. Compounds of formula (VI) are commercially available or may be synthesized by those skilled in the art.

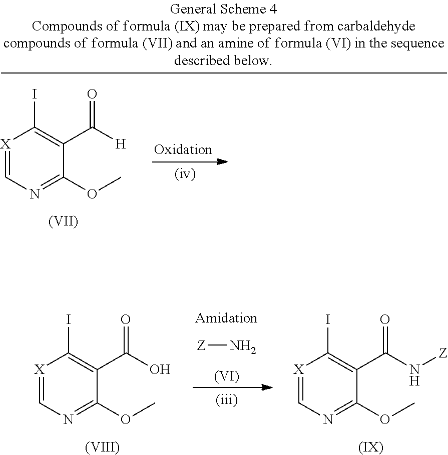

The oxidation of the aldehyde (VII) to the corresponding acid (VIII) is typically carried out with a strong oxidant such as KMnO₄, sodium perborate or methyl trioxorhenium, in a suitable solvent such as pyridine, water, acetic acid or methanol typically at room temperature for between 1 and 24 h. It will be appreciated that aldehydes of formula (VII) may also be converted into acids of formula (VIII) via corresponding ester intermediates using reactions which may be known to those skilled in the art such as with Br₂ and an alcoholic solvent. The acid (VIII) may then be converted into amides of formula (IX) using similar methods to those described in General Scheme 3. Thus, preferred amide coupling agents are either EDCI with HOBt, T3P, HATU, HBTU or BOP. Preferred organic bases comprise either DIPEA or TEA in a suitable organic solvent such as DCM, DMF, DMA, THF, MeOH or MeCN. Compound of formula (IX) can also be prepared from corresponding acid via acid chloride route using chlorinating reagents such as SOCl₂, SO₂C₁₂, POCl₃ or POCl₅. The reaction may be shaken or stirred at room temperature, typically for up to 24 h. Compounds of formula (VI) are commercially available or may be synthesized by those skilled in the art.

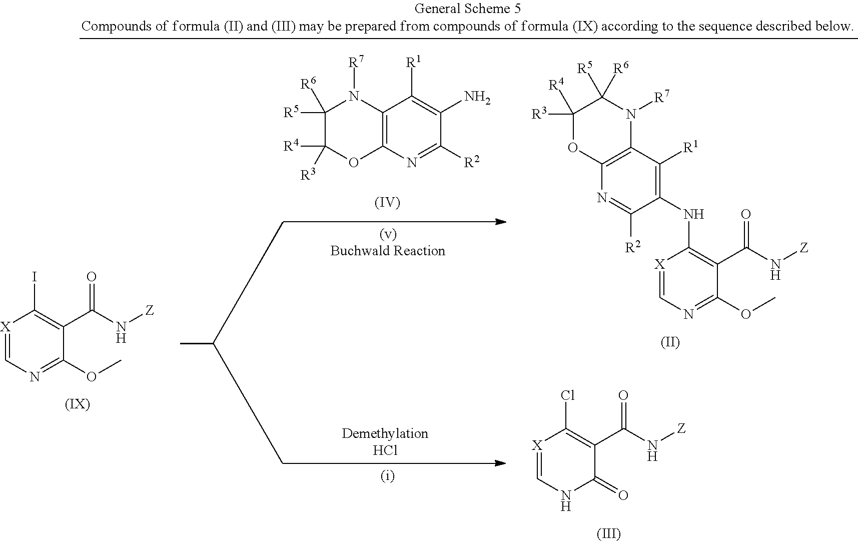

Compounds of formula (IX) may undergo a Buchwald amination reaction with suitable amines of formula (IV) to provide the amines of formula (II). The Buchwald reactions are carried out with a suitable base such as DIPEA, TEA, NaOtBu, Cs₂CO₃, Na₂CO₃ or NaH and a suitable solvent such as n-BuOH, t-BuOH, 1,4-dioxane or EtOH at elevated temperatures, typically 60-110° C. Suitable transition metal catalysts for the reaction include Pd₂(dba)₃, Pd(dppf)Cl₂, Pd(OAc)₂ or Pd(dba)Cl₂ with suitable ligands such as dppf, BINAP, Xantphos or S-Phos and the reactions are typically carried out for 12-24 h.

The compounds of formula (IX) may directly undergo a demethylation reaction using conditions similar to those described in General Scheme 1. Thus, demethylation of (IX) is typically performed in an acidic reaction mixture using, for example, aqueous H₂SO₄ or HCl with or without a co-solvent such as 1,4-dioxane, ethers or alcohols and typically at elevated temperature. Alternatively, a nucleophilic bromine source can also be used as a reagent for the demethylation, for example by using BBr₃, LiBr/pTSA or aqueous HBr with or without a co-solvent such as 1,4-dioxane, DMF or ethers under heating at 60-100° C. to give the pyridones of formula (III).

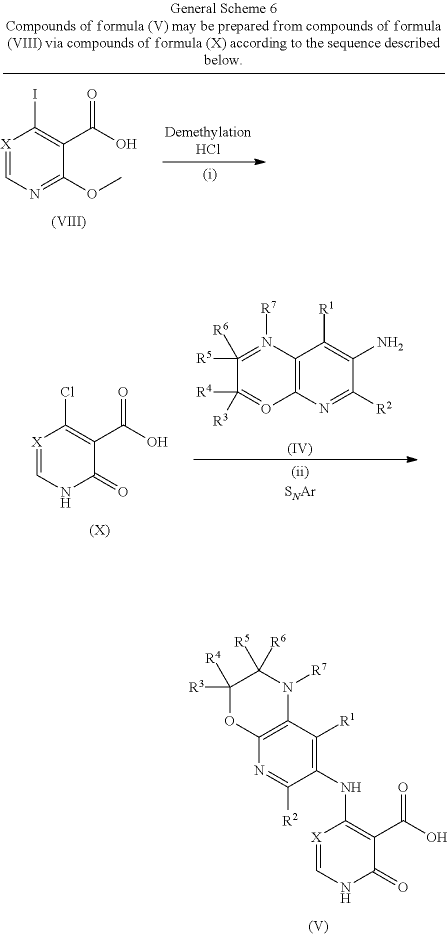

Compounds of formula (VIII) may be converted into the corresponding pyridones of formula (X) in a demethylation reaction similar to that described in General Scheme 1. Thus, the reaction is typically performed with aqueous HCl with or without a co-solvent such as 1,4-dioxane or ethers and typically at 60-100° C. for between 6 and 24 h. During the demethylation reaction, it is typically observed that the iodide group of (VIII) is replaced by a chloride to give the product compounds of formula (X). The acids of formula (X) may then undergo an SNAr reaction with an amine of formula (IV) in conditions similar to those described in General Scheme 2. Thus, the amine is dissolved in a suitable solvent such as EtOH, ⁱPrOH, ⁿBuOH or ^{t}BuOH along with a separate base, typically Et₃N, DIPEA, or NMM and the mixtures heated at 80-120° C. for up to 24 h to give the amine products of formula (V). In cases where either component may be of limited solubility, a phase transfer catalyst such as TBAI in a suitable solvent such as toluene or xylene with a suitable base such as Et₃N, DIPEA, or NMM may also be employed. Amines of formula (IV) are either commercially available or may be synthesized by those skilled in the art.

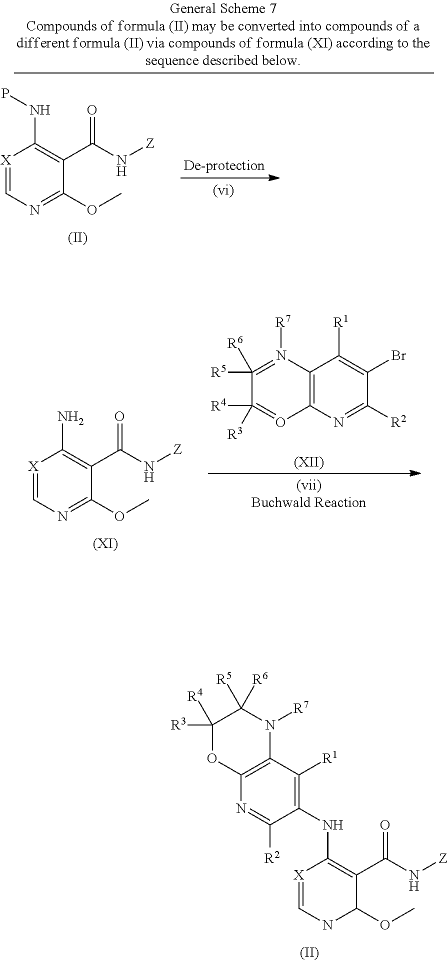

This sequence typically applies to compounds of formula (II) in which Y is a protecting group which will be known as such to those skilled in the art. For example, if the protecting group is para-methoxybenzyl or dimethoxybenzyl, the group Y may be removed with an acidic reagent such as TFA, triflic acid or HCl in a suitable solvent such as DCM, dioxane, DCE or toluene with or without heating at temperatures of between 2° and 80° C. for between 1 and 48 h to give the primary amines of formula (XI). These amines may then be used in a Buchwald amination reaction with a suitable halide of formula (XII) to give the secondary amines of formula (II) using conditions similar to those described in General Scheme 5. Thus, the reactions are carried out with a suitable base such as DIPEA, TEA, NaOtBu, Cs₂CO₃, Na₂CO₃ or NaH and a suitable solvent such as n-BuOH, t-BuOH, 1,4-dioxane, toluene or EtOH at elevated temperatures, typically 60-110° C. Suitable transition metal catalysts for the reaction include Pd₂(dba)₃, Pd(dppf)Cl₂, Pd(OAc)₂ or Pd(dba)Cl₂ with suitable ligands such as dppf, BINAP, Xantphos or S-Phos and the reactions are typically carried out for 12-24 h. Preferred reagents include the BrettPhos-Pd-G3 catalyst system. Halides of formula (XII) are either commercially available or may be synthesized by those skilled in the art.

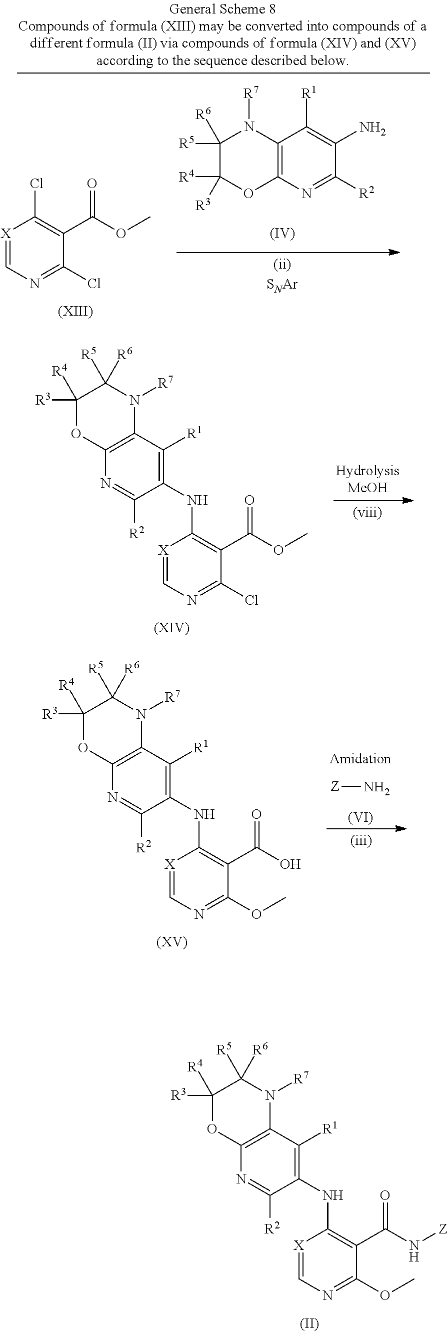

Compounds of formula (XIII) may undergo a Buchwald amination reaction with a suitable amine of formula (IV) to give the amines of formula (XIV) using conditions similar to those described in General Scheme 5. Thus, the reactions are carried out with a suitable base such as DIPEA, TEA, NaOtBu, Cs₂CO₃, Na₂CO₃ or NaH and a suitable solvent such as n-BuOH, t-BuOH, 1,4-dioxane, toluene or EtOH at elevated temperatures, typically 60-110° C. Suitable transition metal catalysts for the reaction include Pd₂(dba)₃, Pd(dppf)Cl₂, Pd(OAc)₂ or Pd (dba) C₁₂ with suitable ligands such as dppf, BINAP, Xantphos or S-Phos and the reactions are typically carried out for 12-24 h. Amines of formula (IV) are either commercially available or may be synthesized by those skilled in the art. The resulting compounds of formula (XIV) may then undergo an ester hydrolysis reaction which typically uses a suitable alkali or base to hydrolyse the ester and provide the acids of formula (XV). The suitable alkali or base may be LiOH, KOH, NaOH or K2CO₃, and the reaction is typically conducted in an aqueous solution or in mixtures of solvents such as water, THF, MeOH or EtOH at room temperature for between 1 and 48 h. The resulting acids of formula (XV) may then undergo an amide bond forming reaction with a suitable amine of formula (VI) using conditions similar to those described in General Scheme 3. Typical conditions employ activation of the carboxylic acid using a suitable organic base and a suitable coupling agent. Preferred coupling agents are either EDCI with HOBt, T3P, HATU, HBTU or BOP. Preferred organic bases comprise either DIPEA or TEA in a suitable organic solvent such as DCM, DMF, DMA, THF, MeOH or MeCN. The reaction may be shaken or stirred at room temperature, typically for up to 24 h. Compounds of formula (VI) are commercially available or may be synthesized by those skilled in the art to give the products of formula (II).

### General Synthetic Procedures

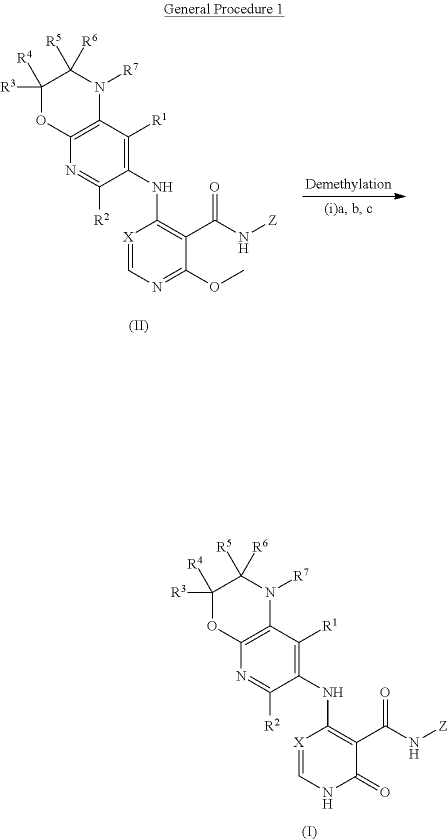

### Method a:

A compound of formula (II) (1.0 eq.) was taken in 4M HCl in 1,4-dioxane (13 mL/mmol) at 0-5° C. and the resulting reaction mixture was refluxed for 2-6 h. Progress of the reaction was monitored by TLC or/and LCMS. After completion the solvent was evaporated under reduced pressure to give crude product which was purified by trituration with ether-pentane mixtures or column chromatography to afford the compounds of formula (I) (yield 7-80%) as solids.

### Method b:

To a stirred solution of a compound of formula (II) (1.0 eq.) in DMF (9 mL/mmol) was added PTSA.H₂O (5.0 eq.) followed by LiBr (5.0 eq.) at RT and the resulting reaction mixture was stirred at 100-120° C. for 10-15 mins. Completion of the reaction was confirmed by UPLC-MS or/and TLC. Thereafter, the reaction mass was diluted with chilled water and extracted with 5-15% methanol in DCM. The combined organic layers were washed with brine, dried over anhydrous sodium sulphate, filtered and concentrated in vacuo to give the crude product which was purified by column chromatography or prep-HPLC to afford the compounds of formula (I) (yield 7-80%) as solids.

### Method c:

To a stirred solution of a compound of formula (II) (1.0 eq. 0.295 mmol) in 1,4-dioxane (6 mL/mmol) was added HBr (40%, 6 mL/mmol) dropwise at 0-5° C. The whole was stirred at 75-80° C. overnight. Progress of the reaction was checked by LCMS and after completion the solvent and excess HBr were evaporated in vacuo by azeotropic distillation using acetonitrile and the remaining residue was washed with diethyl ether to give the crude product which was purified by column chromatography to afford the compounds of formula (I) (yield 10-65%) as solids.

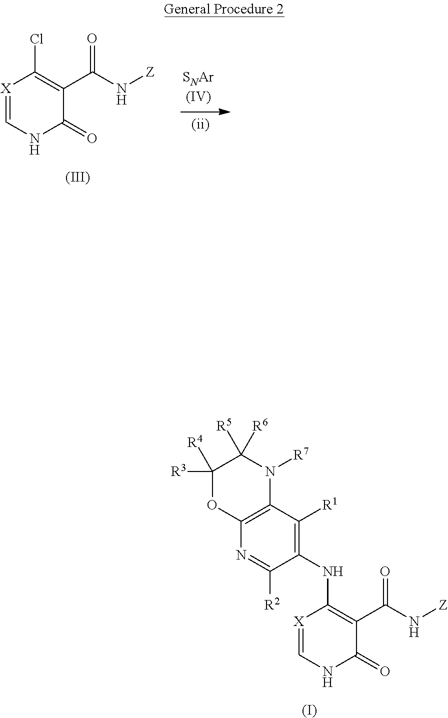

To a stirred solution of a compound of formula (III) (1.0 eq.) in n-butanol or t-BuOH or toluene (11 mL/mmol) was added N,N-diisopropylethylamine or TEA (15.0 eq.) and the mixture was stirred at room temperature for 10-20 mins. Thereafter, a compound of formula (IV) (1.5 eq.) was added to the reaction vessel. The resulting reaction mixture was stirred at 100-120° C. for 10-16 h. Progress of the reaction was monitored by LCMS/TLC and after completion of the reaction the solvent was evaporated under reduced pressure to give a crude mass which was purified by column chromatography or preparative reverse phase HPLC to afford the compounds of formula (I) (yield 10-65%) as solids.

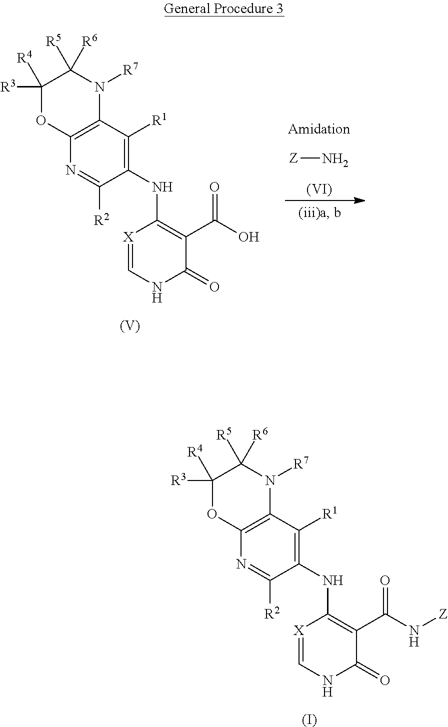

### Method a:

To a stirred solution of a compound of formula (V) (1.0 eq.) in THF (3.5 mL/mmol) and a few drops of DMF were added HATU (1.5 eq.) and TEA (3.0 eq.1) at room temperature and the resulting reaction mixture was allowed to stir at RT for 10-15 mins. Then a compound of formula (VI) Z—NH₂ (1.2 eq.) was added into the reaction vessel and the mixture was stirred at room temperature for 2-5 h. Progress of the reaction was monitored by TLC and LCMS which confirmed formation of the desired product. Thereafter, the reaction mixture was evaporated in vacuo to give a residue which was diluted with 5-10% MeOH in DCM and washed with water and brine repeatedly. The organic portion was dried over anhydrous sodium sulphate and concentrated under vacuum to give the crude product which was purified by Combi-flash column chromatography using 5-10% MeOH in DCM as eluent to afford the compounds of formula (I) (yield 10-80%) as solids.

### Method b:

To a stirred solution of a compound of formula (V) (1.0 eq.) in toluene (6.5 mL/mmol) was added POCl₃ (0.30 mL/mmol) and the combined mixture was refluxed for 2-4 h to give the corresponding acid chloride intermediate. Thereafter, the solvents were evaporated under vacuum and the crude acid chloride was taken up in DCM (6.5 mL/mmol) and triethylamine (5.0 eq.) was added followed by a compound of formula (VI) Z—NH₂ (1.5 eq.) at 0-5° C. The resulting reaction mixture was stirred at room temperature for 10-16 h. Progress of the reaction was monitored by LCMS or/and TLC and after completion the solvents were evaporated under vacuum to give the crude product which was purified by column chromatography or prep-HPLC to afford the compounds of formula (I) (yield 5-65%) as solids.

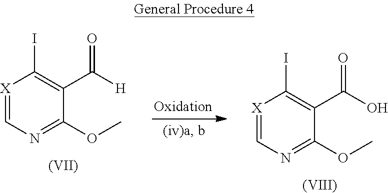

### Method a:

To a stirred solution of a compound of formula (VII) (1.0 eq.) in a mixture of tert-butanol (5.2 mL/mmol) and water (2.6 mL/mmol) at 0-5° C. was added 2-methyl-2-butene (3.0 eq.) followed by sodium dihydrogen phosphate (2.5 eq.) and sodium chlorite (2.0 eq.) and the whole mixture was stirred at 0-5° C. for 1-2 h. Progress of the reaction was monitored by LCMS and after completion the reaction mass was quenched with IN formic acid solution. The product was extracted with ethyl acetate. The combined organic layers were washed with brine, dried over anhydrous sodium sulphate and concentrated under vacuum to afford the compounds of formula (VIII) (yield 70-80%) as solids.

### Method b:

To a stirred solution of a compound of formula (VII) (1.0 eq.) in aqueous pyridine (50%, 5.2 mL/mmol) was added KMnO₄ (1.0 eq.) portionwise at 0-5° C. The resulting reaction mixture was allowed to stir at RT for 1-2 h. Progress of the reaction was monitored by TLC/LCMS and after completion the reaction mixture was filtered and washed with acetonitrile and water. The filtrate was distilled under reduced pressure using excess acetonitrile to give the crude product which was purified by trituration with diethyl ether to afford the compounds of formula (VIII) (yield 65-85%) as their potassium salts.

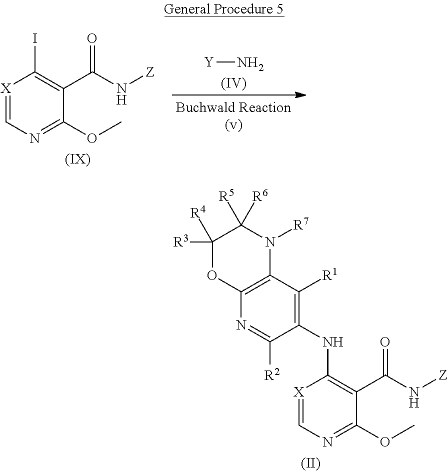

To a stirred solution of a compound of formula (IX) (1.0 eq.) in 1,4-dioxane (15 mL/mmol) in a sealed tube was added a compound of formula (IV) Y—NH₂ (1.2 eq.) and Cs₂CO₃ (3.0 eq.). The combined reaction mixture was degassed for 10-20 mins. with a nitrogen balloon. Then, Pd₂ (dba)₃ (0.1 eq.) and Xantphos (0.2 eq.) were added into the reaction vessel and the resulting reaction mixture was stirred at 100° C. overnight. Progress of the reaction was monitored by LCMS/TLC and after completion the reaction mixture was diluted with water and extracted with 10% methanol in DCM. The combined organic layers were washed with brine, dried over anhydrous sodium sulphate, filtered and concentrated under vacuum to give the crude product which was purified by column chromatography on silica gel using 3-5% methanol in DCM as eluent to afford the compounds of formula (II) (yield 70-80%) as solids.

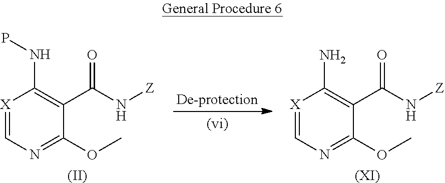

To a stirred solution of a compound of formula (II) (1.0 eq.) in DCE (8 mL/mmol) was added TFA (2 mL/mmol) dropwise at 0-5° C. and the combined mixture was allowed to stir at room temperature overnight. Progress of the reaction was monitored by LCMS/TLC and after completion the reaction mixture was evaporated to dryness to give a residue which was neutralized with TEA and extracted with 15% methanol in DCM. The combined organic layers were washed with brine, dried over anhydrous sodium sulphate, filtered and concentrated under vacuum to give the crude product which was purified by column chromatography on silica gel using 5-10% methanol in DCM as eluent to afford the compounds of formula (XI) (yield 70-75%) as solids.

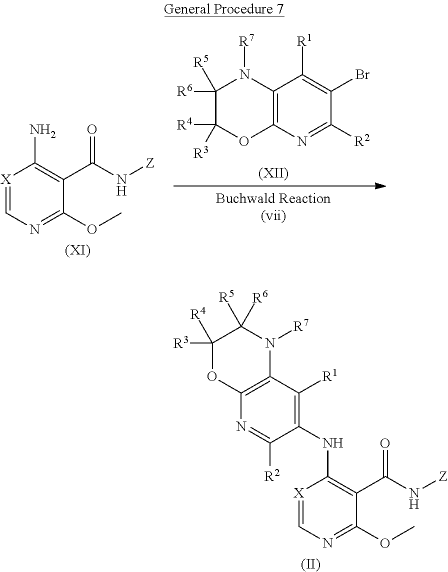

To a stirred solution of a compound of formula (XI) (1.0 eq.) in toluene (40 mL/mmol) was added a compound of formula (XII) (1.2 eq.) followed by NaO′Bu (2.5 eq.). The reaction mixture was purged for 10-20 mins. with a nitrogen balloon. Then, BretPhos-Pd-G3 (0.2 eq.) was added into the reaction vessel and the resulting reaction mixture was stirred at 100° C. overnight. Progress of the reaction was monitored by LCMS/TLC and after completion the reaction mixture was diluted with water and extracted with 10% methanol in DCM. The combined organic layers were washed with brine, dried over anhydrous sodium sulphate, filtered and concentrated under vacuum to give a crude compound which was purified by column chromatography on silica gel using 4-5% methanol in DCM as eluent to afford the compounds of formula (II) (yield 40-50%) as solids.

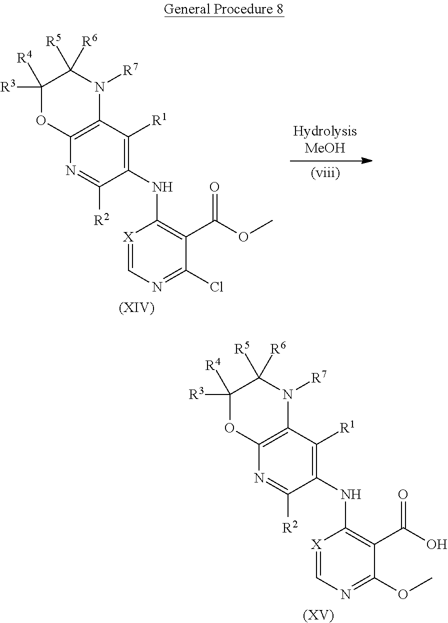

To a stirred solution of a compound of formula (XIV) (1.0 eq., 2.1 mmol) in THF (2 mL/mmol) was added a solution of LiOH.H₂O (4.0 eq.) in water (1 mL/mmol) followed by MeOH (2 mL/mmol) at room temperature and the whole allowed to stir at RT overnight. Progress of the reaction was monitored by UPLC-MS/TLC which showed formation of the desired product. Thereafter, the reaction mixture was concentrated in vacuo and slightly acidified with a solution of citric acid to give a white solid precipitate which was filtered and washed with water followed by hexane and dried under vacuum to afford the compounds of formula (XV) (yield 70-80%) as solids.

### General Purification and Analytical Methods

All final compounds were purified by either Combi-flash or prep-HPLC purification, and analysed for purity and product identity by UPLC or LCMS according to one of the below conditions.

### Prep-HPLC

Preparative HPLC was carried out on a Waters auto purification instrument using either a YMC Triart C18 column (250×20 mm, 5 μm) or a Phenyl Hexyl column (250×21.2 mm, 5 μm) operating at between ambient temperature and 50° C. with a flow rate of 16.0-50.0 mL/min.

Mobile phase 1: A=20 mM Ammonium Bicarbonate in water, B=Acetonitrile; Gradient Profile: Mobile phase initial composition of 80% A and 20% B, then to 60% A and 40% B after 3 min., then to 30% A and 70% B after 20 min., then to 5% A and 95% B after 21 min., held at this composition for 1 min. for column washing, then returned to initial composition for 3 min.

Mobile phase 2: A=10 mM Ammonium Acetate in water, B=Acetonitrile; Gradient Profile: Mobile phase initial composition of 90% A and 10% B, then to 70% A and 30% B after 2 min., then to 20% A and 80% B after 20 min., then to 5% A and 95% B after 21 min., held at this composition for 1 min. for column washing, then returned to initial composition for 3 min.

### LCMS Method

General 5 min method: Zorbax Extend C18 column (50×4.6 mm, 5 μm) operating at ambient temperature and a flow rate of 1.2 mL/min. Mobile phase: A=10 mM Ammonium Acetate in water, B=Acetonitrile; Gradient profile: from 90% A and 10% B to 70% A and 30 B in 1.5 min, and then to 10% A and 90% B in 3.0 min, held at this composition for 1.0 min, and finally back to initial composition for 2.0 min.

### UPLC Method

UPLC was carried out on a Waters auto purification instrument using a Zorbax Extend C18 column (50×4.6 mm, 5 μm) at ambient temperature and a flow rate of 1.5 ml/min. Mobile phase 1: A=5 mM Ammonium Acetate in water, B=5 mM Ammonium Acetate in 90:10 Acetonitrile/water; Gradient profile from 95% A and 5% B to 65% A and 35% B in 2 min., then to 10% A and 90% B in 3.0 min., held at this composition for 4.0 min. and finally back to the initial composition for 5.0 min.

Mobile phase 2: A=0.05% formic acid in water, B=Acetonitrile; Gradient profile from 98% A and 2% B over 1 min., then 90% A and 10% B for 1 min., then 2% A and 98% B for 2 min. and then back to the initial composition for 3 min.

### EXAMPLES

Nuclear magnetic resonance (NMR) spectra were in all cases consistent with the proposed structures. Characteristic chemical shifts (8) are given in parts-per-million downfield from tetramethylsilane (for 1H-NMR) and upfield from trichloro-fluoro-methane (for 19F NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d6-DMSO, deuterodimethylsulphoxide; and CD3OD, deuteromethanol.

Mass spectra, MS (m/z), were recorded using electrospray ionisation (ESI). Where relevant and unless otherwise stated the m/z data provided are for isotopes ¹⁹F, ³⁵Cl, ⁷⁹Br and ¹²⁷I.

All chemicals, reagents and solvents were purchased from commercial sources and used without further purification. All reactions were performed under an atmosphere of nitrogen unless otherwise noted.

Flash column chromatography was carried out using pre-packed silica gel cartridges in a Combi-Flash platform. Prep-HPLC purification was carried out according to the General purification and analytical methods described above. Thin layer chromatography (TLC) was carried out on Merck silica gel 60 plates (5729). All final compounds were >95% pure as judged by the LCMS or UPLC analysis methods described in the General purification and analytical methods above unless otherwise stated.

### Example 45:4-((8-Methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide

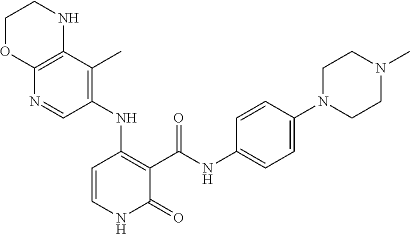 [SMILES: Cc1c(Nc2cc[nH]c(=O)c2C(=O)Nc2ccc(N3CCN(C)CC3)cc2)cnc2c1NCCO2]

Example 45 was prepared according to the methods described in General Procedures 1-7, and the methods described below.

### Preparation 1:4-Chloro-N-(4-(4-methylpiperazin-1-yl)phenyl)-2-oxo-1,2-dihydropyridine-HCl

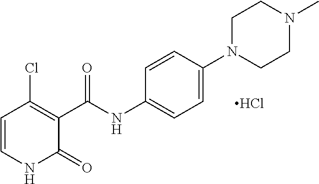 [SMILES: CN1CCN(c2ccc(NC(=O)c3c(Cl)cc[nH]c3=O)cc2)CC1 ; Cl]

### Step 1:4-Iodo-2-methoxynicotinic acid

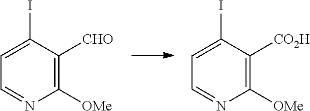 [SMILES: COc1nccc(I)c1C=O ; COc1nccc(I)c1C(=O)O]

To a stirred solution of commercially available 4-iodo-2-methoxynicotinaldehyde (5.0 g, 19.01 mmol) in a mixture of tert-butanol (100 mL) and water (50 mL) at 0-5° C. was added 2-methyl-2-butene (6.03 mL, 57.03 mmol) followed by sodium dihydrogen phosphate (6.55 g, 47.52 mmol) and sodium chlorite (3.43 g, 38.02 mmol) and the combined mixture was stirred at 0-5° C. for 1 h. Progress of the reaction was monitored by LCMS and after completion the reaction mass was quenched with IN formic acid solution and the product was extracted with ethyl acetate. The combined organic layers were washed with brine, dried over anhydrous sodium sulphate and concentrated under vacuum to give the titled compound (4.2 g, yield 79.17%) as an off-white solid. LCMS m/z: 280.0 [M+H].

### Step 2:4-Iodo-2-methoxy-N-(4-(4-methylpiperazin-1-yl)phenyl) nicotinamide

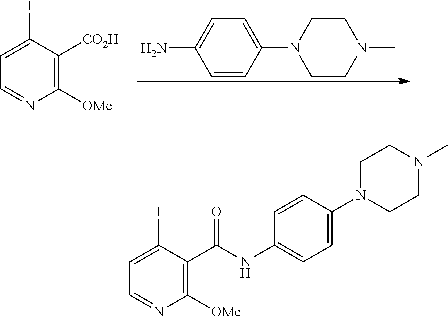 [SMILES: COc1nccc(I)c1C(=O)O ; CN1CCN(c2ccc(N)cc2)CC1 ; COc1nccc(I)c1C(=O)Nc1ccc(N2CCN(C)CC2)cc1]

To a stirred solution of 4-iodo-2-methoxynicotinic acid (Preparation-1, Step-1) (4.0 g, 14.33 mmol) in THF (50 mL) and a few drops of DMF were added HATU (8.17 g, 21.50 mmol) and TEA (5.90 mL, 43.01 mmol) at room temperature and the resulting reaction mixture was allowed to stir at RT for 10-15 mins. Then commercially available 4-(4-methylpiperazin-1-yl) aniline (3.29 g, 17.20 mmol) was added into the reaction vessel and the mixture was stirred at room temperature for 2-3 h. TLC and LCMS showed complete formation of the desired product. Thereafter, the reaction mixture was evaporated in vacuo to give a residue which was diluted with 5-10% MeOH/DCM and washed with water and brine repeatedly. The organic portion was dried over anhydrous sodium sulphate and concentrated under vacuum to give the crude product which was purified by Combi-flash column chromatography using 5-10% MeOH in DCM as eluent to afford the titled compound (5.0 g, yield 77.1%) as an off-white solid. LCMS m/z: 452.8 [M+H].

### Step 3:4-Chloro-N-(4-(4-methylpiperazin-1-yl)phenyl)-2-oxo-1,2-dihydropyridine-3-carboxamide.HCl

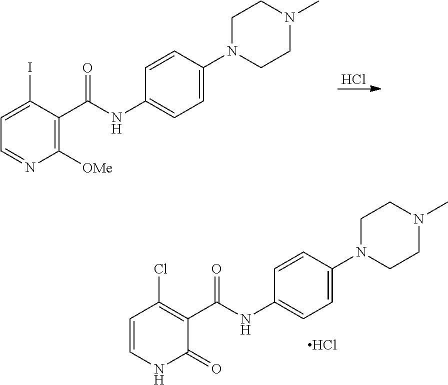 [SMILES: COc1nccc(I)c1C(=O)Nc1ccc(N2CCN(C)CC2)cc1 ; Cl ; CN1CCN(c2ccc(NC(=O)c3c(Cl)cc[nH]c3=O)cc2)CC1 ; Cl]

4-Iodo-2-methoxy-N-(4-(4-methylpiperazin-1-yl)phenyl) nicotinamide (Preparation-1, Step-2) (500 mg, 1.11 mmol) was taken up in 4M HCl in 1,4-dioxane (15 mL) at 0-5° C. and the reaction mixture was refluxed for 2 h. TLC and LCMS showed formation of the desired product and so the solvent was evaporated under reduced pressure to give the solid crude product which was purified by trituration with ether-pentane mixture and dried under vacuum to afford the titled compound (250 mg, yield 65.17%) as an off-white solid. LCMS m/z: 347.18 [M+H].

### Preparation 2:8-Methyl-2,3-dihydro-1H-pyrido[2,3-b][1,4]oxazin-7-amine

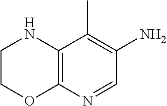

### Step 1: Ethyl 2-((5-bromo-4-methyl-3-nitropyridin-2-yl)oxy)acetate

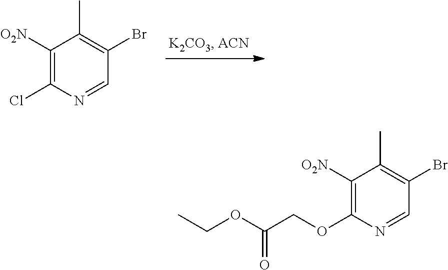 [SMILES: Cc1c(Br)cnc(Cl)c1[N+](=O)[O-] ; CCOC(=O)COc1ncc(Br)c(C)c1[N+](=O)[O-]]

To a stirred solution of commercially available 5-bromo-2-chloro-4-methyl-3-nitropyridine (25.0 g, 99.41 mmol) in acetonitrile (400 mL) was added oven dried potassium carbonate (41.21 g, 298.26 mmol) and ethyl glycolate (28.22 mL, 298.26 mmol). The resulting reaction mass was heated at 80° C. for 12 h under a nitrogen atmosphere. Progress of the reaction was monitored by LCMS/TLC and after completion of the reaction, it was cooled to RT, filtered through a sintered funnel and the filtrate was evaporated in vacuo to give the crude material which was purified via column chromatography using 5-7% ethyl acetate in hexane as eluent to afford the titled compound (15.0 g, yield 47.0%) as an off-white solid compound. LCMS m/z: 318.8 [M+H].

### Step 2:7-Bromo-8-methyl-1H-pyrido[2,3-b][1,4]oxazin-2 (3H)-one

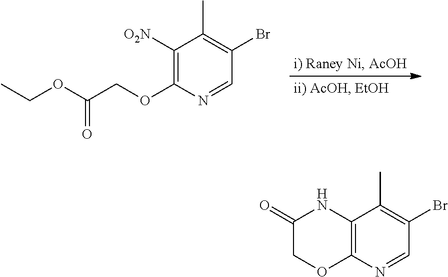 [SMILES: CCOC(=O)COc1ncc(Br)c(C)c1[N+](=O)[O-] ; Cc1c(Br)cnc2c1NC(=O)CO2]

A stirred solution of ethyl 2-((5-bromo-4-methyl-3-nitropyridin-2-yl)oxy)acetate (Preparation-2, Step-1) (25.0 g, 78.34 mmol) in methanol (300.0 mL) was charged to a clean dry par shaker vessel and purged with argon, then glacial acetic acid (20.0 mL) was added followed by Raney Ni (˜13.12 g). The whole mixture was again purged with argon for 10 mins. and hydrogenated at room temperature under 40 psi hydrogen for 12 h. TLC showed complete consumption of the starting material and formation of the desired product along with some uncyclized intermediate amine-ester. The reaction mixture was filtered through a short celite bed and after several washes of the celite bed, the obtained filtrate was concentrated in vacuo to give a crude material which was taken up in a round bottomed flask (500 mL) and ethanol (150 mL) was added to it followed by glacial acetic acid (30 mL). The resulting reaction mixture was heated at 80° C. for 12 h, the solvents were evaporated in vacuo to afford the crude product which was purified by washing with MTBE and pentane to afford the titled compound (13.3 g, yield 70%) as an off-white solid. LCMS m/z: 243.0 [M+H].

### Step 3:7-Bromo-8-methyl-2,3-dihydro-1H-pyrido[2,3-b][1,4]oxazine

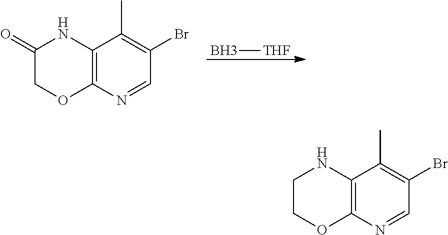

To a stirred solution of 7-bromo-8-methyl-1H-pyrido[2,3-b][1,4]oxazin-2 (3H)-one (Preparation-2, Step-2) (10.0 g, 41.32 mmol) in THF was added BH₃.THF solution (170.0 mL) dropwise at 0-5° C. under a nitrogen atmosphere. The mixture was then heated at 70° C. for 3 h. After completion (confirmed by TLC), the reaction mixture was cooled to room temperature and quenched by adding MeOH (200 mL) followed by conc. HCl. The mixture was concentrated under vacuum to give the crude material which was basified with aq. NaHCO₃ solution and extracted with EtOAc (3×100 mL). The combined organic layers were dried over anhydrous Na₂SO₄, filtered and evaporated in vacuo to afford the titled compound (7.5 g, yield 80%) as an off-white crude solid which was used as such in the next step without any further purification. LCMS m/z: 228.8 [M+H].

### Step 4: tert-Butyl 7-bromo-8-methyl-2,3-dihydro-1H-pyrido[2,3-b][1,4]oxazine-1-carboxylate

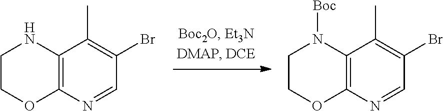 [SMILES: Cc1c(Br)cnc2c1NCCO2 ; Cc1c(Br)cnc2c1N(C(=O)OC(C)(C)C)CCO2]

To a stirred solution of 7-bromo-8-methyl-2,3-dihydro-1H-pyrido[2,3-b][1,4]oxazine (Preparation-2, Step-3) (15.0 g, 65.78 mmol) in DCE (200 mL) was added triethylamine (46.23 mL, 328.94 mmol), DMAP (8.03 g, 65.78 mmol) and Boc-anhydride (60.52 mL, 263.15 mmol) at RT under a nitrogen atmosphere. The reaction mass was heated at 70° C. for 16 h. Progress of the reaction was monitored by LCMS/TLC and after completion of the reaction water was added and the organics were extracted with ethyl acetate. The combined organic layers were washed with water followed by brine, dried over anhydrous sodium sulphate and evaporated under reduced pressure to afford the crude compound which was purified by column chromatography using 20-30% ethyl acetate in hexane to afford the titled compound (9.7 g, yield 45%) as an off white solid. LCMS m/z: 329.0 [M+H].

### Step 5: tert-Butyl 7-((4-methoxybenzyl)amino)-8-methyl-2,3-dihydro-1H-pyrido[2,3-b][1,4]oxazine-1-carboxylate

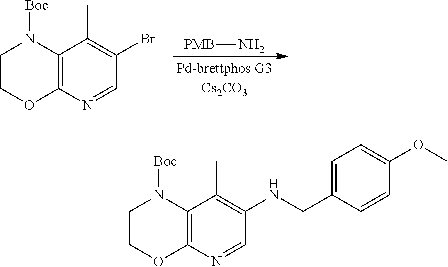

To a stirred solution of tert-butyl 7-bromo-8-methyl-2,3-dihydro-1H-pyrido[2,3-b][1,4]oxazine-1-carboxylate (Preparation-2, Step-4) (200 mg, 0.61 mmol) in 1,4-dioxane was added 4-methoxybenzylamine (0.16 mL, 1.22 mmol) and cesium carbonate (497 mg, 1.524 mmol). The resulting reaction mixture was degassed with nitrogen for 20 mins. then BrettphosPdG3 (112 mg, 0.12 mmol) was added and the mixture was again degassed for 10 mins. The whole reaction mass was heated at 100° C. for 16 h. Progress of the reaction was monitored by LCMS/TLC and after completion the mixture was filtered through a celite bed, washed with ethyl acetate and the combined washings were concentrated under reduced pressure to afford the crude product which was purified by column chromatography using 30-40% ethyl acetate in hexane as eluent to afford the titled compound (120 mg, yield 51.06%) as a pale yellow solid. LCMS m/z: 386 [M+H].

### Step 6:8-Methyl-2,3-dihydro-1H-pyrido[2,3-b][1,4]oxazin-7-amine.TFA

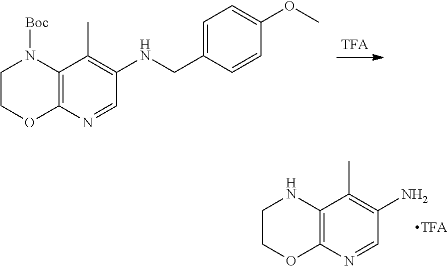 [SMILES: COc1ccc(CNc2cnc3c(c2C)N(C(=O)OC(C)(C)C)CCO3)cc1 ; Cc1c(N)cnc2c1NCCO2]

A solution of tert-butyl 7-((4-methoxybenzyl)amino)-8-methyl-2,3-dihydro-1H-pyrido[2,3-b][1,4]oxazine-1-carboxylate (Preparation-2, Step-5) (120 mg, 0.31 mmol) in TFA (4 mL) was stirred at room temperature for 2 h. After completion of the reaction (monitored by LCMS), the reaction mixture was evaporated in vacuo to afford the titled compound as its TFA salt (130 mg, crude) which was used as such in the next step without any further purification. LCMS m/z: 166 [M+H].

### Preparation 3:4-((8-Methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide (Example 45)

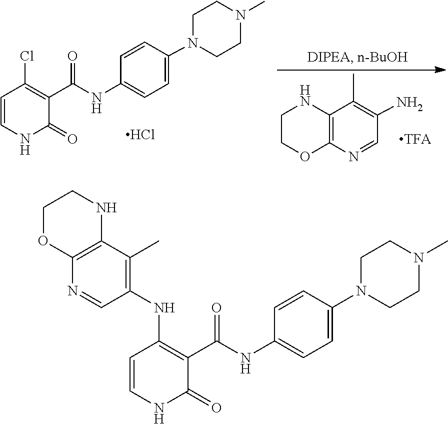 [SMILES: CN1CCN(c2ccc(NC(=O)c3c(Cl)cc[nH]c3=O)cc2)CC1 ; Cl ; Cc1c(N)cnc2c1NCCO2 ; Cc1c(Nc2cc[nH]c(=O)c2C(=O)Nc2ccc(N3CCN(C)CC3)cc2)cnc2c1NCCO2]

To a stirred solution of 4-chloro-N-(4-(4-methylpiperazin-1-yl)phenyl)-2-oxo-1,2-dihydropyridine-3-carboxamide.HCl (Preparation-1, Step-2) (150 mg, 0.43 mmol) in n-butanol (5 mL) was added N,N-diisopropylethylamine (1.13 mL, 6.50 mmol) and the whole was stirred at room temperature for 20 mins. Thereafter, 8-methyl-2,3-dihydro-1H-pyrido[2,3-b][1,4]oxazin-7-amine.TFA (Preparation-2, Step-6) (126 mg, 0.65 mmol) was added into the reaction mixture and the whole stirred at 120° C. for 16 h. Progress of the reaction was monitored by LCMS/TLC and after completion of the reaction, the solvent was evaporated under reduced pressure to give the crude product which was purified by preparative reverse phase HPLC to afford the titled compound (25 mg, yield 9.14%, qualitative HPLC purity 99.22% by area normalization) as a white solid. ¹H NMR (400 MHz; DMSO-d₆): δ 1.92 (s, 3H), 2.21 (s, 3H), 2.43-2.45 (m, 4H), 3.07-3.09 (m, 4H), 3.31 (bs, 2H), 4.25 (t, J=3.8 Hz, 2H), 5.55 (d, J=7.36 Hz, 1H), 5.79 (s, 1H), 6.90 (d, J=8.8 Hz, 2H), 7.27 (d, J=7.6 Hz, 1H), 7.30 (s, 1H), 7.46 (d, J=8.8 Hz, 2H), 11.35 (s, 1H), 11.90 (s, 1H), 12.86 (s, 1H); LCMS m/z: 476.46 [M+H].

### Example 167: N-(4-(4-Cyclopropylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide

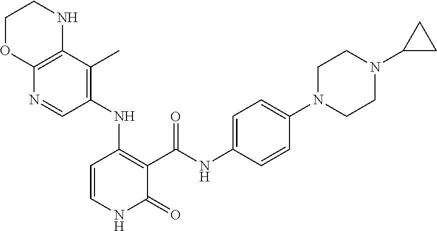 [SMILES: Cc1c(Nc2cc[nH]c(=O)c2C(=O)Nc2ccc(N3CCN(C4CC4)CC3)cc2)cnc2c1NCCO2]

Example 167 was prepared according to the methods described in General Procedures 1-7, and the methods described below.

### Preparation 9:8-Methyl-2,3-dihydro-1H-pyrido[2,3-b][1,4]oxazin-7-amine dihydrochloride

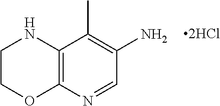

### Step 1:7-Bromo-8-methyl-1H-pyrido[2,3-b][1,4]oxazin-2 (3H)-one

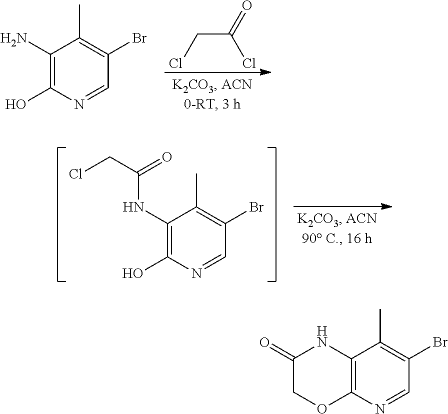

To a stirred solution of commercially available 3-amino-5-bromo-4-methylpyridin-2-ol (50.0 g, 0.246 mol) in acetonitrile (1.0 L) in an ice bath at 0-5° C. was added 2-chloroacetyl chloride (55.66 g, 0.495 mol). Then after 2-5 mins. of stirring, K2CO₃ (85.4 g, 0.618 mol, 2.5 eq) was added and the combined mixture was allowed to warm slowly to RT for 3 h. After 3 h, the reaction vessel was transferred to an oil bath and the contents refluxed at ˜90° C. for 3 h. LC-MS showed complete consumption of 3-amino-5- bromo-4-methylpyridin-2-ol and formation of the acylated intermediate. A further 2.5 equivalents of K2CO₃ (85.4 g, 0.618 mmol, 2.5 eq) was added into the reaction mixture in 0.5 equivalent increments to make the pH˜8. Then the resulting reaction mixture was stirred at reflux for 16 h. Complete consumption of the intermediate was confirmed by LC-MS. The reaction mixture was cooled to room temperature, the solvent was decanted and concentrated under reduced pressure to afford the crude material which was quenched with ice-cold water (1.0 L) to maintain an internal temperature of 5-10° C. The obtained suspension was stirred for 30 mins. and filtered. The leftover reaction mass was diluted with more ice-cold water (1.0 L). The obtained suspension was stirred for 30 mins. and filtered through the same Buchner funnel. The filter cake was washed with ice-cold water till the pH of the filtrate became neutral. The obtained wet material was dried in a vacuum oven at 50° C. overnight to afford the title compound (53.0 g, 88.5% yield, 97% pure by HPLC) as a white solid. LCMS m/z: 242.97 [M+H].

### Step 2:7-Bromo-8-methyl-2,3-dihydro-1H-pyrido[2,3-b][1,4]oxazine

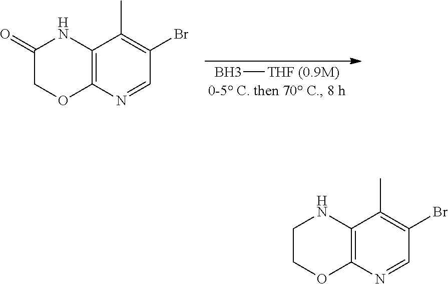

To a stirred solution of 7-bromo-8-methyl-1H-pyrido[2,3-b][1,4]oxazin-2 (3H)-one (Preparation 9, Step 1) (54 g, 0.222 mol) in dry THF (0.27 L, 5 V.) was added BH₃.THF solution (0.98 L, 0.888 mol, 0.9M solution in THF) dropwise via a dropping funnel under an inert atmosphere at 0-5° C. and the combined mixture was further stirred for 15 mins. The resulting reaction mixture was allowed to warm to RT then the reaction vessel was transferred to a pre-heated oil bath and the contents stirred at 70° C. for 8 h. Progress of the reaction was monitored by LC-MS until complete consumption of the starting material and formation of desired product was observed. The reaction mass was cooled to RT then transferred to an ice-bath and quenched with dropwise addition of ice-cold MeOH (0.27 L, 5V.) followed by IN HCl (0.98 L) and the whole further stirred overnight. Progress of the reaction was monitored by LC-MS which confirmed the formation of the desired product along with traces of impurities. After completion of the reaction the solvent was evaporated from the reaction mixture under reduced pressure to give a residue which was cooled on an ice-bath and neutralized with saturated NaHCO₃ solution (3 L) to achieve a PH˜8. The neutralized mass was diluted with water (1.0 L) and extracted with EtOAc (2×2.5 L). The combined organic layers were washed with brine solution (2.0 L), dried over anhydrous Na₂SO₄, filtered and concentrated under reduced pressure to afford the crude product (48.0 g) which was purified by column chromatography on silica gel (60-120 mess) (200 g for slurry and 2.5 Kg for column bed) using 10-50% EtOAc in DCM as eluent to afford the title compound (44.5 g). This material was again dissolved in ethyl acetate (0.9 L), followed by addition of activated charcoal (2.2 g) and stirred at room temperature for 30 mins. The charcoal was filtered through a celite bed, the bed was washed with ethyl acetate (0.1 L) and the resulting filtrate was evaporated in vacuo to afford the title compound (44.0 g, 86.8% yield, 99% pure by HPLC) as a white solid. LCMS m/z: 229.0 [M+H].

### Step 3: tert-Butyl (8-methyl-2,3-dihydro-1H-pyrido[2,3-b][1,4]oxazin-7-yl) carbamate

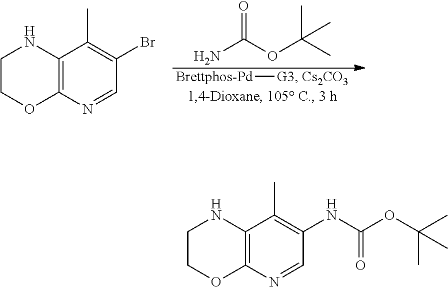

To a stirred solution of tert-butyl carbamate (63.9 g, 0.546 mol) in 1,4-dioxane (1.0 L) was added Cs₂CO₃ (177.79 g, 0.55 mol) and 7-bromo-8-methyl-2,3-dihydro-1H-pyrido[2,3-b][1,4]oxazine (Preparation 9, Step 2) (50.0 g, 0.218 mol) at RT. The reaction mixture was purged with nitrogen gas for 30 mins. Brettphos-Pd-G3 (5.94 g, 0.007 mol) was added and the mixture was again purged with nitrogen gas for 15 mins. The resulting reaction mixture was transferred to a pre-heated oil bath and stirred at 105° C. for 3 h under an inert atmosphere. Progress of the reaction was monitored by LC-MS and after completion the reaction mixture was cooled to RT, filtered through a celite bed and the bed was washed with 1,4-dioxane (200 mL). The filtrate was combined with the filtrate of another 75 g batch reaction as well and concentrated under reduced pressure to give a residue which was diluted with water (2.0 L) and extracted with EtOAc (2×2.5 L). The combined organic layers were washed with brine (2×2.0 L), dried over anhydrous Na₂SO₄, filtered and concentrated under reduced pressure to afford the crude product (250 g) which was purified by column chromatography on 60-120 mesh silica gel (500 g for the slurry and 5 kg for the column bed) using 5-50% EtOAc in DCM as eluent to give the title compound (135 g, 93% yield, 99% pure by HPLC) as a pale yellow solid. LCMS m/z: 266.24 [M+H].

### Step 4:8-Methyl-2,3-dihydro-1H-pyrido[2,3-b][1,4]oxazin-7-amine dihydrochloride

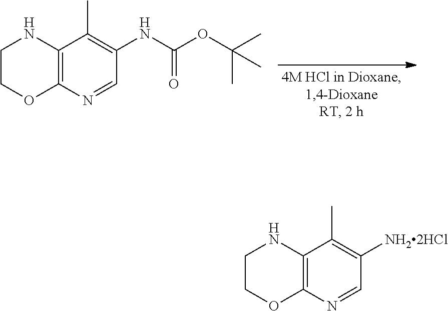

To a stirred suspension of tert-butyl (8-methyl-2,3-dihydro-1H-pyrido[2,3-b][1,4]oxazin-7-yl) carbamate (Preparation 9, Step 3) (135.0 g, 0.508 mol) in 1,4-dioxane (1.38 L) was added 4M HCl in 1,4-dioxane (1.38 L) at ice-cold temperature and the combined mixture allowed to stir at RT for 2 h. As the 4M HCl was added the suspension began to dissolve and lumps began to appear in the reaction mixture. Slowly, the lumps broke down and the reaction mixture became a thick suspension. After 2 h stirring at RT, LC-MS and TLC confirmed completion of the reaction in which the starting material was fully consumed. The solvent from the reaction mixture was evaporated under reduced pressure to obtain the crude material which was triturated with heptane, filtered and dried in vacuo at 40° C. for 30 mins. to afford the title compound (119.0 g, 99% yield, 99% pure by HPLC) as a light brown solid. LCMS m/z: 166.14 [M+H].

### Preparation 10:4-(4-Cyclopropylpiperazin-1-yl) aniline-methane

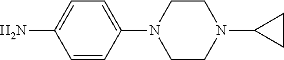 [SMILES: Nc1ccc(N2CCN(C3CC3)CC2)cc1]

### Step 1:1-Cyclopropyl-4-(4-nitrophenyl) piperazine

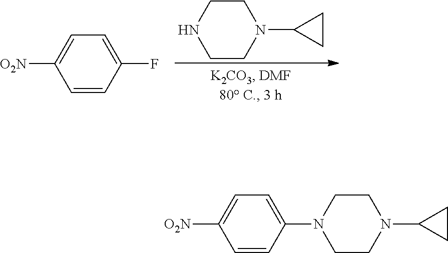 [SMILES: O=[N+]([O-])c1ccc(F)cc1 ; C1CN(C2CC2)CCN1 ; O=[N+]([O-])c1ccc(N2CCN(C3CC3)CC2)cc1]

To a stirred solution of commercially available 1-cyclopropylpiperazine (107.33 g, 0.9 mol) in DMF (1.0 L) was added K₂CO₃ (293.83 g, 2.12 mol) and the resulting reaction mixture was stirred at room temperature for 10 mins. Commercially available 1-fluoro-4-nitrobenzene (100.0 g, 0.709 mol) was added into the reaction mixture which was then stirred at 80° C. for 3 h. Progress of the reaction was monitored by HPLC and TLC. After complete consumption of the starting material the reaction mixture was cooled to RT and quenched with ice-cold water (5.0 L). The resulting suspension was stirred at RT for 30 mins. The precipitated solid was filtered and washed with water (2.5 L) until the pH of the filtrate became neutral. Finally, the solid was washed with n-heptane (1.0 L) and dried in a vacuum oven at 50° C. for 16 h to afford the title compound (172.0 g, 98% yield, 98.83% pure by HPLC) as a yellow solid. LCMS m/z: 248.18 [M+H].

### Step 2:4-(4-Cyclopropylpiperazin-1-yl) aniline

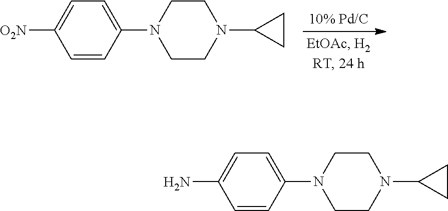 [SMILES: O=[N+]([O-])c1ccc(N2CCN(C3CC3)CC2)cc1 ; Nc1ccc(N2CCN(C3CC3)CC2)cc1]

A stirred solution of 1-cyclopropyl-4-(4-nitrophenyl) piperazine (Preparation 10, Step 1) (6.0 g, 0.024 mol) in ethyl acetate (60 mL) was purged with nitrogen followed by addition of 10% Pd/C (50% wet; 1.2 g, 20% w/w) under an inert atmosphere and the mixture was maintained under hydrogen balloon pressure at room temperature for 24 h. Progress of the reaction was monitored by UPLC and TLC. After complete consumption of the starting material the reaction mixture was filtered through a celite bed under an inert atmosphere and the bed was washed with ethyl acetate (15 mL) under a nitrogen gas atmosphere. The filtrate was evaporated under reduced pressure to afford the title compound (5.105 g, 98% yield, 77.49% pure by HPLC) as a light brown solid. LCMS m/z: 218.2 [M+H].

### Preparation 11: N-(4-(4-Cyclopropylpiperazin-1-yl)phenyl)-4-iodo-2-methoxynicotinamide

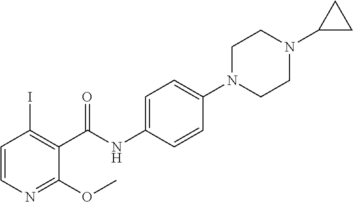 [SMILES: COc1nccc(I)c1C(=O)Nc1ccc(N2CCN(C3CC3)CC2)cc1]

### Step 1:4-Iodo-2-methoxynicotinic acid

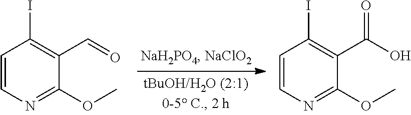

To a stirred solution of commercially available 4-iodo-2-methoxynicotinaldehyde (50.0 g, 0.19 mol) in t-BuOH (1.0 L) was added an aqueous solution of NaH₂PO₄ (114.0 g, 0.95 mol) and NaClO₂ (55.85 g, 0.6 mol) portionwise over 30 mins. at 0-5° C. . . . After the addition was complete the reaction mixture was maintained at 0-5° C. for 2 h. Progress of the reaction was monitored by HPLC and TLC. After complete consumption of the starting material the reaction mixture was quenched with an aqueous solution of Na₂SO₃ (250.0 g in 1.0 L of water) at 0-5° C. during which an exotherm was observed up to ˜ 10° C. and the colour of the mixture changed from yellow to light green. A saturated solution of K2CO₃ (500 mL) was then added to make the reaction mass basic (pH=14). The basic aqueous solution was washed with EtOAc (1.0 L) and acidified (pH=2) using 12N HCl (500 mL) at 5-10° C. Finally, the compound was extracted using 20% t-BuOH in ethyl acetate (2×2.0 L). The combined organic layers were dried over anhydrous Na₂SO₄, filtered and evaporated under reduced pressure to afford the title compound (50.0 g, 94% yield, 99% pure by HPLC) as a white solid. LCMS m/z: 279.94 [M+H].

### Step 2: N-(4-(4-Cyclopropylpiperazin-1-yl)phenyl)-4-iodo-2-methoxynicotinamide

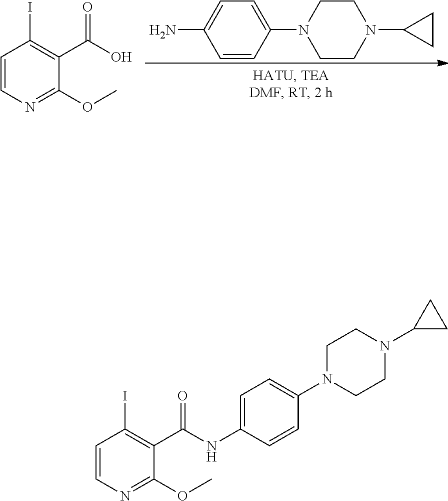 [SMILES: COc1nccc(I)c1C(=O)O ; Nc1ccc(N2CCN(C3CC3)CC2)cc1 ; COc1nccc(I)c1C(=O)Nc1ccc(N2CCN(C3CC3)CC2)cc1]

To a stirred solution of 4-iodo-2-methoxynicotinic acid (Preparation 11, Step 1) (100.0 g, 0.358 mol) in DMF (1.0 L) was added HATU (204.4 g, 0.538 mol) followed by dropwise addition of triethylamine (149.3 mL, 1.07 mol) at room temperature and the combined mixture was allowed to stir at RT for 10 mins. Further, 4-(4-cyclopropylpiperazin-1-yl) aniline (Preparation 10, Step 2) (125.6 g, 0.573 mol) was added and the resulting reaction mixture was allowed to stir at room temperature for 2 h. Progress of the reaction was monitored by UPLC and TLC. After complete consumption of the starting material the reaction mixture was poured into ice cooled water (4.0 L) and stirred for 30 mins. The precipitated solid was filtered and washed with water until the pH of the filtrate became neutral. Then the solid was dried in a vacuum oven at 50° C. for 16 h to afford the title compound (160.0 g, 94% yield, 99% pure by HPLC) as a light brown solid. LCMS m/z: 479.16 [M+H].

### Preparation 12: Step 1: N-(4-(4-Cyclopropylpiperazin-1-yl)phenyl)-2-methoxy-4-((8-methyl-2,3-dihydro-1H-pyrido[2,3-b][1,4]oxazin-7-yl)amino) nicotinamide

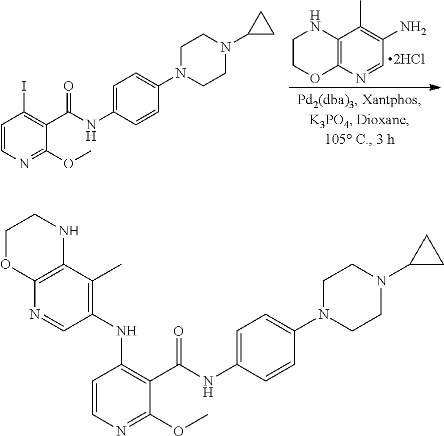

To a stirred solution of N-(4-(4-cyclopropylpiperazin-1-yl)phenyl)-4-iodo-2-methoxynicotinamide (Preparation 11, Step 2) (35.0 g, 0.073 mol) in 1,4-dioxane (0.35 L) was added K3PO₄ (62.13 g, 0.29 mol) and 8-methyl-2,3-dihydro-1H-pyrido[2,3-b][1,4]oxazin-7-amine dihydrochloride (Preparation 9, Step 4) (18.2 g, 0.077 mol) at room temperature. The reaction mixture was purged with nitrogen gas for 30 mins. After that, Pd₂ (dba)₃ (2.01 g, 0.0022 mol) and Xantphos (2.54 g, 0.0044 mol) were added to it. The resulting reaction mixture was transferred to a pre-heated oil bath and stirred at 105° C. for 3 h under an inert atmosphere. Progress of the reaction was monitored by LCMS and after completion the reaction mixture was cooled to RT and filtered through a celite bed and the bed was washed with DCM (0.2 L). The filtrate was concentrated under reduced pressure to give a residue (45.0 g) as a yellow solid which was diluted with water (0.7 L) and extracted with DCM (2×0.7 L). The combined organic layers were washed with brine (0.5 L), dried over anhydrous Na₂SO₄, filtered and concentrated under reduced pressure to afford the crude product (38.0 g) which was purified by column chromatography on silica gel[90.0 g (60-120 mesh silica gel) for the slurry and 1.35 kg (100-200 mesh) for the column bed], using 2-2.5% MeOH in DCM as eluent[0.5% of (total silica used)triethylamine was used to make the silica gel basic] to afford the title compound (31.0 g, 63% yield, 98.72% pure by HPLC) as a yellow solid. LCMS m/z: 516.35 [M+H].

### Step 2: N-(4-(4-Cyclopropylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide

### Example 167

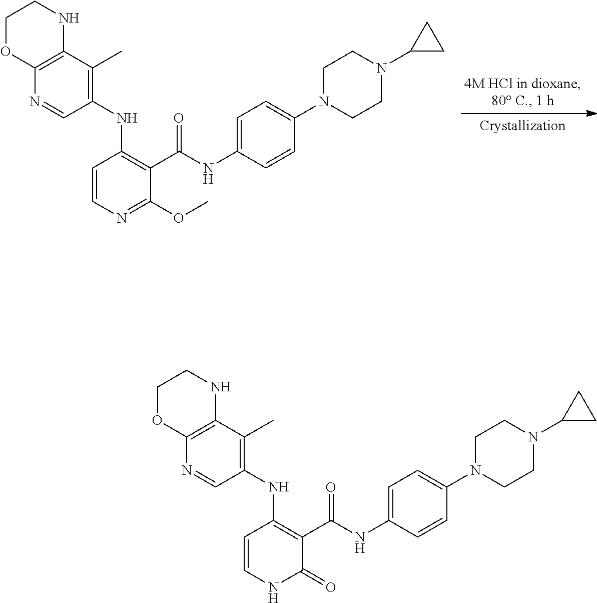 [SMILES: COc1nccc(Nc2cnc3c(c2C)NCCO3)c1C(=O)Nc1ccc(N2CCN(C3CC3)CC2)cc1 ; Cc1c(Nc2cc[nH]c(=O)c2C(=O)Nc2ccc(N3CCN(C4CC4)CC3)cc2)cnc2c1NCCO2]

A stirred solution of N-(4-(4-cyclopropylpiperazin-1-yl)phenyl)-2-methoxy-4-((8-methyl-2,3-dihydro-1H-pyrido[2,3-b][1,4]oxazin-7-yl)amino) nicotinamide (Preparation 12, Step 1) (12.5 g, 0.024 mol) in 4M HCl-1,4-dioxane (0.25 L) was heated at 80° C. for 1 h. Initially, the compound dissolved in the 4M HCl in 1,4-dioxane but as the temperature reached 80° C. lumps appeared in the reaction mixture. Slowly, the lumps broke down and the reaction mixture became a thick suspension. Progress of the reaction was monitored by LCMS. After complete consumption of the starting material the solvent from the reaction mixture was evaporated under reduced pressure to obtain a residue as a pale yellow solid which was dissolved in Milli-Q water (0.75 L, 60 V) and washed with ethyl acetate (2×0.3 L, HPLC grade). The acidic aqueous layer was transferred to a 2 L RBF and cooled to 0-5° C. with external cooling by an ice-salt mixture as the solution was neutralized by a saturated solution of NaHCO₃[0.2 L, 16 V (saturated NaHCO₃ was filtered through a sintered funnel prior to use)] by dropwise addition. After neutralization the pH of the solution was around 7-8 and a solid compound precipitated from the solution which was left to stir at RT for 30 mins. The solid was then filtered off using a Buchner funnel and washed with water until the pH of the filtrate became neutral. Finally, a heptane (0.1 L) wash was given to afford the wet cake (17.0 g, 97% pure by HPLC) as a pale yellow solid.

Crystallization 1: The wet product (17.0 g 0.034 mol) was dissolved in DCM (2.5 L, 200 V, HPLC grade) and refluxed to ensure maximum dissolution before Mili-Q water (0.6 L) was added and the whole filtered through a cotton plug. The solid collected on the cotton was washed with DCM (0.1 L) and mixed with the main original layer. The layers were separated and the organic portion was washed with brine (0.6 L), dried over anhydrous sodium sulphate, filtered and transferred to a 3 neck 5 L round bottomed flask. Activated charcoal (0.6 g) was added and the combined mixture was refluxed for 30 mins. The charcoal was filtered through a celite bed and the bed was washed with DCM (0.525 L, total DCM used was 250 V). To this clear yellow solution heptane [1.1 L, (⅓ volumes of total DCM)] was added slowly through a dropping funnel and the whole was refluxed. The clear solution became slightly turbid and the mixture was left to stir at RT overnight. The suspension was cooled with an external ice-salt mixture to 0-5° C. for 1 h and filtered through a Buchner funnel. Finally, the combined wet cake was washed with n-heptane (2×0.1 L) and left to suck dry for 1 h. The solid was dried in a vacuum oven at 50° C. overnight to afford the title compound (28.0 g, 76% yield, 99.78% pure by HPLC) as a pale yellow solid.

Crystallization 2: The wet product (10 g) was dissolved in DMA (160 mL) at 50° C., then charcoal (500 mg) was added and the whole was stirred at 50° C. for 45 mins. The mixture was filtered through a celite (15 g) bed and the bed was washed with hot DMA (5×20 mL). The filtrate was collected and chilled water (420 mL) added dropwise at 4-5° C. to obtain a precipitate and the suspension was kept cold for 3 hours. The suspension was filtered and washed with water (420 mL). The wet cake was given a slurry wash with water (2×300 mL), suck dried for 4 h and finally dried in a vacuum oven at 50° C. for 18 h then 75° C. for 10 h to afford the title compound (8.55 g, 99.02% pure by HPLC) as a light yellow solid. ¹H NMR (400 MHz; DMSO-d₆): δ 0.43-0.46 (m, 2H), 0.84-0.88 (m, 2H), 1.62-1.67 (m, 1H), 1.93 (s, 3H), 2.67 (t, J=4.84 Hz, 4H), 3.05 (t, J=4.56 Hz, 4H), 3.33 (s, 2H), 4.26 (t, J=4.12 Hz, 2H), 5.54-5.57 (m, 1H), 5.81 (s, 1H), 6.90 (d, J=9.08 Hz, 2H), 7.26-7.30 (m, 2H), 7.47 (d, J=9.0 Hz, 2H), 11.35 (d, J=6.0 Hz, 1H), 11.91 (s, 1H), 12.85 (s, 1H); LCMS m/z: 502.22 [M+H].

### Example 168: N-(3-Methyl-1,2,3,4,4a,5-hexahydrobenzo[b]pyrazino[1,2-d][1,4]oxazin-8-yl)-4-((8-methyl-2,3-dihydro-1H-pyrido[2,3-b][1,4]oxazin-7-yl)amino)-2-oxo-1,2-dihydropyridine-3-carboxamide

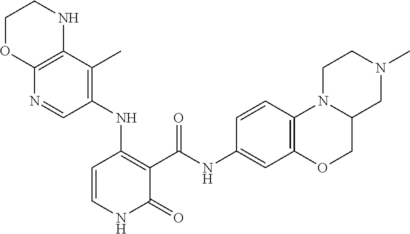 [SMILES: Cc1c(Nc2cc[nH]c(=O)c2C(=O)Nc2ccc3c(c2)OCC2CN(C)CCN32)cnc2c1NCCO2]

Example 168 was prepared according to the methods described in General Procedures 1-7, and the methods described below.

### Preparation 13:3-Methyl-1,2,3,4,4a,5-hexahydrobenzo[b]pyrazino[1,2-d][1,4]oxazin-8-amine

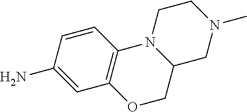 [SMILES: CN1CCN2c3ccc(N)cc3OCC2C1]

### Step 1: tert-Butyl 3-(hydroxymethyl) piperazine-1-carboxylate

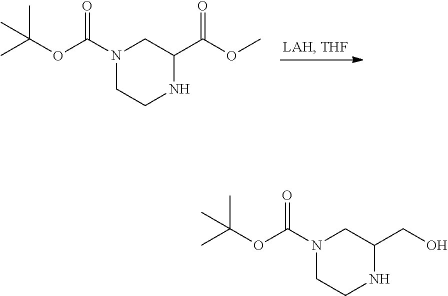

To a stirred solution of commercially available 1-(tert-butyl)3-methyl piperazine-1,3-dicarboxylate (1000 mg, 4.093 mmol) in dry THF (10 mL) was added a 1M solution of LiAlH₄ in THF (4.912 mL, 4.912 mmol) at 0-5° C. and the reaction mass was stirred at this temperature for 2 h. Progress of the reaction was monitored by TLC and after completion it was quenched by dropwise addition of a saturated solution of sodium sulphate and further stirred at RT for 1 h. The aqueous solution was filtered through a celite bed. The filtrate was evaporated in vacuo to afford the title compound (1050 mg, crude) as a yellowish solid which was used in the next step without any further purification. LCMS m/z: 217.10 [M+H].

### Step 2: tert-Butyl 8-nitro-1,2,4a,5-tetrahydrobenzo[b]pyrazino[1,2-d][1,4]oxazine-3 (4H)-carboxylate

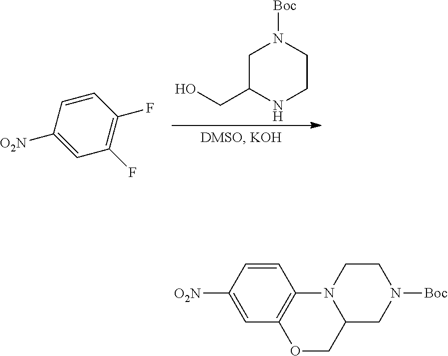

To a stirred solution of tert-butyl 3-(hydroxymethyl) piperazine-1-carboxylate (Preparation 13, Step 1) (780 mg, 3.606 mmol) in DMSO (10 mL) was added commercially available 1,2-difluoro-4-nitrobenzene (745.87 mg, 4.688 mmol) followed by KOH (627.30 mg, 11.179 mmol) and the whole was stirred at 30° C. for 18 h. The progress of the reaction was monitored by LCMS and after completion it was diluted with chilled water and extracted with 10% methanol in DCM. The combined organic layers were distilled off to obtain the crude compound which was purified by column chromatography on a silica gel bed using methanol and DCM as eluent to afford the title compound (280 mg, 99% yield) as a yellow semi-solid. LCMS m/z: 336.11 [M+H].

### Step 3:8-Nitro-1,2,3,4,4a,5-hexahydrobenzo[b]pyrazino[1,2-d][1,4]oxazine.HCl

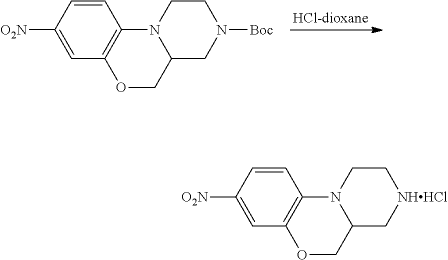

To a stirred solution of tert-butyl 8-nitro-1,2,4a,5-tetrahydrobenzo[b]pyrazino[1,2-d][1,4]oxazine-3 (4H)-carboxylate (Preparation 13, Step 2) (280 mg, 0.834 mmol) in 1,4-dioxane (3 mL) was added 4M HCl-dioxane (3 mL) at 0-5° C. and the resulting reaction mixture was stirred at RT for 3 h. The reaction was monitored by LCMS and after completion it was evaporated in vacuo to afford the title compound (270 mg, crude) as a yellowish solid which was used in the next step without any further purification. LCMS m/z: 236.02 [M+H].

### Step 4:3-Methyl-8-nitro-1,2,3,4,4a,5-hexahydrobenzo[b]pyrazino[1,2-d][1,4]oxazine

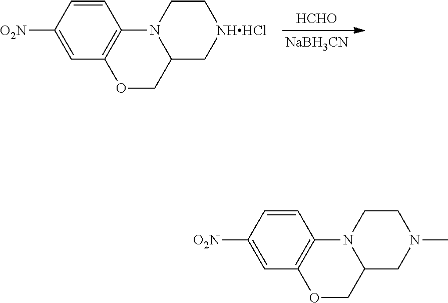

To a stirred solution of 8-nitro-1,2,3,4,4a,5-hexahydrobenzo[b]pyrazino[1,2-d][1,4]oxazine.HCl (Preparation 13, Step 3) (270 mg, 1.148 mmol) in MeOH (5 mL) was added a 40% solution of formaldehyde (0.430 mL, 5.739 mmol) and NaBH₃CN (129.83 mg, 2.066 mmol) at RT. The resulting reaction mixture was stirred at RT for 16 h. Progress of the reaction was monitored by UPLC-MS and after completion it was diluted with water and extracted with 10% methanol in DCM. The combined organic layers were evaporated in vacuo to afford the title compound (235 mg, crude) as a yellowish semi-solid which was used in the next step without any further purification. LCMS m/z: 250.05 [M+H].

### Step 5:3-Methyl-1,2,3,4,4a,5-hexahydrobenzo[b]pyrazino[1,2-d][1,4]oxazin-8-amine

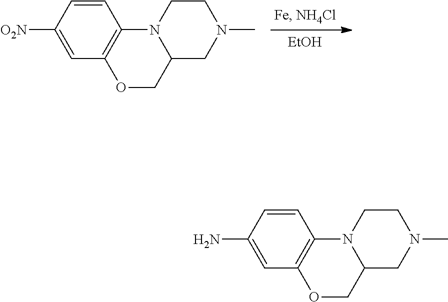

To a stirred solution of 3-methyl-8-nitro-1,2,3,4,4a,5-hexahydrobenzo[b]pyrazino[1,2-d][1,4]oxazine (Preparation 13, Step 4) (225 mg, 0.903 mmol) in ethanol (5 mL) was added Fe powder (252.02 mg, 4.513 mmol) followed by NH₄Cl (241.41 mg, 4.513 mmol) as a solution in water (1 mL). The resulting reaction mixture was refluxed at 85° C. for 2 h. Progress of the reaction was monitored by TLC and LC-MS. After completion, the reaction mixture was diluted with water (20 mL) and extracted with 10% methanol in DCM solution (2×100 mL). The organic layer was dried over anhydrous sodium sulphate, concentrated under reduced pressure to afford the crude compound which was purified by Combi-flash using 4% methanol in DCM as eluent to afford the title compound (180 mg, 90% yield) as a pale brown sticky liquid. LCMS m/z: 220.06 [M+H].

### Preparation 14, Step 1:4-Iodo-2-methoxy-N-(3-methyl-1,2,3,4,4a,5-hexahydrobenzo[b]pyrazino[1,2-d][1,4]oxazin-8-yl) nicotinamide

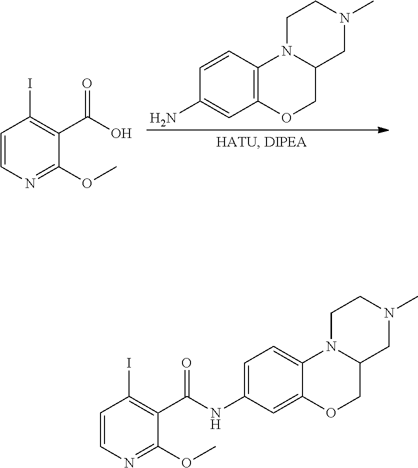 [SMILES: COc1nccc(I)c1C(=O)O ; CN1CCN2c3ccc(N)cc3OCC2C1 ; COc1nccc(I)c1C(=O)Nc1ccc2c(c1)OCC1CN(C)CCN21]

To a stirred solution of 4-iodo-2-methoxynicotinic acid (Preparation 11, Step 1) (170 mg, 0.609 mmol) in THF (10 mL) was added HATU (278.05 mg, 0.731 mmol) at RT. The resulting reaction mixture was stirred at RT for 15 mins. Then DIPEA (0.319 mL, 1.828 mmol) and 3-methyl-1,2,3,4,4a,5-hexahydrobenzo[b]pyrazino[1,2-d][1,4]oxazin-8-amine (Preparation 13, Step 5) (133.54 mg, 0.609 mmol) were added into the reaction vessel and the whole was stirred at room temperature for 18 h. Progress of the reaction was monitored by TLC/LC-MS and after completion the reaction mixture was diluted with water (20 mL) and extracted with 10% methanol in DCM (2×100 mL). The combined organic layers were dried over anhydrous sodium sulphate, filtered and concentrated under reduced pressure to give the crude product which was purified by Combi-flash on a silica gel (12 g) column using 5% MeOH in DCM as eluent to afford the title compound (150 mg, 51% yield, HPLC purity 98%) as a pale brown sticky liquid. LCMS m/z: 481.04 [M+H].

### Step 2:4-Iodo-N-(3-methyl-1,2,3,4,4a,5-hexahydrobenzo[b]pyrazino[1,2-d][1,4]oxazin-8-yl)-2-oxo-1,2-dihydropyridine-3-carboxamide hydrochloride

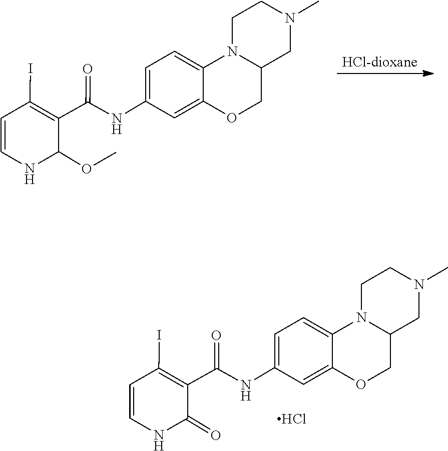 [SMILES: COC1NC=CC(I)=C1C(=O)Nc1ccc2c(c1)OCC1CN(C)CCN21 ; CN1CCN2c3ccc(NC(=O)c4c(I)cc[nH]c4=O)cc3OCC2C1 ; Cl]

A stirred suspension of 4-iodo-2-methoxy-N-(3-methyl-1,2,3,4,4a,5-hexahydrobenzo[b]pyrazino[1,2-d][1,4]oxazin-8-yl) nicotinamide (Preparation 14, Step 1) (140 mg, 0.291 mmol) in 4M HCl in dioxane (5 mL) was heated at 80° C. for 2 h. After completion of the reaction (monitored by LCMS) the reaction mass was evaporated in vacuo to afford the title compound (140 mg, crude) as a yellowish sticky solid which was used in the next step without any further purification. LCMS m/z: 375.05_[M+H].

### Step 3: N-(3-Methyl-1,2,3,4,4a,5-hexahydrobenzo[b]pyrazino[1,2-d][1,4]oxazin-8-yl)-4-((8-methyl-2,3-dihydro-1H-pyrido[2,3-b][1,4]oxazin-7-yl)amino)-2-oxo-1,2-dihydropyridine-3-carboxamide (Example 168)

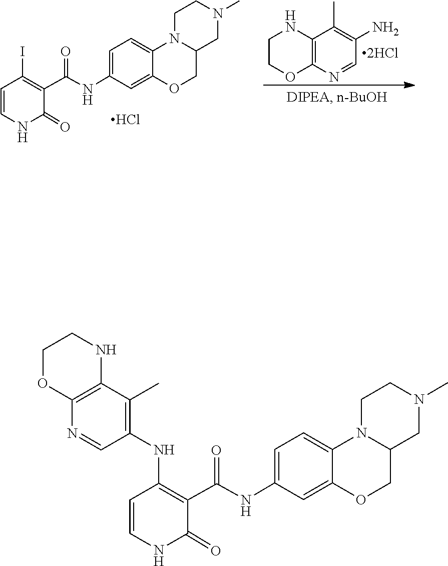 [SMILES: CN1CCN2c3ccc(NC(=O)c4c(I)cc[nH]c4=O)cc3OCC2C1 ; Cl ; Cc1c(N)cnc2c1NCCO2 ; Cc1c(Nc2cc[nH]c(=O)c2C(=O)Nc2ccc3c(c2)OCC2CN(C)CCN32)cnc2c1NCCO2]

To a stirred solution of 4-iodo-N-(3-methyl-1,2,3,4,4a,5-hexahydrobenzo[b]pyrazino[1,2-d][1,4]oxazin-8-yl)-2-oxo-1,2-dihydropyridine-3-carboxamide hydrochloride (Preparation 14, Step 2) (140 mg, 0.421 mmol) in n-BuOH (10 mL) was added 8-methyl-2,3-dihydro-1H-pyrido[2,3-b][1,4]oxazin-7-amine dihydrochloride (Preparation 9, Step 4) (83.40 mg, 0.505 mmol) followed by DIPEA (0.367 mL, 2.103 mmol) at RT. The resulting reaction mixture was stirred at 110° C. for 18 h. Progress of the reaction was monitored by LC-MS and after completion the reaction mixture was diluted with water (25 mL) and extracted with 10% methanol in DCM solution (2×100 mL). The organic layer was dried over anhydrous sodium sulphate, filtered and concentrated under reduced pressure to give the crude product which was purified by prep-HPLC to afford the title compound (25 mg, 13% yield, 96.82% pure by HPLC) as a brown solid. ¹H NMR (500 MHz; DMSO-d₆): δ 1.67 (t, J=10.65 Hz, 1H), 1.92 (s, 3H), 2.05-2.10 (m, 2H), 2.21 (s, 3H), 2.57-2.63 (m, 2H), 2.78 (d, J=10.0 Hz, 1H), 2.85 (d, J=10.6 Hz, 1H), 2.99 (t, J=9.65 Hz, 1H), 3.65 (d, J=11.35H, 1H), 3.89 (t, J=9.45 Hz, 1H), 4.21-4.26 (m, 3H), 5.55 (d, J=7.45 Hz, 1H), 5.81 (s, 1H), 6.81 (d, J=8.8 Hz, 1H), 6.92-6.94 (m, 1H), 7.14 (d, J=2.25 Hz, 1H), 7.28 (t, J=10.05 Hz, 2H), 11.31 (s, 1H), 11.88 (s, 1H), 12.82 (s, 1H); LCMS m/z: 504.29 [M+H].

### Example 363:4-((8-Methyl-2,3-dihydro-1H-pyrido[2,3-b][1,4]oxazin-7-yl)amino)-N-(4-(4-(6-methylpyridin-3-yl) piperazin-1-yl)phenyl)-2-oxo-1,2-dihydropyridine-3-carboxamide

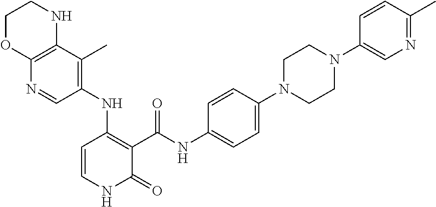 [SMILES: Cc1ccc(N2CCN(c3ccc(NC(=O)c4c(Nc5cnc6c(c5C)NCCO6)cc[nH]c4=O)cc3)CC2)cn1]

Example 363 was prepared according to the methods described in General Procedures 1-7, and the methods described below.

### Preparation 15:4-(4-(6-Methylpyridin-3-yl) piperazin-1-yl) aniline

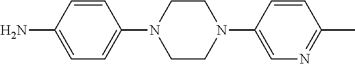 [SMILES: Cc1ccc(N2CCN(c3ccc(N)cc3)CC2)cn1]

### Step 1:1-(6-Methylpyridin-3-yl)-4-(4-nitrophenyl) piperazine

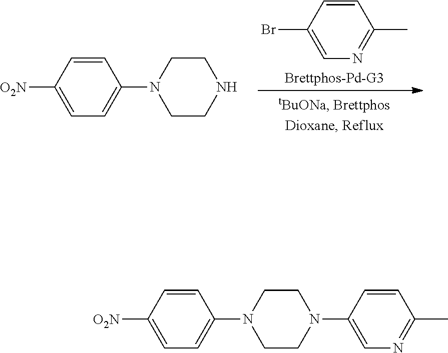

To a degassed solution of commercially available 1-(4-nitrophenyl) piperazine (300 mg, 1.43 mmol) in 1,4-dioxane (5 mL) was added 5-bromo-2-methyl-pyridine (296 mg, 1.72 mmol), ^{t}BuONa (402 mg, 3.58 mmol), BrettPhos (154 mg, 0.29 mmol) and Brettphos-Pd-G3 (130 mg, 0.14 mmol) at RT. The resulting mixture was heated at 100° C. under a nitrogen atmosphere for 16 h. Progress of the reaction was monitored by LCMS and after completion the reaction mixture was cooled to RT and filtered through a celite bed. The filtrate was evaporated under reduced pressure to obtain the crude product which was then purified by column chromatography to afford the title compound (400 mg, 93.5% yield) as a yellow solid. LCMS m/z: 299.1 [M+H].

### Step 2:4-(4-(6-Methylpyridin-3-yl) piperazin-1-yl) aniline

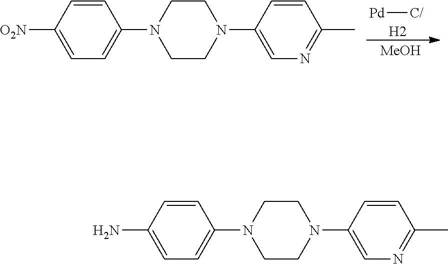

To a stirred solution of 1-(6-methylpyridin-3-yl)-4-(4-nitrophenyl) piperazine (Preparation 15, Step 1) (250 mg, 0.88 mmol) in methanol (5 mL) was added 10% Pd—C(50 mg) under an inert atmosphere at RT and the reaction mixture was then placed under hydrogen balloon pressure for 2 h. TLC and LC-MS confirmed completion of the reaction, after which the reaction mass was filtered through a short celite bed and the filtrate was evaporated under reduced pressure to obtain the crude product which was purified by column chromatography to afford the title compound (200 mg, 85% yield) as an off-white solid. LCMS m/z: 269.2 [M+H].

### Preparation 16:2-Methoxy-4-((8-methyl-2,3-dihydro-1H-pyrido[2,3-b][1,4]oxazin-7-yl)amino) nicotinic acid

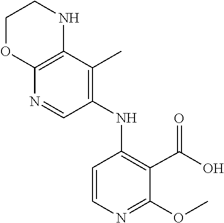 [SMILES: COc1nccc(Nc2cnc3c(c2C)NCCO3)c1C(=O)O]

### Step 1: Methyl 4-iodo-2-methoxynicotinate

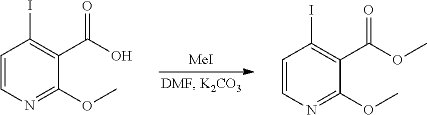

To a stirred solution of commercially available 4-iodo-2-methoxynicotinic acid (300 mg, 1.075 mmol) in DMF (6 mL) was added K₂CO₃ (371.50 mg, 2.688 mmol) followed by MeI (0.1 mL, 1.613 mmol) at ice-cold temperature and the whole was allowed to stir at room temperature for 2 h. UPLC and TLC showed the desired mass had formed and the starting material was fully consumed. The reaction mixture was diluted with water (20 mL) and extracted with ethyl acetate (2×100 mL). The combined organic layers were dried over anhydrous sodium sulphate, filtered and concentrated under reduced pressure to afford the crude product which was purified by Combi-flash chromatography on silica gel (12.0 g) using 20% ethyl acetate in hexane as eluent to afford the title compound (190 mg, 60% yield) as an off-white solid. LCMS m/z: 294.02 [M+H].

### Step 2: Methyl 2-methoxy-4-((8-methyl-2,3-dihydro-1H-pyrido[2,3-b][1,4]oxazin-7-yl)amino) nicotinate

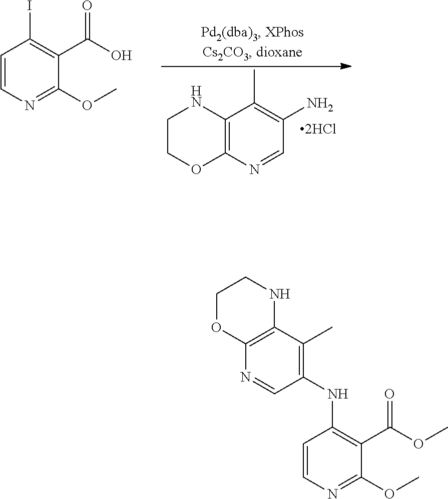 [SMILES: COc1nccc(I)c1C(=O)O ; Cc1c(N)cnc2c1NCCO2 ; COC(=O)c1c(Nc2cnc3c(c2C)NCCO3)ccnc1OC]

To a stirred solution of 8-methyl-2,3-dihydro-1H-pyrido[2,3-b][1,4]oxazin-7-amine dihydrochloride (Preparation 9, Step 4) (121.64 mg, 0.737 mmol) in 1,4-dioxane (15 mL) was added Cs₂CO₃ (1000.81 mg, 3.072 mmol) followed by methyl 4-iodo-2-methoxynicotinate (Preparation 16, Step 1) (180 mg, 0.614 mmol) at RT. The resulting mixture was degassed for 15 mins. and then Pd₂ (dba)₃ (56.22 mg, 0.0614 mmol) and XPhos (71.05 mg, 0.1228 mmol) were added and the whole was further stirred at 100° C. overnight. LCMS and TLC showed the desired compound had formed and the starting material was fully consumed. The crude mixture was diluted with water and extracted with 10% methanol in DCM. The combined organic layers were dried over anhydrous Na₂SO₄, filtered and evaporated in vacuo to obtain the crude product which was purified by Combi-flash to afford the title compound (150 mg, 77% yield) as a white solid. LCMS m/z: 331.22 [M+H].

### Step 3:2-Methoxy-4-((8-methyl-2,3-dihydro-1H-pyrido[2,3-b][1,4]oxazin-7-yl)amino) nicotinic acid

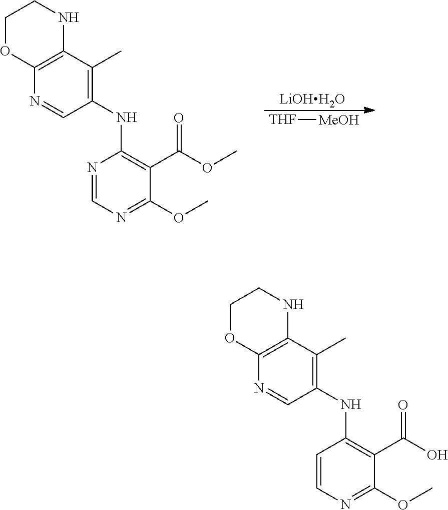

To a stirred solution of methyl 4-((8-methyl-2,3-dihydro-1H-pyrido[2,3-b][1,4]oxazin-7-yl)amino)-2-oxo-1,2-dihydropyridine-3-carboxylate (Preparation 16, Step 2) (140 mg, 0.424 mmol) in THF (2 mL) and MeOH (1 mL) was added LiOH.H₂O (89 mg, 2.12 mmol) dissolved in H₂O (1 mL) and the whole was stirred at RT for 4 h. UPLC and TLC showed the desired mass had formed and the starting material was fully consumed. The solvents were evaporated in vacuo to obtain the crude product which was diluted with a small amount of water and the pH of the mixture was adjusted to ˜5 by slow addition of citric acid solution. The resulting mass was diluted with water and extracted with 10% methanol in DCM. The combined organic layers were dried over anhydrous Na₂SO₄, filtered and evaporated in vacuo to afford the title compound (130 mg, crude) which was used in the next step without any further purification. LCMS m/z: 317.19 [M+H].

### Preparation 17, Step 1:2-Methoxy-4-((8-methyl-2,3-dihydro-1H-pyrido[2,3-b][1,4]oxazin-7-yl)amino)-N-(4-(4-(6-methylpyridin-3-yl) piperazin-1-yl)phenyl) nicotinamide

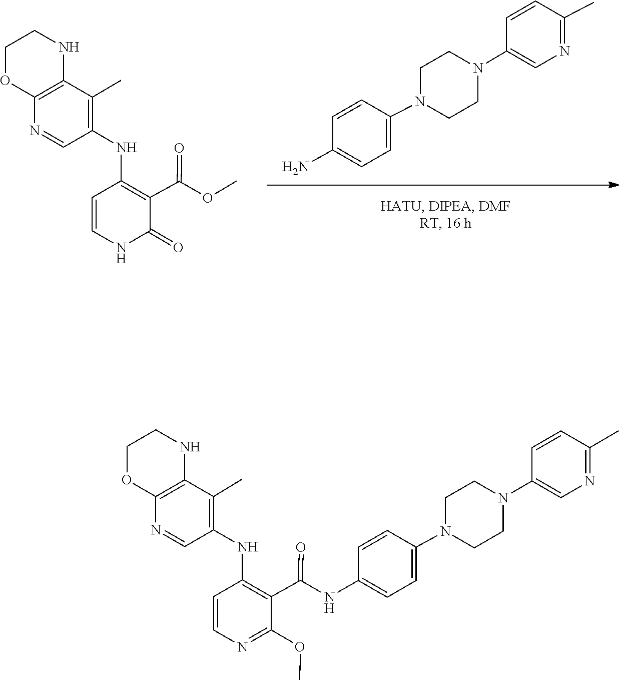 [SMILES: COC(=O)c1c(Nc2cnc3c(c2C)NCCO3)cc[nH]c1=O ; Cc1ccc(N2CCN(c3ccc(N)cc3)CC2)cn1 ; COc1nccc(Nc2cnc3c(c2C)NCCO3)c1C(=O)Nc1ccc(N2CCN(c3ccc(C)nc3)CC2)cc1]

To a stirred solution of 2-methoxy-4-((8-methyl-2,3-dihydro-1H-pyrido[2,3-b][1,4]oxazin-7-yl)amino) nicotinic acid (Preparation 16, Step 3) (100 mg, 0.32 mmol) in DMF (3 mL) was added DIPEA (0.17 mL, 0.95 mmol) and HATU (360 mg, 0.95 mmol) and the whole was stirred at RT for 30 mins. Then 4-(4-(6-methylpyridin-3-yl) piperazin-1-yl) aniline (preparation 15, Step 2) (84.8 mg, 0.32 mmol) was added into the reaction vessel and stirring continued at RT for 16 h. Progress of the reaction was monitored by LCMS and after completion the reaction mass was quenched with ice-cold water and extracted with ethyl acetate. The combined organic layers were washed with brine, dried over anhydrous sodium sulphate, filtered and concentrated under reduced pressure to obtain the crude product which was purified by column chromatography to afford the title compound (100 mg, 55.8% yield) as a brown solid. LCMS m/z: 567.3 [M+H].

### Step 2:4-((8-Methyl-2,3-dihydro-1H-pyrido[2,3-b][1,4]oxazin-7-yl)amino)-N-(4-(4-(6-methylpyridin-3-yl) piperazin-1-yl)phenyl)-2-oxo-1,2-dihydropyridine-3-carboxamide

### Example 363

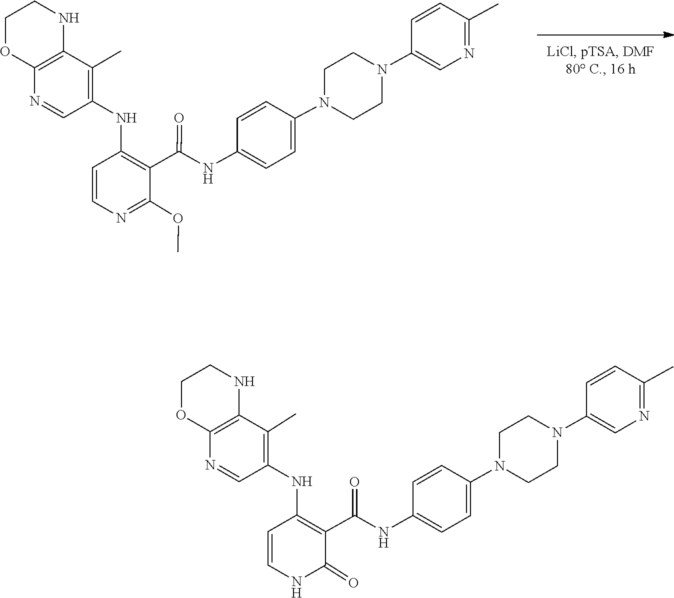 [SMILES: COc1nccc(Nc2cnc3c(c2C)NCCO3)c1C(=O)Nc1ccc(N2CCN(c3ccc(C)nc3)CC2)cc1 ; Cc1ccc(N2CCN(c3ccc(NC(=O)c4c(Nc5cnc6c(c5C)NCCO6)cc[nH]c4=O)cc3)CC2)cn1]

To a stirred solution of 2-methoxy-4-((8-methyl-2,3-dihydro-1H-pyrido[2,3-b][1,4]oxazin-7-yl)amino)-N-(4-(4-(6-methylpyridin-3-yl) piperazin-1-yl)phenyl) nicotinamide (Preparation 17, Step 1) (40 mg, 0.07 mmol) in DMF (2 mL) was added LiCl (14.9 mg, 0.35 mmol) and pTSA (67.2 mg, 0.35 mmol) under an inert atmosphere at RT. The resulting reaction mixture was stirred at 80° C. for 16 h. Progress of the reaction was monitored by LCMS and after completion the reaction mass was directly purified by prep-HPLC to afford the title compound (12 mg, 30.8% yield, 99.65% purity by HPLC) as an off-white solid. ¹H NMR (400 MHz; DMSO-d₆): δ 1.93 (s, 3H), 2.36 (s, 3H), 3.25-3.31 (m, 10H, merged in DMSO water), 4.25 (s, 2H), 5.56 (d, J=7.36 Hz, 1H), 5.79 (s, 1H), 6.99 (d, J=8.96 Hz, 2H), 7.10 (d, J=8.4 Hz, 1H), 7.26-7.33 (m, 3H), 7.51 (d, J=8.88 Hz, 2H), 8.21 (s, 1H), 11.34 (s, 1H), 11.90 (s, 1H), 12.87 (s, 1H); LCMS m/z: 553.2 [M+H].

### Example 371:4-((8-Methyl-2,3-dihydro-1H-pyrido[2,3-b][1,4]oxazin-7-yl)amino)-2-oxo-N-(4-(4-(thiazol-2-yl) piperazin-1-yl)phenyl)-1,2-dihydropyridine-3-carboxamide

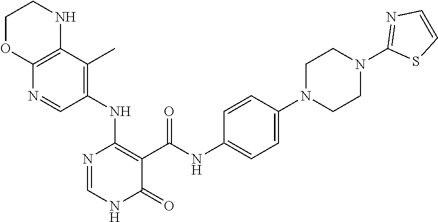 [SMILES: Cc1c(Nc2nc[nH]c(=O)c2C(=O)Nc2ccc(N3CCN(c4nccs4)CC3)cc2)cnc2c1NCCO2]

Example 371 was prepared according to the methods described in General Procedures 1-7, and the methods described below.

### Preparation 18:4-(4-(Thiazol-2-yl) piperazin-1-yl) aniline

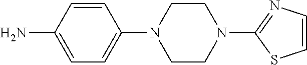 [SMILES: Nc1ccc(N2CCN(c3nccs3)CC2)cc1]

### Step 1:2-(4-(4-Nitrophenyl) piperazin-1-yl) thiazole

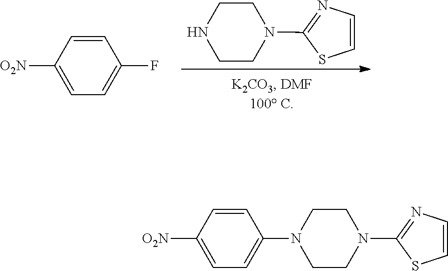 [SMILES: O=[N+]([O-])c1ccc(F)cc1 ; c1csc(N2CCNCC2)n1 ; O=[N+]([O-])c1ccc(N2CCN(c3nccs3)CC2)cc1]

To a stirred solution of commercially available 1-fluoro-4-nitro-benzene (400 mg, 2.34 mmol) in DMF (10 mL) was added K₂CO₃ (978 mg, 7.09 mmol) and commercially available 2-piperazin-1-ylthiazole (400 mg, 2.84 mmol) and stirring was continued at RT for 16 h. LCMS confirmed the formation of product. The reaction mixture was quenched with ice-cold water and extracted with ethyl acetate. The combined organic layers were washed with brine, dried over sodium sulphate, filtered and concentrated under reduced pressure to obtain the crude product which was purified by column chromatography to afford the title compound (500 mg, 72.9% yield) as a yellow solid. LCMS m/z: 291.0 [M+H].

### Step 2:4-(4-(Thiazol-2-yl) piperazin-1-yl) aniline

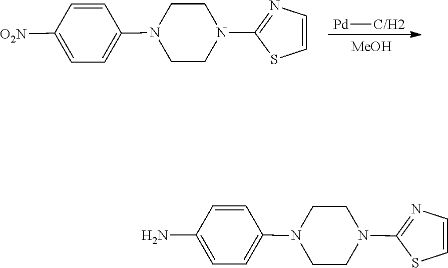

To a stirred solution of 2-(4-(4-nitrophenyl) piperazin-1-yl) thiazole (Preparation 18, Step 1) (250 mg, 0.877 mmol) in methanol (5 mL) was added 10% Pd—C(50 mg) under an inert atmosphere at RT. The resulting mixture was stirred at RT for 2 h. LCMS confirmed formation of the product. The reaction mass was filtered through a short celite bed and the filtrate was evaporated under reduced pressure to afford the title compound (210 mg, 92% yield) as a solid which was used in the next step without any further purification. LCMS m/z: 261.1 [M+H].

### Preparation 19:4-((8-Methyl-2,3-dihydro-1H-pyrido[2,3-b][1,4]oxazin-7-yl)amino)-2-oxo-N-(4-(4-(thiazol-2-yl) piperazin-1-yl)phenyl)-1,2-dihydropyridine-3-carboxamide

### Example 371

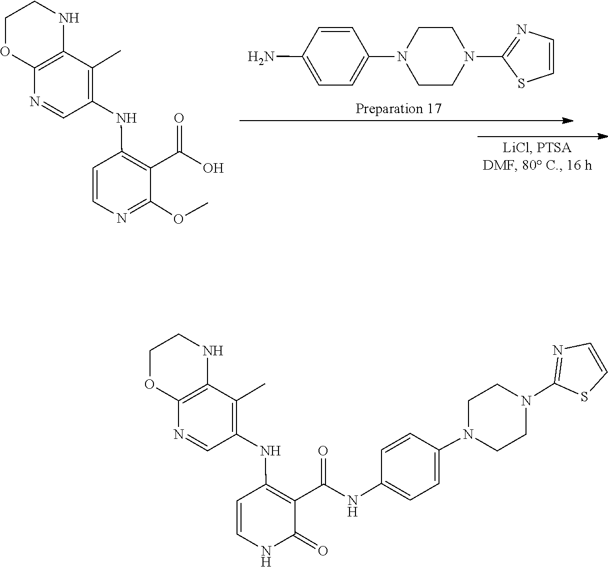 [SMILES: COc1nccc(Nc2cnc3c(c2C)NCCO3)c1C(=O)O ; Nc1ccc(N2CCN(c3nccs3)CC2)cc1 ; Cc1c(Nc2cc[nH]c(=O)c2C(=O)Nc2ccc(N3CCN(c4nccs4)CC3)cc2)cnc2c1NCCO2]

The title compound was prepared using exactly the same methods as described in Preparation 17, Steps 1-2 wherein the coupling amine was 4-(4-(thiazol-2-yl) piperazin-1-yl) aniline (Preparation 18, Steps 2). HPLC purity: 99.10%; ¹H NMR (400 MHz; DMSO-d₆): δ 1.93 (s, 3H), 3.23 (t, J=5.12 Hz, 4H), 3.33 (s, 2H, merged in DMSO water)3.53 (t, J=4.8 Hz, 4H), 4.24 (d, J=3.65 Hz, 2H), 5.55 (d, J=7.4 Hz, 1H), 5.81 (s, 1H), 6.88 (d, J=3.6 Hz, 1H), 6.99 (d, J=9.04 Hz, 2H), 7.19 (d, J=3.6 Hz, 1H), 7.29 (d, J=11.92 Hz, 2H), 7.51 (d, J=8.92 Hz, 2H), 11.37 (d, J=7.56 Hz, 1H), 11.89 (s, 1H), 12.89 (s, 1H); LCMS m/z: 545.39 [M+H].

### Example 197:4-((3,8-Dimethyl-2,3-dihydro-1H-pyrido[2,3-b][1,4]oxazin-7-yl)amino)-N-(4-(2-(methoxymethyl)-4-methylpiperazin-1-yl)phenyl)-2-oxo-1,2-dihydropyridine-3-carboxamide

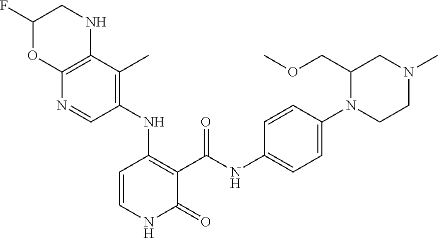 [SMILES: COCC1CN(C)CCN1c1ccc(NC(=O)c2c(Nc3cnc4c(c3C)NCC(F)O4)cc[nH]c2=O)cc1]

Example 197 was prepared according to the methods described in General Procedures 1-7, and the methods described below.

### Preparation 20:4-(2-(Methoxymethyl)-4-methylpiperazin-1-yl) aniline

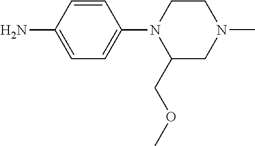 [SMILES: COCC1CN(C)CCN1c1ccc(N)cc1]

### Step 1: tert-Butyl 3-(hydroxymethyl)-4-(4-nitrophenyl) piperazine-1-carboxylate

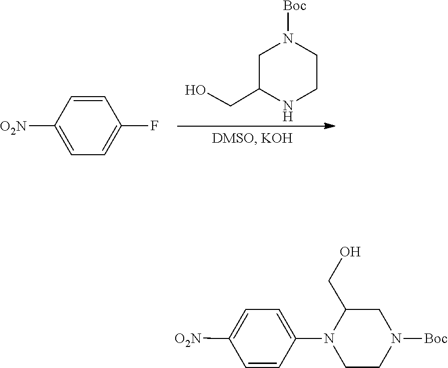 [SMILES: O=[N+]([O-])c1ccc(F)cc1 ; CC(C)(C)OC(=O)N1CCNC(CO)C1 ; CC(C)(C)OC(=O)N1CCN(c2ccc([N+](=O)[O-])cc2)C(CO)C1]

To a stirred solution of commercially available 1-fluoro-4-nitrobenzene (500 mg, 0.046 mmol) in DMSO (8 mL) was added tert-butyl 3-(hydroxymethyl) piperazine-1-carboxylate (Preparation 13, Step 1) (342.51 mg, 2.427 mmol) followed by KOH (389.15 mg, 6.935 mmol) at RT, then it was further stirred at 30° C. for 18 h. Progress of the reaction was monitored by TLC/LCMS and after completion the reaction mixture was diluted with chilled water (20 mL) and extracted with ethyl acetate (2×100 mL). The combined organic layers were dried over anhydrous sodium sulphate, filtered and concentrated under reduced pressure to give the crude product which was purified by Combi-flash using 3% MeOH-DCM as eluent to afford the title compound (415 mg, 51% yield) as a pale brown sticky liquid. LCMS m/z: 338.12 [M+H].

### Step 2:2-(Methoxymethyl)-4-methyl-1-(4-nitrophenyl) piperazine

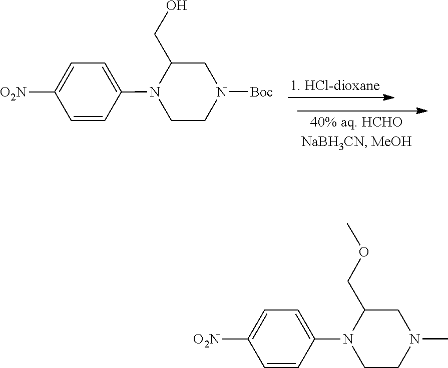 [SMILES: CC(C)(C)OC(=O)N1CCN(c2ccc([N+](=O)[O-])cc2)C(CO)C1 ; COCC1CN(C)CCN1c1ccc([N+](=O)[O-])cc1]

To a stirred solution of tert-butyl 3-(hydroxymethyl)-4-(4-nitrophenyl) piperazine-1-carboxylate (Preparation 20, Step 1) (400 mg, 1.186 mmol) in 1,4-dioxane (4 mL) was added 4M HCl in dioxane (4 mL) at 0-5° C. and the whole was stirred at 30° C. for 5 h. Progress of the reaction was monitored by LC-MS and after completion the solvent was evaporated to dryness to afford the intermediate (1-(4-nitrophenyl) piperazin-2-yl) methanol hydrochloride (432 g, crude) as a yellow solid which was dissolved in MeOH (5 mL) and formaldehyde 40% solution (0.66 mL, 8.851 mmol) added along with NaBH₃CN (200.23 mg, 3.186 mmol) at RT. The resulting mixture was stirred at RT for 18 h. Progress of the reaction was monitored by UPLC-MS and after completion the reaction mixture was diluted with water (20 mL) and extracted with 10% methanol in DCM solution (2×100 mL). The combined organic layers were dried over anhydrous sodium sulphate, filtered and concentrated under reduced pressure to afford the crude product which was purified by Combi-flash using 4% MeOH in DCM as eluent to afford the title compound (235 mg, 50% yield) as a pale brown sticky liquid. LCMS m/z: 266.09 [M+H].

### Step 3:4-(2-(Methoxymethyl)-4-methylpiperazin-1-yl) aniline

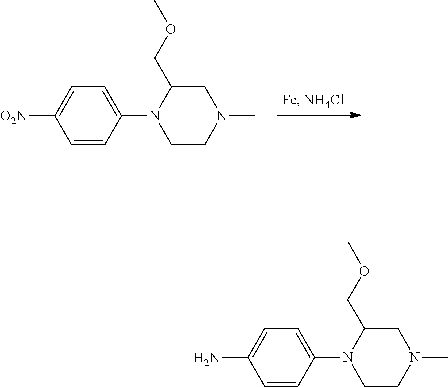 [SMILES: COCC1CN(C)CCN1c1ccc([N+](=O)[O-])cc1 ; COCC1CN(C)CCN1c1ccc(N)cc1]

To a stirred solution of 2-(methoxymethyl)-4-methyl-1-(4-nitrophenyl) piperazine (Preparation 20, Step 2) (200 mg, 0.754 mmol) in EtOH (8 mL) was added Fe powder (210.47 mg, 3.769 mmol) and aq. NH₄Cl solution (201.61 mg, 3.769 mmol in 1 ML water) at RT. The resulting reaction mixture was refluxed at 80° C. for 2 h. After completion of the reaction it was cooled to RT, diluted with water (20 mL) and extracted with 10% methanol in DCM solution (2×100 mL). The combined organic layers were dried over anhydrous sodium sulphate, filtered and concentrated under reduced pressure to afford the title compound (250 mg, crude) as a brown sticky solid which was used in the next step without further purification. LCMS m/z: 236.31 [M+H].

### Preparation 21:3,8-Dimethyl-2,3-dihydro-1H-pyrido[2,3-b][1,4]oxazin-7-amine. 2HCl

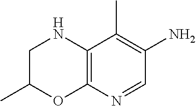

### Step 1: N-(5-Bromo-2-hydroxy-4-methylpyridin-3-yl)-2-chloropropanamide

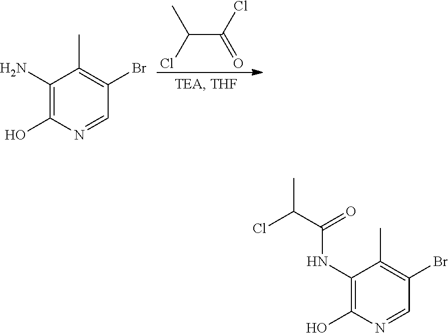

To a stirred solution of commercially available 3-amino-5-bromo-4-methylpyridin-2-ol (2000 mg, 9.852 mmol) in THF (50 mL) was added TEA (4.15 mL, 29.556 mmol) at 0-5° C. Thereafter 2-chloropropanoyl chloride (1.17 ml, 11.822 mmol) was added dropwise and the resulting reaction mixture was stirred at RT for 3 h. Progress of the reaction was monitored by TLC and LC-MS which showed conversion of the starting material to the desired product. The reaction mixture was diluted with water (200 mL) and extracted with 10% methanol in DCM solution (10×100 mL). The combined organic layers were dried over anhydrous sodium sulphate, filtered and concentrated under reduced pressure to afford the title compound (3.0 g, crude) as a pale white solid which was used in the next step without any further purification. LCMS m/z: 292.84 [M+H].

### Step 2:7-Bromo-3,8-dimethyl-1H-pyrido[2,3-b][1,4]oxazin-2 (3H)-one

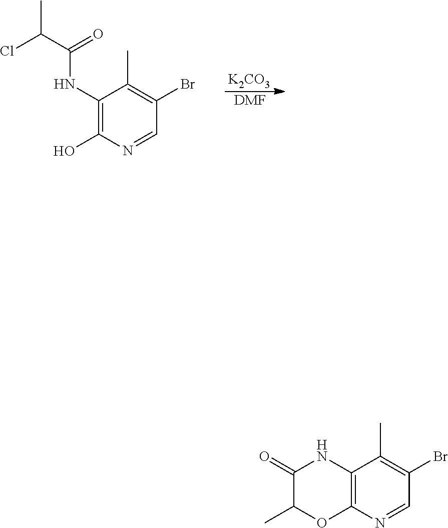

To a stirred solution of N-(5-bromo-2-hydroxy-4-methylpyridin-3-yl)-2-chloropropanamide (Preparation 21, Step 1) (6000 mg, 20.439 mmol) in DMF (60 mL) was added K₂CO₃ (8474 mg, 61.318 mmol) and the whole was stirred for a few mins. at room temperature, then heated at 70° C. overnight. Progress of the reaction was monitored by TLC and LC-MS which showed conversion of the starting material to the desired product. The reaction mixture was poured into chilled water (300 mL) and stirred for 30 mins. to produce a solid precipitate which was collected in a Buchner funnel and washed with hexane, suck dried for 30 mins. followed by oven drying overnight to afford the title compound (4.1 g, crude) as a pale brown solid which was used in the next step without further purification. LCMS m/z: 256.94 [M+H].

### Step 3:7-Bromo-3,8-dimethyl-2,3-dihydro-1H-pyrido[2,3-b][1,4]oxazine

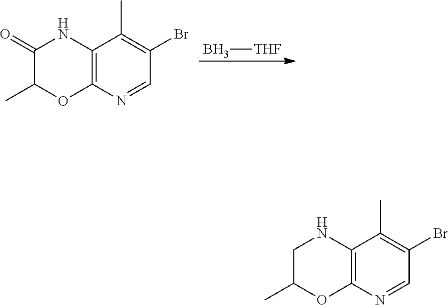

A stirred solution of 7-bromo-3,8-dimethyl-1H-pyrido[2,3-b][1,4]oxazin-2 (3H)-one (Preparation 21, Step 2) (2970 mg, 11.552 mmol) in BH₃. THF (46.2 mL, 1M solution in THF) was stirred at 0-5° C. for a few mins. then at room temperature for 3 h. After completion of the reaction (monitored by LC-MS and TLC) the reaction mixture was quenched by the dropwise addition of MeOH (100 mL) until the effervescence ceased followed by addition of 1N HCl (46.2 mL) and further stirred for 1 h. Next, MeOH was evaporated under reduced pressure to give a residue which was neutralized with an aqueous solution of saturated NaHCO₃. Then, it was diluted with water (200 mL) and extracted with EtOAc (4×200 mL). The combined organic layers were dried over anhydrous Na₂SO₄, filtered and evaporated in vacuo to afford the crude product which was purified by column chromatography on silica gel (80 g) using 5-7% ethyl acetate in DCM as eluent to afford the title compound (2.1 g, 75% yield) as a white solid. LCMS m/z: 242 [M+H].

### Step 4: tert-Butyl 7-bromo-3,8-dimethyl-2,3-dihydro-1H-pyrido[2,3-b][1,4]oxazine-1-carboxylate

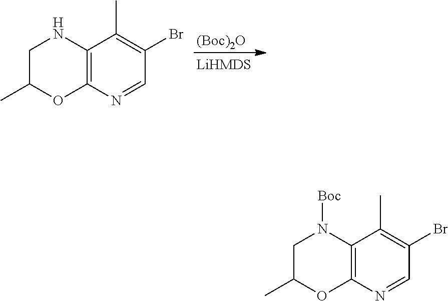 [SMILES: "(Boc)2O" ; Cc1c(Br)cnc2c1NCC(C)O2 ; Cc1c(Br)cnc2c1N(C(=O)OC(C)(C)C)CC(C)O2]

To a stirred solution of 7-bromo-3,8-dimethyl-2,3-dihydro-1H-pyrido[2,3-b][1,4]oxazine (Preparation 21, Step 3) (2100 mg, 8.638 mmol) in THF (42 mL) was added 1.3M LiHMDS (9.96 mL, 1.5 eq) at 0-5° C. under an inert atmosphere. Then Boc-anhydride (3.96 mL, 17.277 mmol) was added into the reaction vessel and stirring was continued at RT for 7 h. Progress of the reaction was monitored by TLC/LCMS and after completion the reaction mixture was diluted with water and extracted with EtOAc (8×100 mL). The combined organic layers were dried over anhydrous sodium sulphate, filtered and concentrated under reduced pressure to give the crude product which was purified by column chromatography using 25-30% EtOAc in hexane as eluent to afford the title compound (2.4 g, 81% yield) as a pale white solid. LCMS m/z: 342.92 [M+H].

### Step 5: tert-Butyl 7-((tert-butoxycarbonyl)amino)-3,8-dimethyl-2,3-dihydro-1H-pyrido[2,3-b][1,4]oxazine-1-carboxylate

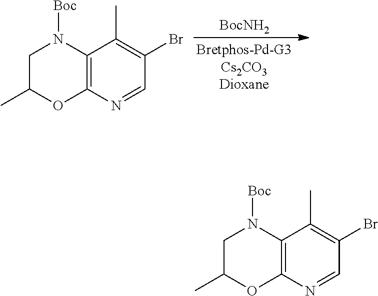

To a stirred solution of Boc-amine (1574 mg, 13.442 mmol) in 1,4-dioxane (46 mL) was added Cs₂CO₃ (6569 mg, 20.16 mmol) and tert-butyl 7-bromo-3,8-dimethyl-2,3-dihydro-1H-pyrido[2,3-b][1,4]oxazine-1-carboxylate (Preparation 21, Step 4) (2306 mg, 6.721 mmol) at RT. The resulting reaction mixture was purged with nitrogen for 20 mins. Thereafter, Brettphos-Pd-G3 (1218 mg, 1.344 mmol) was added into the reaction vessel and the whole was stirred at 105° C. for 3 h. Progress of the reaction was monitored by LCMS and after completion the reaction mixture was diluted with water (200 mL) and extracted with EtOAc (6×200 mL). The combined organic layers were washed with brine, dried over anhydrous Na₂SO₄, filtered and concentrated in vacuo to obtain the crude product which was purified by column chromatography using 25% EtOAc in DCM as eluent to afford the title compound (2100 mg, 82% yield) as a brown solid. LCMS m/z: 380.40 [M+H].

### Step 6:3,8-Dimethyl-2,3-dihydro-1H-pyrido[2,3-b][1,4]oxazin-7-amine. 2HCl

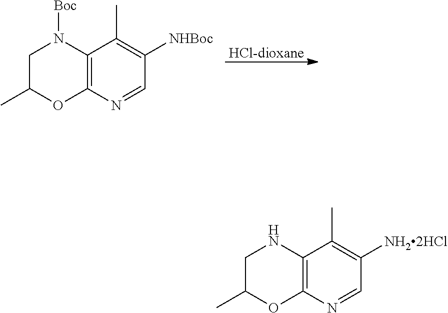

A solution of tert-butyl 7-((tert-butoxycarbonyl)amino)-3,8-dimethyl-2,3-dihydro-1H-pyrido[2,3-b][1,4]oxazine-1-carboxylate (Preparation 21, Step 5) (1900 mg, 5.013 mmol) in 2M HCl in dioxane (38 mL) was stirred at RT for 4 h. Progress of the reaction was monitored by LCMS and after completion the solvent was evaporated in vacuo to give a residue which was triturated with diethyl ether and dried to afford the title compound (1330 mg, crude) as a pale brown solid. LCMS m/z: 180.22 [M+H].

### Preparation 22, Step 1:4-Iodo-2-methoxy-N-(4-(2-(methoxymethyl)-4-methylpiperazin-1-yl)phenyl) nicotinamide

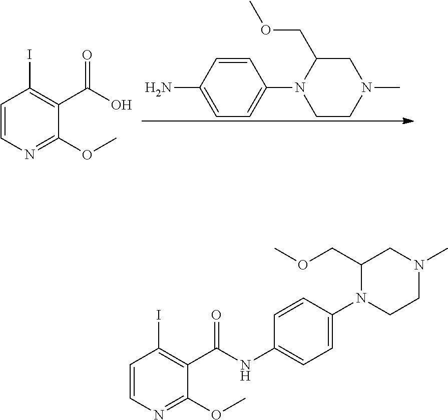 [SMILES: COc1nccc(I)c1C(=O)O ; COCC1CN(C)CCN1c1ccc(N)cc1 ; COCC1CN(C)CCN1c1ccc(NC(=O)c2c(I)ccnc2OC)cc1]

To a stirred solution of 4-iodo-2-methoxynicotinic acid (Preparation 11, Step 1) (280 mg, 1.004 mmol) in THF (10 mL) was added HBTU (456.82 mg, 1.205 mmol) and stirred at RT for 15 mins. Then DIPEA (0.525 ml, 3.011 mmol) and 4-(2-(methoxymethyl)-4-methylpiperazin-1-yl) aniline (Preparation 20, Step 3) (236.22 mg, 1.004 mmol) were added to the reaction vessel and the whole was further stirred at RT for 18 h. Progress of the reaction was monitored by LC-MS and after completion the reaction mixture was diluted with water (20 mL) and extracted with 10% methanol in DCM solution (2×100 mL). The combined organic layers were dried over anhydrous sodium sulphate, filtered and concentrated under reduced pressure to afford the crude product which was purified by Combi-flash using 4% MeOH in DCM as eluent to afford the title compound (155 mg, 37% yield) as a pale brown sticky solid. LCMS m/z: 497.16 [M+H].

### Step 2:4-((3,8-Dimethyl-2,3-dihydro-1H-pyrido[2,3-b][1,4]oxazin-7-yl)amino)-2-methoxy-N-(4-(2-(methoxymethyl)-4-methylpiperazin-1-yl)phenyl) nicotinamide

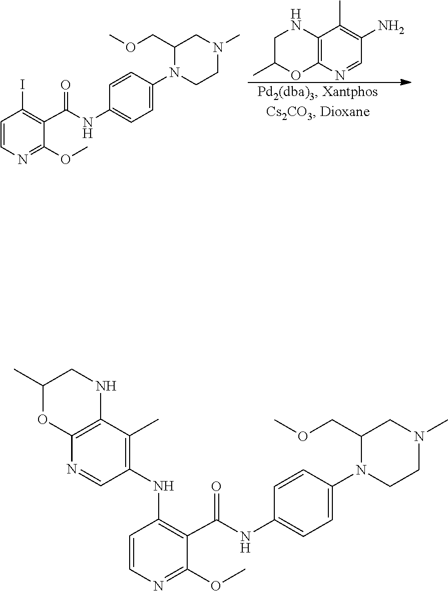

To a stirred solution of 4-iodo-2-methoxy-N-(4-(2-(methoxymethyl)-4-methylpiperazin-1-yl)phenyl) nicotinamide (Preparation 22, Step 1) (145 mg, 0.292 mmol) in 1,4-dioxane (10 mL) was added 3,8-dimethyl-2,3-dihydro-1H-pyrido[2,3-b][1,4]oxazin-7-amine. 2HCl (Preparation 21, Step 6) (62.827 mg, 0.351 mmol) followed by Cs₂CO₃ (428.32 mg, 1.315 mmol) at RT. This mixture was purged with nitrogen gas for 15 min. then Pd₂ (dba)₃ (26.75 mg,0.029 mmol) and Xantphos (33.81 mg, 0.058 mmol) were added into the reaction vessel and heated at 100° C. overnight. Progress of the reaction was monitored by TLC/LC-MS and after completion the reaction mixture was diluted with water (20 mL) and extracted with 10% methanol in DCM solution (3×100 mL). The combined organic layers were dried over anhydrous sodium sulphate, filtered and concentrated under reduced pressure to give the crude product which was purified by Combi-flash using 4-5% MeOH-DCM as eluent to afford the title compound (140 mg, 87% yield) as a brown sticky solid. LCMS m/z: 548.38 [M+H].

### Step 3:4-((3,8-Dimethyl-2,3-dihydro-1H-pyrido[2,3-b][1,4]oxazin-7-yl)amino)-N-(4-(2-(methoxymethyl)-4-methylpiperazin-1-yl)phenyl)-2-oxo-1,2-dihydropyridine-3-carboxamide (Example 197)

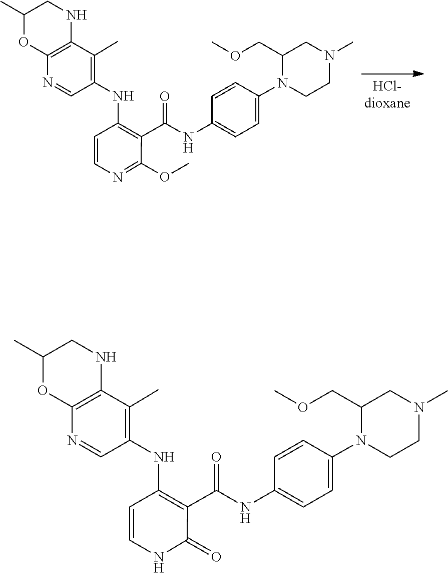

A stirred suspension of 4-((3,8-dimethyl-2,3-dihydro-1H-pyrido[2,3-b][1,4]oxazin-7-yl)amino)-2-methoxy-N-(4-(2-(methoxymethyl)-4-methylpiperazin-1-yl)phenyl) nicotinamide (Preparation 22, Step 2) (130 mg, 0.237 mmol) in 4M HCl in dioxane (5 mL) was stirred at 80° C. for 3 h. The progress of the reaction was monitored by LC-MS and after completion the reaction mixture was distilled off to obtain the crude product which was purified by prep-HPLC to afford the title compound (20 mg, 16% yield) as a white solid. HPLC purity: 99.84%; ¹H NMR (500 MHz; DMSO-d₆): δ 1.26 (d, J=6.25 Hz, 3H), 1.84-1.88 (m, 4H), 1.97-2.01 (m, 1H), 2.13-2.18 (m, 1H), 2.19 (s, 3H), 2.29-2.33 (m, 1H), 2.47-2.50 (m, 1H), 2.59-2.62 (m, 1H), 2.69-2.71 (m, 1H), 2.86-2.91 (m, 1H), 3.24 (s, 3H), 3.31-3.39 (m, 1H), 3.75-3.78 (m, 1H), 4.00-4.03 (m, 1H), 4.16-4.18 (m, 1H), 5.47-5.49 (m, 1H), 5.75 (s, 1H), 6.86 (d, J=8.95 Hz, 2H), 7.21-7.24 (m, 2H), 7.46 (d, J=8.95 Hz, 2H), 11.31 (s, 1H), 11.79 (s, 1H), 12.85 (s, 1H); LCMS m/z: 534.35 [M+H].

### Example 263: N-(3-Amino-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide

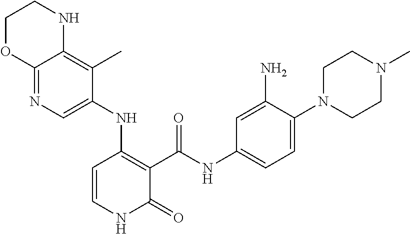 [SMILES: Cc1c(Nc2cc[nH]c(=O)c2C(=O)Nc2ccc(N3CCN(C)CC3)c(N)c2)cnc2c1NCCO2]

Example 263 was prepared according to the methods described in General Procedures 1-7, and the methods described below.

### Preparation 23, Step 1:4-Iodo-2-methoxy-N-(4-(4-methylpiperazin-1-yl)-3-nitrophenyl) nicotinamide

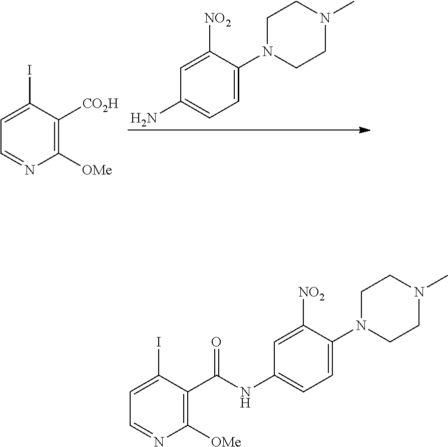 [SMILES: COc1nccc(I)c1C(=O)O ; CN1CCN(c2ccc(N)cc2[N+](=O)[O-])CC1 ; COc1nccc(I)c1C(=O)Nc1ccc(N2CCN(C)CC2)c([N+](=O)[O-])c1]

To a stirred solution of 4-iodo-2-methoxynicotinic acid (Preparation 11, Step 1) (200 mg, 0.71 mmol) in DCM (5 mL) was added thionyl chloride (0.10 mL, 1.41 mmol) and the whole was stirred at 45° C. for 1 h. Thereafter, the reaction was cooled to room temperature and concentrated in vacuo to obtain the crude corresponding acid chloride which was dissolved in DCM (5 mL) and DIPEA (0.38 mL, 2.14 mmol) added followed by a solution of commercially available 4-(4-methylpiperazin-1-yl)-3-nitro-aniline (202 mg, 0.85 mmol) in DCM (2 mL) dropwise at 0-5° C. The resulting reaction mixture was stirred at RT for 16 h. Progress of the reaction was monitored by LCMS and after completion the reaction mixture was quenched with water and extracted with DCM. The combined organic layers were washed with brine, dried over anhydrous sodium sulphate, filtered and concentrated under reduced pressure to obtain the crude product which was purified by column chromatography to afford the title compound (250 mg, 71% yield) as an off-white solid. LCMS m/z: 497.97 [M+H].

### Step 2:4-Chloro-N-(4-(4-methylpiperazin-1-yl)-3-nitrophenyl)-2-oxo-1,2-dihydropyridine-3-carboxamide

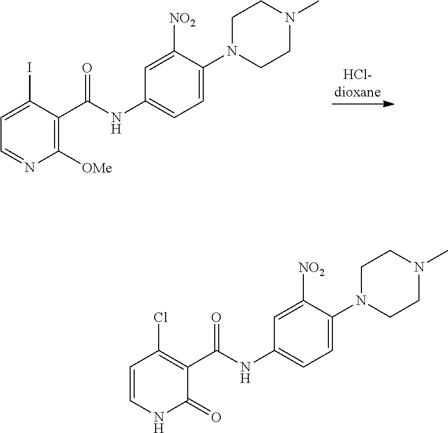 [SMILES: COc1nccc(I)c1C(=O)Nc1ccc(N2CCN(C)CC2)c([N+](=O)[O-])c1 ; CN1CCN(c2ccc(NC(=O)c3c(Cl)cc[nH]c3=O)cc2[N+](=O)[O-])CC1]

A mixture of 4-iodo-2-methoxy-N-(4-(4-methylpiperazin-1-yl)-3-nitrophenyl) nicotinamide (Preparation 23, Step 1) (250 mg, 0.503 mmol) in 4M HCl in dioxane (5 mL) was stirred at 80° C. for 2 h. Then the reaction was monitored by LCMS and after completion the reaction mixture was evaporated under reduced pressure to give a residue which was triturated with diethyl ether to afford the title compound (200 mg, crude) as a brown solid which was used in the next step without any further purification. LCMS m/z: 392.1 [M+H].

### Step 3:4-((8-Methyl-2,3-dihydro-1H-pyrido[2,3-b][1,4]oxazin-7-yl)amino)-N-(4-(4-methylpiperazin-1-yl)-3-nitrophenyl)-2-oxo-1,2-dihydropyridine-3-carboxamide

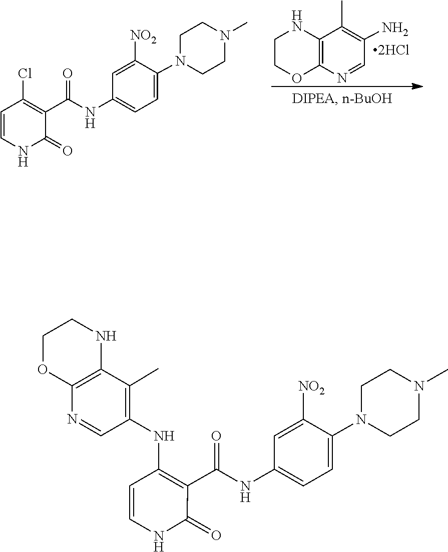

To a stirred solution of 4-chloro-N-(4-(4-methylpiperazin-1-yl)-3-nitrophenyl)-2-oxo-1,2-dihydropyridine-3-carboxamide (Preparation 23, Step 2) (200 mg, 0.51 mmol) in n-BuOH (4 mL) was added DIPEA (0.27 mL, 1.53 mmol) and 8-methyl-2,3-dihydro-1H-pyrido[2,3-b][1,4]oxazin-7-amine. 2HCl (Preparation 9, Step 4) (101 mg, 0.61 mmol) at RT. The resulting reaction mixture was stirred at 120° C. for 16 h in a sealed tube. The progress of the reaction was checked by LC-MS, thereafter the solvent was evaporated in vacuo to give the crude product which was purified by column chromatography on silica gel (100-200 mesh) to afford the title compound (130 mg, 48.9% yield) as an off-white solid. LCMS m/z: 521.2 [M+H].

### Preparation 24: N-(3-Amino-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide

### Example 263

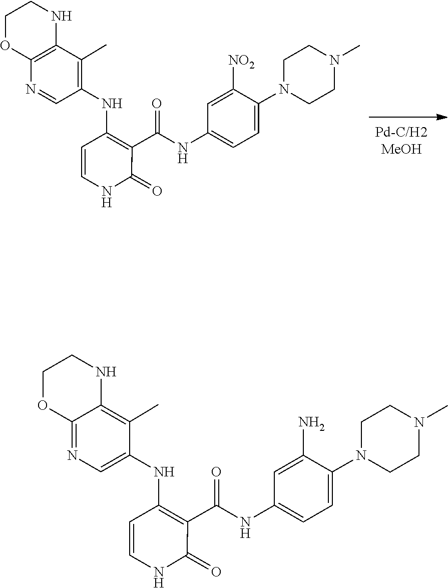 [SMILES: Cc1c(Nc2cc[nH]c(=O)c2C(=O)Nc2ccc(N3CCN(C)CC3)c([N+](=O)[O-])c2)cnc2c1NCCO2 ; Cc1c(Nc2cc[nH]c(=O)c2C(=O)Nc2ccc(N3CCN(C)CC3)c(N)c2)cnc2c1NCCO2]

To a stirred solution of 4-((8-methyl-2,3-dihydro-1H-pyrido[2,3-b][1,4]oxazin-7-yl)amino)-N-(4-(4-methylpiperazin-1-yl)-3-nitrophenyl)-2-oxo-1,2-dihydropyridine-3-carboxamide (Preparation 23, Step 3) (100 mg, 0.19 mmol) in methanol (5 mL) was added 10% Pd—C(30 mg) under an inert atmosphere and the whole was stirred under hydrogen balloon pressure for 2 h. Progress of the reaction was monitored by LC-MS and after completion, the reaction mixture was filtered through a short celite bed and the bed washed with 10% MeOH-DCM. The filtrate was then concentrated under reduced pressure to obtain the crude compound which was purified by prep-HPLC to afford the title compound (13 mg, 14% yield) as an off-white solid. HPLC purity: 99.82%; ¹H NMR (400 MHZ; DMSO-d₆): δ 1.92 (s, 3H), 2.22 (s, 3H), 2.76 (s, 4H), 3.31 (s, 4H), 4.25 (s, 2H), 4.77 (s, 2H), 5.55 (d, J=7.4 Hz, 1H), 5.79 (s, 1H), 6.77-6.85 (m, 2H), 7.03 (d, J=2.08 Hz, 1H), 7.24-7.30 (m, 2H), 11.33 (d, J=6.08 Hz, 1H), 11.93 (s, 1H), 12.77 (s, 1H); LCMS m/z: 491.34 [M+H].

### Example 132:4-((8-Methoxy-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide

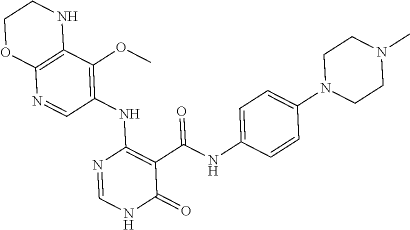 [SMILES: COc1c(Nc2nc[nH]c(=O)c2C(=O)Nc2ccc(N3CCN(C)CC3)cc2)cnc2c1NCCO2]

Example 132 was prepared according to the methods described in General Procedures 1-7, and the methods described below.

### Preparation 25: tert-Butyl 7-bromo-8-methoxy-2,3-dihydro-1H-pyrido[2,3-b][1,4]oxazine-1-carboxylate

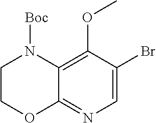 [SMILES: COc1c(Br)cnc2c1N(C(=O)OC(C)(C)C)CCO2]

### Step 1:5-Bromo-2-chloro-4-methoxy-3-nitropyridine

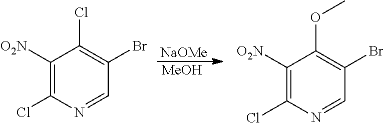

To a stirred solution of commercially available 5-bromo-2,4-dichloro-3-nitropyridine (1650 mg, 6.069 mol) in MeOH (20 mL) was added NaOMe (327.84 mg, 6.069 mmol) at RT and the combined reaction mixture was stirred at 60° C. for 2 h. Progress of the reaction was monitored by TLC and LCMS and after completion the solvent was evaporated in vacuo to dryness to obtain a residue which was purified by Combi-flash over silica gel using ethyl acetate and hexane as eluent to afford the title compound (1550 mg, 95% yield) as a yellowish liquid.

### Step 2: Methyl 2-((5-bromo-4-methoxy-3-nitropyridin-2-yl)oxy)acetate

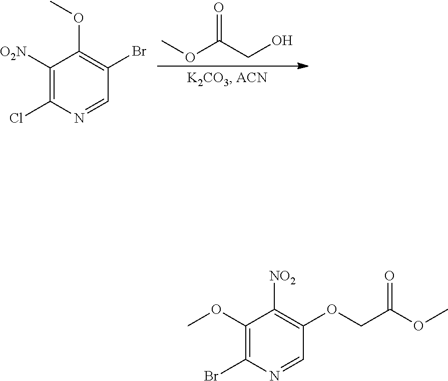

To a stirred solution of methyl 2-hydroxyacetate (3132.22 mg, 34.772 mmol) in acetonitrile (60 mL) was added K₂CO₃ (4805.78 mg, 34.772 mmol) and after 10 mins. 5-bromo-2-chloro-4-methoxy-3-nitropyridine (Preparation 25, Step 1) (3100 mg, 11.591 mmol) was added into the reaction mixture at RT. The resulting mixture was stirred at 80° C. overnight. Progress of the reaction was monitored by TLC and LCMS and after completion it was diluted with water and extracted with ethyl acetate. The combined organic layers were evaporated in vacuo to dryness to obtain the crude compound which was purified by Combi-flash over silica gel using ethyl acetate and hexane as eluent to afford the title compound (360 mg, 10% yield) as a yellowish sticky solid. LCMS m/z: 320.98 [M+H].

### Step 3:7-Bromo-8-methoxy-1H-pyrido[2,3-b][1,4]oxazin-2 (3H)-one

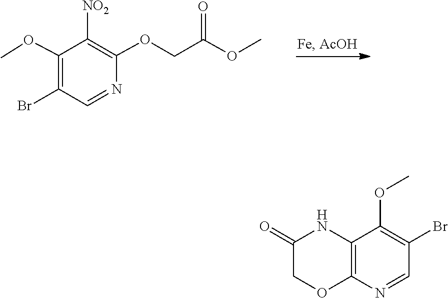

To a stirred solution of methyl 2-((5-bromo-4-methoxy-3-nitropyridin-2-yl)oxy)acetate (Preparation 25, Step 2) (360 mg, 1.121 mmol) in AcOH (5 mL) was added Fe powder (250.43 mg, 4.484 mmol) at RT, thereafter the reaction mixture was refluxed at 90° C. for 1.5 h. LCMS and TLC showed the desired product had formed. The acetic acid was evaporated in vacuo to give a residue which was neutralized with a saturated solution of sodium bicarbonate and diluted with water. The neutralised aqueous mass was extracted with ethyl acetate. The combined organic layers were washed with brine, dried over anhydrous sodium sulphate, filtered and concentrated under vacuum to obtain the title compound (300 mg, crude) as a white solid which was used in the next step without any further purification. LCMS m/z: 258.94 [M+H].

### Step 4:7-Bromo-8-methoxy-2,3-dihydro-1H-pyrido[2,3-b][1,4]oxazine

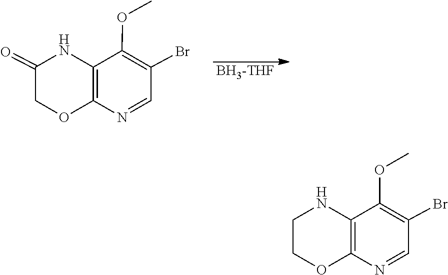

A suspension of 7-bromo-8-methoxy-1H-pyrido[2,3-b][1,4]oxazin-2 (3H)-one (Preparation 25, Step 3) (300 mg, 1.158 mmol) in 0.9M BH₃-THF solution (6.43 mL, 5.790 mmol) at 0-5° C. was stirred at RT for 2 h in a Reactive-vial. Progress of the reaction was monitored by TLC and LCMS, and after completion the reaction mass was quenched with methanol followed by 1N HCl (0.2 mL). After 1 h, it was evaporated in vacuo to dryness which was neutralized with a saturated solution of sodium bicarbonate and diluted with water. The neutralised aqueous mass was extracted with ethyl acetate. The combined organic layers were washed with brine, dried over anhydrous sodium sulphate, filtered and concentrated under vacuum to afford the title compound (360 mg, crude) as a yellowish sticky liquid which was used in the next step without any further purification. LCMS m/z: 244.96 [M+H].

### Step 5: tert-Butyl 7-bromo-8-methoxy-2,3-dihydro-1H-pyrido[2,3-b][1,4]oxazine-1-carboxylate

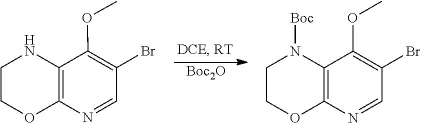 [SMILES: "Boc2O" ; COc1c(Br)cnc2c1NCCO2 ; COc1c(Br)cnc2c1N(C(=O)OC(C)(C)C)CCO2]

To a stirred solution of 7-bromo-8-methoxy-2,3-dihydro-1H-pyrido[2,3-b][1,4]oxazine (Preparation 25, Step 4) (360 mg, 1.468 mmol) in DCE (10 mL) was added TEA (1.02 mL, 7.340 mmol), DMAP (179.34 mg, 1.468 mmol) and Boc-anhydride (1.349 mL, 5.878 mmol) at RT. The resulting reaction mixture was stirred at RT for 6 h. Progress of the reaction was monitored by TLC and LCMS, and after completion the reaction mass was diluted with water and extracted with ethyl acetate. The combined organic layers were distilled off to obtain the crude product which was purified by Combi-flash over silica gel using ethyl acetate and hexane as eluent to afford the title compound (300 mg, 59% yield) as a yellowish sticky solid. LCMS m/z: 345.06 [M+H].

### Preparation 26:4-Amino-2-methoxy-N-(4-(4-methylpiperazin-1-yl)phenyl) nicotinamide

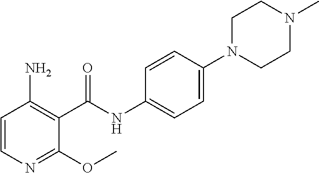 [SMILES: COc1nccc(N)c1C(=O)Nc1ccc(N2CCN(C)CC2)cc1]

### Step 1: tert-Butyl (2-methoxy-3-((4-(4-methylpiperazin-1-yl)phenyl) carbamoyl)pyridin-4-yl) carbamate

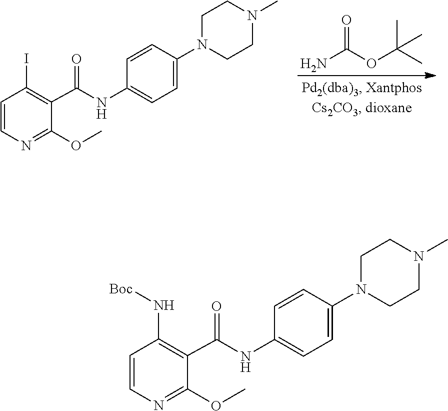

To a stirred solution of 4-iodo-2-methoxy-N-(4-(4-methylpiperazin-1-yl)phenyl) nicotinamide (Preparation 1, Step 2) (353 mg, 0.780 mmol) in dioxane (10 mL) was added tert-butyl carbamate (182.78 mg, 1.560 mmol) followed by Cs₂CO₃ (635.3 mg, 1.949 mmol) at RT, The resulting reaction mixture was purged with nitrogen gas for 15 mins. Then, Pd₂ (dba)₃ (71.42 mg,0.077 mmol) and Xantphos (90.26 mg,0.155 mmol) were added into the reaction vessel which was heated at 105° C. overnight. The progress of the reaction was monitored by TLC and LCMS and after completion the reaction mixture was diluted with water and extracted with 10% MeOH in DCM solution (5×100 mL). The combined organic layers were dried over anhydrous sodium sulphate, filtered and concentrated under reduced pressure to afford the crude compound which was purified by Combi-flash on a silica gel column (12 g) using 3-4% MeOH in DCM as eluent to yield the title compound (315 mg, 49% yield) as a sticky solid. LCMS m/z: 442.55 [M+H].

### Step 2:4-Amino-2-methoxy-N-(4-(4-methylpiperazin-1-yl)phenyl) nicotinamide

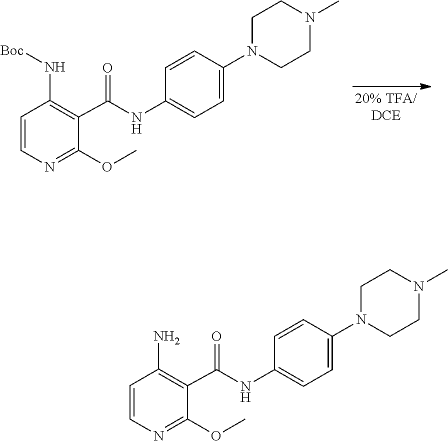 [SMILES: COc1nccc(NC(=O)OC(C)(C)C)c1C(=O)Nc1ccc(N2CCN(C)CC2)cc1 ; COc1nccc(N)c1C(=O)Nc1ccc(N2CCN(C)CC2)cc1]

To a stirred solution of tert-butyl (2-methoxy-3-((4-(4-methylpiperazin-1-yl)phenyl) carbamoyl)pyridin-4-yl) carbamate (Preparation 26, Step 1) (300 mg, 0.679 mmol) in DCE (6 mL) was added TFA (1.2 mL) with ice cooling and the combined mixture was stirred for a few mins. Before being allowed to warm slowly to room temperature over 2 h. Progress of the reaction was monitored by TLC and LCMS which showed conversion of the starting material to the desired product along with the de-methylated product as a minor impurity. The reaction mixture was diluted with water and neutralized with a saturated sodium bicarbonate solution. The product was extracted with 10% MeOH in DCM (3×100 mL) and the combined organic layers were dried over anhydrous sodium sulphate, filtered and concentrated under reduced pressure to give the crude product which was purified by Combi-flash on a silica gel column (12 g) using 7-9% MeOH in DCM as eluent to afford the title compound (191 mg, 82% yield) as a yellow sticky solid. LCMS m/z: 342.13 [M+H].

### Preparation 27, Step 1: tert-Butyl 8-methoxy-7-((2-methoxy-3-((4-(4-methylpiperazin-1-yl)phenyl) carbamoyl)pyridin-4-yl)amino)-2,3-dihydro-1H-pyrido[2,3-b][1,4]oxazine-1-carboxylate

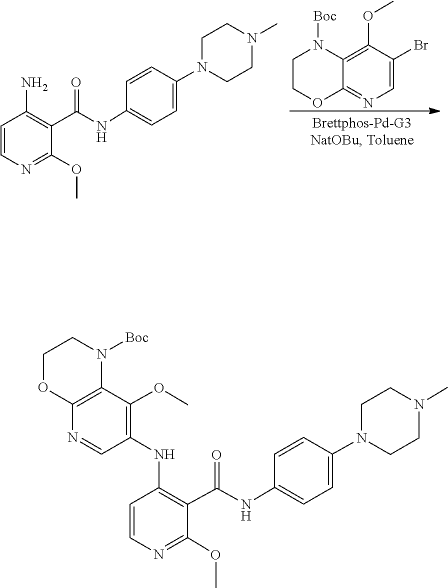

To a stirred solution of 4-amino-2-methoxy-N-(4-(4-methylpiperazin-1-yl)phenyl) nicotinamide (Preparation 26, Step 2) (80 mg, 0.234 mmol) in toluene (10 mL) was added tert-butyl 7-bromo-8-methoxy-2,3-dihydro-1H-pyrido[2,3-b][1,4]oxazine-1-carboxylate (Preparation 25, Step 5) (96.80 mg, 0.281 mmol) and NaO′Bu (56.33 mg, 0.586 mmol) at RT. The resulting reaction mixture was purged with nitrogen for 15 mins. after which BrettPhos-Pd-G3 (42.51 mg, 0.047 mmol) was added into the reaction vessel and the whole heated at 100° C. overnight. Progress of the reaction was checked by LCMS and after completion the reaction mass was diluted with water and extracted with 10% methanol in DCM. The combined organic layers were distilled off to obtain the crude compound which was purified by Combi-flash over silica gel to afford the title compound (200 mg, crude) as a brown sticky liquid. LCMS m/z: 606.27 [M+H].

### Step 2:4-((8-Methoxy-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide (Example 132)

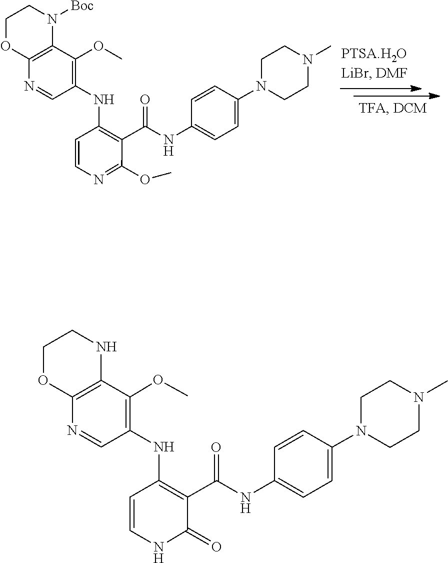 [SMILES: COc1nccc(Nc2cnc3c(c2OC)N(C(=O)OC(C)(C)C)CCO3)c1C(=O)Nc1ccc(N2CCN(C)CC2)cc1 ; COc1c(Nc2cc[nH]c(=O)c2C(=O)Nc2ccc(N3CCN(C)CC3)cc2)cnc2c1NCCO2]

To a stirred solution of tert-butyl 8-methoxy-7-((2-methoxy-3-((4-(4-methylpiperazin-1-yl)phenyl) carbamoyl)pyridin-4-yl)amino)-2,3-dihydro-1H-pyrido[2,3-b][1,4]oxazine-1-carboxylate (Preparation 27, Step 1) (30 mg, 0.049 mmol) in DMF (3 mL) was added PTSA.H₂O (9.41 mg,0.049 mmol) followed by LiBr (4.25 mg, 0.049 mmol). The whole was heated at 65° C. for 40 mins. LCMS showed formation of the desired mass. After completion the reaction mixture was diluted with water and extracted with 10% MeOH in DCM. The combined organic layers were evaporated under reduced pressure to afford the crude de-methylated compound tert-butyl 8-methoxy-7-((2-methoxy-3-((4-(4-methylpiperazin-1-yl)phenyl) carbamoyl)pyridin-4-yl)amino)-2,3-dihydro-1H-pyrido[2,3-b][1,4]oxazine-1-carboxylate (66 mg, crude) as a brown sticky solid which was suspended in 20% TFA in DCM (2 mL) and stirred at RT for 3 h under an inert atmosphere. Progress of the reaction was monitored by LCMS and after completion the solvent was evaporated under reduced pressure to give the crude product which was purified by prep-HPLC to afford the title compound (2.8 mg, 11% yield) as a white solid. HPLC purity: 95.24%; 1H NMR (500 MHz; DMSO-d₆): δ 2.17 (s, 3H), 2.43 (s, 4H), 3.03 (s, 4H), 3.22 (d, J=3.7 Hz, 2H), 3.62 (s, 3H), 4.21 (t, J=3.85 Hz, 2H), 5.68 (d, J=7.1 Hz, 1H), 5.84 (s, 1H), 6.85 (d, J=9.05 Hz, 2H), 7.23-7.26 (m, 2H), 7.40 (d, J=9.0 Hz, 2H), 11.35 (d, J=5.95 Hz, 1H), 11.93 (s, 1H), 12.80 (s, 1H); LCMS m/z: 492.25 [M+H].

### Example 163: N-(2-Methyl-1,2,3,4-tetrahydroisoquinolin-7-yl)-4-((8-methyl-2,3-dihydro-1H-pyrido[2,3-b][1,4]oxazin-7-yl)amino)-2-oxo-1,2-dihydropyridine-3-carboxamide

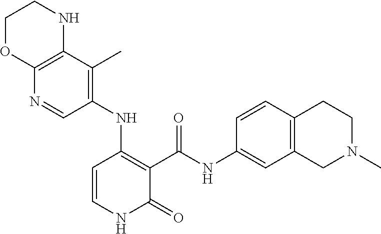 [SMILES: Cc1c(Nc2cc[nH]c(=O)c2C(=O)Nc2ccc3c(c2)CN(C)CC3)cnc2c1NCCO2]

Example 132 was prepared according to the methods described in General Procedures 1-7, and the methods described below.

### Preparation 28:2-Methyl-1,2,3,4-tetrahydroisoquinolin-7-amine.TFA

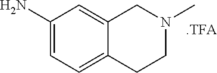

### Step 1:7-Bromo-1,2,3,4-tetrahydroisoquinoline

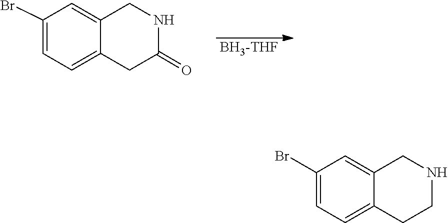 [SMILES: O=C1Cc2ccc(Br)cc2CN1 ; "BH3-THF" ; Brc1ccc2c(c1)CNCC2]

A suspension of commercially available 7-bromo-1,4-dihydroisoquinolin-3 (2H)-one (340 mg, 1.503 mmol) in 0.9M BH₃-THF solution (5.01 mL, 4.511 mmol) at 0-5° C. was stirred at RT for 1 h and then heated at 60° C. for 1 h. The progress of the reaction was monitored by TLC and LCMS and after completion it was quenched with methanol followed by IN HCl and neutralized with a saturated solution of sodium bicarbonate. The aqueous mass was extracted with 10% methanol in DCM and the combined organic layers were concentrated in vacuo to give a crude compound which was purified over silica gel using methanol in DCM as eluent to afford the title compound (460 mg, crude) as a yellowish solid. LCMS m/z: 211.92 [M+H].

### Step 2:7-Bromo-2-methyl-1,2,3,4-tetrahydroisoquinoline

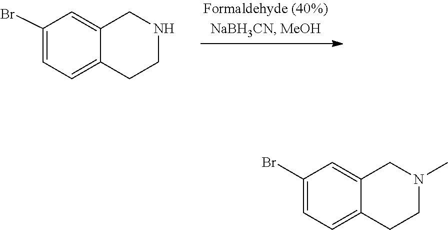

To a stirred solution of 7-bromo-1,2,3,4-tetrahydroisoquinoline (Preparation 28, Step 1) (460 mg, 1.428 mmol) in MeOH (10 mL) was added a 40% solution of formaldehyde (0.813 mL, 10.844 mmol) and NaBH₃CN (245.33 mg, 3.904 mmol) at RT. After the addition was complete, the whole was stirred at RT for a further 16 h. After completion of the reaction (checked by LCMS) the solvent was evaporated in vacuo to give a residue which was purified by Combi-flash using 5% MeOH in DCM to afford the title compound (475 mg, 97% yield) as a yellowish sticky solid. LCMS m/z: 225.92 [M+H].

### Step 3: N-(4-Methoxybenzyl)-2-methyl-1,2,3,4-tetrahydroisoquinolin-7-amine

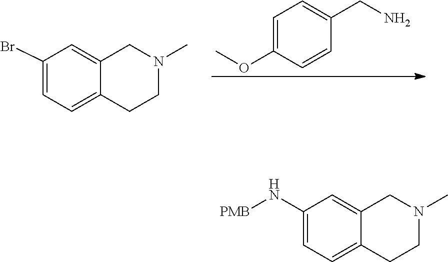

To a stirred solution of 7-bromo-2-methyl-1,2,3,4-tetrahydroisoquinoline (Preparation 28, Step 2) (475 mg, 2.100 mmol) in 1,4-dioxane (10 mL) was added (4-methoxyphenyl) methanamine (0.411 mL, 3.150 mmol) and NaO′Bu (605.43 mg, 6.300 mmol) at RT. The resulting mixture was purged with nitrogen gas for 10 mins. Then Pd₂ (dba)₃ (192.36 mg, 0.210 mmol) and Brettphos (225.44 mg, 0.420 mmol) were added to the reaction mixture and the whole was heated at 100° C. for 18 h. Progress of the reaction was monitored by LCMS and after completion it was diluted with water and extracted with 10% methanol in DCM. The combined organic layers were distilled off to obtain the crude compound which was purified on silica gel using methanol in DCM as eluent to afford the title compound (355 mg, 60% yield) as a brown solid. LCMS m/z: 282.86 [M+H].

### Step 4:2-Methyl-1,2,3,4-tetrahydroisoquinolin-7-amine.TFA

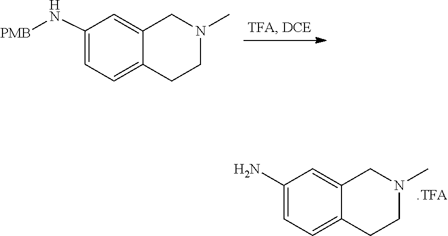

To a stirred solution of N-(4-methoxybenzyl)-2-methyl-1,2,3,4-tetrahydroisoquinolin-7-amine (Preparation 28, Step 3) (345 mg, 1.221 mmol) in DCE (5 mL) was added TFA (1 mL) at 0-5° C. and the reaction mixture was stirred at RT for 3 h. The reaction was monitored by LCMS and after completion the solvent was evaporated in vacuo to afford the title compound (500 mg, crude) as a blackish sticky solid which was used in the next step without any further purification. LCMS m/z: 163.27 [M+H].

### Preparation 29, Step 1:4-Iodo-2-methoxy-N-(2-methyl-1,2,3,4-tetrahydroisoquinolin-7-yl) nicotinamide

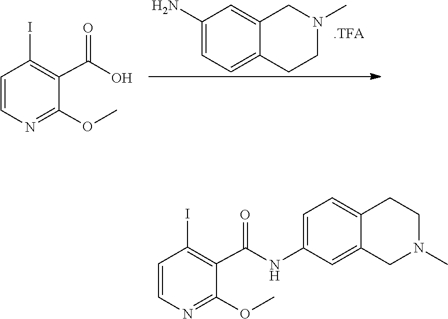 [SMILES: COc1nccc(I)c1C(=O)O ; CN1CCc2ccc(N)cc2C1 ; COc1nccc(I)c1C(=O)Nc1ccc2c(c1)CN(C)CC2]

To a stirred solution of 4-iodo-2-methoxynicotinic acid (Preparation 11, Step 1) (280 mg, 1.003 mmol) in THF (10 mL) was added HATU (457.97 mg, 1.204 mmol) and DIPEA (0.7 mL, 4.012 mmol) followed by 2-methyl-1,2,3,4-tetrahydroisoquinolin-7-amine.TFA (Preparation 28, Step 4) (195.27 mg, 1.204 mmol) at RT. The resulting reaction mixture was stirred at RT for 3 h. After completion of the reaction (checked by LCMS) the reaction mass was diluted with water and extracted with 10% methanol in DCM. The combined organic layers were concentrated in vacuo to give a crude compound which was purified on silica gel using methanol in DCM as eluent to afford the title compound (185 mg) as a brown solid. LCMS m/z: 424.06 [M+H].

### Step 2:4-Chloro-N-(2-methyl-1,2,3,4-tetrahydroisoquinolin-7-yl)-2-oxo-1,2-dihydropyridine-3-carboxamide.HCl

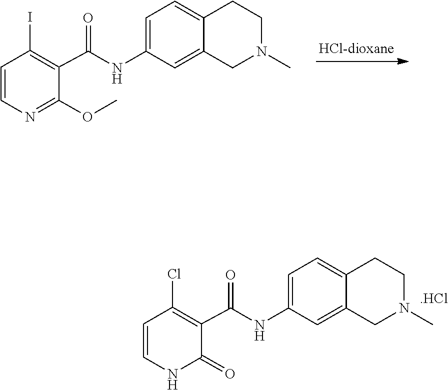 [SMILES: COc1nccc(I)c1C(=O)Nc1ccc2c(c1)CN(C)CC2 ; CN1CCc2ccc(NC(=O)c3c(Cl)cc[nH]c3=O)cc2C1 ; ".HCl"]

A stirred suspension of 4-iodo-2-methoxy-N-(2-methyl-1,2,3,4-tetrahydroisoquinolin-7-yl) nicotinamide (Preparation 29, Step 1) (165 mg, 0.390 mmol) in 4M HCl in dioxane (5 mL) was stirred at 80° C. for 18 h. The progress of the reaction was monitored by LCMS and after completion the solvent was evaporated in vacuo to afford the title compound (135 mg, crude) as a brown semi solid which was used in the next step without any further purification. LCMS m/z: 318.07 [M+H].

### Step 3: N-(2-Methyl-1,2,3,4-tetrahydroisoquinolin-7-yl)-4-((8-methyl-2,3-dihydro-1H-pyrido[2,3-b][1,4]oxazin-7-yl)amino)-2-oxo-1,2-dihydropyridine-3-carboxamide

### Example 163

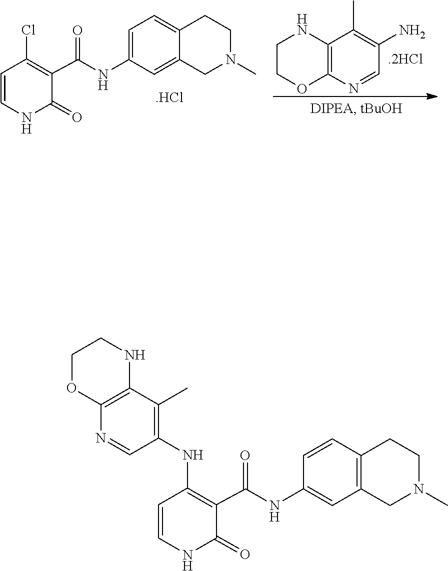

To a stirred suspension of 4-chloro-N-(2-methyl-1,2,3,4-tetrahydroisoquinolin-7-yl)-2-oxo-1,2-dihydropyridine-3-carboxamide.HCl (Preparation 29, Step 2) (125 mg, 0.393 mmol) in t-BuOH (5 mL) was added 8-methyl-2,3-dihydro-1H-pyrido[2,3-b][1,4]oxazin-7-amine dihydrochloride (Preparation 9, Step 4) (77.98 mg, 0.472 mmol) followed by DIPEA (0.35 mL, 1.965 mmol) at RT. The resulting reaction mixture was heated at 80° C. for 18 h. The progress of the reaction was monitored by LCMS and after completion the reaction mass was diluted with water and extracted with 10% methanol in DCM. The combined organic layers were evaporated in vacuo to give a crude product which was purified on silica gel using methanol in DCM as eluent followed by prep-HPLC to afford the title compound (8 mg, 5% yield) as a faint brown solid. HPLC purity: 99.18%; ¹H NMR (500 MHz; DMSO-d₆): δ 1.85 (s, 3H), 2.25 (s, 3H), 2.43 (s, 2H merged in DMSO), 2.50 (t, J=5.65 Hz, 2H), 2.70 (t, J=5.25 Hz, 2H), 3.38 (s, 2H), 4.19 (s, 2H), 5.47 (d, J=7.45 Hz, 1H), 5.75 (s, 1H), 6.98 (d, J=8.7 Hz, 1H), 7.22 (d, J=9.95 Hz, 2H), 7.28 (d, J=6.75 Hz, 2H), 11.34 (s, 1H), 11.76 (s, 1H), 12.92 (s, 1H); LCMS m/z: 445.21 [M−H].

### Example 315: N-(4-(4-Isobutyrylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide

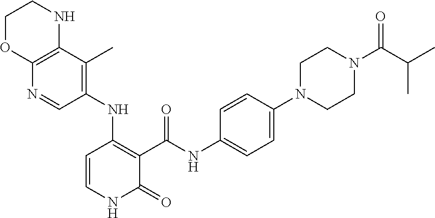 [SMILES: Cc1c(Nc2cc[nH]c(=O)c2C(=O)Nc2ccc(N3CCN(C(=O)C(C)C)CC3)cc2)cnc2c1NCCO2]

Example 315 was prepared according to the methods described in General Procedures 1-7, and the methods described below.

### Preparation 30:4-((8-Methyl-2,3-dihydro-1H-pyrido[2,3-b][1,4]oxazin-7-yl)amino)-2-oxo-N-(4-(piperazin-1-yl)phenyl)-1,2-dihydropyridine-3-carboxamide

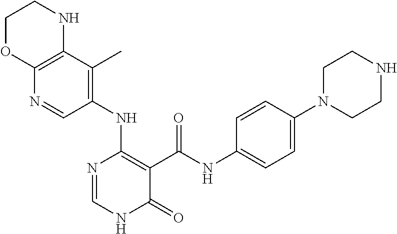 [SMILES: Cc1c(Nc2nc[nH]c(=O)c2C(=O)Nc2ccc(N3CCNCC3)cc2)cnc2c1NCCO2]

### Step 1: tert-Butyl 4-(4-(4-iodo-2-methoxynicotinamido)phenyl) piperazine-1-carboxylate

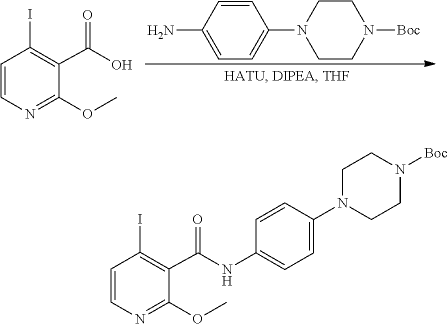 [SMILES: COc1nccc(I)c1C(=O)O ; CC(C)(C)OC(=O)N1CCN(c2ccc(N)cc2)CC1 ; COc1nccc(I)c1C(=O)Nc1ccc(N2CCN(C(=O)OC(C)(C)C)CC2)cc1]

To a stirred solution of 4-iodo-2-methoxynicotinic acid (Preparation 11, Step 1) (2000 mg, 7.168 mmol) in THF (30 mL) was added HATU (4088.17 mg, 10.75 mmol) followed by DIPEA (3.68 mL, 21.50 mmol) and after 10 mins. commercially available tert-butyl 4-(4-aminophenyl) piperazine-1-carboxylate (1789.56 mg, 6.451 mmol) was added into the reaction vessel. The whole was allowed to stir at RT for 12 h. UPLC and TLC showed that the desired product had formed and the starting materials were fully consumed. The reaction mixture was diluted with water and extracted with 15% methanol in DCM. The combined organic layers were washed with brine, dried over anhydrous Na₂SO₄, filtered through a celite pad and the filtrate was evaporated in vacuo to obtain the crude product which was purified by Combi-flash (40 g column) using 60% ethyl acetate in hexane as eluent to afford the title compound (3220 mg, 78% yield) as a black solid. LCMS m/z: 539.16 [M+H].

### Step 2: tert-Butyl 4-(4-(2-methoxy-4-((8-methyl-2,3-dihydro-1H-pyrido[2,3-b][1,4]oxazin-7-yl)amino) nicotinamido)phenyl) piperazine-1-carboxylate

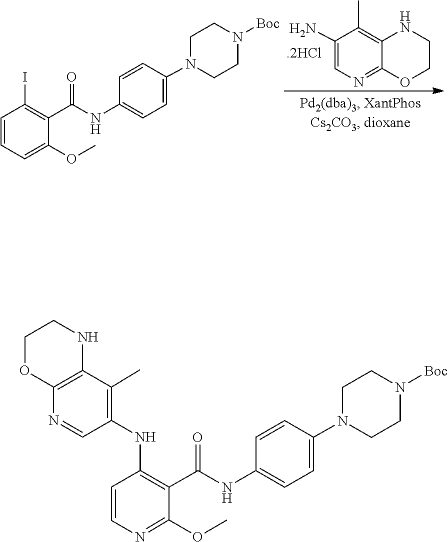 [SMILES: COc1cccc(I)c1C(=O)Nc1ccc(N2CCN(C(=O)OC(C)(C)C)CC2)cc1 ; Cc1c(N)cnc2c1NCCO2 ; COc1nccc(Nc2cnc3c(c2C)NCCO3)c1C(=O)Nc1ccc(N2CCN(C(=O)OC(C)(C)C)CC2)cc1]

To a stirred solution of tert-butyl 4-(4-(4-iodo-2-methoxynicotinamido)phenyl) piperazine-1-carboxylate (Preparation 30, Step 1) (153.28 mg, 0.929 mmol) in 1,4-dioxane (15 mL) was added Cs₂CO₃ (1514.03 mg, 4.647 mmol) followed by 8-methyl-2,3-dihydro-1H-pyrido[2,3-b][1,4]oxazin-7-amine dihydrochloride (Preparation 9, Step 4) (500 mg, 0.929 mmol) at RT. The resulting reaction mixture was purged with nitrogen gas for 15 mins. then Pd₂ (dba)₃ (85.06 mg, 0.0929 mmol) and XantPhos (107.56 mg, 0.1859 mmol) were added into the reaction vessel. The whole was allowed to stir at 100° C. overnight under an inert atmosphere. LCMS and TLC showed that the desired compound had formed and the starting materials were fully consumed. The reaction mass was cooled to RT, diluted with water and extracted with 5-6% methanol in DCM. The combined organic layers were dried over anhydrous Na₂SO₄, filtered through a celite bed and the filtrate was evaporated in vacuo to obtain a crude product which was purified by Combi-flash (80 g column) using 10% methanol in DCM as eluent to afford the title compound (330 mg, 63% yield) as a yellow solid. LCMS m/z: 576.67 [M+H].

### Step 3:4-((8-Methyl-2,3-dihydro-1H-pyrido[2,3-b][1,4]oxazin-7-yl)amino)-2-oxo-N-(4-(piperazin-1-yl)phenyl)-1,2-dihydropyridine-3-carboxamide.HCl

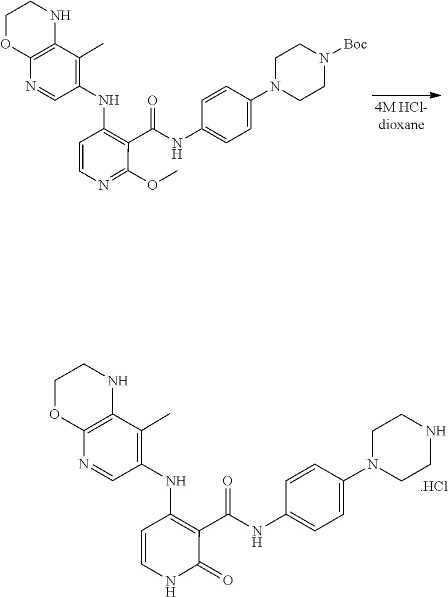 [SMILES: COc1nccc(Nc2cnc3c(c2C)NCCO3)c1C(=O)Nc1ccc(N2CCN(C(=O)OC(C)(C)C)CC2)cc1 ; Cc1c(Nc2cc[nH]c(=O)c2C(=O)Nc2ccc(N3CCNCC3)cc2)cnc2c1NCCO2 ; ".HCl"]

A suspension of tert-butyl 4-(4-(2-methoxy-4-((8-methyl-2,3-dihydro-1H-pyrido[2,3-b][1,4]oxazin-7-yl)amino) nicotinamido)phenyl) piperazine-1-carboxylate (Preparation 30, Step 2) (500 mg, 0.869 mmol) in 4M HCl in dioxane (10 mL) was stirred at 80° C. for 3 h. UPLC and TLC showed that the desired mass had formed and the starting material was fully consumed. The solvent from the reaction mixture was evaporated in vacuo to give a crude product which was triturated with diethyl ether to afford the title compound (570 mg, crude) as a yellow solid. LCMS m/z: 462.47 [M+H].

### Preparation 31: N-(4-(4-Isobutyrylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide

### Example 315

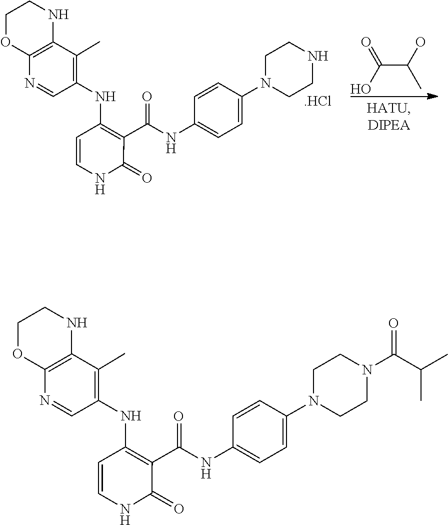

To a stirred solution of isobutyric acid (12 mg, 0.136 mmol) in DMF (4 mL) was added HATU (62.14 mg, 0.238 mmol) followed by DIPEA (0.119 ml, 0.681 mmol) at RT. The resulting reaction mixture was stirred at RT for 10 mins. then 4-((8-methyl-2,3-dihydro-1H-pyrido[2,3-b][1,4]oxazin-7-yl)amino)-2-oxo-N-(4-(piperazin-1-yl)phenyl)-1,2-dihydropyridine-3-carboxamide.HCl (Preparation 30, Step 3) (69.14 mg, 0.150 mmol) was added into the reaction vessel and stirring was continued at RT for 18 h. Progress of the reaction was monitored by LCMS and after completion the reaction mixture was diluted with water (20 mL) and extracted with 10% methanol in DCM solution (2×50 mL). The combined organic layer was dried over anhydrous sodium sulphate, filtered through a celite pad and the filtrate was concentrated under reduced pressure to give a crude product which was purified by prep-HPLC to afford the title compound (35 mg. 44% yield) as a white solid. HPLC purity: 99.26%; ¹H NMR (400 MHz; DMSO-d₆): δ 1.02 (d, J=6.72 Hz, 6H), 1.93 (s, 3H), 2.88-2.95 (m, 1H), 3.04-3.10 (m, 4H), 3.33 (9s, 2H), 3.60-3.64 (m, 4H), 4.26 (t, J=3.92 Hz, 2H), 5.56 (d, J=7.4 Hz, 1H), 5.81 (s, 1H), 6.95 (d, J=9.08 Hz, 2H), 7.29 (t, J=5.44 Hz, 2H), 7.50 (d, J=9.0 Hz, 2H), 11.37 (s, 1H), 11.89 (s, 1H), 12.92 (s, 1H); LCMS m/z: 532.05 [M+H].

### Example 389: N-(3-Chloro-4-(4-ethylpiperazin-1-yl)-5-fluorophenyl)-4-((8-methyl-2,3-dihydro-1H-pyrido[2,3-b][1,4]oxazin-7-yl)amino)-2-oxo-1,2-dihydropyridine-3-carboxamide

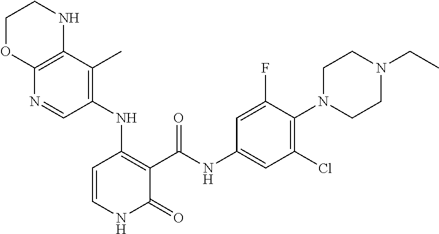 [SMILES: CCN1CCN(c2c(F)cc(NC(=O)c3c(Nc4cnc5c(c4C)NCCO5)cc[nH]c3=O)cc2Cl)CC1]

Example 389 was prepared according to the methods described in General Procedures 1-7, and the methods described below.

### Preparation 32:3-Chloro-4-(4-ethylpiperazin-1-yl)-5-fluoroaniline

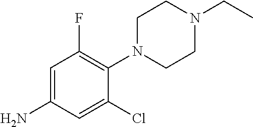 [SMILES: CCN1CCN(c2c(F)cc(N)cc2Cl)CC1]

### Step 1:1-(2-Chloro-6-fluoro-4-nitrophenyl)-4-ethylpiperazine

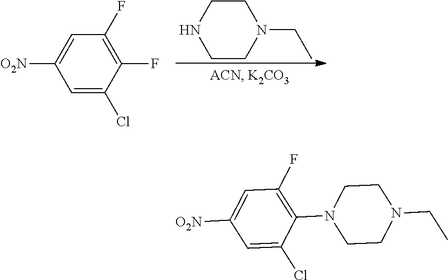

To a stirred solution of commercially available 1-chloro-2,3-difluoro-5-nitrobenzene (300 mg, 1.55 mmol) in ACN (8 mL) was added 1-ethylpiperazine (194.75 mg, 1.705 mmol) at RT. The resulting reaction mixture was stirred at 80° C. for 16 h. Progress of the reaction was monitored by UPLC-MS and after completion the reaction mass was diluted with 10% MeOH in DCM and washed with brine. The organic layer was concentrated in vacuo to give the title compound (486 mg, crude) as a yellow sticky solid. LCMS m/z: 288.11 [M+H].

### Step 2:3-Chloro-4-(4-ethylpiperazin-1-yl)-5-fluoroaniline

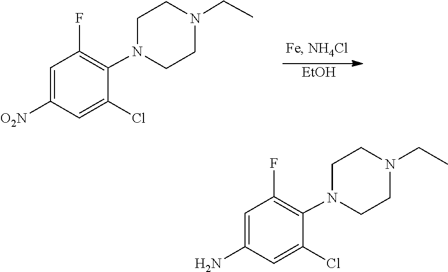 [SMILES: "EtOH" ; CCN1CCN(c2c(F)cc([N+](=O)[O-])cc2Cl)CC1 ; CCN1CCN(c2c(F)cc(N)cc2Cl)CC1]

To a stirred solution of 1-(2-chloro-6-fluoro-4-nitrophenyl)-4-ethylpiperazine (Preparation 32, Step 1) (409 mg, 1.425 mmol) in a mixture of solvents EtOH (6 mL) and H₂O (2 mL) was added Fe powder (397.73 mg, 7.123 mmol) followed by NH₄Cl (380.99 mg, 7.123 mmol) at RT. The resulting reaction mixture was allowed to stir at 80° C. for 16 h. Progress of the reaction was monitored by UPLC-MS and after completion the reaction mixture was filtered through a celite bed and the bed was washed with 10% MeOH in DCM. The obtained filtrate was washed with water followed by brine and concentrated in vacuo to afford the title compound (422 mg, crude) as a yellow sticky solid. LCMS m/z: 258.11 [M+H].

### Preparation 33, Step 1: N-(3-Chloro-4-(4-ethylpiperazin-1-yl)-5-fluorophenyl)-4-iodo-2-methoxynicotinamide

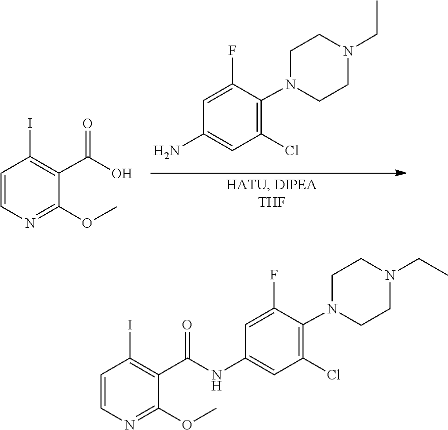 [SMILES: COc1nccc(I)c1C(=O)O ; CCN1CCN(c2c(F)cc(N)cc2Cl)CC1 ; CCN1CCN(c2c(F)cc(NC(=O)c3c(I)ccnc3OC)cc2Cl)CC1]

To a stirred solution of 4-iodo-2-methoxynicotinic acid (Preparation 11, Step 1) (372 mg, 1.333 mmol) in THF (10 mL) was added HATU (760.37 mg, 1.999 mmol) followed by DIPEA (516.99 mg, 3.999 mmol) at RT and after 10 min. 3-chloro-4-(4-ethylpiperazin-1-yl)-5-fluoroaniline (Preparation 32, Step 2) (343 mg, 1.333 mmol) was added into the reaction vessel and stirring continued at RT for 16 h. Progress of the reaction was monitored by UPLC-MS and after completion the reaction mass was diluted with 10% MeOH in DCM, washed with water followed by brine and concentrated in vacuo to give the crude product which was purified by column chromatography using 2-3% MeOH in DCM as eluent to afford the title compound (269 mg, 39% yield) as a yellow solid. LCMS m/z: 519.21 [M+H].

### Step 2: N-(3-Chloro-4-(4-ethylpiperazin-1-yl)-5-fluorophenyl)-2-methoxy-4-((8-methyl-2,3-dihydro-1H-pyrido[2,3-b][1,4]oxazin-7-yl)amino) nicotinamide

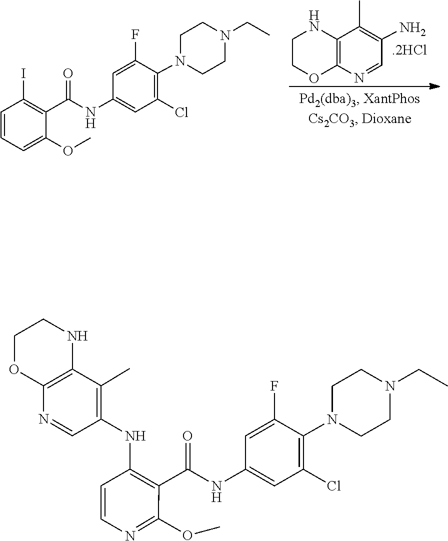

A stirred mixture of N-(3-chloro-4-(4-ethylpiperazin-1-yl)-5-fluorophenyl)-4-iodo-2-methoxynicotinamide (Preparation 33, Step 1) (200 mg, 0.386 mmol), 8-methyl-2,3-dihydro-1H-pyrido[2,3-b][1,4]oxazin-7-amine dihydrochloride (Preparation 9, Step 4) (76.43 mg, 0.463 mmol) and Cs₂CO₃ (565.97 mg, 1.737 mmol) in dioxane (8 mL) was purged with nitrogen gas for 10 mins. then Pd₂ (dba)₃ (35.35 mg, 0.039 mmol) and Xantphos (44.67 mg, 0.077 mmol) were added into the reaction vessel and the whole heated at 100° C. for 16 h. Progress of the reaction was monitored by UPLC-MS and after completion the reaction mass was diluted with 10% MeOH in DCM, filtered through a celite bed and the bed was washed with MeOH-DCM. The obtained filtrate was washed with water followed by brine. The organic layer was evaporated in vacuo to give the crude product which was purified by column chromatography using 4-5% MeOH in DCM as eluent to afford the title compound (210 mg, 97% yield) as a dark yellow sticky liquid. LCMS m/z: 555.98 [M+H].

### Step 3: N-(3-Chloro-4-(4-ethylpiperazin-1-yl)-5-fluorophenyl)-4-((8-methyl-2,3-dihydro-1H-pyrido[2,3-b][1,4]oxazin-7-yl)amino)-2-oxo-1,2-dihydropyridine-3-carboxamide

### Example 389

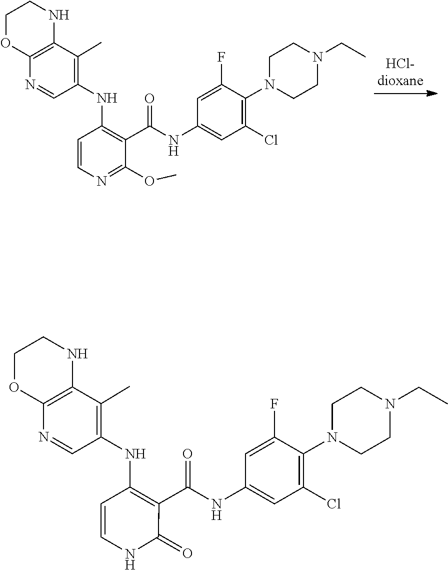 [SMILES: CCN1CCN(c2c(F)cc(NC(=O)c3c(Nc4cnc5c(c4C)NCCO5)ccnc3OC)cc2Cl)CC1 ; CCN1CCN(c2c(F)cc(NC(=O)c3c(Nc4cnc5c(c4C)NCCO5)cc[nH]c3=O)cc2Cl)CC1]

A suspension of N-(3-chloro-4-(4-ethylpiperazin-1-yl)-5-fluorophenyl)-2-methoxy-4-((8-methyl-2,3-dihydro-1H-pyrido[2,3-b][1,4]oxazin-7-yl)amino) nicotinamide (Preparation 33, Step 2) (200 mg, 0.360 mmol) in 4M HCl-dioxane (6 mL) was stirred at 80° C. for 1 h. After completion of the reaction (monitored by UPLC-MS) the solvent was evaporated in vacuo to obtain the crude compound which was purified by prep-HPLC to afford the title compound (51 mg, 26% yield) as a pale yellow solid. HPLC purity: 99.85%; ¹H NMR (400 MHz; DMSO-d₆): δ 1.04 (t, J=7.16 Hz, 3H), 1.93 (s, 3H), 2.33-2.40 (m, 2H), 2.50-2.51 (m, 2H merged with DMSO), 3.06 (s, 4H), 3.30-3.32 (m, 4H merged in DMSO water), 4.26 (t, J=4.0 Hz, 2H), 5.56 (d, J=7.44 Hz, 1H), 5.81 (s, 1H), 7.32 (d, J=5.28 Hz, 2H), 7.48-7.52 (m, 1H), 7.59 (d, J=1.88 Hz, 1H), 11.45 (s, 1H), 11.60 (s, 1H), 13.26 (s, 1H); LCMS m/z: 542.11 [M+H].

### Example 396: N-(4-(2-Hydroxypropan-2-yl)-1-methoxyisoquinolin-6-yl)-4-((8-methyl-2,3-dihydro-1H-pyrido[2,3-b][1,4]oxazin-7-yl)amino)-2-oxo-1,2-dihydropyridine-3-carboxamide

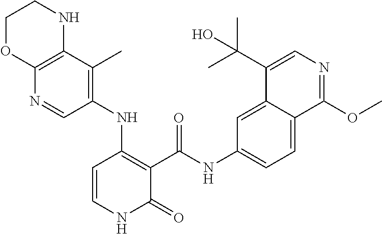 [SMILES: COc1ncc(C(C)(C)O)c2cc(NC(=O)c3c(Nc4cnc5c(c4C)NCCO5)cc[nH]c3=O)ccc12]

Example 396 was prepared according to the methods described in General Procedures 1-7, and the methods described below.

### Preparation 34:2-(6-Amino-1-methoxyisoquinolin-4-yl) propan-2-ol

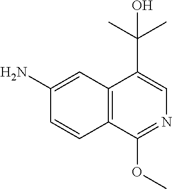 [SMILES: COc1ncc(C(C)(C)O)c2cc(N)ccc12]

### Step 1: tert-Butyl (1-oxo-1,2-dihydroisoquinolin-6-yl) carbamate

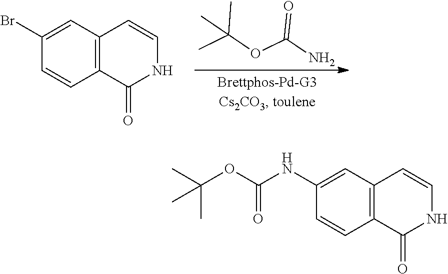

To a stirred suspension of commercially available 6-bromoisoquinolin-1 (2H)-one (1120.25 mg, 5.00 mmol) in toluene (20 mL) was added tert-butyl carbamate (1464.25 mg, 12.50 mmol) and Cs₂CO₃ (4887.30 mg, 15.00 mmol) at RT. The resulting reaction mixture was purged with nitrogen gas for 10 min. then Brettphos-Pd-G3 (906.50 mg, 1.00 mmol) was added into the reaction vessel and the whole was heated at 100° C. for 16 h. Progress of the reaction was monitored by LCMS and after completion the reaction mixture was diluted with water and extracted with 10% MeOH in DCM. The combined organic layers were washed with brine, dried over anhydrous sodium sulphate, filtered and concentrated under reduced pressure to obtain the crude product which was purified by Combi-flash using 2% MeOH in DCM as eluent to afford the title compound (780 mg, 60% yield) as a white solid. LCMS m/z: 261.22 [M+H].

### Step 2: tert-Butyl (4-iodo-1-oxo-1,2-dihydroisoquinolin-6-yl) carbamate

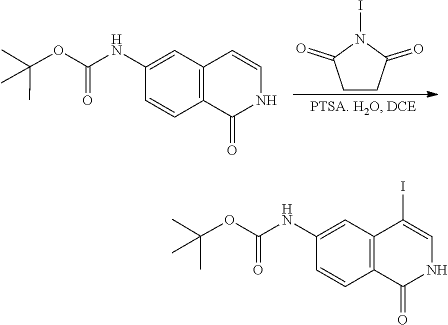

To a stirred suspension of tert-butyl (1-oxo-1,2-dihydroisoquinolin-6-yl) carbamate (Preparation 34, Step 1) (780.87 mg, 3.00 mmol) in DCE (20 mL) was added 1-iodopyrrolidine-2,5-dione (742.43 mg, 3.30 mmol) and PTSA.H₂O (57.06 mg, 0.30 mmol) at RT. The resulting reaction mixture was allowed to stir at RT for 2 h. After completion of the reaction (checked by LCMS) the reaction mixture was treated with an aqueous solution of NaHSO₃ (100 mL) and extracted with 10% MeOH in DCM. The combined organic layers were washed with brine solution, dried over anhydrous sodium sulphate, filtered and concentrated under reduced pressure to afford the title compound (1004 mg, 86% yield) as an off-white solid which was used in the next step without any further purification. LCMS m/z: 387.17 [M+H].

### Step 3: tert-Butyl (4-iodo-1-methoxyisoquinolin-6-yl) carbamate

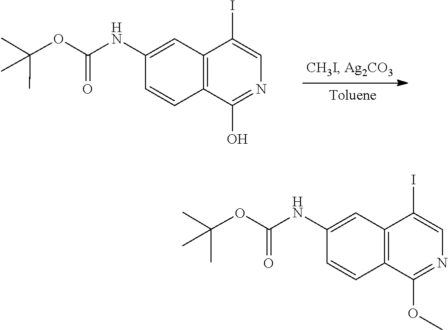 [SMILES: CC(C)(C)OC(=O)Nc1ccc2c(O)ncc(I)c2c1 ; COc1ncc(I)c2cc(NC(=O)OC(C)(C)C)ccc12]

To a stirred solution of tert-butyl (4-iodo-1-oxo-1,2-dihydroisoquinolin-6-yl) carbamate (Preparation 34, Step 2) (502.03 mg, 1.30 mmol) in toluene (20 mL) was added Ag2CO₃ (3584.0 mg, 13.00 mmol) and MeI (0.40 mL, 6.50 mmol) at RT in a sealed tube. Thewhole was allowed to stir at 110° C. for 16 h. Transformation was confirmed by UP-LCMS, then the reaction mixture was cooled to RT, diluted with ethyl acetate and passed through a celite bed, which was then further washed with ethyl acetate. The filtrate was dried over anhydrous sodium sulphate, filtered and concentrated under reduced pressure to give the crude product which was purified by Combi-flash using 5% EtOAc in hexane as eluent to afford the title compound (440.23 mg, 85% yield) as a white solid. LCMS m/z: 401.18 [M+H].

### Step 4: tert-Butyl (4-acetyl-1-methoxyisoquinolin-6-yl) carbamate

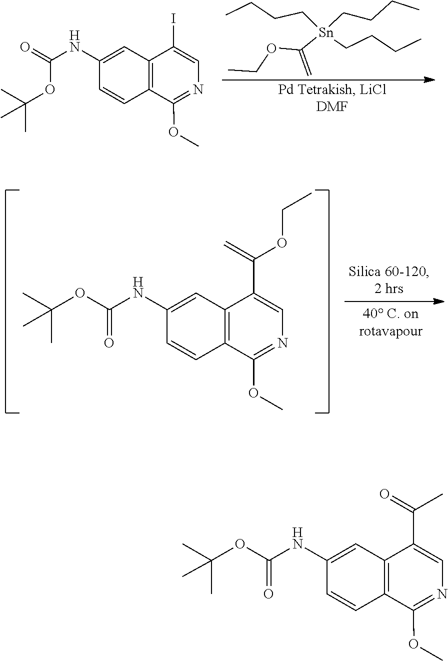

To a stirred degassed solution of tert-butyl (4-iodo-1-methoxyisoquinolin-6-yl) carbamate (Preparation 34, Step 3) (440.23 mg, 1.10 mmol), tributyl (1-ethoxyvinyl) stannane (595.89 mg, 1.65 mmol) and LiCl (139.88 mg, 3.30 mmol) in DMF (10 mL) was added Pd(PPh₃)₄ (127.11 mg, 0.11 mmol) at RT in a sealed tube. The resulting reaction mixture was stirred at 100° C. for 16 h. Progress of the reaction was monitored by UP-LCMS and after completion the reaction mixture was poured into ice-water and extracted with ethyl acetate. The combined organic layers were washed with brine, dried over anhydrous sodium sulphate, filtered and concentrated under reduced pressure to give the crude product which was adsorbed on silica gel and gently rotated on a Rotary at 40° C. for 2 h. UP-LCMS showed complete conversion of the ether group into the desired acetone group after rearrangement. The adsorbed compound was purified by Combi-flash using 10% EtOAc in hexane as eluent to afford the title compound (284 mg, 81% yield) as a white solid. LCMS m/z: 317.19 [M+H].

### Step 5: tert-Butyl (4-(2-hydroxypropan-2-yl)-1-methoxyisoquinolin-6-yl) carbamate

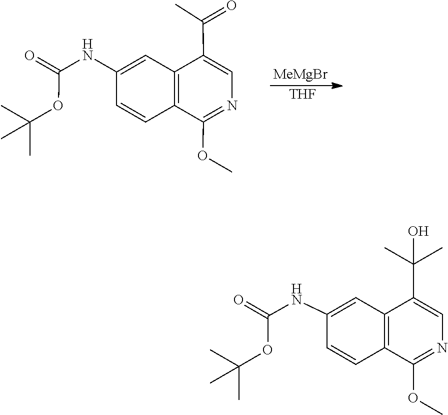 [SMILES: "MeMgBr" ; COc1ncc(C(C)=O)c2cc(NC(=O)OC(C)(C)C)ccc12 ; COc1ncc(C(C)(C)O)c2cc(NC(=O)OC(C)(C)C)ccc12 ; "THF"]

To a stirred solution of tert-butyl (4-acetyl-1-methoxyisoquinolin-6-yl) carbamate (Preparation 34, Step 4) (474.21 mg, 1.50 mmol) in dry THF (10 mL) was added methyl magnesium bromide (0.95 mL, 2.70 mmol) dropwise at 0-5° C. and the combined mixture was allowed to stir at RT for 4 h. Complete conversion was confirmed by UP-LCMS thereafter the reaction mixture was quenched with a saturated ammonium chloride solution (50 mL) and extracted with ethyl acetate. The combined organic layers were washed with brine, dried over anhydrous sodium sulphate, filtered and concentrated under reduced pressure to give the crude product which was purified by Combi-flash using 30% EtOAc in hexane as eluent to afford the title compound (265 mg, 88% yield) as a pale yellow solid. LCMS m/z: 333.27 [M+H].

### Step 6:2-(6-Amino-1-methoxyisoquinolin-4-yl) propan-2-ol.HCl

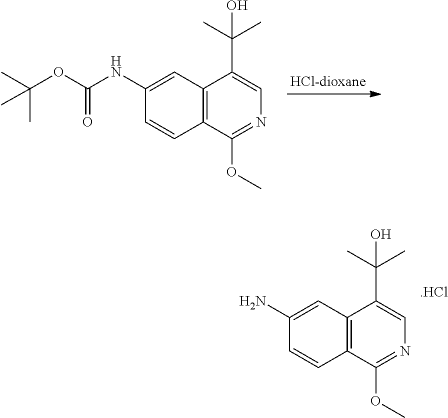 [SMILES: COc1ncc(C(C)(C)O)c2cc(NC(=O)OC(C)(C)C)ccc12 ; COc1ncc(C(C)(C)O)c2cc(N)ccc12 ; ".HCl"]

To a stirred solution of tert-butyl (4-(2-hydroxypropan-2-yl)-1-methoxyisoquinolin-6-yl) carbamate (Preparation 34, Step 5) (99.70 mg, 0.30 mmol) in dioxane (2 mL) was added 4M HCl-dioxane (2 mL) with ice cooling and the whole allowed to stir at RT for 16 h. Completion of the reaction was confirmed by LCMS and after that the reaction mixture was concentrated under reduced pressure to give a residue which was triturated with diethyl ether to afford the title compound (106 mg, crude) as a white solid. LCMS m/z: 233.27 [M+H].

### Preparation 35, Step 1:4-Chloro-2-oxo-1,2-dihydropyridine-3-carboxylic acid

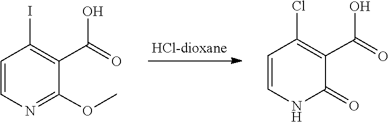

A suspension of 4-iodo-2-methoxynicotinic acid (Preparation 11, Step 1) (558.06 mg, 2 mmol) in 4M HCl in dioxane (8 mL) was allowed to stir at 80° C. for 2 h. UP-LCMS and TLC showed the complete conversion of starting material into the desired product. The solvent from the reaction mixture was evaporated in vacuo to give a residue which was triturated with diethyl ether to afford the title compound (399 mg, crude) as an off-white solid. LCMS m/z: 173.98 [M+H].

### Step 2:4-((8-Methyl-2,3-dihydro-1H-pyrido[2,3-b][1,4]oxazin-7-yl)amino)-2-oxo-1,2-dihydropyridine-3-carboxylic acid

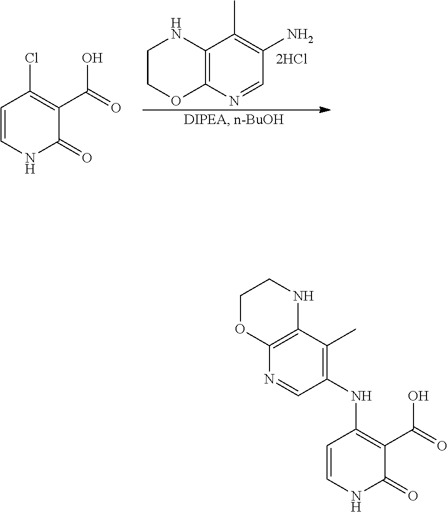

To a stirred suspension of 4-chloro-2-oxo-1,2-dihydropyridine-3-carboxylic acid (Preparation 35, Step 1) (399.16 mg, 2.00 mmol) in n-butanol (10 mL) was added 8-methyl-2,3-dihydro-1H-pyrido[2,3-b][1,4]oxazin-7-amine dihydrochloride (Preparation 9, Step 4) (396.46 mg, 2.40 mmol) followed by DIPEA (2 mL, 12.00 mmol) at RT. The resulting reaction mixture was stirred at 130° C. for 22 h. Progress of the reaction was monitored by TLC and UP-LCMS and after completion the solvent was evaporated in vacuo to give a residue which was triturated with diethyl ether to afford the title compound (408 mg, 67% yield) as a greyish solid that was used in the next step without any further purification. LCMS m/z: 303.15 [M+H].

### Step 3: N-(4-(2-Hydroxypropan-2-yl)-1-methoxyisoquinolin-6-yl)-4-((8-methyl-2,3-dihydro-1H-pyrido[2,3-b][1,4]oxazin-7-yl)amino)-2-oxo-1,2-dihydropyridine-3-carboxamide (Example 396)

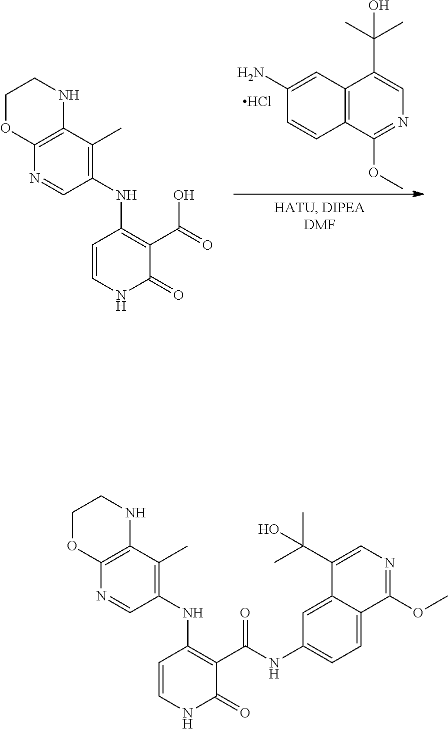 [SMILES: Cc1c(Nc2cc[nH]c(=O)c2C(=O)O)cnc2c1NCCO2 ; Cl ; COc1ncc(C(C)(C)O)c2cc(N)ccc12 ; COc1ncc(C(C)(C)O)c2cc(NC(=O)c3c(Nc4cnc5c(c4C)NCCO5)cc[nH]c3=O)ccc12]

To a stirred suspension of 4-((8-methyl-2,3-dihydro-1H-pyrido[2,3-b][1,4]oxazin-7-yl)amino)-2-oxo-1,2-dihydropyridine-3-carboxylic acid (Preparation 35, Step 2) (120.91 mg, 0.40 mmol) in DMF (3 mL) was added HATU (228.13 mg, 0.60 mmol), DIPEA (0.20 mL, 0.12 mmol) and 2-(6-amino-1-methoxyisoquinolin-4-yl) propan-2-ol.HCl (Preparation 34, Step 6) (92.91 mg, 0.40 mmol) at RT. The resulting reaction mixture was allowed to stir at 50° C. for 16 h. Progress of the reaction was monitored by TLC and UP-LCMS and after completion the reaction mixture was poured into ice-water and extracted with 10% MeOH in DCM. The combined organic layers were washed with brine, dried over anhydrous Na₂SO₄, filtered and concentrated in vacuo to give the crude product which was purified by Combi-flash using 10% MeOH in DCM as eluent followed by prep-HPLC to afford the title compound (5 mg, 3% yield) as a pale yellow solid. HPLC purity: 99.79%; ¹H NMR (400 MHz; DMSO-d₆): δ 1.68 (s, 6H), 1.95 (s, 3H), 3.32 (s, 2H), 4.02 (s, 3H), 4.27 (s, 2H), 5.22 (s, 1H), 5.57 (d, J=7.28 Hz, 1H), 5.84 (s, 1H), 7.33 (t, J=4.24 Hz, 2H), 7.87-7.90 (m, 1H), 7.98 (s, 1H), 8.18 (d, J=4.24 Hz, 1H), 8.98 (s, 1H), 11.46 (d, J=6.04 Hz, 1H), 11.78 (s, 1H), 13.46 (s, 1H); LCMS m/z: 517.33 [M+H].

### Example 198:4-((3-Fluoro-8-methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide

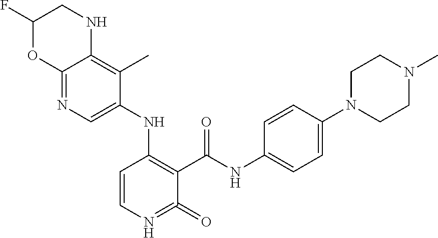

Example 198 was prepared according to the methods described in General Procedures 1-7, and the methods described below.

### Preparation 36, Step 1: tert-Butyl 7-((tert-butoxycarbonyl)amino)-3-fluoro-8-methyl-2,3-dihydro-1H-pyrido[2,3-b][1,4]oxazine-1-carboxylate

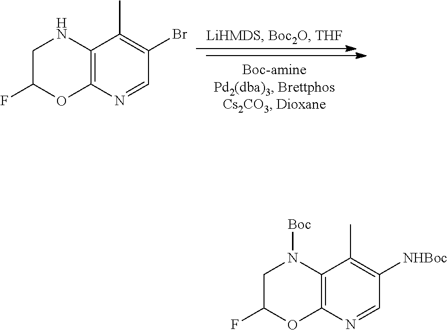 [SMILES: Cc1c(Br)cnc2c1NCC(F)O2 ; Cc1c(NC(=O)OC(C)(C)C)cnc2c1N(C(=O)OC(C)(C)C)CC(F)O2]

To a solution of commercially available 7-bromo-3-fluoro-8-methyl-2,3-dihydro-1H-pyrido[2,3-b][1,4]oxazine (100 mg, 0.41 mmol) in dry THF (2 mL) was added LiHMDS (1M, 0.61 mL) followed by Boc-anhydride (134 mg, 0.61 mmol) at RT. The resulting reaction mixture was refluxed for 6 h. TLC showed completion of the reaction. The reaction mixture was quenched with saturated ammonium chloride solution and the organic layer was extracted with ethyl acetate. The combined organic layers were washed with water followed by brine, dried over sodium sulphate, filtered and concentrated under vacuum to obtain the Boc protected intermediate (80 mg, 0.23 mmol) which was immediately taken up in degassed dioxane (2 mL) and Boc-amine (135 mg, 1.16 mmol), Cs₂CO₃ (188 mg, 0.58 mmol), Brettphos (49.6 mg, 0.09 mmol) and Pd₂ (dba)₃ (42.3 mg, 0.05 mmol) added and the whole was heated at 110° C. under a nitrogen atmosphere for 16 h. Progress of the reaction was monitored by LCMS and after completion, the reaction mixture was filtered through a celite bed and the filtrate was evaporated under reduced pressure to obtain the crude product which was purified by column chromatography to afford the title compound (50 mg, 56.4% yield) as an off-white solid. LCMS m/z: 384.0 [M+H].

### Step 2:4-((3-Fluoro-8-methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide (Example 198)

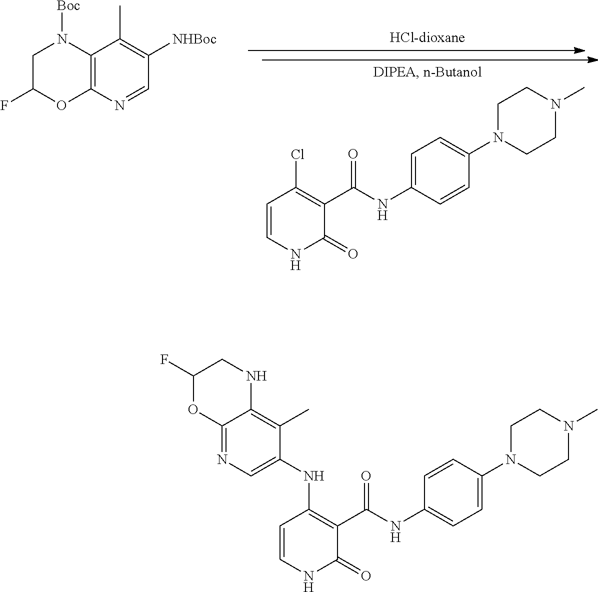 [SMILES: Cc1c(NC(=O)OC(C)(C)C)cnc2c1N(C(=O)OC(C)(C)C)CC(F)O2 ; CN1CCN(c2ccc(NC(=O)c3c(Cl)cc[nH]c3=O)cc2)CC1 ; Cc1c(Nc2cc[nH]c(=O)c2C(=O)Nc2ccc(N3CCN(C)CC3)cc2)cnc2c1NCC(F)O2]

A suspension of tert-butyl 7-((tert-butoxycarbonyl)amino)-3-fluoro-8-methyl-2,3-dihydro-1H-pyrido[2,3-b][1,4]oxazine-1-carboxylate (Preparation 36, Step 1) (50 mg, 0.13 mmol) in 4M HCl-dioxane (1 mL) was stirred at 0-5° C. for 1.5 h. TLC showed completion of the reaction, the solvent was evaporated under reduced pressure to obtain a residue which was immediately taken up in n-butanol (1 mL) and DIPEA (0.15 mL, 0.86 mmol) and 4-chloro-N-(4-(4-methylpiperazin-1-yl)phenyl)-2-oxo-1,2-dihydropyridine-3-carboxamide (Preparation 1, Step 3) (60 mg, 0.17 mmol) were added at RT. The resulting mixture was heated at 120° C. for 16 h. After completion of the reaction (monitored by TLC and LCMS) the solvent was evaporated under vacuum to obtain a residue which was then purified by prep-HPLC to afford the title compound (10 mg, 16% yield) as a light brown solid. HPLC purity: 99.76%; ¹H NMR (400 MHz; DMSO-d₆): δ 1.98 (s, 3H), 2.22 (s, 3H), 2.45 (s, 4H), 3.07-3.08 (m, 4H), 3.24 (d, J=13.28 Hz, 1H), 3.55 (d, J=12.84 Hz, 1H), 5.56 (d, J=7.4 Hz, 1H), 5.99 (s, 1H), 6.28 (s, 0.5H), 6.42 (s, 0.5H), 6.91 (d, J=8.88 Hz, 2H), 7.28 (t, J=6.84 Hz, 1H), 7.43-7.48 (m, 3H), 11.38 (d, J=6.08 Hz, 1H), 11.98 (s, 1H), 12.85 (s, 1H); LCMS m/z: 394.30 [M+H].

### Example 137: N,N-Dimethyl-4-(4-(4-((8-methyl-2,3-dihydro-1H-pyrido[2,3-b][1,4]oxazin-7-yl)amino)-2-oxo-1,2-dihydropyridine-3-carboxamido)phenyl) piperazine-1-carboxamide

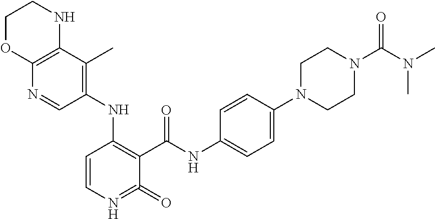

Example 137 was prepared according to the methods described in General Procedures 1-7, and the methods described below.

### Preparation 37: N,N-Dimethyl-4-(4-(4-((8-methyl-2,3-dihydro-1H-pyrido[2,3-b][1.4]oxazin-7-yl)amino)-2-oxo-1,2-dihydropyridine-3-carboxamido)phenyl) piperazine-1-carboxamide (Example 137)

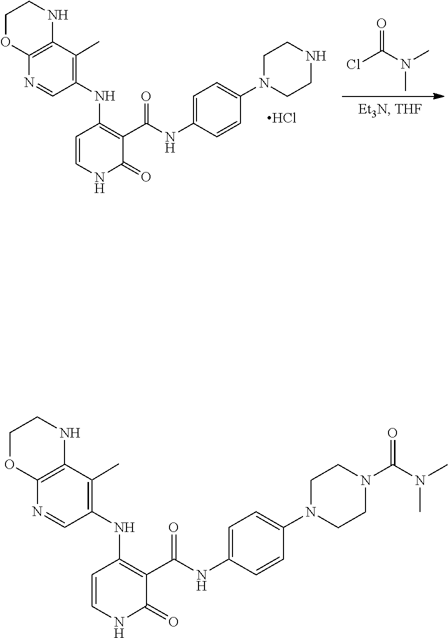

To a stirred solution of 4-((8-methyl-2,3-dihydro-1H-pyrido[2,3-b][1,4]oxazin-7-yl)amino)-2-oxo-N-(4-(piperazin-1-yl)phenyl)-1,2-dihydropyridine-3-carboxamide.HCl (Preparation 30, Step 3) (70 mg, 0.15 mmol) in THF (2 mL) was added TEA (0.11 mL, 0.76 mmol) followed by dimethyl carbamoyl chloride (0.01 ml, 0.15) at 0-5° C. and the combined mixture was further stirred at RT for 3 h. Progress of the reaction was monitored by LCMS and after completion the solvent was evaporated in vacuo to obtain the crude product which was purified by prep-HPLC to afford the title compound (12 mg, 13% yield) as an off-white solid. HPLC purity: 99.64%; ¹H NMR (400 MHz; DMSO-d₆): δ 1.93 (s, 3H), 2.77 (s, 6H), 3.08-3.10 (m, 4H), 3.22-3.25 (m, 4H), 3.32 (bs, 2H merged in DMSO-H₂O), 4.25 (s, 2H), 5.55 (d, J=7.2 Hz, 1H), 5.79 (s, 1H), 6.94 (d, J=8.8 Hz, 2H), 7.28 (t, J=6.4 Hz, 2H), 7.49 (d, J=8.8 Hz, 2H), 11.34 (s, 1H), 11.90 (s, 1H), 12.87 (s, 1H); LCMS m/z: 533.45 [M+H].

### Example 271: N-(4-(1-Isopropyl-1H-pyrazol-5-yl)phenyl)-4-((8-methyl-2,3-dihydro-1H-pyrido[2,3-b][1.4]oxazin-7-yl)amino)-2-oxo-1,2-dihydropyridine-3-carboxamide

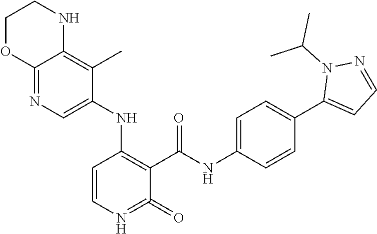

Example 271 was prepared according to the methods described in General Procedures 1-7, and the methods described below.

### Preparation 38:4-Chloro-N-(4-(1-isopropyl-1H-pyrazol-5-yl)phenyl)-2-oxo-1,2-dihydropyridine-3-carboxamide

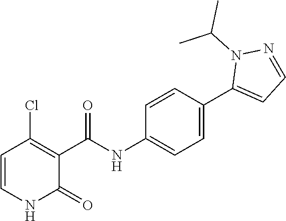 [SMILES: CC(C)n1nccc1-c1ccc(NC(=O)c2c(Cl)cc[nH]c2=O)cc1]

### Step 1:4-Iodo-N-(4-(1-isopropyl-1H-pyrazol-5-yl)phenyl)-2-methoxynicotinamide

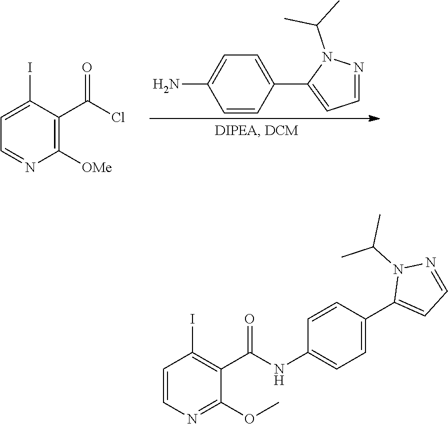 [SMILES: COc1nccc(I)c1C(=O)Cl ; CC(C)n1nccc1-c1ccc(N)cc1 ; COc1nccc(I)c1C(=O)Nc1ccc(-c2ccnn2C(C)C)cc1]

To a stirred solution of commercially available 4-(1-isopropyl-1H-pyrazol-5-yl) aniline (100 mg, 0.49 mmol) in DCM (5 mL) was added DIPEA (0.432 mL, 2.48 mmol) and freshly prepared 4-iodo-2-methoxynicotinoyl chloride (369.40 mg, 1.24 mmol) at 0-5° C. The resulting reaction mixture was stirred at RT for 24 h. After completion of the reaction (monitored by LCMS and TLC) the solvent was evaporated under reduced pressure to obtain a crude product which was purified by column chromatography using 2-5% MeOH in DCM to afford the title compound (120 mg, 52.1% yield) as an off-white solid. LCMS m/z: 463.2 [M+H].

### Step 2:4-Chloro-N-(4-(1-isopropyl-1H-pyrazol-5-yl)phenyl)-2-oxo-1,2-dihydropyridine-3-carboxamide.HCl

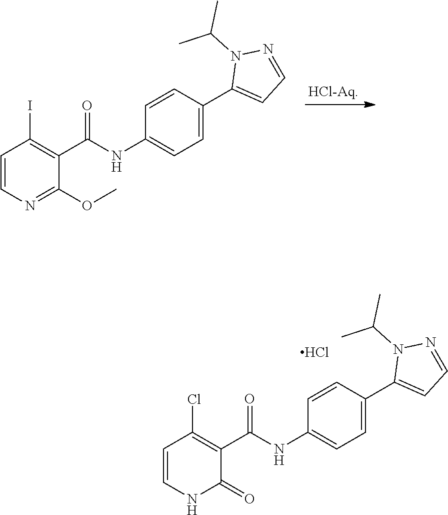 [SMILES: COc1nccc(I)c1C(=O)Nc1ccc(-c2ccnn2C(C)C)cc1 ; CC(C)n1nccc1-c1ccc(NC(=O)c2c(Cl)cc[nH]c2=O)cc1 ; Cl]

A suspension of 4-iodo-N-(4-(1-isopropyl-1H-pyrazol-5-yl)phenyl)-2-methoxynicotinamide (Preparation 38, Step 1) (120 mg, 0.259 mmol) in 6N aq. HCl (3 mL) was refluxed for 16 h. After completion of the reaction (monitored by LCMS and TLC) the solvent was removed in vacuo to afford the title compound (80 mg, crude) as an off-white solid.

### Preparation 39: N-(4-(1-Isopropyl-1H-pyrazol-5-yl)phenyl)-4-((8-methyl-2,3-dihydro-1H-pyrido[2,3-b][1.4]oxazin-7-yl)amino)-2-oxo-1,2-dihydropyridine-3-carboxamide

### Example 271

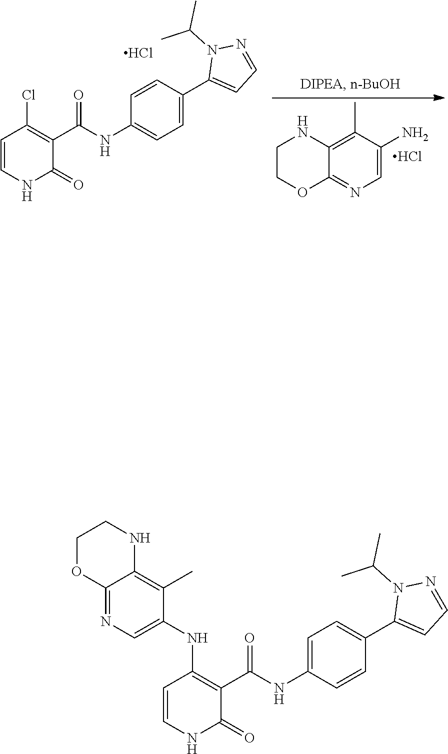 [SMILES: CC(C)n1nccc1-c1ccc(NC(=O)c2c(Cl)cc[nH]c2=O)cc1 ; Cl ; Cc1c(N)cnc2c1NCCO2 ; Cl ; Cc1c(Nc2cc[nH]c(=O)c2C(=O)Nc2ccc(-c3ccnn3C(C)C)cc2)cnc2c1NCCO2]

To a stirred solution of 4-chloro-N-(4-(1-isopropyl-1H-pyrazol-5-yl)phenyl)-2-oxo-1,2-dihydropyridine-3-carboxamide.HCl (80 mg, 0.225 mmol) in n-BuOH (2 mL) was added DIPEA (0.12 mL, 0.67 mmol) followed by 8-methyl-2,3-dihydro-1H-pyrido[2,3-b][1,4]oxazin-7-amine dihydrochloride (Preparation 9, Step 4) (73.95 mg, 0.45 mmol) at RT. The resulting reaction mixture was heated at 120° C. for 16 h. Progress of the reaction was monitored by LCMS and after completion the solvent was removed in vacuo to give a residue which was purified by prep-HPLC to afford the title compound (10 mg, 9% yield) as a light brown solid. HPLC purity: 99.89%; ¹H NMR (400 MHZ; MeOD): δ 1.43 (d, J=6.64 Hz, 6H), 2.03 (d, J=9.36 Hz, 3H), 3.44-3.47 (m, 2H), 4.35 (t, J=4.28 Hz, 2H), 4.57-4.63 (m, 1H), 5.73 (t, J=7.44 Hz, 1H), 6.26 (s, 1H), 7.24 (d, J=7.6 Hz, 1H), 7.34-7.38 (m, 3H), 7.53 (d, J=1.8 Hz, 1H), 7.80 (d, J=8.44 Hz, 2H), 11.96 (s, 1H), 13.01 (s, 1H); LCMS m/z: 486.35 [M+H].

Example 126:4-((8-Methyl-2-oxo-2,3-dihydro-1H-pyrido[2,3-b][1,4]oxazin-7-ylamino)-N-(4-(4-methylpiperazin-1-yl)phenyl)-2-oxo-1,2-dihydropyridine-3-carboxamide

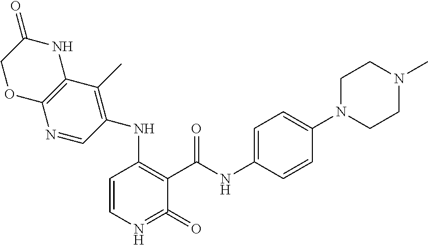

Example 126 was prepared according to the methods described in General Procedures 1-7, and the methods described below.

### Preparation 40:7-Amino-8-methyl-1H-pyrido[2,3-b][1,4]oxazin-2 (3H)-one.HCl

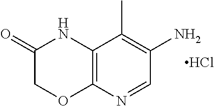

### Step 1: tert-Butyl (8-methyl-2-oxo-2,3-dihydro-1H-pyrido[2,3-b][1,4]oxazin-7-yl) carbamate

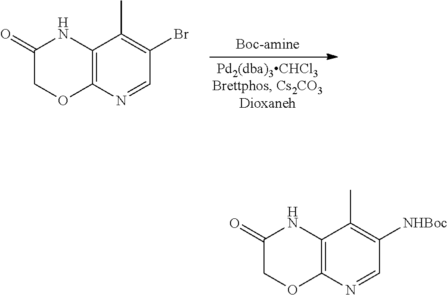 [SMILES: Cc1c(Br)cnc2c1NC(=O)CO2 ; Cc1c(NC(=O)OC(C)(C)C)cnc2c1NC(=O)CO2]

To a degassed solution of 7-bromo-8-methyl-1H-pyrido[2,3-b][1,4]oxazin-2 (3H)-one (Preparation 9, Step 1) (300 mg, 1.24 mmol) in dioxane (10 mL) was added tert-butyl carbamate (433 mg, 3.703 mmol), Cs₂CO₃ (1209 mg, 3.72 mmol), Brettphos (270 mg, 0.49 mmol) and tris(dibenzylideneacetone) dipalladium (0).CHCI₃ (260 mg, 0.25 mmol) at RT. The resulting reaction mixture was heated at 110° C. for 16 h. Progress of the reaction was monitored by LCMS and after completion the reaction mixture was filtered and the filtrate was concentrated under vacuum to obtain the crude product which was purified by column chromatography (70-100% ethyl acetate-hexane) to afford the title compound (250 mg, 72.19% yield) as an off-white solid. LCMS m/z: 279.9 [M+H].

### Step 2:7-Amino-8-methyl-1H-pyrido[2,3-b][1,4]oxazin-2 (3H)-one.HCl

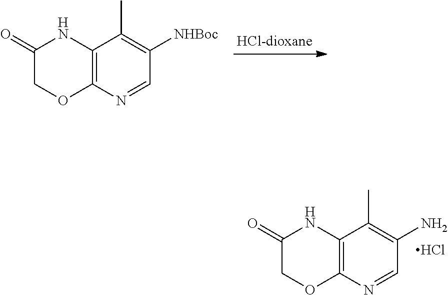 [SMILES: Cc1c(NC(=O)OC(C)(C)C)cnc2c1NC(=O)CO2 ; Cc1c(N)cnc2c1NC(=O)CO2 ; Cl]

A suspension of tert-butyl (8-methyl-2-oxo-2,3-dihydro-1H-pyrido[2,3-b][1,4]oxazin-7-yl) carbamate (Preparation 40, Step 1) (60 mg, 0.21 mmol) in 4M HCl-dioxane (1 mL) was stirred at RT for 1 h. After completion of the reaction (monitored by LCMS) the reaction mass was concentrated in vacuo to afford the title compound (90 mg, crude) as an off-white solid.

### Preparation 41:4-((8-Methyl-2-oxo-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide

### Example 126

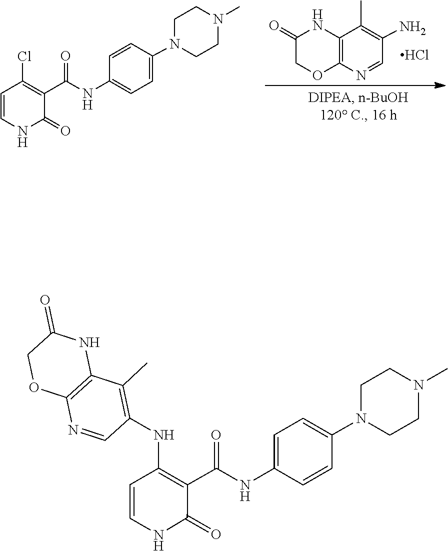

To a stirred solution of 7-amino-8-methyl-1H-pyrido[2,3-b][1,4]oxazin-2 (3H)-one.HCl (Preparation 40, Step 2) (90 mg, 0.503 mmol) in n-BuOH (2 mL) was added DIPEA (0.7 mL. 3.90 mmol) followed by 4-chloro-N-(4-(4-methylpiperazin-1-yl)phenyl)-2-oxo-1,2-dihydropyridine-3-carboxamide (Preparation 1, Step 3) (70 mg,0.39 mmol) at RT. The resulting mixture was stirred at 120° C. for 16 h. Progress of the reaction was monitored by LCMS and after completion the solvent was removed in vacuo to give a residue which was purified by prep-HPLC to afford the title compound (12 mg, 5% yield) as a white solid. HPLC purity: 98.05%; ¹H NMR (400 MHz; DMSO-d₆): δ; 2.04 (s, 3H), 2.21 (s, 3H), 2.44-2.45 (m, 4H), 3.08 (s, 4H), 4.50 (s, 2H), 5.56 (d, J=7.2 Hz, 1H), 6.90 (d, J=8.8 Hz, 2H), 7.29 (d, J=7.2 Hz, 1H), 7.42 (s, 1H), 7.47 (d, J=8.8 Hz, 2H), 11.85 (s, 1H), 13.21 (s, 1H); LCMS m/z: 490.39 [M+H].

The examples in the tables below were prepared according to the above methods used to make Example 45, 126, 132, 137, 163, 167, 168, 197, 198, 263, 271, 315, 363, 371, 389 and 396 as described in General Procedures 1-8 using the appropriate amines. In most cases the amines were commercially available or were synthesized by analogy to the above methods. Purification was as stated in the aforementioned methods.

| | | | Purity | LCMS |
|---|---|---|---|---|
| Ex | Structure | IUPAC Name | (%) | [M + H] |
| 45 | 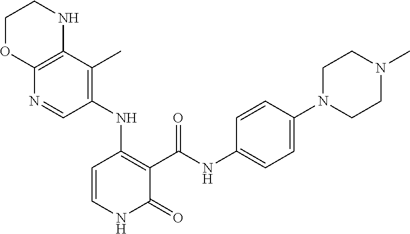 [SMILES: Cc1c(Nc2cc[nH]c(=O)c2C(=O)Nc2ccc(N3CCN(C)CC3)cc2)cnc2c1NCCO2] | 4-((8-methyl-2,3-dihydro-1H- pyrido[2,3-b][1,4]oxazin-7- yl)amino)-N-(4-(4- methylpiperazin-1-yl)phenyl)- 2-oxo-1,2-dihydropyridine-3- carboxamide | 99.22 | 476.46 |
| 93 | 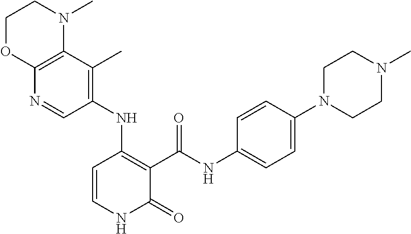 [SMILES: Cc1c(Nc2cc[nH]c(=O)c2C(=O)Nc2ccc(N3CCN(C)CC3)cc2)cnc2c1N(C)CCO2] | 4-((1,8-dimethyl-2,3-dihydro- 1H-pyrido[2,3-b][1,4]oxazin- 7-yl)amino)-N-(4-(4- methylpiperazin-1-yl)phenyl)- 2-oxo-1,2-dihydropyridine-3- carboxamide | 99.89 | 490.5 |
| 96 | 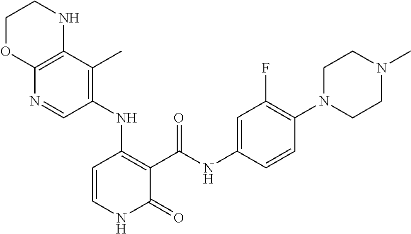 [SMILES: Cc1c(Nc2cc[nH]c(=O)c2C(=O)Nc2ccc(N3CCN(C)CC3)c(F)c2)cnc2c1NCCO2] | N-(3-fluoro-4-(4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.68 | 494.43 |
| 97 | 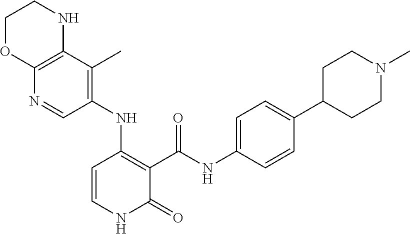 [SMILES: Cc1c(Nc2cc[nH]c(=O)c2C(=O)Nc2ccc(C3CCN(C)CC3)cc2)cnc2c1NCCO2] | 4-((8-methyl-2,3-dihydro-1H- pyrido[2,3-b][1,4]oxazin-7- yl)amino)-N-(4-(1- methylpiperidin-4-yl)phenyl)- 2-oxo-1,2-dihydropyridine-3- carboxamide | 98.53 | 475.42 |
| 105 | 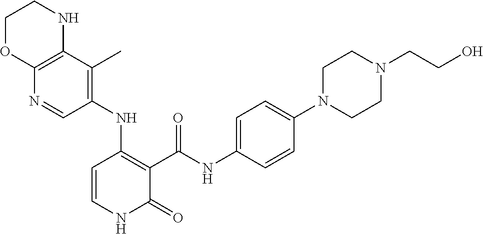 [SMILES: Cc1c(Nc2cc[nH]c(=O)c2C(=O)Nc2ccc(N3CCN(CCO)CC3)cc2)cnc2c1NCCO2] | N-(4-(4-(2- hydroxyethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.78 | 506.42 |
| 106 | 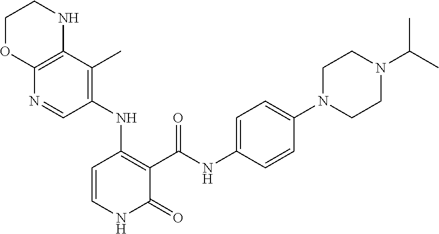 [SMILES: Cc1c(Nc2cc[nH]c(=O)c2C(=O)Nc2ccc(N3CCN(C(C)C)CC3)cc2)cnc2c1NCCO2] | N-(4-(4-isopropylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.0 | 504.42 |
| 115 | 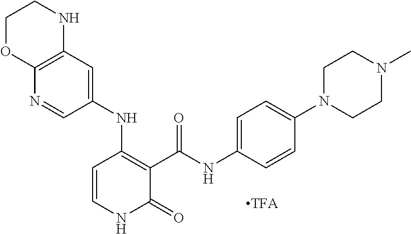 [SMILES: CN1CCN(c2ccc(NC(=O)c3c(Nc4cnc5c(c4)NCCO5)cc[nH]c3=O)cc2)CC1] | 4-((2,3-dihydro-1H- pyrido[2,3-b][1,4]oxazin-7- yl)amino)-N-(4-(4- methylpiperazin-1-yl)phenyl)- 2-oxo-1,2-dihydropyridine-3- carboxamide.TFA | 98.34 | 462.3 |
| 118 | 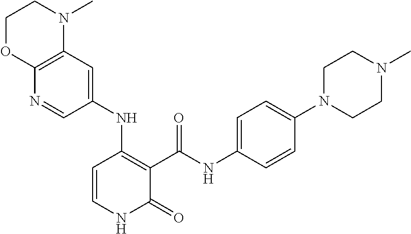 [SMILES: CN1CCN(c2ccc(NC(=O)c3c(Nc4cnc5c(c4)N(C)CCO5)cc[nH]c3=O)cc2)CC1] | 4-((1-methyl-2,3-dihydro-1H- pyrido[2,3-b][1,4]oxazin-7- yl)amino)-N-(4-(4- methylpiperazin-1-yl)phenyl)- 2-oxo-1,2-dihydropyridine-3- carboxamide | 99.76 | 476.29 |
| 125 | 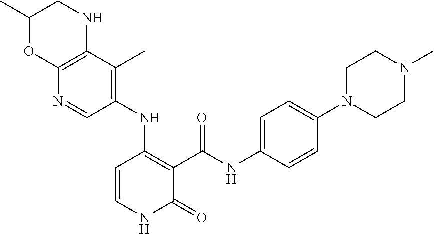 | 4-((3,8-dimethyl-2,3-dihydro- 1H-pyrido[2,3-b][1,4]oxazin- 7-yl)amino)-N-(4-(4- methylpiperazin-1-yl)phenyl)- 2-oxo-1,2-dihydropyridine-3- carboxamide | 97.79 | 490.34 |
| 126 | 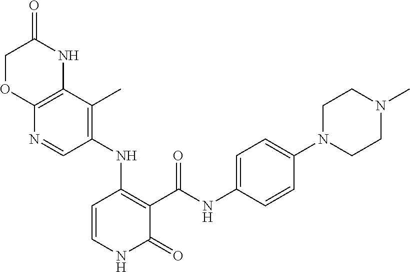 [SMILES: Cc1c(Nc2cc[nH]c(=O)c2C(=O)Nc2ccc(N3CCN(C)CC3)cc2)cnc2c1NC(=O)CO2] | 4-((8-methyl-2-oxo-2,3- dihydro-1H-pyrido[2,3- b][1,4]oxazin-7-yl)amino)-N- (4-(4-methylpiperazin-1- yl)phenyl)-2-oxo-1,2- dihydropyridine-3- carboxamide | 98.05 | 490.39 |
| 130 | 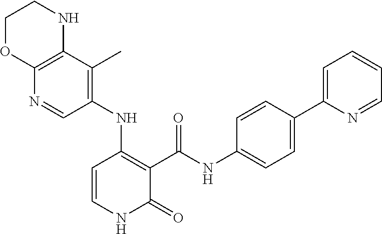 [SMILES: Cc1c(Nc2cc[nH]c(=O)c2C(=O)Nc2ccc(-c3ccccn3)cc2)cnc2c1NCCO2] | 4-((8-methyl-2,3-dihydro-1H- pyrido[2,3-b][1,4]oxazin-7- yl)amino)-2-oxo-N-(4- (pyridin-2-yl)phenyl)-1,2- dihydropyridine-3- carboxamide | 95.43 | 455.34 |
| 132 | 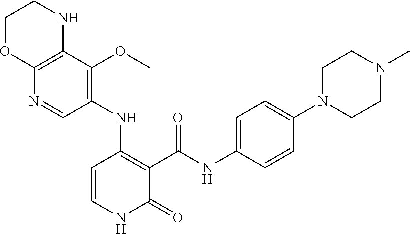 [SMILES: COc1c(Nc2cc[nH]c(=O)c2C(=O)Nc2ccc(N3CCN(C)CC3)cc2)cnc2c1NCCO2] | 4-((8-methoxy-2,3-dihydro- 1H-pyrido[2,3-b][1,4]oxazin- 7-yl)amino)-N-(4-(4- methylpiperazin-1-yl)phenyl)- 2-oxo-1,2-dihydropyridine-3- carboxamide | 95.24 | 492.25 |
| 133 | 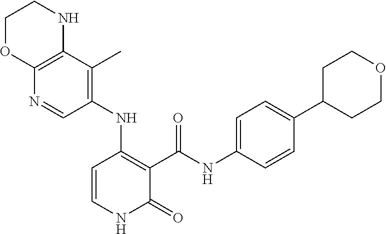 [SMILES: Cc1c(Nc2cc[nH]c(=O)c2C(=O)Nc2ccc(C3CCOCC3)cc2)cnc2c1NCCO2] | 4-((8-methyl-2,3-dihydro-1H- pyrido[2,3-b][1,4]oxazin-7- yl)amino)-2-oxo-N-(4- (tetrahydro-2H-pyran-4- yl)phenyl)-1,2- dihydropyridine-3- carboxamide | 96.57 | 462.23 |
| 134 | 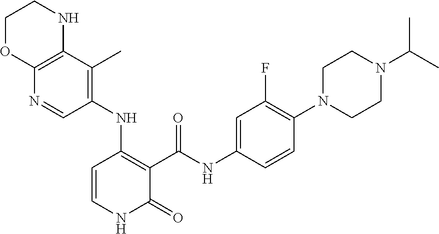 [SMILES: Cc1c(Nc2cc[nH]c(=O)c2C(=O)Nc2ccc(N3CCN(C(C)C)CC3)c(F)c2)cnc2c1NCCO2] | N-(3-fluoro-4-(4- isopropylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.33 | 522.34 |
| 135 | 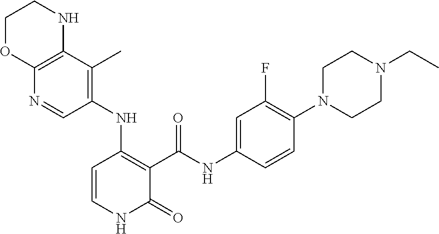 [SMILES: CCN1CCN(c2ccc(NC(=O)c3c(Nc4cnc5c(c4C)NCCO5)cc[nH]c3=O)cc2F)CC1] | N-(4-(4-ethylpiperazin-1-yl)- 3-fluorophenyl)-4-((8-methyl- 2,3-dihydro-1H-pyrido[2,3- b][1,4]oxazin-7-yl)amino)-2- oxo-1,2-dihydropyridine-3- carboxamide | 95.84 | 506.29 [M − H] |
| 136 | 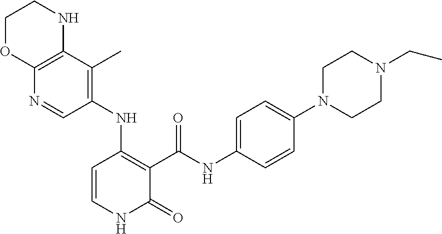 [SMILES: CCN1CCN(c2ccc(NC(=O)c3c(Nc4cnc5c(c4C)NCCO5)cc[nH]c3=O)cc2)CC1] | N-(4-(4-ethylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.66 | 490.29 |
| 137 | 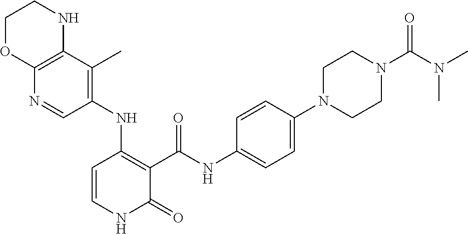 [SMILES: Cc1c(Nc2cc[nH]c(=O)c2C(=O)Nc2ccc(N3CCN(C(=O)N(C)C)CC3)cc2)cnc2c1NCCO2] | N,N-dimethyl-4-(4-(4-((8- methyl-2,3-dihydro-1H- pyrido[2,3-b][1,4]oxazin-7- yl)amino)-2-oxo-1,2- dihydropyridine-3- carboxamido)phenyl) piperazine- 1-carboxamide | 99.64 | 533.45 |
| 138 | 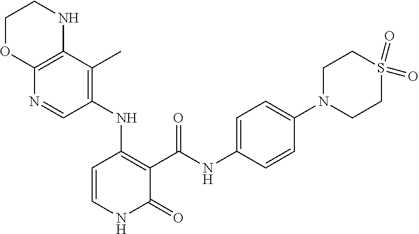 [SMILES: Cc1c(Nc2cc[nH]c(=O)c2C(=O)Nc2ccc(N3CCS(=O)(=O)CC3)cc2)cnc2c1NCCO2] | N-(4-(1,1- dioxidothiomorpholino) phenyl)- 4-((8-methyl-2,3-dihydro- 1H-pyrido[2,3-b][1,4]oxazin- 7-yl)amino)-2-oxo-1,2- dihydropyridine-3- carboxamide | 99.15 | 511.17 |
| 139 | 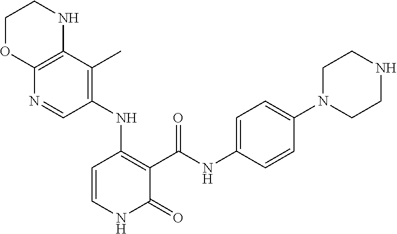 [SMILES: Cc1c(Nc2cc[nH]c(=O)c2C(=O)Nc2ccc(N3CCNCC3)cc2)cnc2c1NCCO2] | 4-((8-methyl-2,3-dihydro-1H- pyrido[2,3-b][1,4]oxazin-7- yl)amino)-2-oxo-N-(4- (piperazin-1-yl)phenyl)-1,2- dihydropyridine-3- carboxamide | 97.64 | 462.33 |
| 140 | 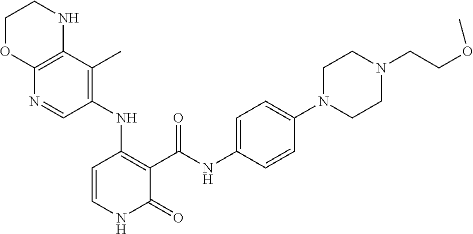 [SMILES: COCCN1CCN(c2ccc(NC(=O)c3c(Nc4cnc5c(c4C)NCCO5)cc[nH]c3=O)cc2)CC1] | N-(4-(4-(2- methoxyethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.27 | 520.28 |
| 143 | 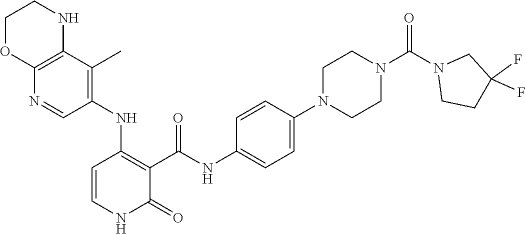 [SMILES: Cc1c(Nc2cc[nH]c(=O)c2C(=O)Nc2ccc(N3CCN(C(=O)N4CCC(F)(F)C4)CC3)cc2)cnc2c1NCCO2] | N-(4-(4-(3,3- difluoropyrrolidine-1- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.16 | 595.44 |
| 144 | 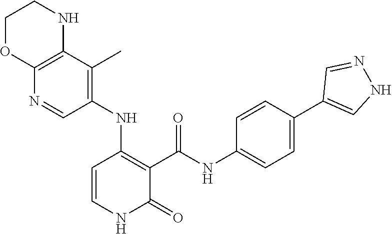 [SMILES: Cc1c(Nc2cc[nH]c(=O)c2C(=O)Nc2ccc(-c3cn[nH]c3)cc2)cnc2c1NCCO2] | N-(4-(1H-pyrazol-4- yl)phenyl)-4-((8-methyl-2,3- dihydro-1H-pyrido[2,3- b][1,4]oxazin-7-yl)amino)-2- oxo-1,2-dihydropyridine-3- carboxamide | 99.64 | 444.35 |
| 145 | 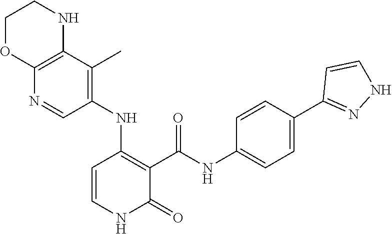 [SMILES: Cc1c(Nc2cc[nH]c(=O)c2C(=O)Nc2ccc(-c3cc[nH]n3)cc2)cnc2c1NCCO2] | N-(4-(1H-pyrazol-3- yl)phenyl)-4-((8-methyl-2,3- dihydro-1H-pyrido[2,3- b][1,4]oxazin-7-yl)amino)-2- oxo-1,2-dihydropyridine-3- carboxamide | 99.2 | 444.1 |
| 146 | 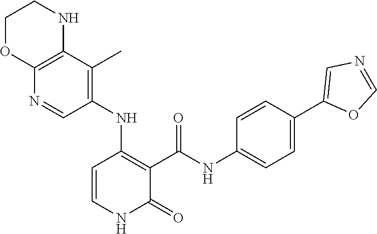 [SMILES: Cc1c(Nc2cc[nH]c(=O)c2C(=O)Nc2ccc(-c3cnco3)cc2)cnc2c1NCCO2] | 4-((8-methyl-2,3-dihydro-1H- pyrido[2,3-b][1,4]oxazin-7- yl)amino)-N-(4-(oxazol-5- yl)phenyl)-2-oxo-1,2- dihydropyridine-3- carboxamide | 99.89 | 445.37 |
| 147 | 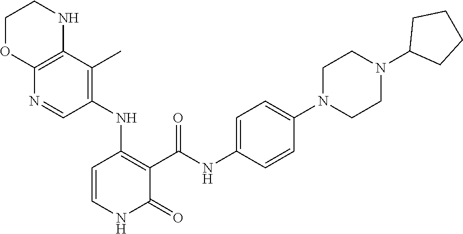 [SMILES: Cc1c(Nc2cc[nH]c(=O)c2C(=O)Nc2ccc(N3CCN(C4CCCC4)CC3)cc2)cnc2c1NCCO2] | N-(4-(4-cyclopentylpiperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.03 | 530.45 |
| 151 | 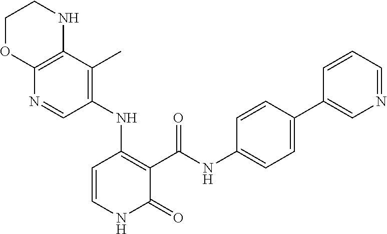 [SMILES: Cc1c(Nc2cc[nH]c(=O)c2C(=O)Nc2ccc(-c3cccnc3)cc2)cnc2c1NCCO2] | 4-((8-methyl-2,3-dihydro-1H- pyrido[2,3-b][1,4]oxazin-7- yl)amino)-2-oxo-N-(4- (pyridin-3-yl)phenyl)-1,2- dihydropyridine-3- carboxamide | 99.61 | 455.40 |
| 152 | 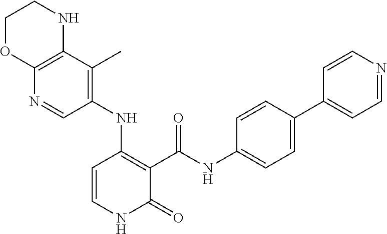 [SMILES: Cc1c(Nc2cc[nH]c(=O)c2C(=O)Nc2ccc(-c3ccncc3)cc2)cnc2c1NCCO2] | 4-((8-methyl-2,3-dihydro-1H- pyrido[2,3-b][1,4]oxazin-7- yl)amino)-2-oxo-N-(4- (pyridin-4-yl)phenyl)-1,2- dihydropyridine-3- carboxamide | 98.78 | 455.2 |
| 153 | 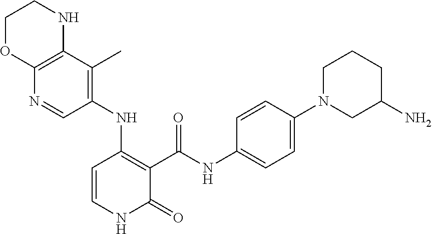 [SMILES: Cc1c(Nc2cc[nH]c(=O)c2C(=O)Nc2ccc(N3CCCC(N)C3)cc2)cnc2c1NCCO2] | N-(4-(3-aminopiperidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.44 | 476.41 |
| 154 | 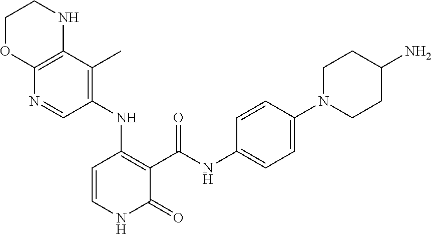 [SMILES: Cc1c(Nc2cc[nH]c(=O)c2C(=O)Nc2ccc(N3CCC(N)CC3)cc2)cnc2c1NCCO2] | N-(4-(4-aminopiperidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.7 | 476.42 |
| 156 | 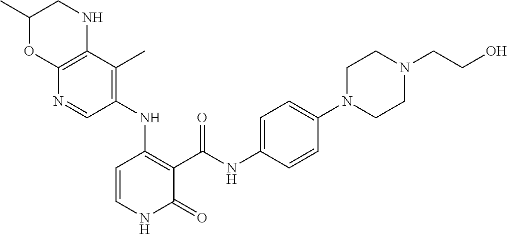 [SMILES: Cc1c(Nc2cc[nH]c(=O)c2C(=O)Nc2ccc(N3CCN(CCO)CC3)cc2)cnc2c1NCC(C)O2] | 4-((3,8-dimethyl-2,3-dihydro- 1H-pyrido[2,3-b][1,4]oxazin- 7-yl)amino)-N-(4-(4-(2- hydroxyethyl)piperazin-1- yl)phenyl)-2-oxo-1,2- dihydropyridine-3- carboxamide | 97.76 | 520.31 |
| 161 | 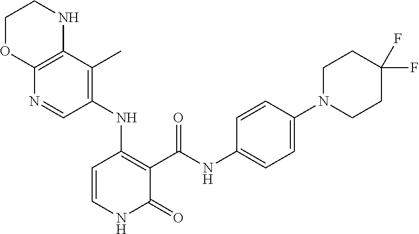 [SMILES: Cc1c(Nc2cc[nH]c(=O)c2C(=O)Nc2ccc(N3CCC(F)(F)CC3)cc2)cnc2c1NCCO2] | N-(4-(4,4-difluoropiperidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.86 | 497.2 |
| 162 | 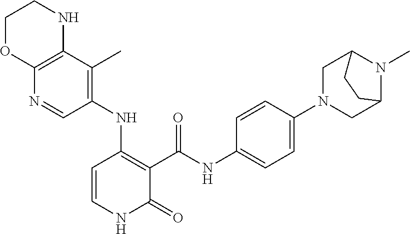 [SMILES: Cc1c(Nc2cc[nH]c(=O)c2C(=O)Nc2ccc(N3CC4CCC(C3)N4C)cc2)cnc2c1NCCO2] | 4-((8-methyl-2,3-dihydro-1H- pyrido[2,3-b][1,4]oxazin-7- yl)amino)-N-(4-(8-methyl- 3,8-diazabicyclo[3.2.1]octan- 3-yl)phenyl)-2-oxo-1,2- dihydropyridine-3- carboxamide | 99.49 | 502.3 |
| 163 | 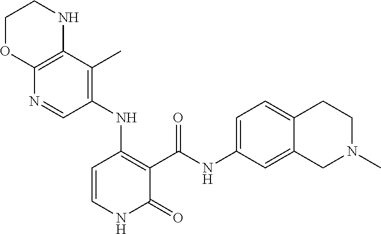 [SMILES: Cc1c(Nc2cc[nH]c(=O)c2C(=O)Nc2ccc3c(c2)CN(C)CC3)cnc2c1NCCO2] | N-(2-methyl-1,2,3,4- tetrahydroisoquinolin-7-yl)-4- ((8-methyl-2,3-dihydro-1H- pyrido[2,3-b][1,4]oxazin-7- yl)amino)-2-oxo-1,2- dihydropyridine-3- carboxamide | 99.18 | 445.21 [M − H] |
| 164 | 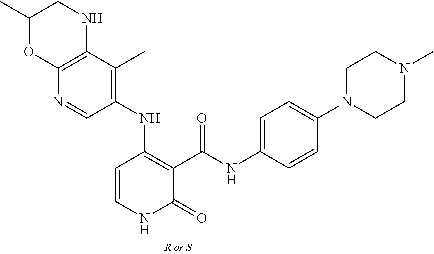 | 4-((3,8-dimethyl-2,3-dihydro- 1H-pyrido[2,3-b][1,4]oxazin- 7-yl)amino)-N-(4-(4- methylpiperazin-1-yl)phenyl)- 2-oxo-1,2-dihydropyridine-3- carboxamide | 99.37 | 488.2 [M − H] |
| 165 | 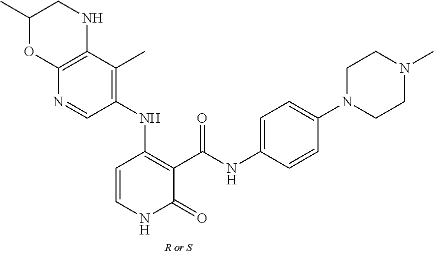 | 4-((3,8-dimethyl-2,3-dihydro- 1H-pyrido[2,3-b][1,4]oxazin- 7-yl)amino)-N-(4-(4- methylpiperazin-1-yl)phenyl)- 2-oxo-1,2-dihydropyridine-3- carboxamide | 98.44 | 488.2 [M − H] |
| 167 | 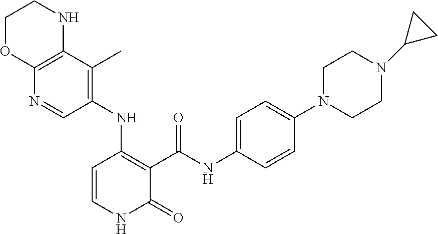 [SMILES: Cc1c(Nc2cc[nH]c(=O)c2C(=O)Nc2ccc(N3CCN(C4CC4)CC3)cc2)cnc2c1NCCO2] | N-(4-(4- cyclopropylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.97 | 502.31 |
| 168 | 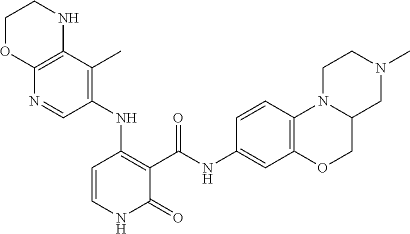 [SMILES: Cc1c(Nc2cc[nH]c(=O)c2C(=O)Nc2ccc3c(c2)OCC2CN(C)CCN32)cnc2c1NCCO2] | N-(3-methyl-1,2,3,4,4a,5- hexahydrobenzo[b]pyrazino [1,2-d][1,4]oxazin-8-yl)-4-((8- methyl-2,3-dihydro-1H- pyrido[2,3-b][1,4]oxazin-7- yl)amino)-2-oxo-1,2- dihydropyridine-3- carboxamide | 96.8 | 504.29 |
| 169 | 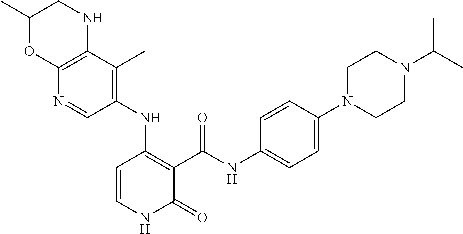 [SMILES: Cc1c(Nc2cc[nH]c(=O)c2C(=O)Nc2ccc(N3CCN(C(C)C)CC3)cc2)cnc2c1NCC(C)O2] | 4-((3,8-dimethyl-2,3-dihydro- 1H-pyrido[2,3-b][1,4]oxazin- 7-yl)amino)-N-(4-(4- isopropylpiperazin-1- yl)phenyl)-2-oxo-1,2- dihydropyridine-3- carboxamide | 99.8 | 518.4 |
| 170 | 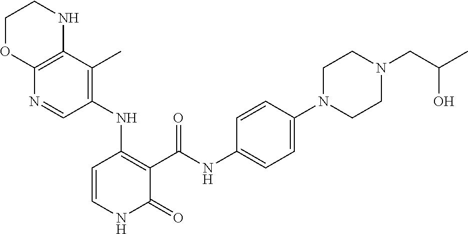 [SMILES: Cc1c(Nc2cc[nH]c(=O)c2C(=O)Nc2ccc(N3CCN(CC(C)O)CC3)cc2)cnc2c1NCCO2] | N-(4-(4-(2- hydroxypropyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.6 | 520.33 |
| 171 | 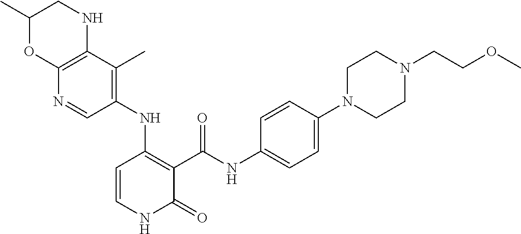 | 4-((3,8-dimethyl-2,3-dihydro- 1H-pyrido[2,3-b][1,4]oxazin- 7-yl)amino)-N-(4-(4-(2- methoxyethyl)piperazin-1- yl)phenyl)-2-oxo-1,2- dihydropyridine-3- carboxamide | 97.4 | 532.36 [M − H] |
| 172 | 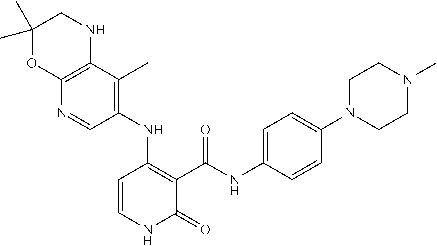 | N-(4-(4-methylpiperazin-1- yl)phenyl)-2-oxo-4-((3,3,8- trimethyl-2,3-dihydro-1H- pyrido[2,3-b][1,4]oxazin-7- yl)amino)-1,2- dihydropyridine-3- carboxamide | 99.2 | 502.36 [M − H] |
| 173 | 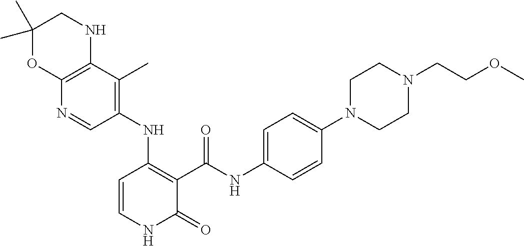 | N-(4-(4-(2- methoxyethyl)piperazin-1- yl)phenyl)-2-oxo-4-((3,3,8- trimethyl-2,3-dihydro-1H- pyrido[2,3-b][1,4]oxazin-7- yl)amino)-1,2- dihydropyridine-3- carboxamide | 96.4 | 548.30 [M − H] |
| 174 | 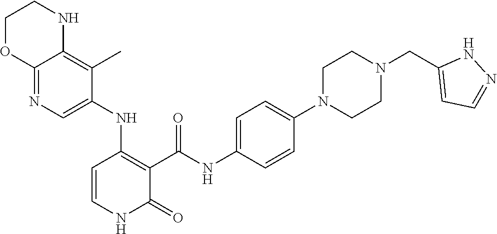 [SMILES: Cc1c(Nc2cc[nH]c(=O)c2C(=O)Nc2ccc(N3CCN(Cc4ccn[nH]4)CC3)cc2)cnc2c1NCCO2] | N-(4-(4-((1H-pyrazol-5- yl)methyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 96.9 | 542.38 |
| 175 | 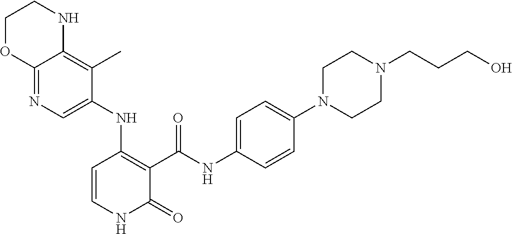 [SMILES: Cc1c(Nc2cc[nH]c(=O)c2C(=O)Nc2ccc(N3CCN(CCCO)CC3)cc2)cnc2c1NCCO2] | N-(4-(4-(3- hydroxypropyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.7 | 520.35 |
| 176 | 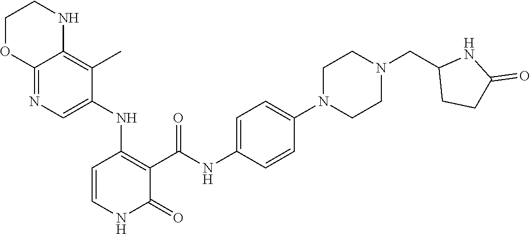 [SMILES: Cc1c(Nc2cc[nH]c(=O)c2C(=O)Nc2ccc(N3CCN(CC4CCC(=O)N4)CC3)cc2)cnc2c1NCCO2] | 4-((8-methyl-2,3-dihydro-1H- pyrido[2,3-b][1,4]oxazin-7- yl)amino)-2-oxo-N-(4-(4-((5- oxopyrrolidin-2- yl)methyl)piperazin-1- yl)phenyl)-1,2- dihydropyridine-3- carboxamide | 99.2 | 559.4 |
| 177 | 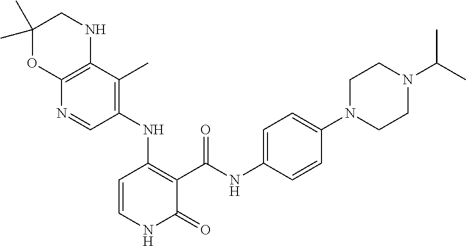 [SMILES: Cc1c(Nc2cc[nH]c(=O)c2C(=O)Nc2ccc(N3CCN(C(C)C)CC3)cc2)cnc2c1NCC(C)(C)O2] | N-(4-(4-isopropylpiperazin-1- yl)phenyl)-2-oxo-4-((3,3,8- trimethyl-2,3-dihydro-1H- pyrido[2,3-b][1,4]oxazin-7- yl)amino)-1,2- dihydropyridine-3- carboxamide | 98.4 | 530.32 [M − H] |
| 178 | 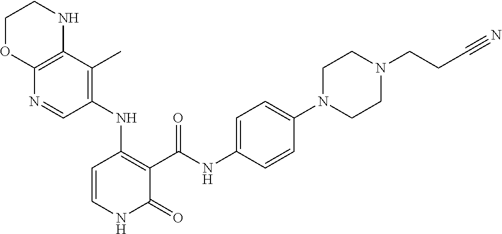 [SMILES: Cc1c(Nc2cc[nH]c(=O)c2C(=O)Nc2ccc(N3CCN(CCC#N)CC3)cc2)cnc2c1NCCO2] | N-(4-(4-(2- cyanoethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.7 | 515.32 |
| 179 | 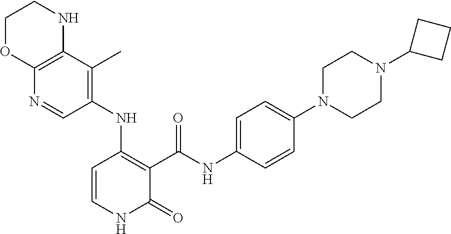 [SMILES: Cc1c(Nc2cc[nH]c(=O)c2C(=O)Nc2ccc(N3CCN(C4CCC4)CC3)cc2)cnc2c1NCCO2] | N-(4-(4-cyclobutylpiperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.1 | 516.36 |
| 180 | 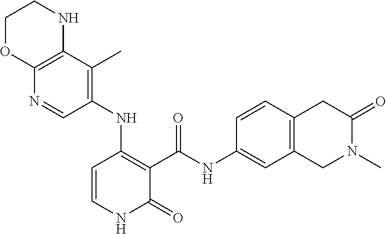 [SMILES: Cc1c(Nc2cc[nH]c(=O)c2C(=O)Nc2ccc3c(c2)CN(C)C(=O)C3)cnc2c1NCCO2] | 4-((8-methyl-2,3-dihydro-1H- pyrido[2,3-b][1,4]oxazin-7- yl)amino)-N-(2-methyl-3- oxo-1,2,3,4- tetrahydroisoquinolin-7-yl)-2- oxo-1,2-dihydropyridine-3- carboxamide | 99.1 | 459.25 [M − H] |
| 181 | 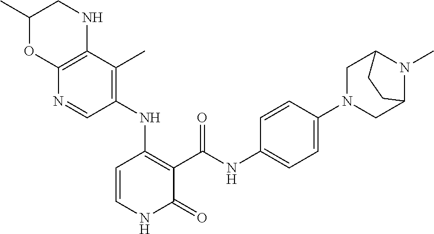 | 4-((3,8-dimethyl-2,3-dihydro- 1H-pyrido[2,3-b][1,4]oxazin- 7-yl)amino)-N-(4-(8-methyl- 3,8-diazabicyclo[3.2.1]octan- 3-yl)phenyl)-2-oxo-1,2- dihydropyridine-3- carboxamide | 98.9 | 516.37 |
| 182 | 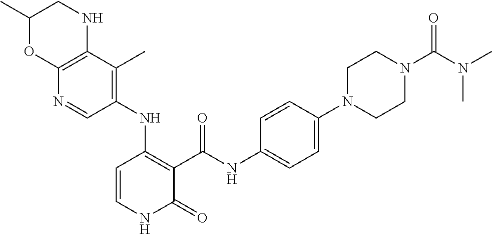 | 4-(4-(4-((3,8-dimethyl-2,3- dihydro-1H-pyrido[2,3- b][1,4]oxazin-7-yl)amino)-2- oxo-1,2-dihydropyridine-3- carboxamido)phenyl)-N,N- dimethylpiperazine-1- carboxamide | 99.7 | 547.35 |
| 183 | 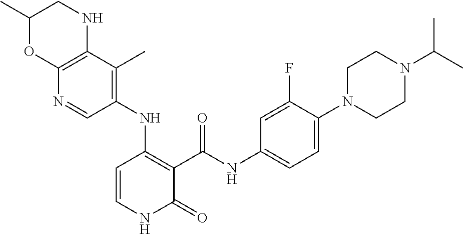 [SMILES: Cc1c(Nc2cc[nH]c(=O)c2C(=O)Nc2ccc(N3CCN(C(C)C)CC3)c(F)c2)cnc2c1NCC(C)O2] | 4-((3,8-dimethyl-2,3-dihydro- 1H-pyrido[2,3-b][1,4]oxazin- 7-yl)amino)-N-(3-fluoro-4-(4- isopropylpiperazin-1- yl)phenyl)-2-oxo-1,2- dihydropyridine-3- carboxamide | 98.6 | 534.35 [M − H] |
| 184 | 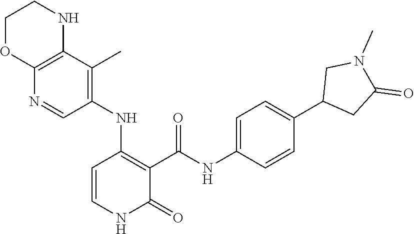 [SMILES: Cc1c(Nc2cc[nH]c(=O)c2C(=O)Nc2ccc(C3CC(=O)N(C)C3)cc2)cnc2c1NCCO2] | 4-((8-methyl-2,3-dihydro-1H- pyrido[2,3-b][1,4]oxazin-7- yl)amino)-N-(4-(1-methyl-5- oxopyrrolidin-3-yl)phenyl)-2- oxo-1,2-dihydropyridine-3- carboxamide | 98.9 | 475.28 |
| 185 | 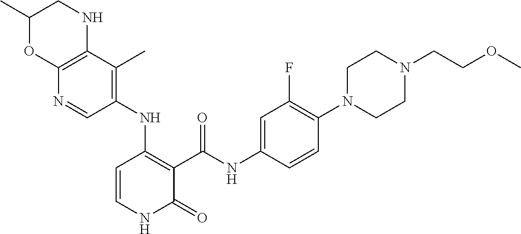 | 4-((3,8-dimethyl-2,3-dihydro- 1H-pyrido[2,3-b][1,4]oxazin- 7-yl)amino)-N-(3-fluoro-4-(4- (2-methoxyethyl)piperazin-1- yl)phenyl)-2-oxo-1,2- dihydropyridine-3- carboxamide | 99.6 | 552.35 |
| 186 | 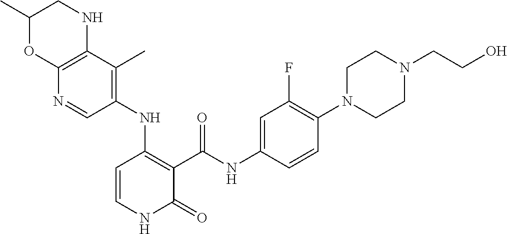 [SMILES: Cc1c(Nc2cc[nH]c(=O)c2C(=O)Nc2ccc(N3CCN(CCO)CC3)c(F)c2)cnc2c1NCC(C)O2] | 4-((3,8-dimethyl-2,3-dihydro- 1H-pyrido[2,3-b][1,4]oxazin- 7-yl)amino)-N-(3-fluoro-4-(4- (2-hydroxyethyl)piperazin-1- yl)phenyl)-2-oxo-1,2- dihydropyridine-3- carboxamide | 99.0 | 538.3 |
| 187 | 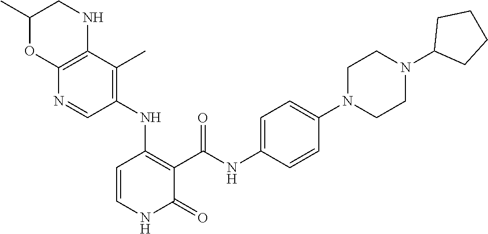 [SMILES: Cc1c(Nc2cc[nH]c(=O)c2C(=O)Nc2ccc(N3CCN(C4CCCC4)CC3)cc2)cnc2c1NCC(C)O2] | N-(4-(4-cyclopentylpiperazin- 1-yl)phenyl)-4-((3,8- dimethyl-2,3-dihydro-1H- pyrido[2,3-b][1,4]oxazin-7- yl)amino)-2-oxo-1,2- dihydropyridine-3- carboxamide | 99.8 | 544.41 |
| 188 | 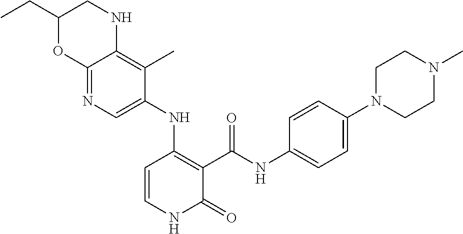 [SMILES: CCC1CNc2c(ncc(Nc3cc[nH]c(=O)c3C(=O)Nc3ccc(N4CCN(C)CC4)cc3)c2C)O1] | 4-((3-ethyl-8-methyl-2,3- dihydro-1H-pyrido[2,3- b][1,4]oxazin-7-yl)amino)-N- (4-(4-methylpiperazin-1- yl)phenyl)-2-oxo-1,2- dihydropyridine-3- carboxamide | 98.7 | 504.36 |
| 189 | 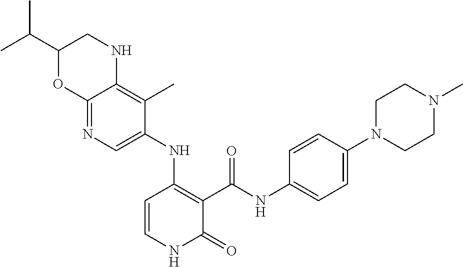 [SMILES: Cc1c(Nc2cc[nH]c(=O)c2C(=O)Nc2ccc(N3CCN(C)CC3)cc2)cnc2c1NCC(C(C)C)O2] | 4-((3-isopropyl-8-methyl-2,3- dihydro-1H-pyrido[2,3- b][1,4]oxazin-7-yl)amino)-N- (4-(4-methylpiperazin-1- yl)phenyl)-2-oxo-1,2- dihydropyridine-3- carboxamide | 98.6 | 518.36 |
| 190 | 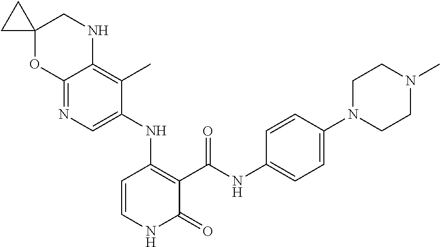 [SMILES: Cc1c(Nc2cc[nH]c(=O)c2C(=O)Nc2ccc(N3CCN(C)CC3)cc2)cnc2c1NCC1(CC1)O2] | 4-((8′-methyl-1′,2′- dihydrospiro[cyclopropane- 1,3′-pyrido[2,3- b][1,4]oxazin]-7′-yl)amino)- N-(4-(4-methylpiperazin-1- yl)phenyl)-2-oxo-1,2- dihydropyridine-3- carboxamide | 99.7 | 502.34 |
| 191 | 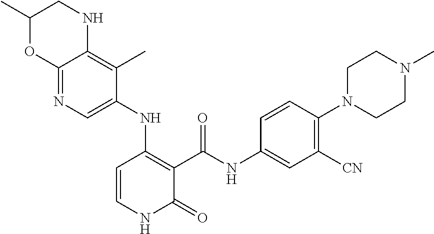 | N-(3-cyano-4-(4- methylpiperazin-1-yl)phenyl)- 4-((3,8-dimethyl-2,3-dihydro- 1H-pyrido[2,3-b][1,4]oxazin- 7-yl)amino)-2-oxo-1,2- dihydropyridine-3- carboxamide | 99.6 | 515.3 |
| 192 | 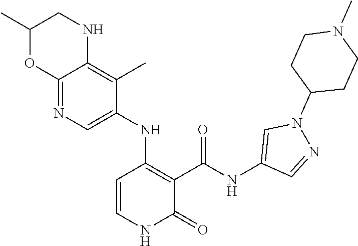 [SMILES: Cc1c(Nc2cc[nH]c(=O)c2C(=O)Nc2cnn(C3CCN(C)CC3)c2)cnc2c1NCC(C)O2] | 4-((3,8-dimethyl-2,3-dihydro- 1H-pyrido[2,3-b][1,4]oxazin- 7-yl)amino)-N-(1-(1- methylpiperidin-4-yl)-1H- pyrazol-4-yl)-2-oxo-1,2- dihydropyridine-3- carboxamide | 99.8 | 479.31 |
| 193 | 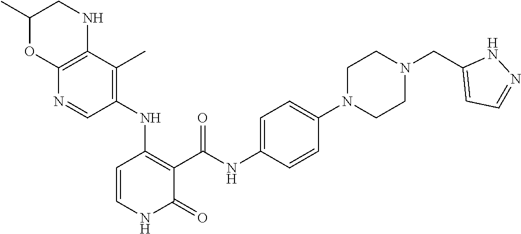 [SMILES: Cc1c(Nc2cc[nH]c(=O)c2C(=O)Nc2ccc(N3CCN(Cc4ccn[nH]4)CC3)cc2)cnc2c1NCC(C)O2] | N-(4-(4-((1H-pyrazol-5- yl)methyl)piperazin-1- yl)phenyl)-4-((3,8-dimethyl- 2,3-dihydro-1H-pyrido[2,3- b][1,4]oxazin-7-yl)amino)-2- oxo-1,2-dihydropyridine-3- carboxamide | 98.6 | 556.35 |
| 194 | 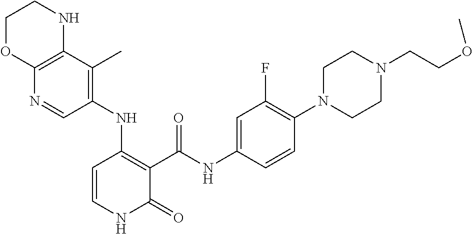 [SMILES: COCCN1CCN(c2ccc(NC(=O)c3c(Nc4cnc5c(c4C)NCCO5)cc[nH]c3=O)cc2F)CC1] | N-(3-fluoro-4-(4-(2- methoxyethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.2 | 536.35 |
| 195 | 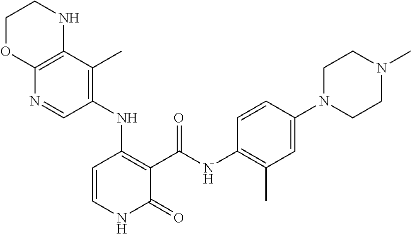 [SMILES: Cc1cc(N2CCN(C)CC2)ccc1NC(=O)c1c(Nc2cnc3c(c2C)NCCO3)cc[nH]c1=O] | 4-((8-methyl-2,3-dihydro-1H- pyrido[2,3-b][1,4]oxazin-7- yl)amino)-N-(2-methyl-4-(4- methylpiperazin-1-yl)phenyl)- 2-oxo-1,2-dihydropyridine-3- carboxamide | 98.61 | 488.29 [M − H] |
| 196 | 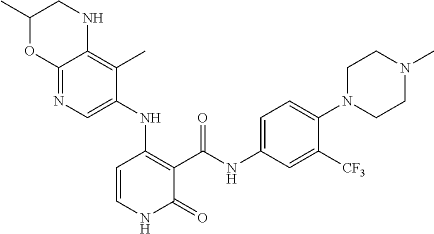 | 4-((3,8-dimethyl-2,3-dihydro- 1H-pyrido[2,3-b][1,4]oxazin- 7-yl)amino)-N-(4-(4- methylpiperazin-1-yl)-3- (trifluoromethyl)phenyl)-2- oxo-1,2-dihydropyridine-3- carboxamide | 99.2 | 558.33 |
| 197 | 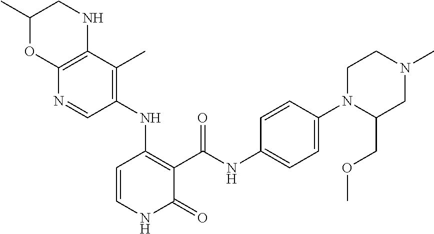 | 4-((3,8-dimethyl-2,3-dihydro- 1H-pyrido[2,3-b][1,4]oxazin- 7-yl)amino)-N-(4-(2- (methoxymethyl)-4- methylpiperazin-1-yl)phenyl)- 2-oxo-1,2-dihydropyridine-3- carboxamide | 99.8 | 534.35 |
| 198 | 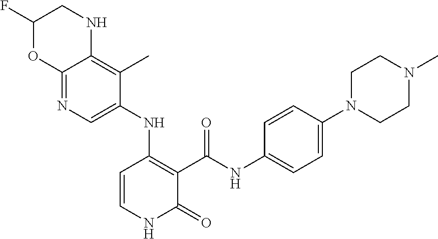 [SMILES: Cc1c(Nc2cc[nH]c(=O)c2C(=O)Nc2ccc(N3CCN(C)CC3)cc2)cnc2c1NCC(F)O2] | 4-((3-fluoro-8-methyl-2,3- dihydro-1H-pyrido[2,3- b][1,4]oxazin-7-yl)amino)-N- (4-(4-methylpiperazin-1- yl)phenyl)-2-oxo-1,2- dihydropyridine-3- carboxamide | 99.8 | 494.3 |
| 199 | 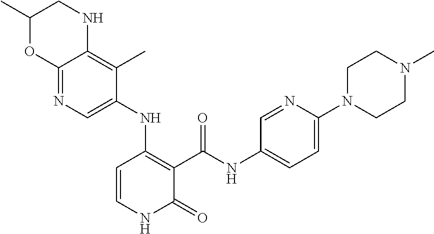 | 4-((3,8-dimethyl-2,3-dihydro- 1H-pyrido[2,3-b][1,4]oxazin- 7-yl)amino)-N-(4-(4- methylpiperazin-1-yl)phenyl)- 2-oxo-1,2-dihydropyridine-3- carboxamide | 99.6 | 491.33 |
| 200 | 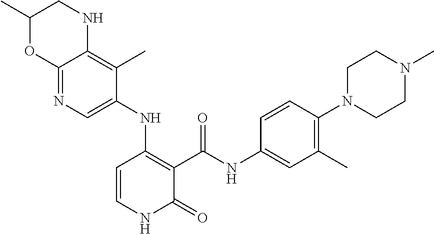 | 4-((3,8-dimethyl-2,3-dihydro- 1H-pyrido[2,3-b][1,4]oxazin- 7-yl)amino)-N-(3-methyl-4- (4-methylpiperazin-1- yl)phenyl)-2-oxo-1,2- dihydropyridine-3- carboxamide | 99.6 | 504.33 |
| 201 | 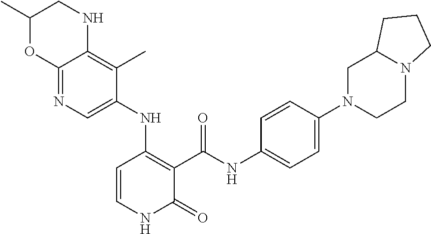 [SMILES: Cc1c(Nc2cc[nH]c(=O)c2C(=O)Nc2ccc(N3CCN4CCCC4C3)cc2)cnc2c1NCC(C)O2] | 4-((3,8-dimethyl-2,3-dihydro- 1H-pyrido[2,3-b][1,4]oxazin- 7-yl)amino)-N-(4- (hexahydropyrrolo[1,2- a]pyrazin-2(1H)-yl)phenyl)-2- oxo-1,2-dihydropyridine-3- carboxamide | 98.8 | 516.34 |
| 202 | 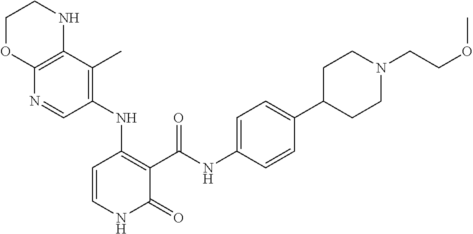 [SMILES: COCCN1CCC(c2ccc(NC(=O)c3c(Nc4cnc5c(c4C)NCCO5)cc[nH]c3=O)cc2)CC1] | N-(4-(1-(2- methoxyethyl)piperidin-4- yl)phenyl)-4-((8-methyl-2,3- dihydro-1H-pyrido[2,3- b][1,4]oxazin-7-yl)amino)-2- oxo-1,2-dihydropyridine-3- carboxamide | 99.1 | 519.38 |
| 203 | 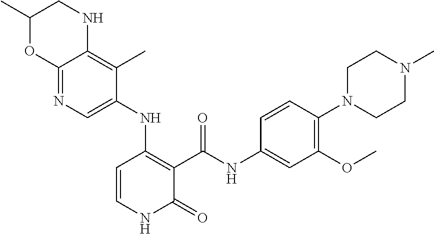 | 4-((3,8-dimethyl-2,3-dihydro- 1H-pyrido[2,3-b][1,4]oxazin- 7-yl)amino)-N-(3-methoxy-4- (4-methylpiperazin-1- yl)phenyl)-2-oxo-1,2- dihydropyridine-3- carboxamide | 93.2 | 520.38 |
| 204 | 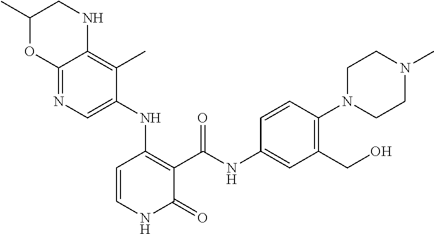 | 4-((3,8-dimethyl-2,3-dihydro- 1H-pyrido[2,3-b][1,4]oxazin- 7-yl)amino)-N-(3- (hydroxymethyl)-4-(4- methylpiperazin-1-yl)phenyl)- 2-oxo-1,2-dihydropyridine-3- carboxamide | 97.5 | 520.35 |
| 205 | 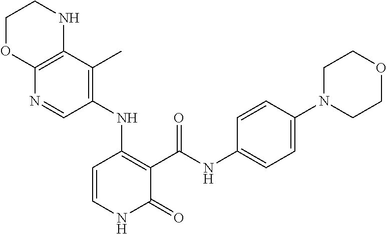 [SMILES: Cc1c(Nc2cc[nH]c(=O)c2C(=O)Nc2ccc(N3CCOCC3)cc2)cnc2c1NCCO2] | 4-((8-methyl-2,3-dihydro-1H- pyrido[2,3-b][1,4]oxazin-7- yl)amino)-N-(4- morpholinophenyl)-2-oxo- 1,2-dihydropyridine-3- carboxamide | 98.2 | 463.27 |
| 206 | 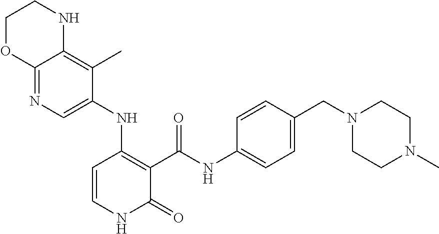 [SMILES: Cc1c(Nc2cc[nH]c(=O)c2C(=O)Nc2ccc(CN3CCN(C)CC3)cc2)cnc2c1NCCO2] | 4-((8-methyl-2,3-dihydro-1H- pyrido[2,3-b][1,4]oxazin-7- yl)amino)-N-(4-((4- methylpiperazin-1- yl)methyl)phenyl)-2-oxo-1,2- dihydropyridine-3- carboxamide | 98.3 | 490.37 |
| 207 | 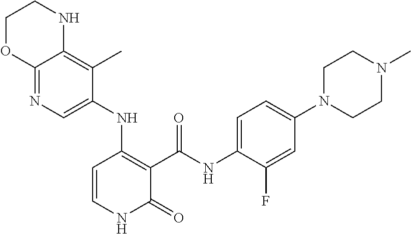 [SMILES: Cc1c(Nc2cc[nH]c(=O)c2C(=O)Nc2ccc(N3CCN(C)CC3)cc2F)cnc2c1NCCO2] | N-(2-fluoro-4-(4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.8 | 494.27 |
| 208 | 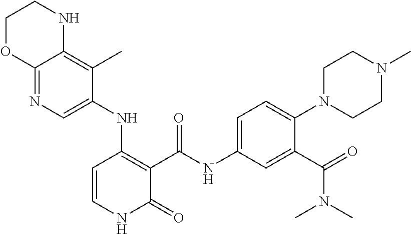 [SMILES: Cc1c(Nc2cc[nH]c(=O)c2C(=O)Nc2ccc(N3CCN(C)CC3)c(C(=O)N(C)C)c2)cnc2c1NCCO2] | N-(3-(dimethylcarbamoyl)-4- (4-methylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.07 | 547.31 |
| 209 | 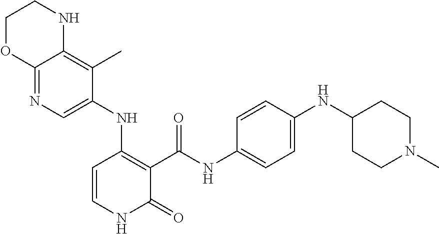 [SMILES: Cc1c(Nc2cc[nH]c(=O)c2C(=O)Nc2ccc(NC3CCN(C)CC3)cc2)cnc2c1NCCO2] | 4-((8-methyl-2,3-dihydro-1H- pyrido[2,3-b][1,4]oxazin-7- yl)amino)-N-(4-((1- methylpiperidin-4- yl)amino)phenyl)-2-oxo-1,2- dihydropyridine-3- carboxamide | 97.7 | 490.37 |
| 210 | 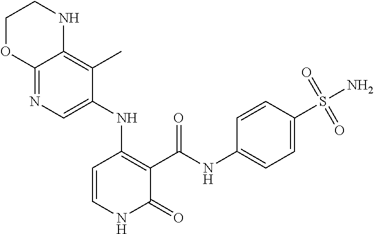 [SMILES: Cc1c(Nc2cc[nH]c(=O)c2C(=O)Nc2ccc(S(N)(=O)=O)cc2)cnc2c1NCCO2] | 4-((8-methyl-2,3-dihydro-1H- pyrido[2,3-b][1,4]oxazin-7- yl)amino)-2-oxo-N-(4- sulfamoylphenyl)-1,2- dihydropyridine-3- carboxamide | 95.1 | 457.24 |
| 211 | 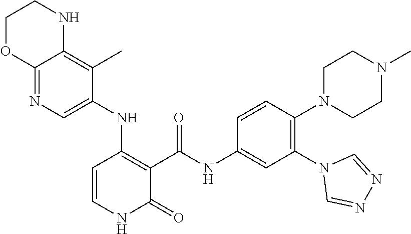 [SMILES: Cc1c(Nc2cc[nH]c(=O)c2C(=O)Nc2ccc(N3CCN(C)CC3)c(-n3cnnc3)c2)cnc2c1NCCO2] | 4-((8-methyl-2,3-dihydro-1H- pyrido[2,3-b][1,4]oxazin-7- yl)amino)-N-(4-(4- methylpiperazin-1-yl)-3-(4H- 1,2,4-triazol-4-yl)phenyl)-2- oxo-1,2-dihydropyridine-3- carboxamide | 97.7 | 543.41 |
| 212 | 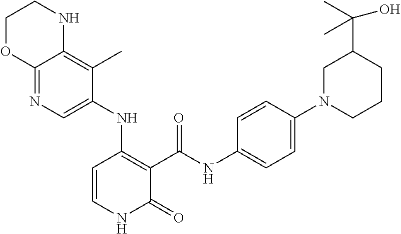 [SMILES: Cc1c(Nc2cc[nH]c(=O)c2C(=O)Nc2ccc(N3CCCC(C(C)(C)O)C3)cc2)cnc2c1NCCO2] | N-(4-(3-(2-hydroxypropan-2- yl)piperidin-1-yl)phenyl)-4- ((8-methyl-2,3-dihydro-1H- pyrido[2,3-b][1,4]oxazin-7- yl)amino)-2-oxo-1,2- dihydropyridine-3- carboxamide | 96.9 | 519.38 |
| 213 | 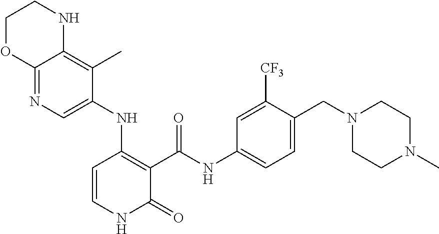 [SMILES: Cc1c(Nc2cc[nH]c(=O)c2C(=O)Nc2ccc(CN3CCN(C)CC3)c(C(F)(F)F)c2)cnc2c1NCCO2] | 4-((8-methyl-2,3-dihydro-1H- pyrido[2,3-b][1,4]oxazin-7- yl)amino)-N-(4-((4- methylpiperazin-1- yl)methyl)-3- (trifluoromethyl)phenyl)-2- oxo-1,2-dihydropyridine-3- carboxamide | 99.1 | 558.38 |
| 214 | 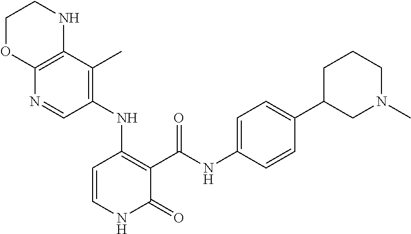 [SMILES: Cc1c(Nc2cc[nH]c(=O)c2C(=O)Nc2ccc(C3CCCN(C)C3)cc2)cnc2c1NCCO2] | 4-((8-methyl-2,3-dihydro-1H- pyrido[2,3-b][1,4]oxazin-7- yl)amino)-N-(4-(1- methylpiperidin-3-yl)phenyl)- 2-oxo-1,2-dihydropyridine-3- carboxamide | 99.1 | 475.32 |
| 215 | 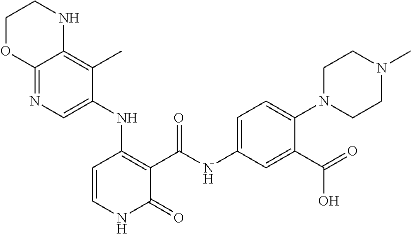 [SMILES: Cc1c(Nc2cc[nH]c(=O)c2C(=O)Nc2ccc(N3CCN(C)CC3)c(C(=O)O)c2)cnc2c1NCCO2] | 5-(4-((8-methyl-2,3-dihydro- 1H-pyrido[2,3-b][1,4]oxazin- 7-yl)amino)-2-oxo-1,2- dihydropyridine-3- carboxamido)-2-(4- methylpiperazin-1-yl)benzoic acid | 99.45 | 520.32 |
| 216 | 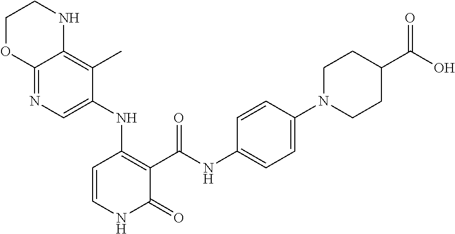 [SMILES: Cc1c(Nc2cc[nH]c(=O)c2C(=O)Nc2ccc(N3CCC(C(=O)O)CC3)cc2)cnc2c1NCCO2] | 1-(4-(4-((8-methyl-2,3- dihydro-1H-pyrido[2,3- b][1,4]oxazin-7-yl)amino)-2- oxo-1,2-dihydropyridine-3- carboxamido)phenyl) piperidine- 4-carboxylic acid | 99.5 | 505.32 |
| 217 | 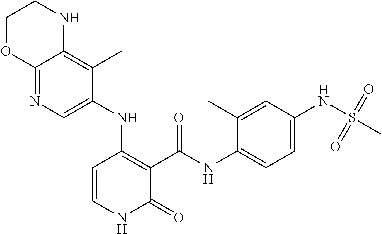 [SMILES: Cc1cc(NS(C)(=O)=O)ccc1NC(=O)c1c(Nc2cnc3c(c2C)NCCO3)cc[nH]c1=O] | 4-((8-methyl-2,3-dihydro-1H- pyrido[2,3-b][1,4]oxazin-7- yl)amino)-N-(2-methyl-4- (methylsulfonamido)phenyl)- 2-oxo-1,2-dihydropyridine-3- carboxamide | 98.27 | 438.32 |
| 218 | 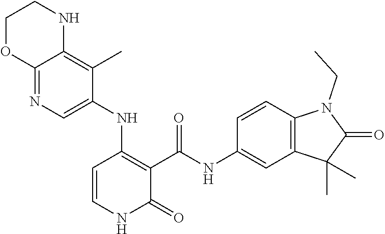 [SMILES: CCN1C(=O)C(C)(C)c2cc(NC(=O)c3c(Nc4cnc5c(c4C)NCCO5)cc[nH]c3=O)ccc21] | N-(1-ethyl-3,3-dimethyl-2- oxoindolin-5-yl)-4-((8- methyl-2,3-dihydro-1H- pyrido[2,3-b][1,4]oxazin-7- yl)amino)-2-oxo-1,2- dihydropyridine-3- carboxamide | 99.33 | 489.31 |
| 219 | 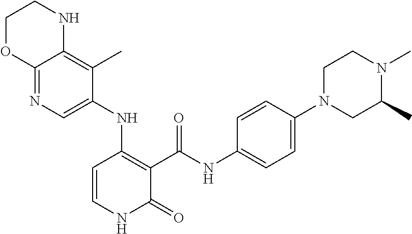 [SMILES: Cc1c(Nc2cc[nH]c(=O)c2C(=O)Nc2ccc(N3CCN(C)[C@@H](C)C3)cc2)cnc2c1NCCO2] | (S)-N-(4-(3,4- dimethylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.6 | 490.35 |
| 220 | 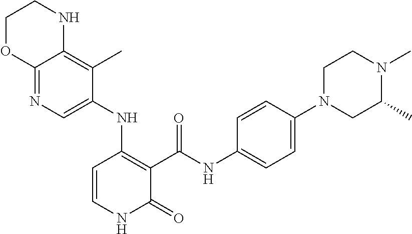 [SMILES: Cc1c(Nc2cc[nH]c(=O)c2C(=O)Nc2ccc(N3CCN(C)[C@H](C)C3)cc2)cnc2c1NCCO2] | (R)-N-(4-(3,4- dimethylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.8 | 490.32 |
| 221 | 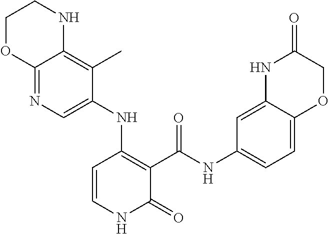 [SMILES: Cc1c(Nc2cc[nH]c(=O)c2C(=O)Nc2ccc3c(c2)NC(=O)CO3)cnc2c1NCCO2] | 4-((8-methyl-2,3-dihydro-1H- pyrido[2,3-b][1,4]oxazin-7- yl)amino)-2-oxo-N-(3-oxo- 3,4-dihydro-2H- benzo[b][1,4]oxazin-6-yl)- 1,2-dihydropyridine-3- carboxamide | 99.52 | 449.31 |
| 222 | 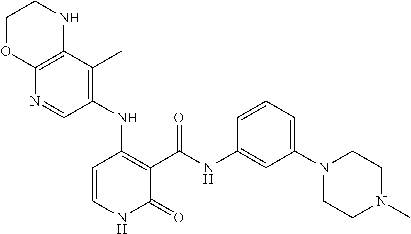 [SMILES: Cc1c(Nc2cc[nH]c(=O)c2C(=O)Nc2cccc(N3CCN(C)CC3)c2)cnc2c1NCCO2] | 4-((8-methyl-2,3-dihydro-1H- pyrido[2,3-b][1,4]oxazin-7- yl)amino)-N-(3-(4- methylpiperazin-1-yl)phenyl)- 2-oxo-1,2-dihydropyridine-3- carboxamide | 95.69 | 476.34 |
| 223 | 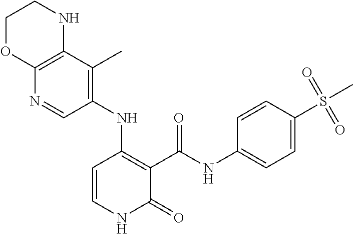 [SMILES: Cc1c(Nc2cc[nH]c(=O)c2C(=O)Nc2ccc(S(C)(=O)=O)cc2)cnc2c1NCCO2] | 4-((8-methyl-2,3-dihydro-1H- pyrido[2,3-b][1,4]oxazin-7- yl)amino)-N-(4- (methylsulfonyl)phenyl)-2- oxo-1,2-dihydropyridine-3- carboxamide | 96.0 | 456.25 |
| 224 | 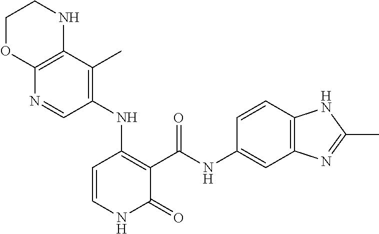 [SMILES: Cc1nc2cc(NC(=O)c3c(Nc4cnc5c(c4C)NCCO5)cc[nH]c3=O)ccc2[nH]1] | N-(2-methyl-1H- benzo[d]imidazol-5-yl)-4-((8- methyl-2,3-dihydro-1H- pyrido[2,3-b][1,4]oxazin-7- yl)amino)-2-oxo-1,2- dihydropyridine-3- carboxamide | 99.7 | 432.32 |
| 225 | 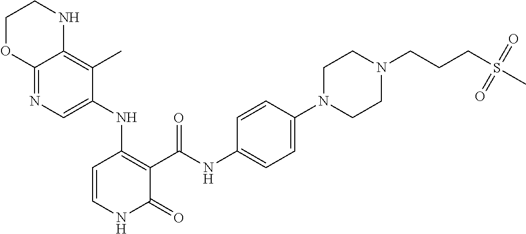 [SMILES: Cc1c(Nc2cc[nH]c(=O)c2C(=O)Nc2ccc(N3CCN(CCCS(C)(=O)=O)CC3)cc2)cnc2c1NCCO2] | 4-((8-methyl-2,3-dihydro-1H- pyrido[2,3-b][1,4]oxazin-7- yl)amino)-N-(4-(4-(3- (methylsulfonyl)propyl) piperazin-1-yl)phenyl)-2- oxo-1,2-dihydropyridine-3- carboxamide | 98.6 | 582.46 |
| 226 | 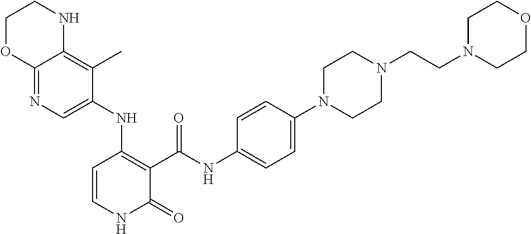 [SMILES: Cc1c(Nc2cc[nH]c(=O)c2C(=O)Nc2ccc(N3CCN(CCN4CCOCC4)CC3)cc2)cnc2c1NCCO2] | 4-((8-methyl-2,3-dihydro-1H- pyrido[2,3-b][1,4]oxazin-7- yl)amino)-N-(4-(4-(2- morpholinoethyl)piperazin-1- yl)phenyl)-2-oxo-1,2- dihydropyridine-3- carboxamide | 98.5 | 575.50 |
| 227 | 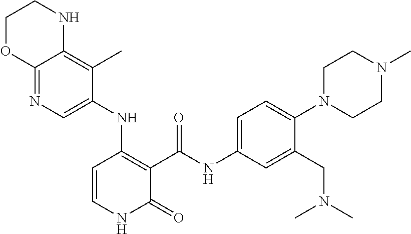 [SMILES: Cc1c(Nc2cc[nH]c(=O)c2C(=O)Nc2ccc(N3CCN(C)CC3)c(CN(C)C)c2)cnc2c1NCCO2] | N-(3- ((dimethylamino)methyl)-4- (4-methylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.6 | 533.46 |
| 228 | 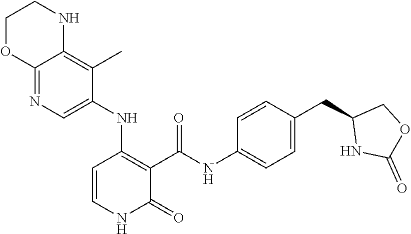 [SMILES: Cc1c(Nc2cc[nH]c(=O)c2C(=O)Nc2ccc(C[C@H]3COC(=O)N3)cc2)cnc2c1NCCO2] | (S)-4-((8-methyl-2,3-dihydro- 1H-pyrido[2,3-b][1,4]oxazin- 7-yl)amino)-2-oxo-N-(4-((2- oxooxazolidin-4- yl)methyl)phenyl)-1,2- dihydropyridine-3- carboxamide | 96.5 | 477.2 |
| 229 | 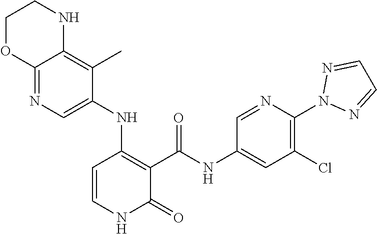 [SMILES: Cc1c(Nc2cc[nH]c(=O)c2C(=O)Nc2cnc(-n3nccn3)c(Cl)c2)cnc2c1NCCO2] | N-(5-chloro-6-(2H-1,2,3- triazol-2-yl)pyridin-3-yl)-4- ((8-methyl-2,3-dihydro-1H- pyrido[2,3-b][1,4]oxazin-7- yl)amino)-2-oxo-1,2- dihydropyridine-3- carboxamide | 96.64 | 480.24 |
| 230 | 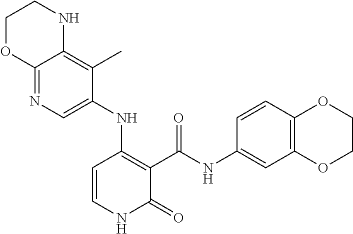 [SMILES: Cc1c(Nc2cc[nH]c(=O)c2C(=O)Nc2ccc3c(c2)OCCO3)cnc2c1NCCO2] | N-(2,3- dihydrobenzo[b][1,4]dioxin- 6-yl)-4-((8-methyl-2,3- dihydro-1H-pyrido[2,3- b][1,4]oxazin-7-yl)amino)-2- oxo-1,2-dihydropyridine-3- carboxamide | 96.9 | 436.27 |
| 231 | 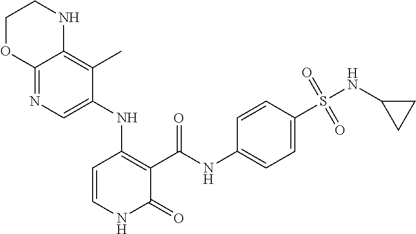 [SMILES: Cc1c(Nc2cc[nH]c(=O)c2C(=O)Nc2ccc(S(=O)(=O)NC3CC3)cc2)cnc2c1NCCO2] | N-(4-(N- cyclopropylsulfamoyl) phenyl)- 4-((8-methyl-2,3-dihydro- 1H-pyrido[2,3-b][1,4]oxazin- 7-yl)amino)-2-oxo-1,2- dihydropyridine-3- carboxamide | 99.2 | 495.1 [M − H] |
| 232 | 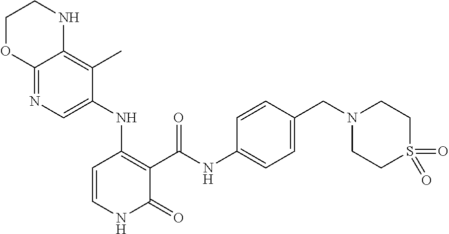 [SMILES: Cc1c(Nc2cc[nH]c(=O)c2C(=O)Nc2ccc(CN3CCS(=O)(=O)CC3)cc2)cnc2c1NCCO2] | N-(4-((1,1- dioxidothiomorpholino) methyl) phenyl)-4-((8-methyl-2,3- dihydro-1H-pyrido[2,3- b][1,4]oxazin-7-yl)amino)-2- oxo-1,2-dihydropyridine-3- carboxamide | 99.8 | 525.2 |
| 233 | 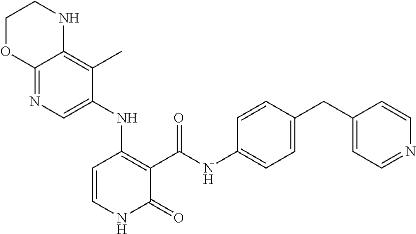 [SMILES: Cc1c(Nc2cc[nH]c(=O)c2C(=O)Nc2ccc(Cc3ccncc3)cc2)cnc2c1NCCO2] | 4-((8-methyl-2,3-dihydro-1H- pyrido[2,3-b][1,4]oxazin-7- yl)amino)-2-oxo-N-(4- (pyridin-4-ylmethyl)phenyl)- 1,2-dihydropyridine-3- carboxamide | 99.6 | 469.29 |
| 234 | 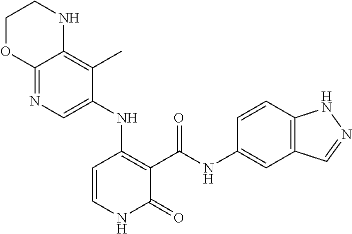 [SMILES: Cc1c(Nc2cc[nH]c(=O)c2C(=O)Nc2ccc3[nH]ncc3c2)cnc2c1NCCO2] | N-(1H-indazol-5-yl)-4-((8- methyl-2,3-dihydro-1H- pyrido[2,3-b][1,4]oxazin-7- yl)amino)-2-oxo-1,2- dihydropyridine-3- carboxamide | 99.3 | 418.27 |
| 235 | 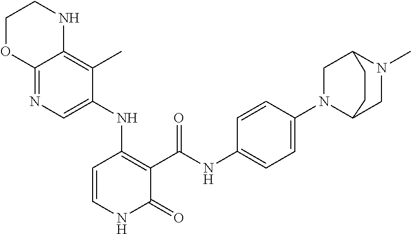 [SMILES: Cc1c(Nc2cc[nH]c(=O)c2C(=O)Nc2ccc(N3CC4CCC3CN4C)cc2)cnc2c1NCCO2] | 4-((8-methyl-2,3-dihydro-1H- pyrido[2,3-b][1,4]oxazin-7- yl)amino)-N-(4-(5-methyl- 2,5-diazabicyclo[2.2.2]octan- 2-yl)phenyl)-2-oxo-1,2- dihydropyridine-3- carboxamide | 99.3 | 502.4 |
| 236 | 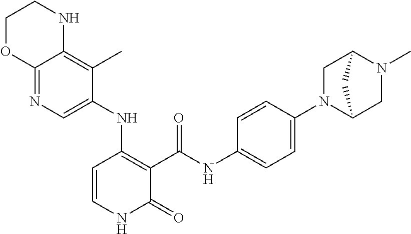 [SMILES: Cc1c(Nc2cc[nH]c(=O)c2C(=O)Nc2ccc(N3C[C@@H]4C[C@H]3CN4C)cc2)cnc2c1NCCO2] | 4-((8-methyl-2,3-dihydro-1H- pyrido[2,3-b][1,4]oxazin-7- yl)amino)-N-(4-((1S,4S)-5- methyl-2,5- diazabicyclo[2.2.1]heptan-2- yl)phenyl)-2-oxo-1,2- dihydropyridine-3- carboxamide | 99.6 | 488.41 |
| 237 | 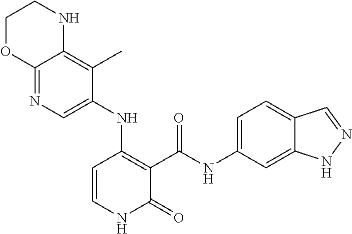 [SMILES: Cc1c(Nc2cc[nH]c(=O)c2C(=O)Nc2ccc3cn[nH]c3c2)cnc2c1NCCO2] | N-(1H-indazol-6-yl)-4-((8- methyl-2,3-dihydro-1H- pyrido[2,3-b][1,4]oxazin-7- yl)amino)-2-oxo-1,2- dihydropyridine-3- carboxamide | 99.8 | 418.37 |
| 238 | 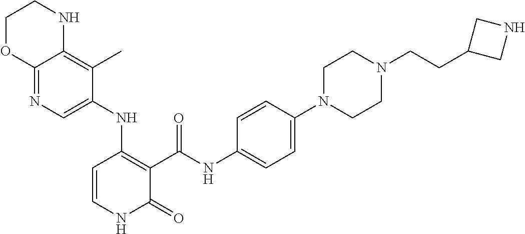 [SMILES: Cc1c(Nc2cc[nH]c(=O)c2C(=O)Nc2ccc(N3CCN(CCC4CNC4)CC3)cc2)cnc2c1NCCO2] | N-(4-(4-(2-(azetidin-3- yl)ethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.2 | 545.44 |
| 239 | 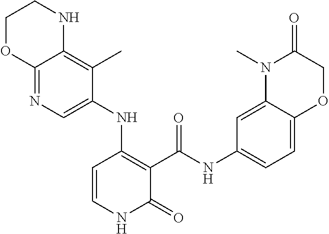 [SMILES: Cc1c(Nc2cc[nH]c(=O)c2C(=O)Nc2ccc3c(c2)N(C)C(=O)CO3)cnc2c1NCCO2] | 4-((8-methyl-2,3-dihydro-1H- pyrido[2,3-b][1,4]oxazin-7- yl)amino)-N-(4-methyl-3- oxo-3,4-dihydro-2H- benzo[b][1,4]oxazin-6-yl)-2- oxo-1,2-dihydropyridine-3- carboxamide | 96.56 | 463.3 |
| 240 | 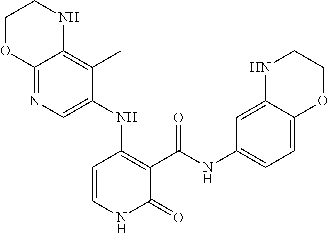 [SMILES: Cc1c(Nc2cc[nH]c(=O)c2C(=O)Nc2ccc3c(c2)NCCO3)cnc2c1NCCO2] | N-(3,4-dihydro-2H- benzo[b][1,4]oxazin-6-yl)-4- ((8-methyl-2,3-dihydro-1H- pyrido[2,3-b][1,4]oxazin-7- yl)amino)-2-oxo-1,2- dihydropyridine-3- carboxamide | 95.2 | 435.36 |
| 241 | 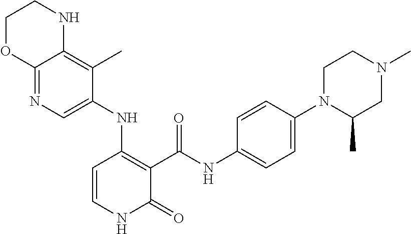 [SMILES: Cc1c(Nc2cc[nH]c(=O)c2C(=O)Nc2ccc(N3CCN(C)C[C@H]3C)cc2)cnc2c1NCCO2] | (R)-N-(4-(2,4- dimethylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.7 | 490.37 |
| 242 | 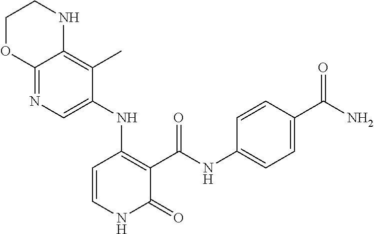 [SMILES: Cc1c(Nc2cc[nH]c(=O)c2C(=O)Nc2ccc(C(N)=O)cc2)cnc2c1NCCO2] | N-(4-carbamoylphenyl)-4-((8- methyl-2,3-dihydro-1H- pyrido[2,3-b][1,4]oxazin-7- yl)amino)-2-oxo-1,2- dihydropyridine-3- carboxamide | 96.5 | 419.2 [M − H] |
| 243 | 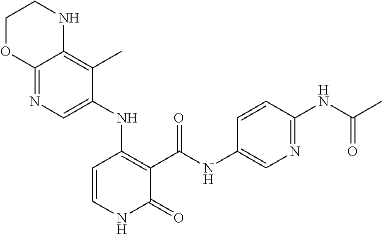 [SMILES: CC(=O)Nc1ccc(NC(=O)c2c(Nc3cnc4c(c3C)NCCO4)cc[nH]c2=O)cn1] | N-(6-acetamidopyridin-3-yl)- 4-((8-methyl-2,3-dihydro-1H- pyrido[2,3-b][1,4]oxazin-7- yl)amino)-2-oxo-1,2- dihydropyridine-3- carboxamide | 92.17 | 436.3 |
| 244 | 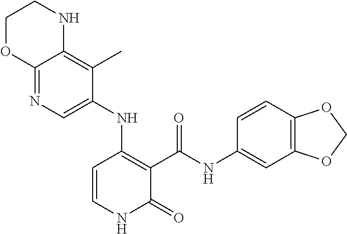 [SMILES: Cc1c(Nc2cc[nH]c(=O)c2C(=O)Nc2ccc3c(c2)OCO3)cnc2c1NCCO2] | N-(benzo[d][1,3]dioxol-5-yl)- 4-((8-methyl-2,3-dihydro-1H- pyrido[2,3-b][1,4]oxazin-7- yl)amino)-2-oxo-1,2- dihydropyridine-3- carboxamide | 98.37 | 422.27 |
| 245 | 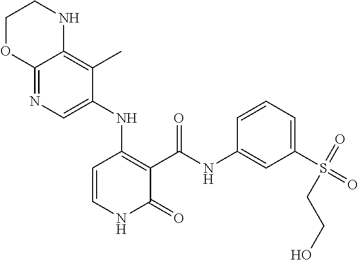 [SMILES: Cc1c(Nc2cc[nH]c(=O)c2C(=O)Nc2cccc(S(=O)(=O)CCO)c2)cnc2c1NCCO2] | N-(3-((2- hydroxyethyl)sulfonyl)phenyl)- 4-((8-methyl-2,3-dihydro- 1H-pyrido[2,3-b][1,4]oxazin- 7-yl)amino)-2-oxo-1,2- dihydropyridine-3- carboxamide | 96.57 | 486.23 |
| 246 | 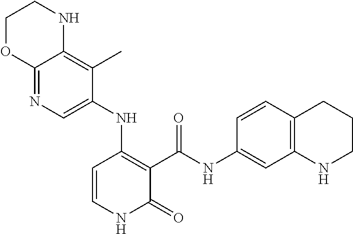 [SMILES: Cc1c(Nc2cc[nH]c(=O)c2C(=O)Nc2ccc3c(c2)NCCC3)cnc2c1NCCO2] | 4-((8-methyl-2,3-dihydro-1H- pyrido[2,3-b][1,4]oxazin-7- yl)amino)-2-oxo-N-(1,2,3,4- tetrahydroquinolin-7-yl)-1,2- dihydropyridine-3- carboxamide | 99.3 | 433.29 |
| 247 | 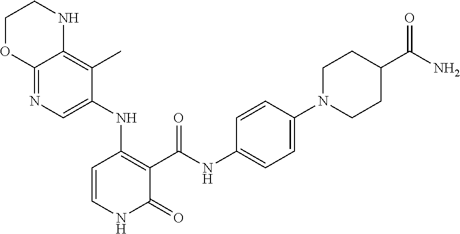 [SMILES: Cc1c(Nc2cc[nH]c(=O)c2C(=O)Nc2ccc(N3CCC(C(N)=O)CC3)cc2)cnc2c1NCCO2] | N-(4-(4-carbamoylpiperidin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 98.6 | 504.33 |
| 248 | 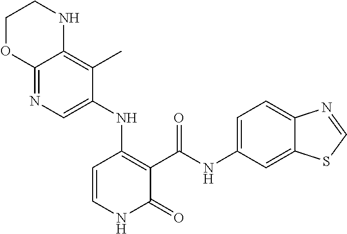 [SMILES: Cc1c(Nc2cc[nH]c(=O)c2C(=O)Nc2ccc3ncsc3c2)cnc2c1NCCO2] | N-(benzo[d]thiazol-6-yl)-4- ((8-methyl-2,3-dihydro-1H- pyrido[2,3-b][1,4]oxazin-7- yl)amino)-2-oxo-1,2- dihydropyridine-3- carboxamide | 99.1 | 435.27 |
| 249 | 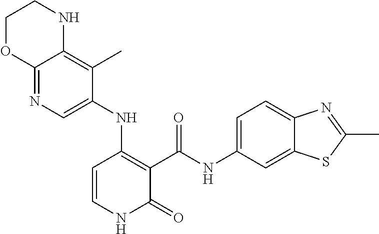 [SMILES: Cc1nc2ccc(NC(=O)c3c(Nc4cnc5c(c4C)NCCO5)cc[nH]c3=O)cc2s1] | 4-((8-methyl-2,3-dihydro-1H- pyrido[2,3-b][1,4]oxazin-7- yl)amino)-N-(2- methylbenzo[d]thiazol-6-yl)- 2-oxo-1,2-dihydropyridine-3- carboxamide | 97.1 | 449.28 |
| 250 | 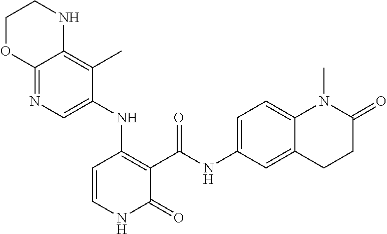 [SMILES: Cc1c(Nc2cc[nH]c(=O)c2C(=O)Nc2ccc3c(c2)CCC(=O)N3C)cnc2c1NCCO2] | 4-((8-methyl-2,3-dihydro-1H- pyrido[2,3-b][1,4]oxazin-7- yl)amino)-N-(1-methyl-2- oxo-1,2,3,4- tetrahydroquinolin-6-yl)-2- oxo-1,2-dihydropyridine-3- carboxamide | 99.4 | 461.31 |
| 251 | 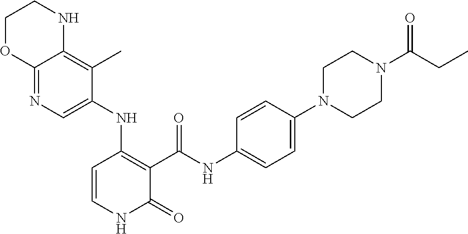 [SMILES: CCC(=O)N1CCN(c2ccc(NC(=O)c3c(Nc4cnc5c(c4C)NCCO5)cc[nH]c3=O)cc2)CC1] | 4-((8-methyl-2,3-dihydro-1H- pyrido[2,3-b][1,4]oxazin-7- yl)amino)-2-oxo-N-(4-(4- propionylpiperazin-1- yl)phenyl)-1,2- dihydropyridine-3- carboxamide | 99.4 | 518.36 |
| 252 | 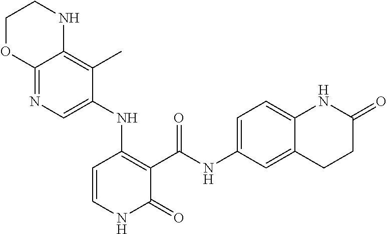 [SMILES: Cc1c(Nc2cc[nH]c(=O)c2C(=O)Nc2ccc3c(c2)CCC(=O)N3)cnc2c1NCCO2] | 4-((8-methyl-2,3-dihydro-1H- pyrido[2,3-b][1,4]oxazin-7- yl)amino)-2-oxo-N-(2-oxo- 1,2,3,4-tetrahydroquinolin-6- yl)-1,2-dihydropyridine-3- carboxamide | 96.0 | 447.38 |
| 253 | 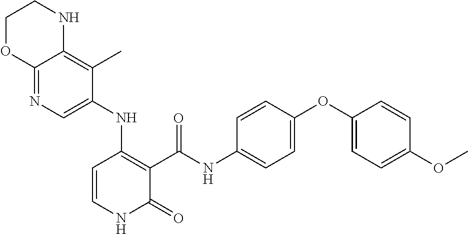 [SMILES: COc1ccc(Oc2ccc(NC(=O)c3c(Nc4cnc5c(c4C)NCCO5)cc[nH]c3=O)cc2)cc1] | N-(4-(4- methoxyphenoxy)phenyl)-4- ((8-methyl-2,3-dihydro-1H- pyrido[2,3-b][1,4]oxazin-7- yl)amino)-2-oxo-1,2- dihydropyridine-3- carboxamide | 96.77 | 500.1 |
| 254 | 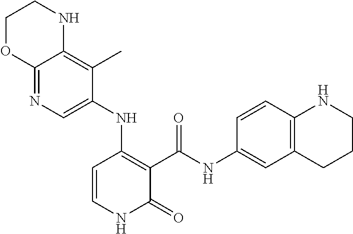 [SMILES: Cc1c(Nc2cc[nH]c(=O)c2C(=O)Nc2ccc3c(c2)CCCN3)cnc2c1NCCO2] | 4-((8-methyl-2,3-dihydro-1H- pyrido[2,3-b][1,4]oxazin-7- yl)amino)-2-oxo-N-(1,2,3,4- tetrahydroquinolin-6-yl)-1,2- dihydropyridine-3- carboxamide | 90.5 | 433.2 |
| 255 | 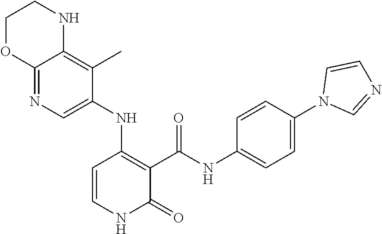 [SMILES: Cc1c(Nc2cc[nH]c(=O)c2C(=O)Nc2ccc(-n3ccnc3)cc2)cnc2c1NCCO2] | N-(4-(1H-imidazol-1- yl)phenyl)-4-((8-methyl-2,3- dihydro-1H-pyrido[2,3- b][1,4]oxazin-7-yl)amino)-2- oxo-1,2-dihydropyridine-3- carboxamide | 97.9 | 444.31 |
| 256 | 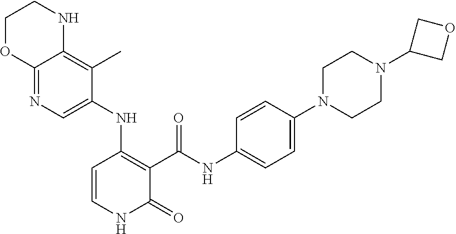 [SMILES: Cc1c(Nc2cc[nH]c(=O)c2C(=O)Nc2ccc(N3CCN(C4COC4)CC3)cc2)cnc2c1NCCO2] | 4-((8-methyl-2,3-dihydro-1H- pyrido[2,3-b][1,4]oxazin-7- yl)amino)-N-(4-(4-(oxetan-3- yl)piperazin-1-yl)phenyl)-2- oxo-1,2-dihydropyridine-3- carboxamide | 98.2 | 518.37 |
| 257 | 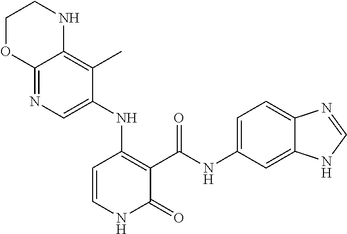 [SMILES: Cc1c(Nc2cc[nH]c(=O)c2C(=O)Nc2ccc3nc[nH]c3c2)cnc2c1NCCO2] | N-(1H-benzo[d]imidazol-6- yl)-4-((8-methyl-2,3-dihydro- 1H-pyrido[2,3-b][1,4]oxazin- 7-yl)amino)-2-oxo-1,2- dihydropyridine-3- carboxamide | 99.4 | 418.37 |
| 258 | 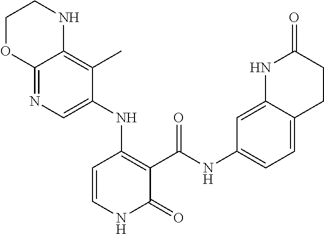 [SMILES: Cc1c(Nc2cc[nH]c(=O)c2C(=O)Nc2ccc3c(c2)NC(=O)CC3)cnc2c1NCCO2] | 4-((8-methyl-2,3-dihydro-1H- pyrido[2,3-b][1,4]oxazin-7- yl)amino)-2-oxo-N-(2-oxo- 1,2,3,4-tetrahydroquinolin-7- yl)-1,2-dihydropyridine-3- carboxamide | 99.8 | 447.34 |
| 259 | 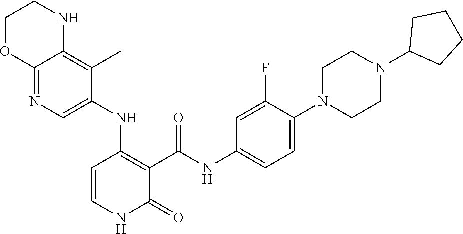 [SMILES: Cc1c(Nc2cc[nH]c(=O)c2C(=O)Nc2ccc(N3CCN(C4CCCC4)CC3)c(F)c2)cnc2c1NCCO2] | N-(4-(4-cyclopentylpiperazin- 1-yl)-3-fluorophenyl)-4-((8- methyl-2,3-dihydro-1H- pyrido[2,3-b][1,4]oxazin-7- yl)amino)-2-oxo-1,2- dihydropyridine-3- carboxamide | 98.9 | 548.41 |
| 260 | 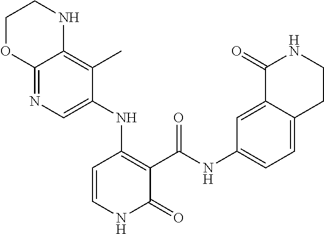 [SMILES: Cc1c(Nc2cc[nH]c(=O)c2C(=O)Nc2ccc3c(c2)C(=O)NCC3)cnc2c1NCCO2] | 4-((8-methyl-2,3-dihydro-1H- pyrido[2,3-b][1,4]oxazin-7- yl)amino)-2-oxo-N-(1-oxo- 1,2,3,4-tetrahydroisoquinolin- 7-yl)-1,2-dihydropyridine-3- carboxamide | 99.59 | 447.34 |
| 261 | 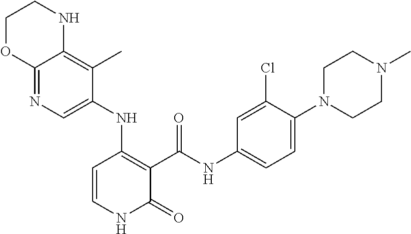 [SMILES: Cc1c(Nc2cc[nH]c(=O)c2C(=O)Nc2ccc(N3CCN(C)CC3)c(Cl)c2)cnc2c1NCCO2] | N-(3-chloro-4-(4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.1 | 510.32 |
| 262 | 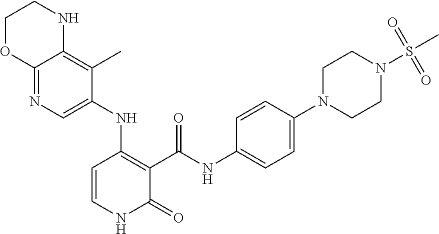 [SMILES: Cc1c(Nc2cc[nH]c(=O)c2C(=O)Nc2ccc(N3CCN(S(C)(=O)=O)CC3)cc2)cnc2c1NCCO2] | 4-((8-methyl-2,3-dihydro-1H- pyrido[2,3-b][1,4]oxazin-7- yl)amino)-N-(4-(4- (methylsulfonyl)piperazin-1- yl)phenyl)-2-oxo-1,2- dihydropyridine-3- carboxamide | 99.6 | 540.33 |
| 263 | 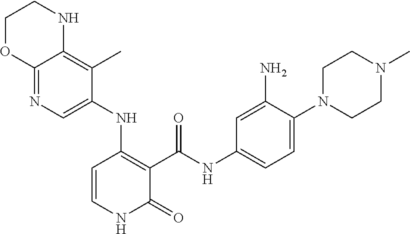 [SMILES: Cc1c(Nc2cc[nH]c(=O)c2C(=O)Nc2ccc(N3CCN(C)CC3)c(N)c2)cnc2c1NCCO2] | N-(3-amino-4-(4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.8 | 491.34 |
| 264 | 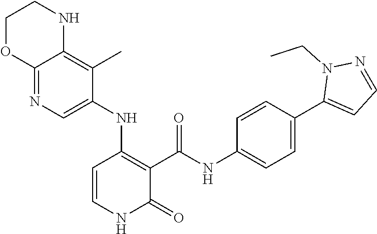 [SMILES: CCn1nccc1-c1ccc(NC(=O)c2c(Nc3cnc4c(c3C)NCCO4)cc[nH]c2=O)cc1] | N-(4-(1-ethyl-1H-pyrazol-5- yl)phenyl)-4-((8-methyl-2,3- dihydro-1H-pyrido[2,3- b][1,4]oxazin-7-yl)amino)-2- oxo-1,2-dihydropyridine-3- carboxamide | 99.2 | 472.3 |
| 265 | 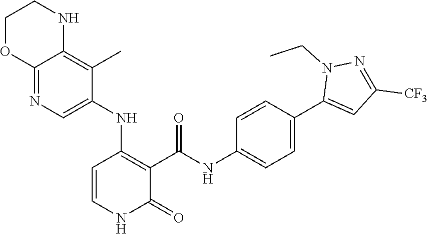 [SMILES: CCn1nc(C(F)(F)F)cc1-c1ccc(NC(=O)c2c(Nc3cnc4c(c3C)NCCO4)cc[nH]c2=O)cc1] | N-(4-(1-ethyl-3- (trifluoromethyl)-1H-pyrazol- 5-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.2 | 540.36 |
| 266 | 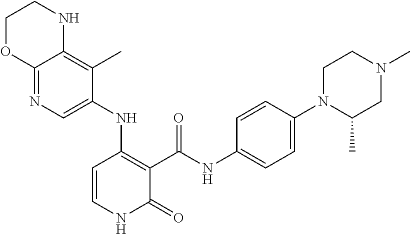 [SMILES: Cc1c(Nc2cc[nH]c(=O)c2C(=O)Nc2ccc(N3CCN(C)C[C@@H]3C)cc2)cnc2c1NCCO2] | (S)-N-(4-(2,4- dimethylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 97.3 | 490.0 |
| 267 | 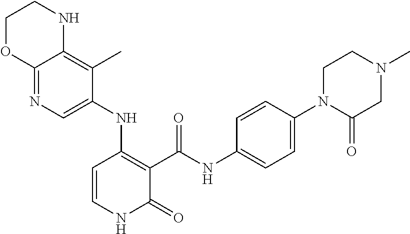 [SMILES: Cc1c(Nc2cc[nH]c(=O)c2C(=O)Nc2ccc(N3CCN(C)CC3=O)cc2)cnc2c1NCCO2] | 4-((8-methyl-2,3-dihydro-1H- pyrido[2,3-b][1,4]oxazin-7- yl)amino)-N-(4-(4-methyl-2- oxopiperazin-1-yl)phenyl)-2- oxo-1,2-dihydropyridine-3- carboxamide | 99.6 | 490.4 |
| 268 | 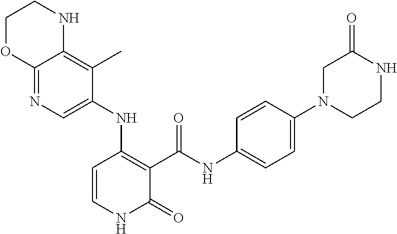 [SMILES: Cc1c(Nc2cc[nH]c(=O)c2C(=O)Nc2ccc(N3CCNC(=O)C3)cc2)cnc2c1NCCO2] | 4-((8-methyl-2,3-dihydro-1H- pyrido[2,3-b][1,4]oxazin-7- yl)amino)-2-oxo-N-(4-(3- oxopiperazin-1-yl)phenyl)- 1,2-dihydropyridine-3- carboxamide | 98.4 | 476.34 |
| 269 | 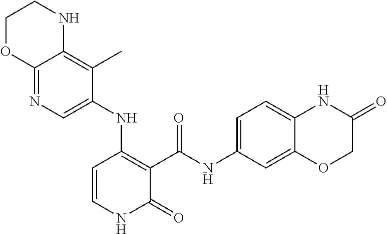 [SMILES: Cc1c(Nc2cc[nH]c(=O)c2C(=O)Nc2ccc3c(c2)OCC(=O)N3)cnc2c1NCCO2] | 4-((8-methyl-2,3-dihydro-1H- pyrido[2,3-b][1,4]oxazin-7- yl)amino)-2-oxo-N-(3-oxo- 3,4-dihydro-2H- benzo[b][1,4]oxazin-7-yl)- 1,2-dihydropyridine-3- carboxamide | 97.6 | 449.24 |
| 270 | 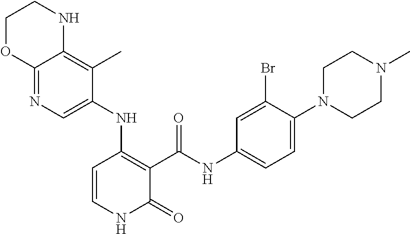 [SMILES: Cc1c(Nc2cc[nH]c(=O)c2C(=O)Nc2ccc(N3CCN(C)CC3)c(Br)c2)cnc2c1NCCO2] | N-(3-bromo-4-(4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.4 | 554.0 |
| 271 | 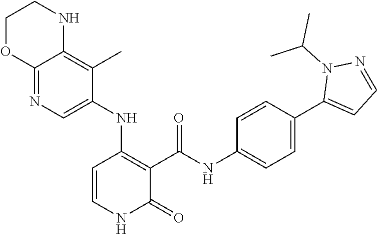 [SMILES: Cc1c(Nc2cc[nH]c(=O)c2C(=O)Nc2ccc(-c3ccnn3C(C)C)cc2)cnc2c1NCCO2] | N-(4-(1-isopropyl-1H- pyrazol-5-yl)phenyl)-4-((8- methyl-2,3-dihydro-1H- pyrido[2,3-b][1,4]oxazin-7- yl)amino)-2-oxo-1,2- dihydropyridine-3- carboxamide | 99.9 | 486.35 |
| 272 | 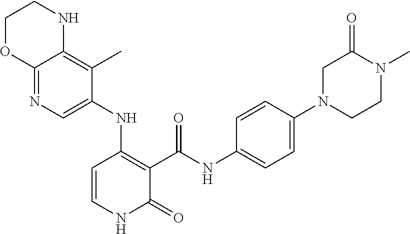 [SMILES: Cc1c(Nc2cc[nH]c(=O)c2C(=O)Nc2ccc(N3CCN(C)C(=O)C3)cc2)cnc2c1NCCO2] | 4-((8-methyl-2,3-dihydro-1H- pyrido[2,3-b][1,4]oxazin-7- yl)amino)-N-(4-(4-methyl-3- oxopiperazin-1-yl)phenyl)-2- oxo-1,2-dihydropyridine-3- carboxamide | 99.0 | 490.47 |
| 273 | 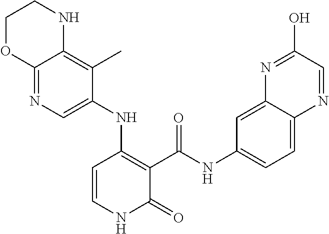 [SMILES: Cc1c(Nc2cc[nH]c(=O)c2C(=O)Nc2ccc3ncc(O)nc3c2)cnc2c1NCCO2] | N-(3-hydroxyquinoxalin-6- yl)-4-((8-methyl-2,3-dihydro- 1H-pyrido[2,3-b][1,4]oxazin- 7-yl)amino)-2-oxo-1,2- dihydropyridine-3- carboxamide | 99.65 | 446.00 |
| 274 | 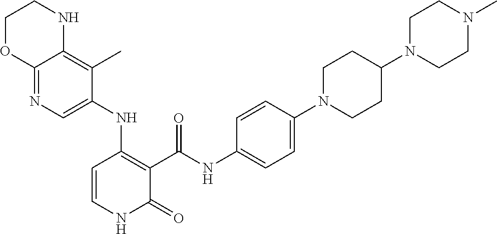 [SMILES: Cc1c(Nc2cc[nH]c(=O)c2C(=O)Nc2ccc(N3CCC(N4CCN(C)CC4)CC3)cc2)cnc2c1NCCO2] | 4-((8-methyl-2,3-dihydro-1H- pyrido[2,3-b][1,4]oxazin-7- yl)amino)-N-(4-(4-(4- methylpiperazin-1- yl)piperidin-1-yl)phenyl)-2- oxo-1,2-dihydropyridine-3- carboxamide | 99.5 | 559.58 |
| 275 | 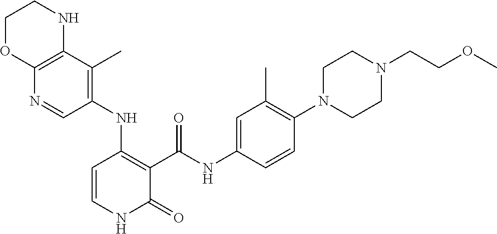 [SMILES: COCCN1CCN(c2ccc(NC(=O)c3c(Nc4cnc5c(c4C)NCCO5)cc[nH]c3=O)cc2C)CC1] | N-(4-(4-(2- methoxyethyl)piperazin-1-yl)- 3-methylphenyl)-4-((8- methyl-2,3-dihydro-1H- pyrido[2,3-b][1,4]oxazin-7- yl)amino)-2-oxo-1,2- dihydropyridine-3- carboxamide | 98.7 | 532.44 |
| 276 | 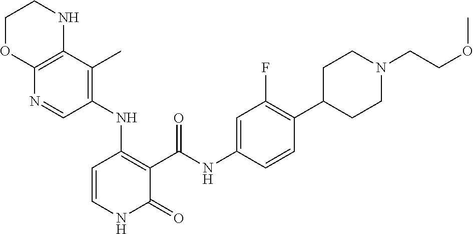 [SMILES: COCCN1CCC(c2ccc(NC(=O)c3c(Nc4cnc5c(c4C)NCCO5)cc[nH]c3=O)cc2F)CC1] | N-(3-fluoro-4-(1-(2- methoxyethyl)piperidin-4- yl)phenyl)-4-((8-methyl-2,3- dihydro-1H-pyrido[2,3- b][1,4]oxazin-7-yl)amino)-2- oxo-1,2-dihydropyridine-3- carboxamide | 99.3 | 537.45 |
| 277 | 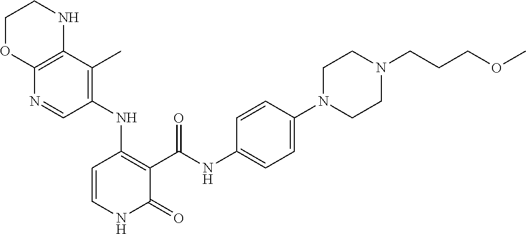 [SMILES: COCCCN1CCN(c2ccc(NC(=O)c3c(Nc4cnc5c(c4C)NCCO5)cc[nH]c3=O)cc2)CC1] | N-(4-(4-(3- methoxypropyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.6 | 532.39 |
| 278 | 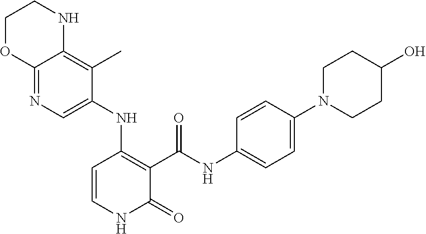 [SMILES: Cc1c(Nc2cc[nH]c(=O)c2C(=O)Nc2ccc(N3CCC(O)CC3)cc2)cnc2c1NCCO2] | N-(4-(4-hydroxypiperidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.5 | 475.36 |
| 279 | 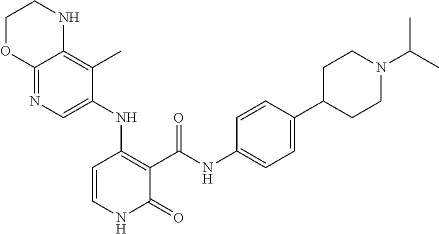 [SMILES: Cc1c(Nc2cc[nH]c(=O)c2C(=O)Nc2ccc(C3CCN(C(C)C)CC3)cc2)cnc2c1NCCO2] | N-(4-(1-isopropylpiperidin-4- yl)phenyl)-4-((8-methyl-2,3- dihydro-1H-pyrido[2,3- b][1,4]oxazin-7-yl)amino)-2- oxo-1,2-dihydropyridine-3- carboxamide | 99.8 | 559.3 |
| 280 | 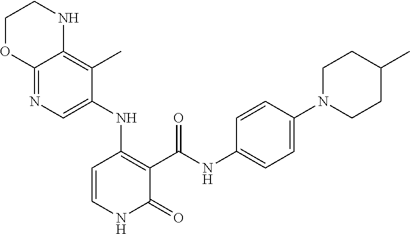 [SMILES: Cc1c(Nc2cc[nH]c(=O)c2C(=O)Nc2ccc(N3CCC(C)CC3)cc2)cnc2c1NCCO2] | 4-((8-methyl-2,3-dihydro-1H- pyrido[2,3-b][1,4]oxazin-7- yl)amino)-N-(4-(4- methylpiperidin-1-yl)phenyl)- 2-oxo-1,2-dihydropyridine-3- carboxamide | 96.4 | 473.33 |
| 281 | 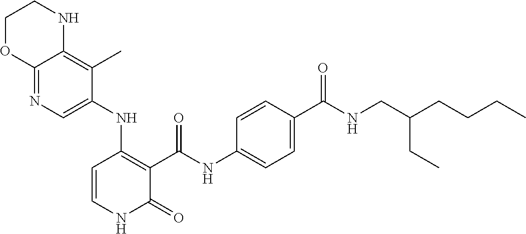 [SMILES: CCCCC(CC)CNC(=O)c1ccc(NC(=O)c2c(Nc3cnc4c(c3C)NCCO4)cc[nH]c2=O)cc1] | N-(4-((2- ethylhexyl)carbamoyl)phenyl)- 4-((8-methyl-2,3-dihydro- 1H-pyrido[2,3-b][1,4]oxazin- 7-yl)amino)-2-oxo-1,2- dihydropyridine-3- carboxamide | 98.34 | 533.4 |
| 282 | 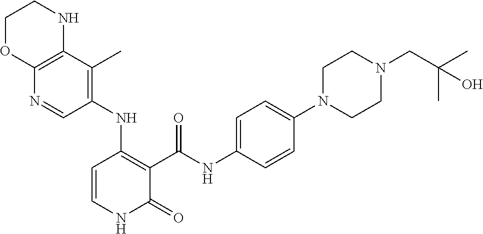 [SMILES: Cc1c(Nc2cc[nH]c(=O)c2C(=O)Nc2ccc(N3CCN(CC(C)(C)O)CC3)cc2)cnc2c1NCCO2] | N-(4-(4-(2-hydroxy-2- methylpropyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.6 | 534.39 |
| 283 | 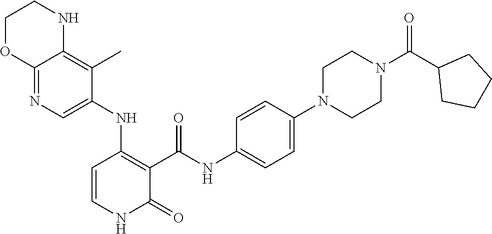 [SMILES: Cc1c(Nc2cc[nH]c(=O)c2C(=O)Nc2ccc(N3CCN(C(=O)C4CCCC4)CC3)cc2)cnc2c1NCCO2] | N-(4-(4- (cyclopentanecarbonyl) piperazin-1-yl)phenyl)-4-((8- methyl-2,3-dihydro-1H- pyrido[2,3-b][1,4]oxazin-7- yl)amino)-2-oxo-1,2- dihydropyridine-3- carboxamide | 99.8 | 558.46 |
| 284 | 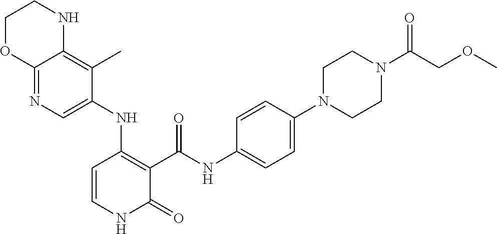 [SMILES: COCC(=O)N1CCN(c2ccc(NC(=O)c3c(Nc4cnc5c(c4C)NCCO5)cc[nH]c3=O)cc2)CC1] | N-(4-(4-(2- methoxyacetyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.6 | 534.46 |
| 285 | 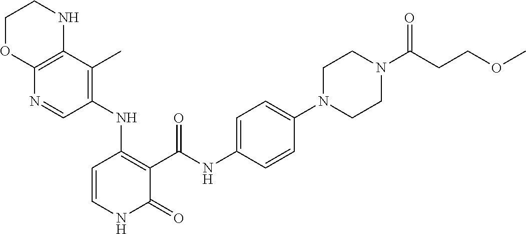 [SMILES: COCCC(=O)N1CCN(c2ccc(NC(=O)c3c(Nc4cnc5c(c4C)NCCO5)cc[nH]c3=O)cc2)CC1] | N-(4-(4-(3- methoxypropanoyl)piperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.7 | 548.49 |
| 286 | 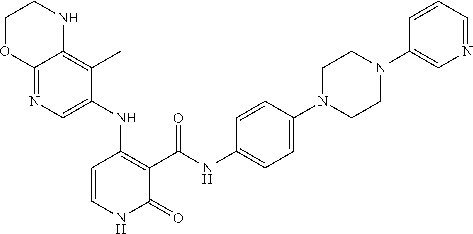 [SMILES: Cc1c(Nc2cc[nH]c(=O)c2C(=O)Nc2ccc(N3CCN(c4cccnc4)CC3)cc2)cnc2c1NCCO2] | 4-((8-methyl-2,3-dihydro-1H- pyrido[2,3-b][1,4]oxazin-7- yl)amino)-2-oxo-N-(4-(4- (pyridin-3-yl)piperazin-1- yl)phenyl)-1,2- dihydropyridine-3- carboxamide | 99.4 | 539.38 |
| 287 | 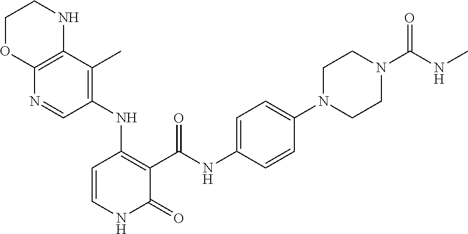 [SMILES: CNC(=O)N1CCN(c2ccc(NC(=O)c3c(Nc4cnc5c(c4C)NCCO5)cc[nH]c3=O)cc2)CC1] | N-methyl-4-(4-(4-((8-methyl- 2,3-dihydro-1H-pyrido[2,3- b][1,4]oxazin-7-yl)amino)-2- oxo-1,2-dihydropyridine-3- carboxamido)phenyl) piperazine-1-carboxamide | 99.8 | 519.42 |
| 288 | 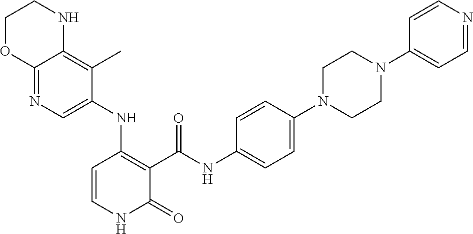 [SMILES: Cc1c(Nc2cc[nH]c(=O)c2C(=O)Nc2ccc(N3CCN(c4ccncc4)CC3)cc2)cnc2c1NCCO2] | 4-((8-methyl-2,3-dihydro-1H- pyrido[2,3-b][1,4]oxazin-7- yl)amino)-2-oxo-N-(4-(4- (pyridin-4-yl)piperazin-1- yl)phenyl)-1,2- dihydropyridine-3- carboxamide | 98.7 | 539.4 |
| 289 | 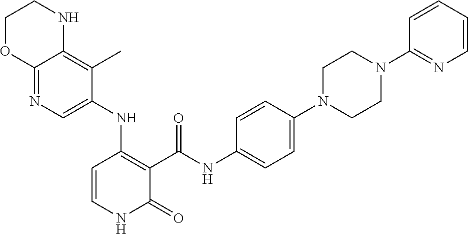 [SMILES: Cc1c(Nc2cc[nH]c(=O)c2C(=O)Nc2ccc(N3CCN(c4ccccn4)CC3)cc2)cnc2c1NCCO2] | 4-((8-methyl-2,3-dihydro-1H- pyrido[2,3-b][1,4]oxazin-7- yl)amino)-2-oxo-N-(4-(4- (pyridin-2-yl)piperazin-1- yl)phenyl)-1,2- dihydropyridine-3- carboxamide | 99.6 | 537.36 |
| 290 | 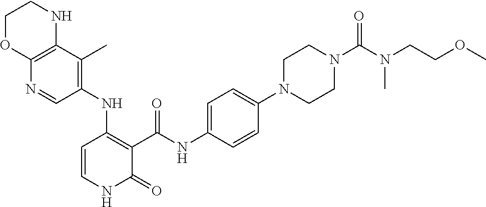 [SMILES: COCCN(C)C(=O)N1CCN(c2ccc(NC(=O)c3c(Nc4cnc5c(c4C)NCCO5)cc[nH]c3=O)cc2)CC1] | N-(2-methoxyethyl)-N- methyl-4-(4-(4-((8-methyl- 2,3-dihydro-1H-pyrido[2,3- b][1,4]oxazin-7-yl)amino)-2- oxo-1,2-dihydropyridine-3- carboxamido)phenyl) piperazine-1-carboxamide | 99.4 | 577.47 |
| 291 | 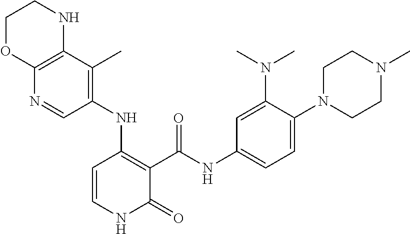 [SMILES: Cc1c(Nc2cc[nH]c(=O)c2C(=O)Nc2ccc(N3CCN(C)CC3)c(N(C)C)c2)cnc2c1NCCO2] | N-(3-(dimethylamino)-4-(4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.6 | 519.47 |
| 292 | 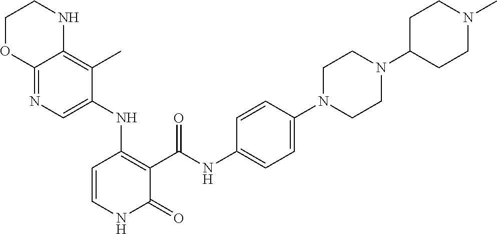 [SMILES: Cc1c(Nc2cc[nH]c(=O)c2C(=O)Nc2ccc(N3CCN(C4CCN(C)CC4)CC3)cc2)cnc2c1NCCO2] | 4-((8-methyl-2,3-dihydro-1H- pyrido[2,3-b][1,4]oxazin-7- yl)amino)-N-(4-(4-(1- methylpiperidin-4- yl)piperazin-1-yl)phenyl)-2- oxo-1,2-dihydropyridine-3- carboxamide | 98.9 | 559.5 |
| 293 | 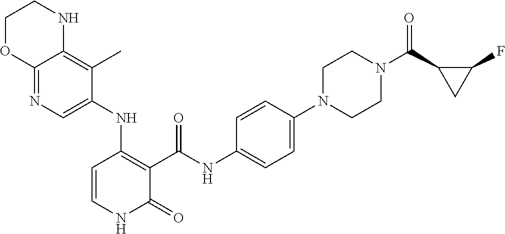 [SMILES: Cc1c(Nc2cc[nH]c(=O)c2C(=O)Nc2ccc(N3CCN(C(=O)[C@@H]4C[C@@H]4F)CC3)cc2)cnc2c1NCCO2] | N-(4-(4-((1S,2S)-2- fluorocyclopropane-1- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.5 | 546.54 [M − H] |
| 294 | 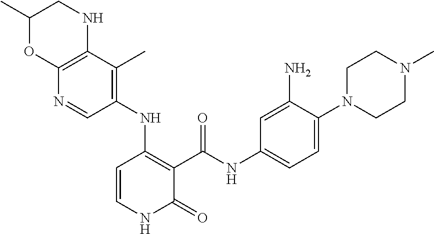 | N-(3-amino-4-(4- methylpiperazin-1-yl)phenyl)- 4-((3,8-dimethyl-2,3-dihydro- 1H-pyrido[2,3-b][1,4]oxazin- 7-yl)amino)-2-oxo-1,2- dihydropyridine-3- carboxamide | 99.0 | 505.4 |
| 295 | 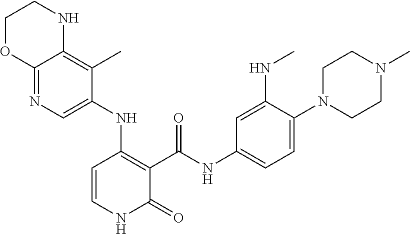 [SMILES: CNc1cc(NC(=O)c2c(Nc3cnc4c(c3C)NCCO4)cc[nH]c2=O)ccc1N1CCN(C)CC1] | 4-((8-methyl-2,3-dihydro-1H- pyrido[2,3-b][1,4]oxazin-7- yl)amino)-N-(3- (methylamino)-4-(4- methylpiperazin-1-yl)phenyl)- 2-oxo-1,2-dihydropyridine-3- carboxamide | 97.3 | 505.35 |
| 296 | 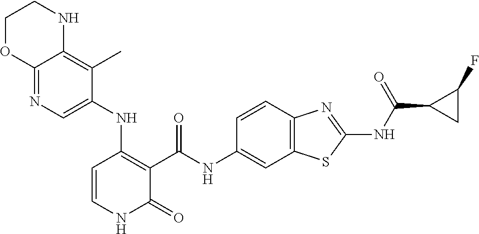 [SMILES: Cc1c(Nc2cc[nH]c(=O)c2C(=O)Nc2ccc3nc(NC(=O)[C@@H]4C[C@@H]4F)sc3c2)cnc2c1NCCO2] | N-(2-((1S,2S)-2- fluorocyclopropane-1- carboxamido)benzo[d]thiazol- 6-yl)-4-((8-methyl-2,3- dihydro-1H-pyrido[2,3- b][1,4]oxazin-7-yl)amino)-2- oxo-1,2-dihydropyridine-3- carboxamide | 98.52 | 536.3 |
| 297 | 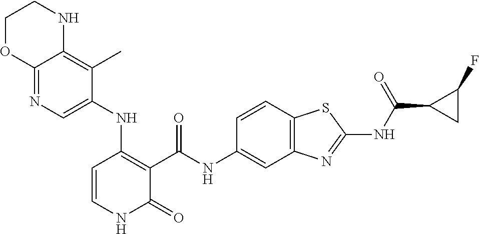 [SMILES: Cc1c(Nc2cc[nH]c(=O)c2C(=O)Nc2ccc3sc(NC(=O)[C@@H]4C[C@@H]4F)nc3c2)cnc2c1NCCO2] | N-(2-((1S,2S)-2- fluorocyclopropane-1- carboxamido)benzo[d]thiazol- 5-yl)-4-((8-methyl-2,3- dihydro-1H-pyrido[2,3- b][1,4]oxazin-7-yl)amino)-2- oxo-1,2-dihydropyridine-3- carboxamide | 94.16 | 536.29 |
| 298 | 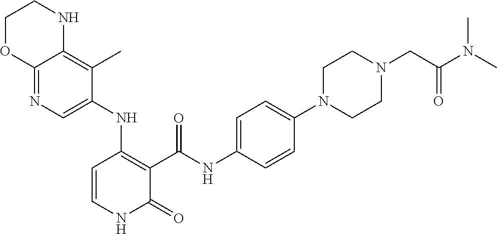 [SMILES: Cc1c(Nc2cc[nH]c(=O)c2C(=O)Nc2ccc(N3CCN(CC(=O)N(C)C)CC3)cc2)cnc2c1NCCO2] | N-(4-(4-(2-(dimethylamino)- 2-oxoethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 97.3 | 547.4 |
| 299 | 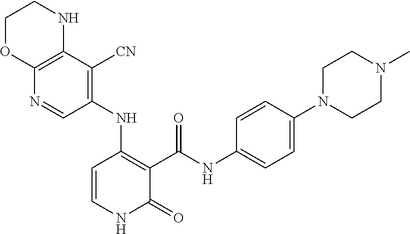 [SMILES: CN1CCN(c2ccc(NC(=O)c3c(Nc4cnc5c(c4C#N)NCCO5)cc[nH]c3=O)cc2)CC1] | 4-((8-cyano-2,3-dihydro-1H- pyrido[2,3-b][1,4]oxazin-7- yl)amino)-N-(4-(4- methylpiperazin-1-yl)phenyl)- 2-oxo-1,2-dihydropyridine-3- carboxamide | 99.2 | 487.31 |
| 300 | 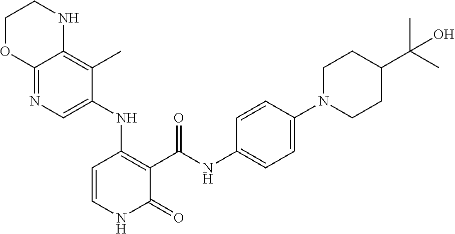 [SMILES: Cc1c(Nc2cc[nH]c(=O)c2C(=O)Nc2ccc(N3CCC(C(C)(C)O)CC3)cc2)cnc2c1NCCO2] | N-(4-(4-(2-hydroxypropan-2- yl)piperidin-1-yl)phenyl)-4- ((8-methyl-2,3-dihydro-1H- pyrido[2,3-b][1,4]oxazin-7- yl)amino)-2-oxo-1,2- dihydropyridine-3- carboxamide | 99.6 | 519.40 |
| 301 | 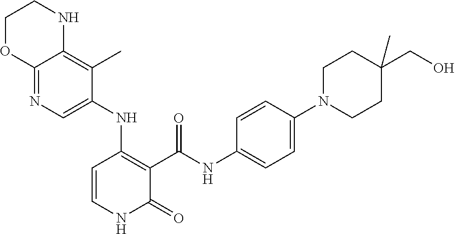 [SMILES: Cc1c(Nc2cc[nH]c(=O)c2C(=O)Nc2ccc(N3CCC(C)(CO)CC3)cc2)cnc2c1NCCO2] | N-(4-(4-(hydroxymethyl)-4- methylpiperidin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.4 | 505.4 |
| 302 | 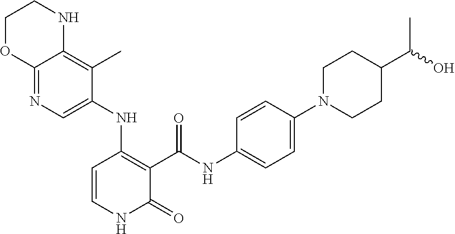 [SMILES: Cc1c(Nc2cc[nH]c(=O)c2C(=O)Nc2ccc(N3CCC(C(C)O)CC3)cc2)cnc2c1NCCO2] | N-(4-(4-(1- hydroxyethyl)piperidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.9 | 505.38 |
| 303 | 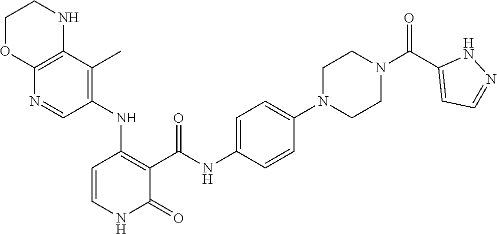 [SMILES: Cc1c(Nc2cc[nH]c(=O)c2C(=O)Nc2ccc(N3CCN(C(=O)c4ccn[nH]4)CC3)cc2)cnc2c1NCCO2] | N-(4-(4-(1H-pyrazole-5- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 96.8 | 556.40 |
| 304 | 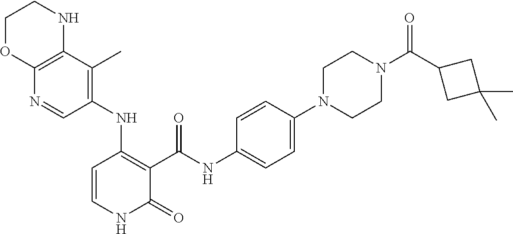 [SMILES: Cc1c(Nc2cc[nH]c(=O)c2C(=O)Nc2ccc(N3CCN(C(=O)C4CC(C)(C)C4)CC3)cc2)cnc2c1NCCO2] | N-(4-(4-(3,3- dimethylcyclobutane-1- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.8 | 572.13 |
| 305 | 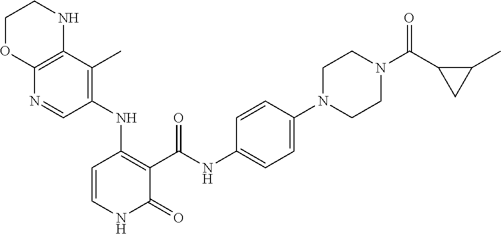 [SMILES: Cc1c(Nc2cc[nH]c(=O)c2C(=O)Nc2ccc(N3CCN(C(=O)C4CC4C)CC3)cc2)cnc2c1NCCO2] | 4-((8-methyl-2,3-dihydro-1H- pyrido[2,3-b][1,4]oxazin-7- yl)amino)-N-(4-(4-(2- methylcyclopropane-1- carbonyl)piperazin-1- yl)phenyl)-2-oxo-1,2- dihydropyridine-3- carboxamide | 99.0 | 544.09 |
| 306 | 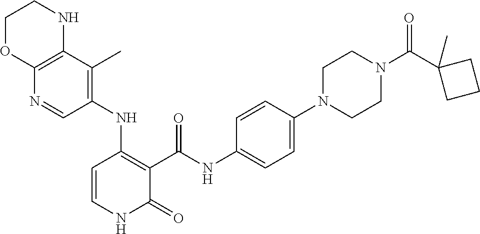 [SMILES: Cc1c(Nc2cc[nH]c(=O)c2C(=O)Nc2ccc(N3CCN(C(=O)C4(C)CCC4)CC3)cc2)cnc2c1NCCO2] | 4-((8-methyl-2,3-dihydro-1H- pyrido[2,3-b][1,4]oxazin-7- yl)amino)-N-(4-(4-(1- methylcyclobutane-1- carbonyl)piperazin-1- yl)phenyl)-2-oxo-1,2- dihydropyridine-3- carboxamide | 99.8 | 558.10 |
| 307 | 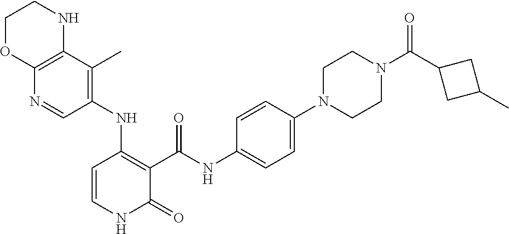 [SMILES: Cc1c(Nc2cc[nH]c(=O)c2C(=O)Nc2ccc(N3CCN(C(=O)C4CC(C)C4)CC3)cc2)cnc2c1NCCO2] | 4-((8-methyl-2,3-dihydro-1H- pyrido[2,3-b][1,4]oxazin-7- yl)amino)-N-(4-(4-(3- methylcyclobutane-1- carbonyl)piperazin-1- yl)phenyl)-2-oxo-1,2- dihydropyridine-3- carboxamide | 99.4 | 558.09 |
| 308 | 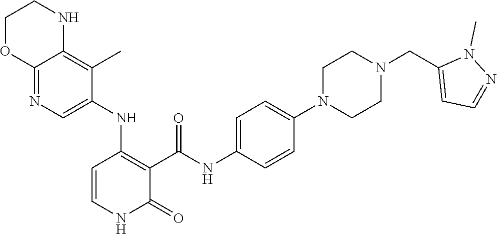 [SMILES: Cc1c(Nc2cc[nH]c(=O)c2C(=O)Nc2ccc(N3CCN(Cc4ccnn4C)CC3)cc2)cnc2c1NCCO2] | N-(4-(4-((1-methyl-1H- pyrazol-5- yl)methyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.0 | 556.10 |
| 309 | 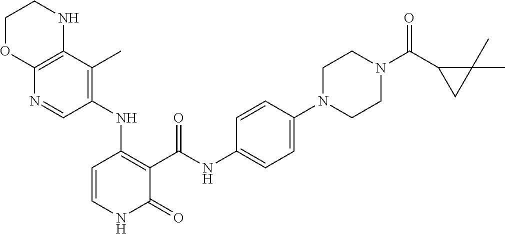 [SMILES: Cc1c(Nc2cc[nH]c(=O)c2C(=O)Nc2ccc(N3CCN(C(=O)C4CC4(C)C)CC3)cc2)cnc2c1NCCO2] | N-(4-(4-(2,2- dimethylcyclopropane-1- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 97.5 | 558.08 |
| 310 | 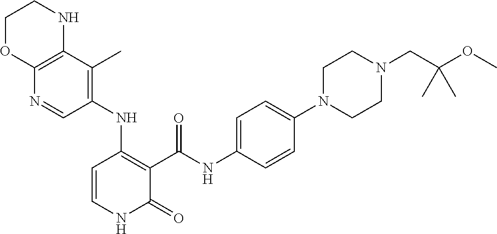 [SMILES: COC(C)(C)CN1CCN(c2ccc(NC(=O)c3c(Nc4cnc5c(c4C)NCCO5)cc[nH]c3=O)cc2)CC1] | N-(4-(4-(2-methoxy-2- methylpropyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.9 | 548.06 |
| 311 | 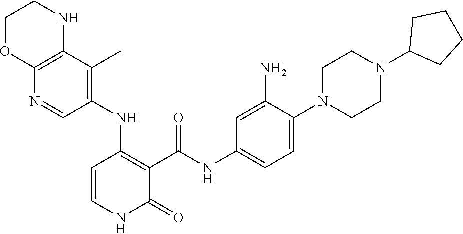 [SMILES: Cc1c(Nc2cc[nH]c(=O)c2C(=O)Nc2ccc(N3CCN(C4CCCC4)CC3)c(N)c2)cnc2c1NCCO2] | N-(3-amino-4-(4- cyclopentylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.8 | 548.06 |
| 312 | 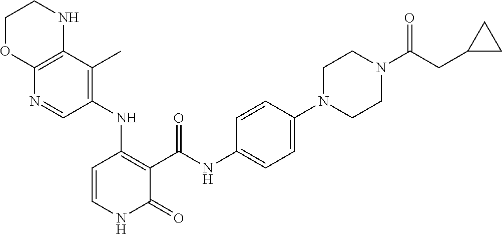 [SMILES: Cc1c(Nc2cc[nH]c(=O)c2C(=O)Nc2ccc(N3CCN(C(=O)CC4CC4)CC3)cc2)cnc2c1NCCO2] | N-(4-(4-(2- cyclopropylacetyl)piperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 97.3 | 544.06 |
| 313 | 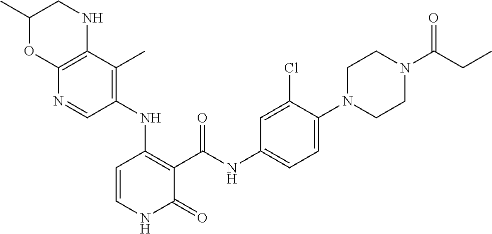 [SMILES: CCC(=O)N1CCN(c2ccc(NC(=O)c3c(Nc4cnc5c(c4C)NCC(C)O5)cc[nH]c3=O)cc2Cl)CC1] | N-(3-chloro-4-(4- propionylpiperazin-1- yl)phenyl)-4-((3,8-dimethyl- 2,3-dihydro-1H-pyrido[2,3- b][1,4]oxazin-7-yl)amino)-2- oxo-1,2-dihydropyridine-3- carboxamide | 99.1 | 566.05 |
| 314 | 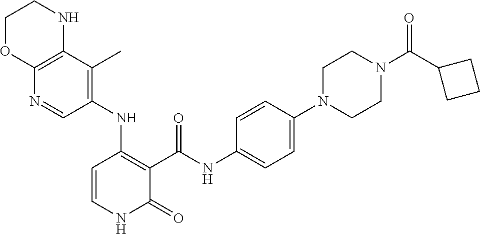 [SMILES: Cc1c(Nc2cc[nH]c(=O)c2C(=O)Nc2ccc(N3CCN(C(=O)C4CCC4)CC3)cc2)cnc2c1NCCO2] | N-(4-(4- (cyclobutanecarbonyl) piperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 98.7 | 566.05 |
| 315 | 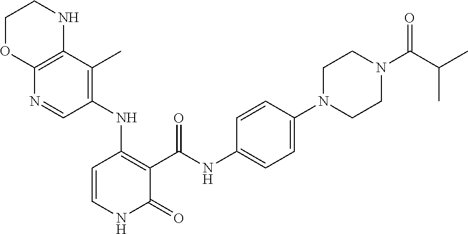 [SMILES: Cc1c(Nc2cc[nH]c(=O)c2C(=O)Nc2ccc(N3CCN(C(=O)C(C)C)CC3)cc2)cnc2c1NCCO2] | N-(4-(4-isobutyrylpiperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.3 | 532.06 |
| 316 | 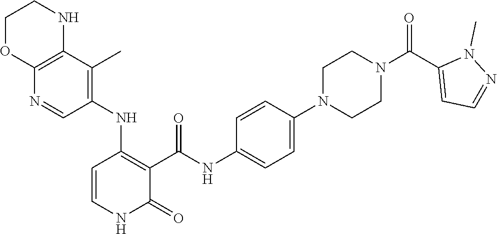 [SMILES: Cc1c(Nc2cc[nH]c(=O)c2C(=O)Nc2ccc(N3CCN(C(=O)c4ccnn4C)CC3)cc2)cnc2c1NCCO2] | N-(4-(4-(1-methyl-1H- pyrazole-5- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.2 | 570.05 |
| 317 | 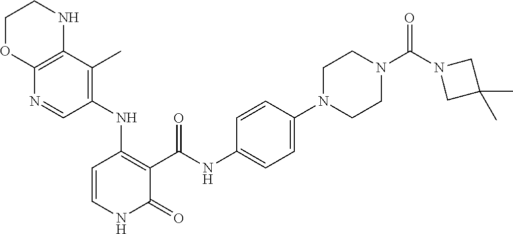 [SMILES: Cc1c(Nc2cc[nH]c(=O)c2C(=O)Nc2ccc(N3CCN(C(=O)N4CC(C)(C)C4)CC3)cc2)cnc2c1NCCO2] | N-(4-(4-(3,3- dimethylazetidine-1- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.0 | 573.11 |
| 318 | 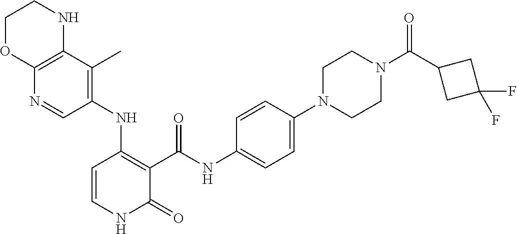 [SMILES: Cc1c(Nc2cc[nH]c(=O)c2C(=O)Nc2ccc(N3CCN(C(=O)C4CC(F)(F)C4)CC3)cc2)cnc2c1NCCO2] | N-(4-(4-(3,3- difluorocyclobutane-1- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 97.6 | 580.06 |
| 319 | 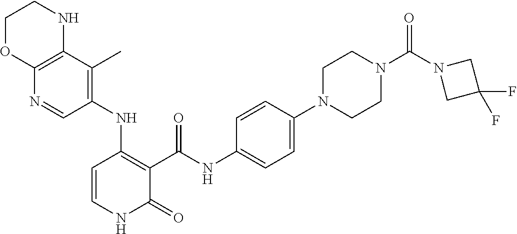 [SMILES: Cc1c(Nc2cc[nH]c(=O)c2C(=O)Nc2ccc(N3CCN(C(=O)N4CC(F)(F)C4)CC3)cc2)cnc2c1NCCO2] | N-(4-(4-(3,3- difluoroazetidine-1- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 97.3 | 581.15 |
| 320 | 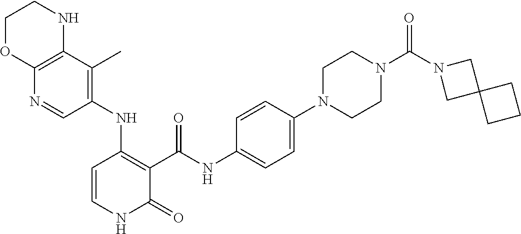 [SMILES: Cc1c(Nc2cc[nH]c(=O)c2C(=O)Nc2ccc(N3CCN(C(=O)N4CC5(CCC5)C4)CC3)cc2)cnc2c1NCCO2] | N-(4-(4-(2- azaspiro[3.3]heptane-2- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 97.1 | 585.11 |
| 321 | 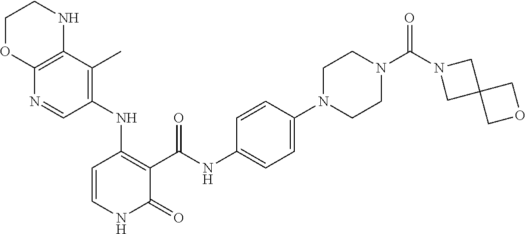 [SMILES: Cc1c(Nc2cc[nH]c(=O)c2C(=O)Nc2ccc(N3CCN(C(=O)N4CC5(COC5)C4)CC3)cc2)cnc2c1NCCO2] | N-(4-(4-(2-oxa-6- azaspiro[3.3]heptane-6- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 96.7 | 587.11 |
| 322 | 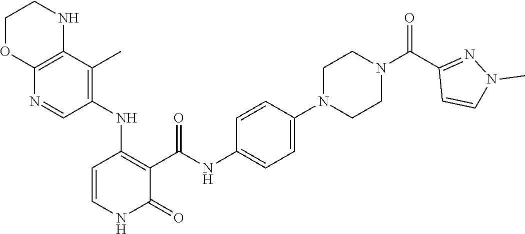 [SMILES: Cc1c(Nc2cc[nH]c(=O)c2C(=O)Nc2ccc(N3CCN(C(=O)c4ccn(C)n4)CC3)cc2)cnc2c1NCCO2] | N-(4-(4-(1-methyl-1H- pyrazole-3- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 95.9 | 570.06 |
| 323 | 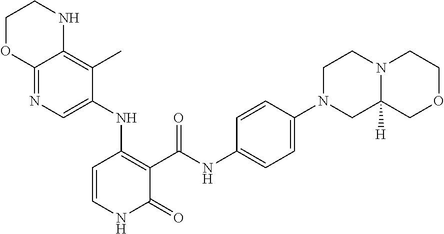 [SMILES: Cc1c(Nc2cc[nH]c(=O)c2C(=O)Nc2ccc(N3CCN4CCOC[C@H]4C3)cc2)cnc2c1NCCO2] | (R)-N-(4- (hexahydropyrazino[2,1- c][1,4]oxazin-8(1H)- yl)phenyl)-4-((8-methyl-2,3- dihydro-1H-pyrido[2,3- b][1,4]oxazin-7-yl)amino)-2- oxo-1,2-dihydropyridine-3- carboxamide | 97.7 | 518.06 |
| 324 | 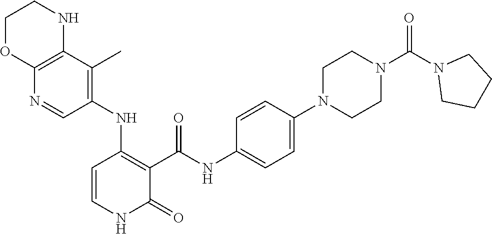 [SMILES: Cc1c(Nc2cc[nH]c(=O)c2C(=O)Nc2ccc(N3CCN(C(=O)N4CCCC4)CC3)cc2)cnc2c1NCCO2] | 4-((8-methyl-2,3-dihydro-1H- pyrido[2,3-b][1,4]oxazin-7- yl)amino)-2-oxo-N-(4-(4- (pyrrolidine-1- carbonyl)piperazin-1- yl)phenyl)-1,2- dihydropyridine-3- carboxamide | 99.0 | 559.08 |
| 325 | 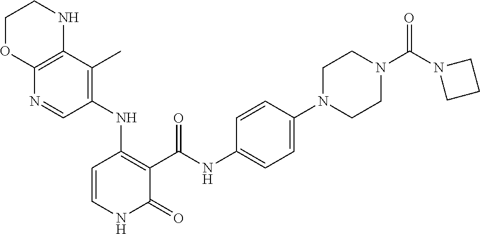 [SMILES: Cc1c(Nc2cc[nH]c(=O)c2C(=O)Nc2ccc(N3CCN(C(=O)N4CCC4)CC3)cc2)cnc2c1NCCO2] | N-(4-(4-(azetidine-1- carbonyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 97.5 | 545.10 |
| 326 | 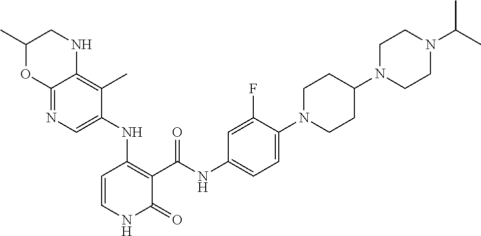 [SMILES: Cc1c(Nc2cc[nH]c(=O)c2C(=O)Nc2ccc(N3CCC(N4CCN(C(C)C)CC4)CC3)c(F)c2)cnc2c1NCC(C)O2] | 4-((3,8-dimethyl-2,3-dihydro- 1H-pyrido[2,3-b][1,4]oxazin- 7-yl)amino)-N-(3-fluoro-4-(4- (4-isopropylpiperazin-1- yl)piperidin-1-yl)phenyl)-2- oxo-1,2-dihydropyridine-3- carboxamide | 99.4 | 619.22 |
| 327 | 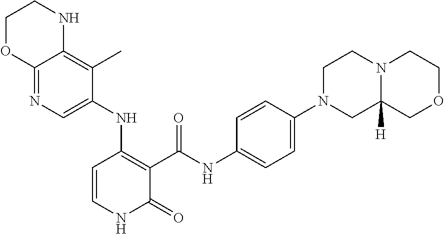 [SMILES: Cc1c(Nc2cc[nH]c(=O)c2C(=O)Nc2ccc(N3CCN4CCOC[C@@H]4C3)cc2)cnc2c1NCCO2] | (S)-N-(4- (hexahydropyrazino[2,1- c][1,4]oxazin-8(1H)- yl)phenyl)-4-((8-methyl-2,3- dihydro-1H-pyrido[2,3- b][1,4]oxazin-7-yl)amino)-2- oxo-1,2-dihydropyridine-3- carboxamide | 97.0 | 518.07 |
| 328 | 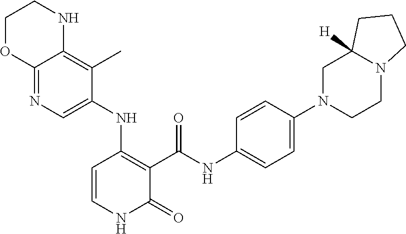 [SMILES: Cc1c(Nc2cc[nH]c(=O)c2C(=O)Nc2ccc(N3CCN4CCC[C@H]4C3)cc2)cnc2c1NCCO2] | (S)-N-(4- (hexahydropyrrolo[1,2- a]pyrazin-2(1H)-yl)phenyl)-4- ((8-methyl-2,3-dihydro-1H- pyrido[2,3-b][1,4]oxazin-7- yl)amino)-2-oxo-1,2- dihydropyridine-3- carboxamide | 99.5 | 502.07 |
| 329 | 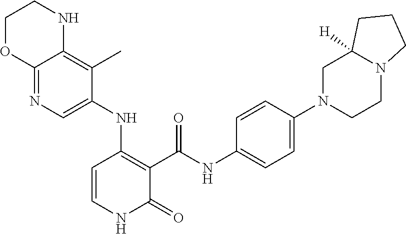 [SMILES: Cc1c(Nc2cc[nH]c(=O)c2C(=O)Nc2ccc(N3CCN4CCC[C@@H]4C3)cc2)cnc2c1NCCO2] | (R)-N-(4- (hexahydropyrrolo[1,2- a]pyrazin-2(1H)-yl)phenyl)-4- ((8-methyl-2,3-dihydro-1H- pyrido[2,3-b][1,4]oxazin-7- yl)amino)-2-oxo-1,2- dihydropyridine-3- carboxamide | 97.5 | 502.04 |
| 330 | 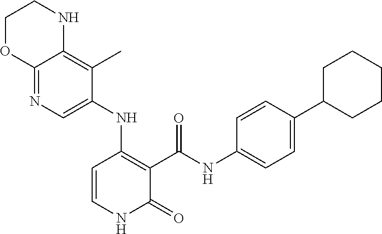 [SMILES: Cc1c(Nc2cc[nH]c(=O)c2C(=O)Nc2ccc(C3CCCCC3)cc2)cnc2c1NCCO2] | N-(4-cyclohexylphenyl)-4- ((8-methyl-2,3-dihydro-1H- pyrido[2,3-b][1,4]oxazin-7- yl)amino)-2-oxo-1,2- dihydropyridine-3- carboxamide | 98.44 | 460.10 |
| 331 | 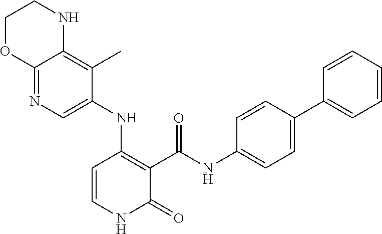 [SMILES: Cc1c(Nc2cc[nH]c(=O)c2C(=O)Nc2ccc(-c3ccccc3)cc2)cnc2c1NCCO2] | N-([1,1′-biphenyl]-4-yl)-4- ((8-methyl-2,3-dihydro-1H- pyrido[2,3-b][1,4]oxazin-7- yl)amino)-2-oxo-1,2- dihydropyridine-3- carboxamide | 99.56 | 454.07 |
| 332 | 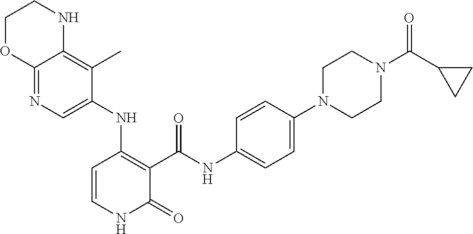 [SMILES: Cc1c(Nc2cc[nH]c(=O)c2C(=O)Nc2ccc(N3CCN(C(=O)C4CC4)CC3)cc2)cnc2c1NCCO2] | N-(4-(4- (cyclopropanecarbonyl) piperazin-1-yl)phenyl)-4-((8- methyl-2,3-dihydro-1H- pyrido[2,3-b][1,4]oxazin-7- yl)amino)-2-oxo-1,2- dihydropyridine-3- carboxamide | 98.5 | 530.06 |
| 333 | 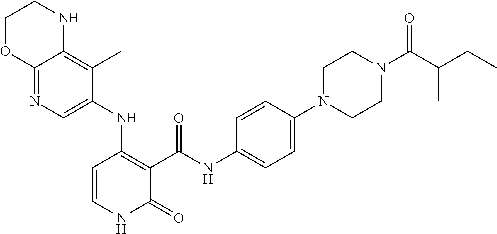 [SMILES: CCC(C)C(=O)N1CCN(c2ccc(NC(=O)c3c(Nc4cnc5c(c4C)NCCO5)cc[nH]c3=O)cc2)CC1] | 4-((8-methyl-2,3-dihydro-1H- pyrido[2,3-b][1,4]oxazin-7- yl)amino)-N-(4-(4-(2- methylbutanoyl)piperazin-1- yl)phenyl)-2-oxo-1,2- dihydropyridine-3- carboxamide | 99.1 | 546.45 |
| 334 | 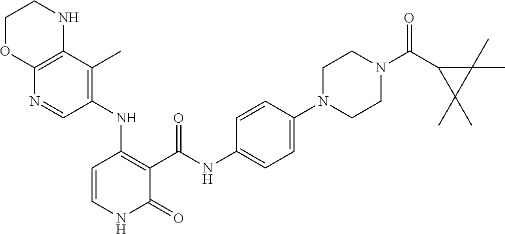 [SMILES: Cc1c(Nc2cc[nH]c(=O)c2C(=O)Nc2ccc(N3CCN(C(=O)C4C(C)(C)C4(C)C)CC3)cc2)cnc2c1NCCO2] | 4-((8-methyl-2,3-dihydro-1H- pyrido[2,3-b][1,4]oxazin-7- yl)amino)-2-oxo-N-(4-(4- (2,2,3,3- tetramethylcyclopropane-1- carbonyl)piperazin-1- yl)phenyl)-1,2- dihydropyridine-3- carboxamide | 98.0 | 586.13 |
| 335 | 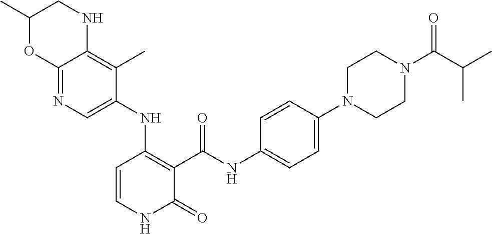 [SMILES: Cc1c(Nc2cc[nH]c(=O)c2C(=O)Nc2ccc(N3CCN(C(=O)C(C)C)CC3)cc2)cnc2c1NCC(C)O2] | 4-((3,8-dimethyl-2,3-dihydro- 1H-pyrido[2,3-b][1,4]oxazin- 7-yl)amino)-N-(4-(4- isobutyrylpiperazin-1- yl)phenyl)-2-oxo-1,2- dihydropyridine-3- carboxamide | 99.3 | 546.41 |
| 336 | 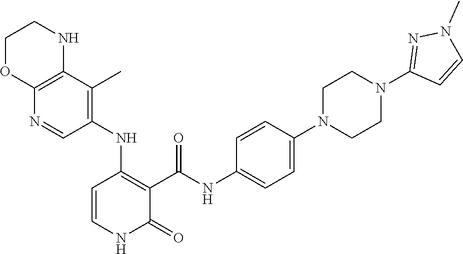 [SMILES: Cc1c(Nc2cc[nH]c(=O)c2C(=O)Nc2ccc(N3CCN(c4ccn(C)n4)CC3)cc2)cnc2c1NCCO2] | N-(4-(4-(1-methyl-1H- pyrazol-3-yl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 100 | 542.48 |
| 337 | 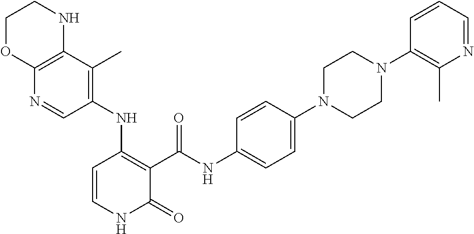 [SMILES: Cc1ncccc1N1CCN(c2ccc(NC(=O)c3c(Nc4cnc5c(c4C)NCCO5)cc[nH]c3=O)cc2)CC1] | 4-((8-methyl-2,3-dihydro-1H- pyrido[2,3-b][1,4]oxazin-7- yl)amino)-N-(4-(4-(2- methylpyridin-3-yl)piperazin- 1-yl)phenyl)-2-oxo-1,2- dihydropyridine-3- carboxamide | 98.4 | 553.3 |
| 338 | 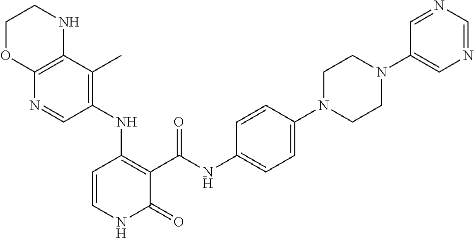 [SMILES: Cc1c(Nc2cc[nH]c(=O)c2C(=O)Nc2ccc(N3CCN(c4cncnc4)CC3)cc2)cnc2c1NCCO2] | 4-((8-methyl-2,3-dihydro-1H- pyrido[2,3-b][1,4]oxazin-7- yl)amino)-2-oxo-N-(4-(4- (pyrimidin-5-yl)piperazin-1- yl)phenyl)-1,2- dihydropyridine-3- carboxamide | 98.4 | 540.46 |
| 339 | 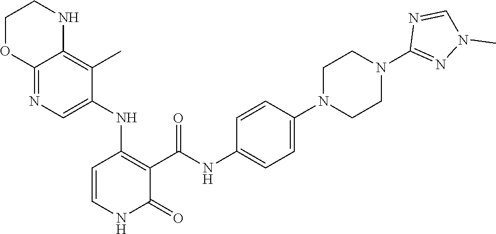 [SMILES: Cc1c(Nc2cc[nH]c(=O)c2C(=O)Nc2ccc(N3CCN(c4ncn(C)n4)CC3)cc2)cnc2c1NCCO2] | N-(4-(4-(1-methyl-1H-1,2,4- triazol-3-yl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.8 | 543.47 |
| 340 | 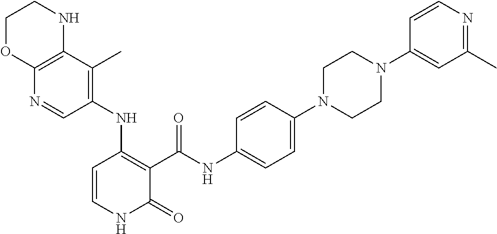 [SMILES: Cc1cc(N2CCN(c3ccc(NC(=O)c4c(Nc5cnc6c(c5C)NCCO6)cc[nH]c4=O)cc3)CC2)ccn1] | 4-((8-methyl-2,3-dihydro-1H- pyrido[2,3-b][1,4]oxazin-7- yl)amino)-N-(4-(4-(2- methylpyridin-4-yl)piperazin- 1-yl)phenyl)-2-oxo-1,2- dihydropyridine-3- carboxamide | 99.8 | 553.47 |
| 341 | 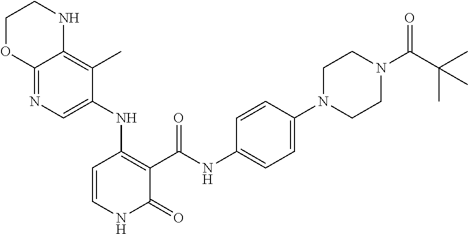 [SMILES: Cc1c(Nc2cc[nH]c(=O)c2C(=O)Nc2ccc(N3CCN(C(=O)C(C)(C)C)CC3)cc2)cnc2c1NCCO2] | 4-((8-methyl-2,3-dihydro-1H- pyrido[2,3-b][1,4]oxazin-7- yl)amino)-2-oxo-N-(4-(4- pivaloylpiperazin-1- yl)phenyl)-1,2- dihydropyridine-3- carboxamide | 98.8 | 546.43 |
| 342 | 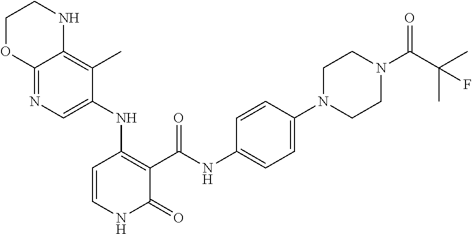 [SMILES: Cc1c(Nc2cc[nH]c(=O)c2C(=O)Nc2ccc(N3CCN(C(=O)C(C)(C)F)CC3)cc2)cnc2c1NCCO2] | N-(4-(4-(2-fluoro-2- methylpropanoyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.4 | 550.44 |
| 343 | 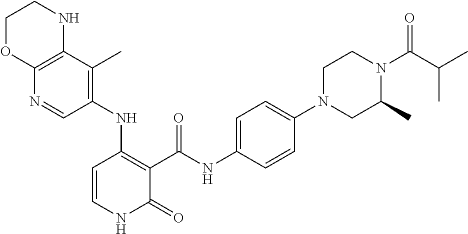 [SMILES: Cc1c(Nc2cc[nH]c(=O)c2C(=O)Nc2ccc(N3CCN(C(=O)C(C)C)[C@@H](C)C3)cc2)cnc2c1NCCO2] | (S)-N-(4-(4-isobutyryl-3- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.7 | 546.45 |
| 344 | 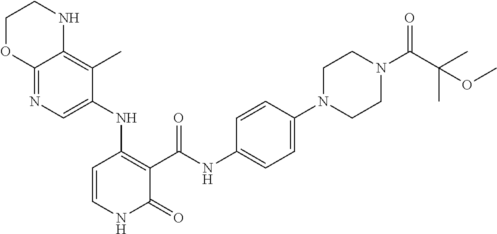 [SMILES: COC(C)(C)C(=O)N1CCN(c2ccc(NC(=O)c3c(Nc4cnc5c(c4C)NCCO5)cc[nH]c3=O)cc2)CC1] | N-(4-(4-(2-methoxy-2- methylpropanoyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.7 | 562.47 |
| 345 | 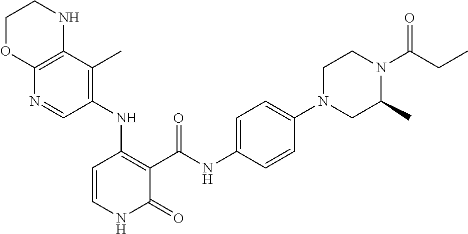 [SMILES: CCC(=O)N1CCN(c2ccc(NC(=O)c3c(Nc4cnc5c(c4C)NCCO5)cc[nH]c3=O)cc2)C[C@@H]1C] | (S)-4-((8-methyl-2,3-dihydro- 1H-pyrido[2,3-b][1,4]oxazin- 7-yl)amino)-N-(4-(3-methyl- 4-propionylpiperazin-1- yl)phenyl)-2-oxo-1,2- dihydropyridine-3- carboxamide | 97.9 | 532.40 |
| 346 | 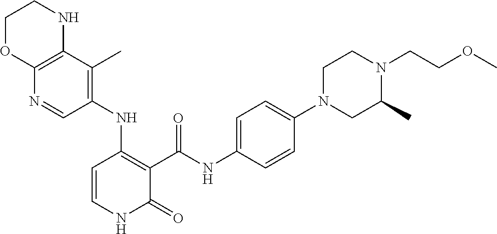 [SMILES: COCCN1CCN(c2ccc(NC(=O)c3c(Nc4cnc5c(c4C)NCCO5)cc[nH]c3=O)cc2)C[C@@H]1C] | (S)-N-(4-(4-(2- methoxyethyl)-3- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.2 | 534.42 |
| 347 | 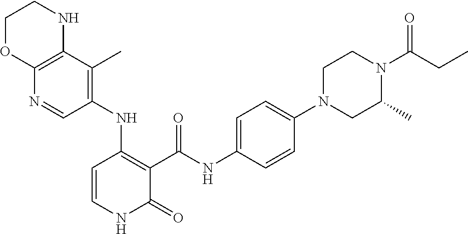 [SMILES: CCC(=O)N1CCN(c2ccc(NC(=O)c3c(Nc4cnc5c(c4C)NCCO5)cc[nH]c3=O)cc2)C[C@H]1C] | (R)-4-((8-methyl-2,3-dihydro- 1H-pyrido[2,3-b][1,4]oxazin- 7-yl)amino)-N-(4-(3-methyl- 4-propionylpiperazin-1- yl)phenyl)-2-oxo-1,2- dihydropyridine-3- carboxamide | 99.7 | 532.4 |
| 348 | 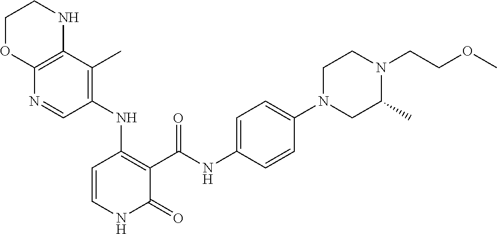 [SMILES: COCCN1CCN(c2ccc(NC(=O)c3c(Nc4cnc5c(c4C)NCCO5)cc[nH]c3=O)cc2)C[C@H]1C] | (R)-N-(4-(4-(2- methoxyethyl)-3- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 98.9 | 534.44 |
| 349 | 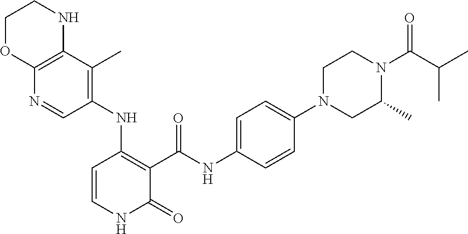 [SMILES: Cc1c(Nc2cc[nH]c(=O)c2C(=O)Nc2ccc(N3CCN(C(=O)C(C)C)[C@H](C)C3)cc2)cnc2c1NCCO2] | (R)-N-(4-(4-isobutyryl-3- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.78 | 546.42 |
| 350 | 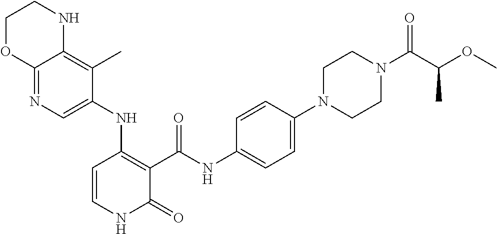 [SMILES: CO[C@@H](C)C(=O)N1CCN(c2ccc(NC(=O)c3c(Nc4cnc5c(c4C)NCCO5)cc[nH]c3=O)cc2)CC1] | (S)-N-(4-(4-(2- methoxypropanoyl)piperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.5 | 548.42 |
| 351 | 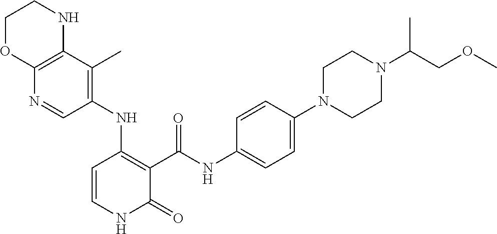 [SMILES: COCC(C)N1CCN(c2ccc(NC(=O)c3c(Nc4cnc5c(c4C)NCCO5)cc[nH]c3=O)cc2)CC1] | N-(4-(4-(1-methoxypropan-2- yl)piperazin-1-yl)phenyl)-4- ((8-methyl-2,3-dihydro-1H- pyrido[2,3-b][1,4]oxazin-7- yl)amino)-2-oxo-1,2- dihydropyridine-3- carboxamide | 99.5 | 534.44 |
| 352 | 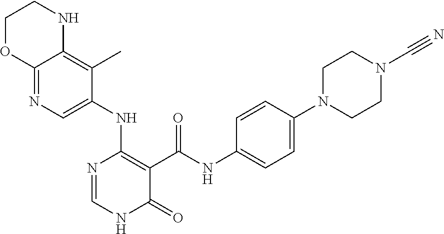 [SMILES: Cc1c(Nc2nc[nH]c(=O)c2C(=O)Nc2ccc(N3CCN(C#N)CC3)cc2)cnc2c1NCCO2] | N-(4-(4-cyanopiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.6 | 487.39 |
| 353 | 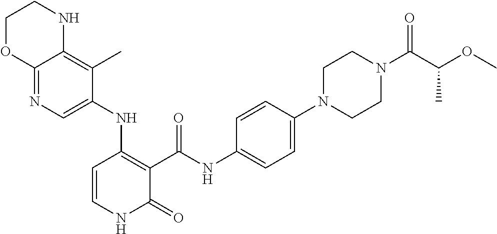 [SMILES: CO[C@H](C)C(=O)N1CCN(c2ccc(NC(=O)c3c(Nc4cnc5c(c4C)NCCO5)cc[nH]c3=O)cc2)CC1] | (R)-N-(4-(4-(2- methoxypropanoyl)piperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.5 | 548.40 |
| 354 | 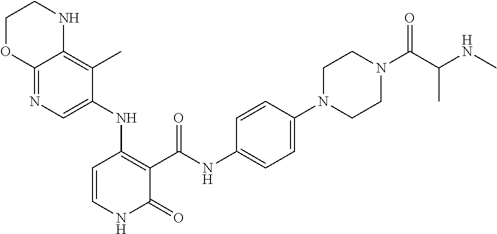 [SMILES: CNC(C)C(=O)N1CCN(c2ccc(NC(=O)c3c(Nc4cnc5c(c4C)NCCO5)cc[nH]c3=O)cc2)CC1] | 4-((8-methyl-2,3-dihydro-1H- pyrido[2,3-b][1,4]oxazin-7- yl)amino)-N-(4-(4- (methylalanyl)piperazin-1- yl)phenyl)-2-oxo-1,2- dihydropyridine-3- carboxamide | 97.8 | 547.42 |
| 355 | 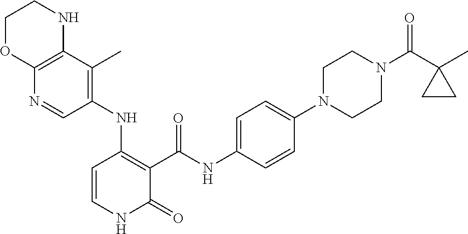 [SMILES: Cc1c(Nc2cc[nH]c(=O)c2C(=O)Nc2ccc(N3CCN(C(=O)C4(C)CC4)CC3)cc2)cnc2c1NCCO2] | 4-((8-methyl-2,3-dihydro-1H- pyrido[2,3-b][1,4]oxazin-7- yl)amino)-N-(4-(4-(1- methylcyclopropane-1- carbonyl)piperazin-1- yl)phenyl)-2-oxo-1,2- dihydropyridine-3- carboxamide | 99.4 | 544.4 |
| 356 | 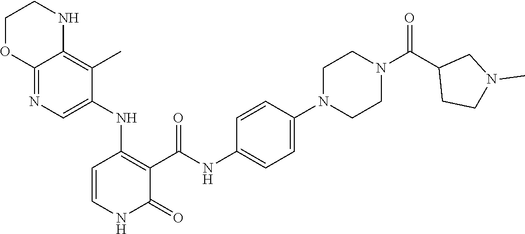 [SMILES: Cc1c(Nc2cc[nH]c(=O)c2C(=O)Nc2ccc(N3CCN(C(=O)C4CCN(C)C4)CC3)cc2)cnc2c1NCCO2] | 4-((8-methyl-2,3-dihydro-1H- pyrido[2,3-b][1,4]oxazin-7- yl)amino)-N-(4-(4-(1- methylpyrrolidine-3- carbonyl)piperazin-1- yl)phenyl)-2-oxo-1,2- dihydropyridine-3- carboxamide | 97.1 | 573.46 |
| 357 | 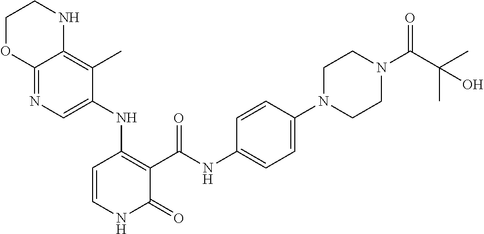 [SMILES: Cc1c(Nc2cc[nH]c(=O)c2C(=O)Nc2ccc(N3CCN(C(=O)C(C)(C)O)CC3)cc2)cnc2c1NCCO2] | N-(4-(4-(2-hydroxy-2- methylpropanoyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 96.2 | 548.42 |
| 358 | 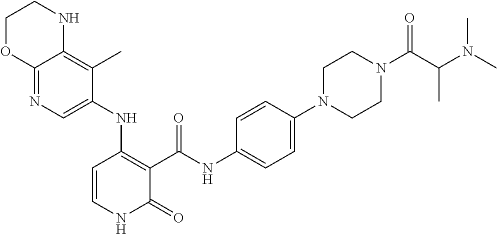 [SMILES: Cc1c(Nc2cc[nH]c(=O)c2C(=O)Nc2ccc(N3CCN(C(=O)C(C)N(C)C)CC3)cc2)cnc2c1NCCO2] | N-(4-(4- (dimethylalanyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.6 | 561.45 |
| 359 | 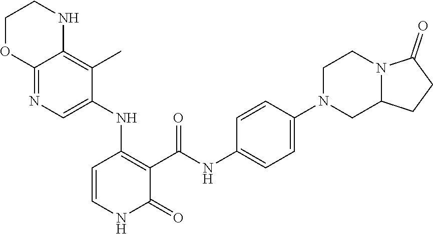 [SMILES: Cc1c(Nc2cc[nH]c(=O)c2C(=O)Nc2ccc(N3CCN4C(=O)CCC4C3)cc2)cnc2c1NCCO2] | (S)-4-((8-methyl-2,3-dihydro- 1H-pyrido[2,3-b][1,4]oxazin- 7-yl)amino)-2-oxo-N-(4-(6- oxohexahydropyrrolo[1,2- a]pyrazin-2(1H)-yl)phenyl)- 1,2-dihydropyridine-3- carboxamide | 99.4 | 516.35 |
| 360 | 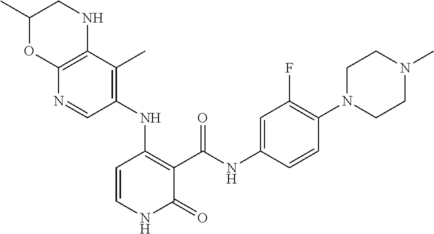 | 4-((3,8-dimethyl-2,3-dihydro- 1H-pyrido[2,3-b][1,4]oxazin- 7-yl)amino)-N-(3-fluoro-4-(4- methylpiperazin-1-yl)phenyl)- 2-oxo-1,2-dihydropyridine-3- carboxamide | 99.5 | 508.37 |
| 361 | 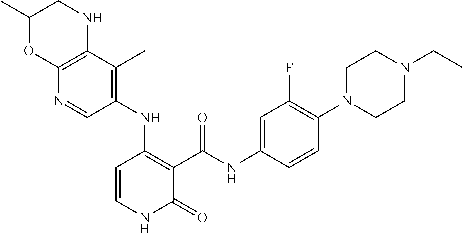 [SMILES: CCN1CCN(c2ccc(NC(=O)c3c(Nc4cnc5c(c4C)NCC(C)O5)cc[nH]c3=O)cc2F)CC1] | 4-((3,8-dimethyl-2,3-dihydro- 1H-pyrido[2,3-b][1,4]oxazin- 7-yl)amino)-N-(4-(4- ethylpiperazin-1-yl)-3- fluorophenyl)-2-oxo-1,2- dihydropyridine-3- carboxamide | 99.4 | 522.37 |
| 362 | 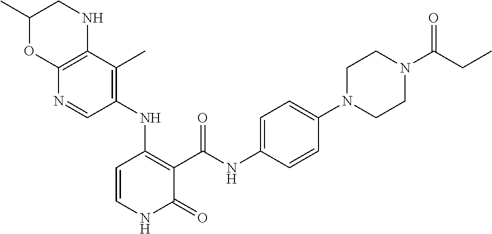 [SMILES: CCC(=O)N1CCN(c2ccc(NC(=O)c3c(Nc4cnc5c(c4C)NCC(C)O5)cc[nH]c3=O)cc2)CC1] | 4-((3,8-dimethyl-2,3-dihydro- 1H-pyrido[2,3-b][1,4]oxazin- 7-yl)amino)-2-oxo-N-(4-(4- propionylpiperazin-1- yl)phenyl)-1,2- dihydropyridine-3- carboxamide | 99.1 | 532.39 |
| 363 | 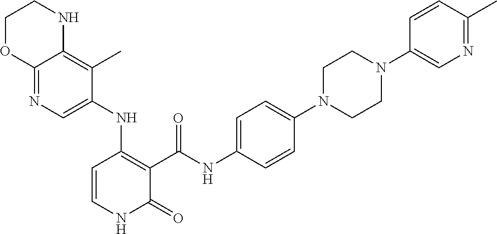 [SMILES: Cc1ccc(N2CCN(c3ccc(NC(=O)c4c(Nc5cnc6c(c5C)NCCO6)cc[nH]c4=O)cc3)CC2)cn1] | 4-((8-methyl-2,3-dihydro-1H- pyrido[2,3-b][1,4]oxazin-7- yl)amino)-N-(4-(4-(6- methylpyridin-3-yl)piperazin- 1-yl)phenyl)-2-oxo-1,2- dihydropyridine-3- carboxamide | 99.7 | 553.2 |
| 364 | 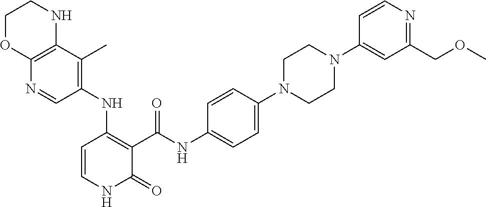 [SMILES: COCc1cc(N2CCN(c3ccc(NC(=O)c4c(Nc5cnc6c(c5C)NCCO6)cc[nH]c4=O)cc3)CC2)ccn1] | N-(4-(4-(2- (methoxymethyl)pyridin-4- yl)piperazin-1-yl)phenyl)-4- ((8-methyl-2,3-dihydro-1H- pyrido[2,3-b][1,4]oxazin-7- yl)amino)-2-oxo-1,2- dihydropyridine-3- carboxamide | 99.3 | 583.2 |
| 365 | 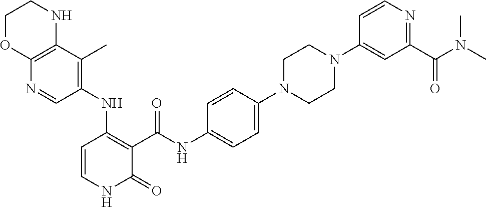 [SMILES: Cc1c(Nc2cc[nH]c(=O)c2C(=O)Nc2ccc(N3CCN(c4ccnc(C(=O)N(C)C)c4)CC3)cc2)cnc2c1NCCO2] | N,N-dimethyl-4-(4-(4-(4-((8- methyl-2,3-dihydro-1H- pyrido[2,3-b][1,4]oxazin-7- yl)amino)-2-oxo-1,2- dihydropyridine-3- carboxamido)phenyl)piperazin- 1-yl)picolinamide | 97.7 | 610.3 |
| 366 | 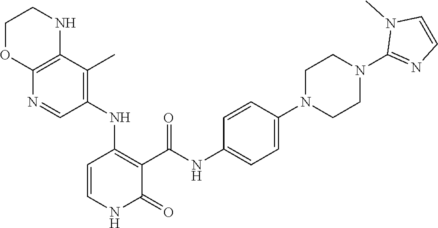 [SMILES: Cc1c(Nc2cc[nH]c(=O)c2C(=O)Nc2ccc(N3CCN(c4nccn4C)CC3)cc2)cnc2c1NCCO2] | N-(4-(4-(1-methyl-1H- imidazol-2-yl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.8 | 542.2 |
| 367 | 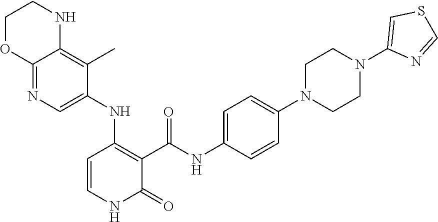 [SMILES: Cc1c(Nc2cc[nH]c(=O)c2C(=O)Nc2ccc(N3CCN(c4cscn4)CC3)cc2)cnc2c1NCCO2] | 4-((8-methyl-2,3-dihydro-1H- pyrido[2,3-b][1,4]oxazin-7- yl)amino)-2-oxo-N-(4-(4- (thiazol-4-yl)piperazin-1- yl)phenyl)-1,2- dihydropyridine-3- carboxamide | 99.3 | 545.1 |
| 368 | 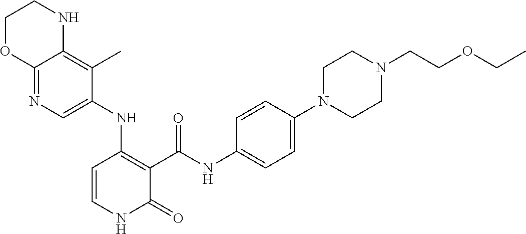 [SMILES: CCOCCN1CCN(c2ccc(NC(=O)c3c(Nc4cnc5c(c4C)NCCO5)cc[nH]c3=O)cc2)CC1] | N-(4-(4-(2- ethoxyethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.7 | 545.1 |
| 369 | 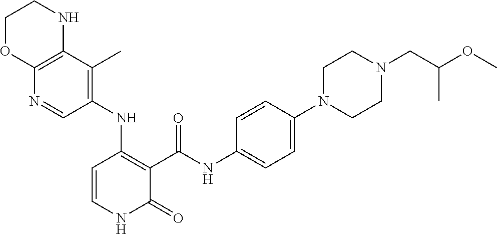 [SMILES: COC(C)CN1CCN(c2ccc(NC(=O)c3c(Nc4cnc5c(c4C)NCCO5)cc[nH]c3=O)cc2)CC1] | N-(4-(4-(2- methoxypropyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.5 | 534.42 |
| 370 | 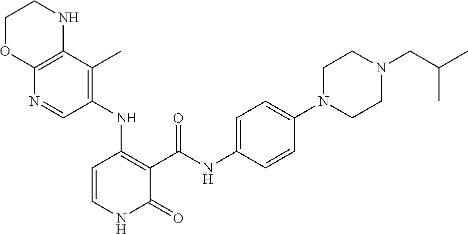 [SMILES: Cc1c(Nc2cc[nH]c(=O)c2C(=O)Nc2ccc(N3CCN(CC(C)C)CC3)cc2)cnc2c1NCCO2] | N-(4-(4-isobutylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.0 | 518.41 |
| 371 | 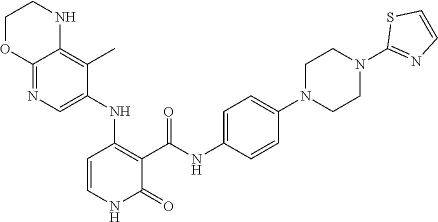 [SMILES: Cc1c(Nc2cc[nH]c(=O)c2C(=O)Nc2ccc(N3CCN(c4nccs4)CC3)cc2)cnc2c1NCCO2] | 4-((8-methyl-2,3-dihydro-1H- pyrido[2,3-b][1,4]oxazin-7- yl)amino)-2-oxo-N-(4-(4- (thiazol-2-yl)piperazin-1- yl)phenyl)-1,2- dihydropyridine-3- carboxamide | 99.1 | 545.39 |
| 372 | 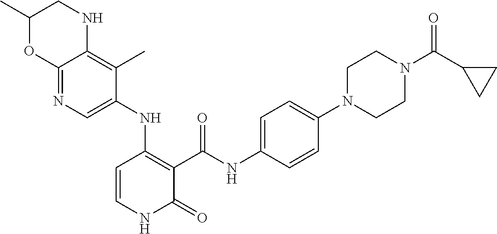 [SMILES: Cc1c(Nc2cc[nH]c(=O)c2C(=O)Nc2ccc(N3CCN(C(=O)C4CC4)CC3)cc2)cnc2c1NCC(C)O2] | N-(4-(4- (cyclopropanecarbonyl) piperazin-1-yl)phenyl)-4- ((3,8-dimethyl-2,3-dihydro- 1H-pyrido[2,3-b][1,4]oxazin- 7-yl)amino)-2-oxo-1,2- dihydropyridine-3- carboxamide | 98.4 | 544.39 |
| 373 | 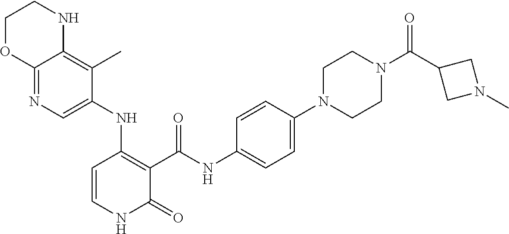 [SMILES: Cc1c(Nc2cc[nH]c(=O)c2C(=O)Nc2ccc(N3CCN(C(=O)C4CN(C)C4)CC3)cc2)cnc2c1NCCO2] | 4-((8-methyl-2,3-dihydro-1H- pyrido[2,3-b][1,4]oxazin-7- yl)amino)-N-(4-(4-(1- methylazetidine-3- carbonyl)piperazin-1- yl)phenyl)-2-oxo-1,2- dihydropyridine-3- carboxamide | 97.3 | 559.41 |
| 374 | 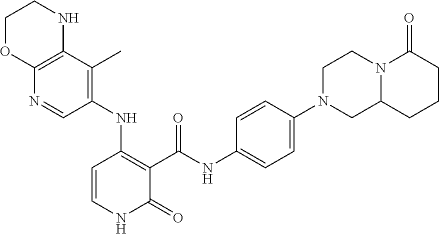 [SMILES: Cc1c(Nc2cc[nH]c(=O)c2C(=O)Nc2ccc(N3CCN4C(=O)CCCC4C3)cc2)cnc2c1NCCO2] | 4-((8-methyl-2,3-dihydro-1H- pyrido[2,3-b][1,4]oxazin-7- yl)amino)-2-oxo-N-(4-(6- oxooctahydro-2H-pyrido[1,2- a]pyrazin-2-yl)phenyl)-1,2- dihydropyridine-3- carboxamide | 99.7 | 530.39 |
| 375 | 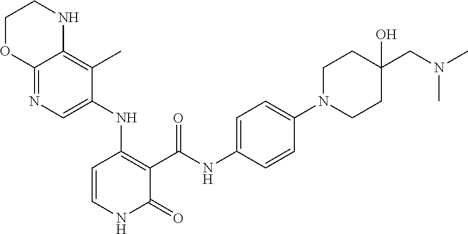 [SMILES: Cc1c(Nc2cc[nH]c(=O)c2C(=O)Nc2ccc(N3CCC(O)(CN(C)C)CC3)cc2)cnc2c1NCCO2] | N-(4-(4- ((dimethylamino)methyl)-4- hydroxypiperidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.3 | 534.42 |
| 376 | 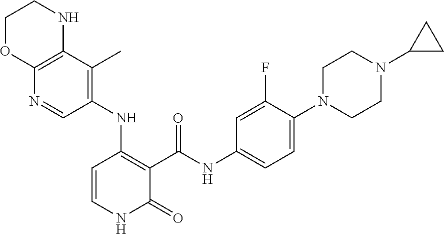 [SMILES: Cc1c(Nc2cc[nH]c(=O)c2C(=O)Nc2ccc(N3CCN(C4CC4)CC3)c(F)c2)cnc2c1NCCO2] | N-(4-(4- cyclopropylpiperazin-1-yl)-3- fluorophenyl)-4-((8-methyl- 2,3-dihydro-1H-pyrido[2,3- b][1,4]oxazin-7-yl)amino)-2- oxo-1,2-dihydropyridine-3- carboxamide | 99.7 | 520.36 |
| 377 | 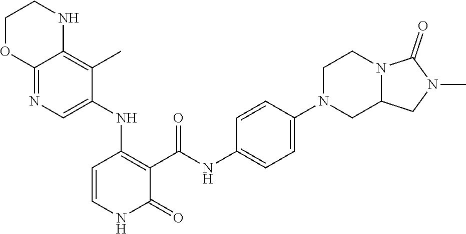 [SMILES: Cc1c(Nc2cc[nH]c(=O)c2C(=O)Nc2ccc(N3CCN4C(=O)N(C)CC4C3)cc2)cnc2c1NCCO2] | 4-((8-methyl-2,3-dihydro-1H- pyrido[2,3-b][1,4]oxazin-7- yl)amino)-N-(4-(2-methyl-3- oxohexahydroimidazo[1,5- a]pyrazin-7(1H)-yl)phenyl)-2- oxo-1,2-dihydropyridine-3- carboxamide | 97.9 | 531.36 |
| 378 | 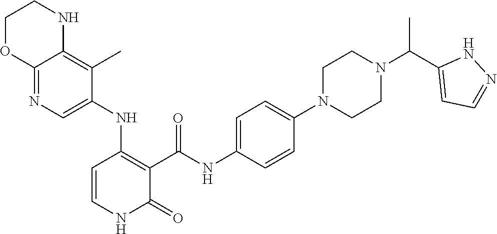 [SMILES: Cc1c(Nc2cc[nH]c(=O)c2C(=O)Nc2ccc(N3CCN(C(C)c4ccn[nH]4)CC3)cc2)cnc2c1NCCO2] | N-(4-(4-(1-(1H-pyrazol-5- yl)ethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.0 | 556.43 |
| 379 | 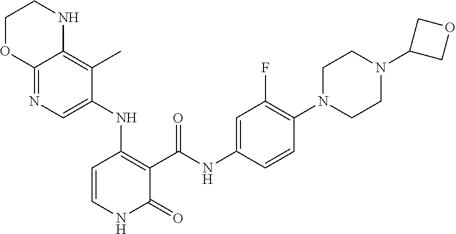 [SMILES: Cc1c(Nc2cc[nH]c(=O)c2C(=O)Nc2ccc(N3CCN(C4COC4)CC3)c(F)c2)cnc2c1NCCO2] | N-(3-fluoro-4-(4-(oxetan-3- yl)piperazin-1-yl)phenyl)-4- ((8-methyl-2,3-dihydro-1H- pyrido[2,3-b][1,4]oxazin-7- yl)amino)-2-oxo-1,2- dihydropyridine-3- carboxamide | 98.5 | 536.37 |
| 380 | 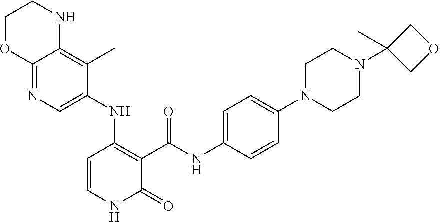 [SMILES: Cc1c(Nc2cc[nH]c(=O)c2C(=O)Nc2ccc(N3CCN(C4(C)COC4)CC3)cc2)cnc2c1NCCO2] | 4-((8-methyl-2,3-dihydro-1H- pyrido[2,3-b][1,4]oxazin-7- yl)amino)-N-(4-(4-(3- methyloxetan-3-yl)piperazin- 1-yl)phenyl)-2-oxo-1,2- dihydropyridine-3- carboxamide | 98.5 | 531.95 |
| 381 | 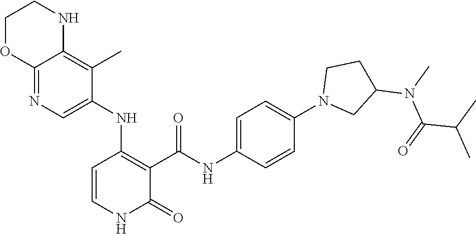 [SMILES: Cc1c(Nc2cc[nH]c(=O)c2C(=O)Nc2ccc(N3CCC(N(C)C(=O)C(C)C)C3)cc2)cnc2c1NCCO2] | 4-((8-methyl-2,3-dihydro-1H- pyrido[2,3-b][1,4]oxazin-7- yl)amino)-N-(4-(3-(N- methylisobutyramido) pyrrolidin- 1-yl)phenyl)-2-oxo-1,2- dihydropyridine-3- carboxamide | 96.2 | 545.93 |
| 382 | 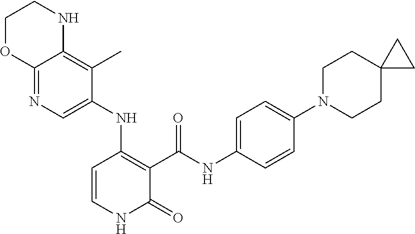 [SMILES: Cc1c(Nc2cc[nH]c(=O)c2C(=O)Nc2ccc(N3CCC4(CC3)CC4)cc2)cnc2c1NCCO2] | N-(4-(6-azaspiro[2.5]octan-6- yl)phenyl)-4-((8-methyl-2,3- dihydro-1H-pyrido[2,3- b][1,4]oxazin-7-yl)amino)-2- oxo-1,2-dihydropyridine-3- carboxamide | 96.4 | 487.36 |
| 383 | 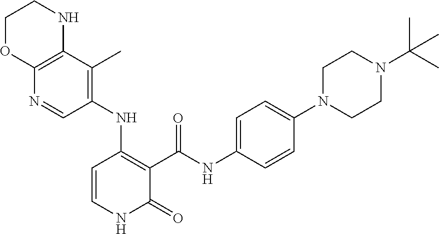 [SMILES: Cc1c(Nc2cc[nH]c(=O)c2C(=O)Nc2ccc(N3CCN(C(C)(C)C)CC3)cc2)cnc2c1NCCO2] | N-(4-(4-(tert-butyl)piperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.0 | 518.39 |
| 384 | 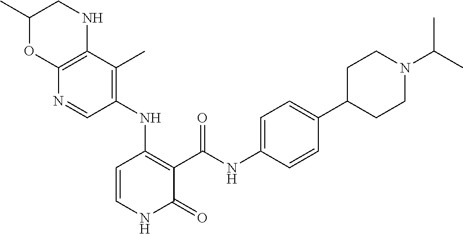 [SMILES: Cc1c(Nc2cc[nH]c(=O)c2C(=O)Nc2ccc(C3CCN(C(C)C)CC3)cc2)cnc2c1NCC(C)O2] | 4-((3,8-dimethyl-2,3-dihydro- 1H-pyrido[2,3-b][1,4]oxazin- 7-yl)amino)-N-(4-(1- isopropylpiperidin-4- yl)phenyl)-2-oxo-1,2- dihydropyridine-3- carboxamide | 99.2 | 517.39 |
| 385 | 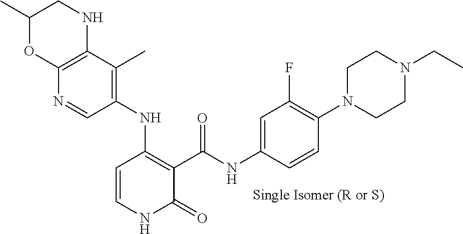 [SMILES: CCN1CCN(c2ccc(NC(=O)c3c(Nc4cnc5c(c4C)NCC(C)O5)cc[nH]c3=O)cc2F)CC1] | 4-((3,8-dimethyl-2,3-dihydro- 1H-pyrido[2,3-b][1,4]oxazin- 7-yl)amino)-N-(4-(4- ethylpiperazin-1-yl)-3- fluorophenyl)-2-oxo-1,2- dihydropyridine-3- carboxamide | 99.1 | 522.2 |
| 386 | 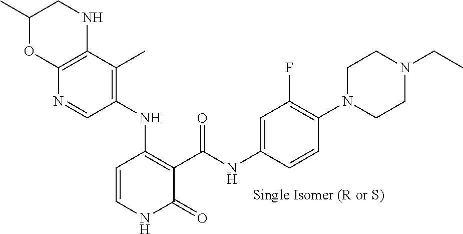 [SMILES: CCN1CCN(c2ccc(NC(=O)c3c(Nc4cnc5c(c4C)NCC(C)O5)cc[nH]c3=O)cc2F)CC1] | 4-((3,8-dimethyl-2,3-dihydro- 1H-pyrido[2,3-b][1,4]oxazin- 7-yl)amino)-N-(4-(4- ethylpiperazin-1-yl)-3- fluorophenyl)-2-oxo-1,2- dihydropyridine-3- carboxamide | 97.8 | 522.2 |
| 387 | 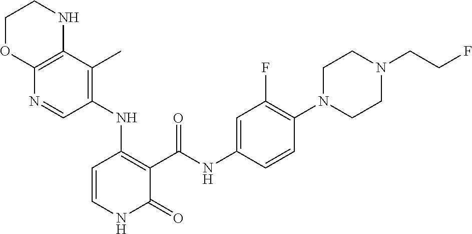 [SMILES: Cc1c(Nc2cc[nH]c(=O)c2C(=O)Nc2ccc(N3CCN(CCF)CC3)c(F)c2)cnc2c1NCCO2] | N-(3-fluoro-4-(4-(2- fluoroethyl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.8 | 526.38 |
| 388 | 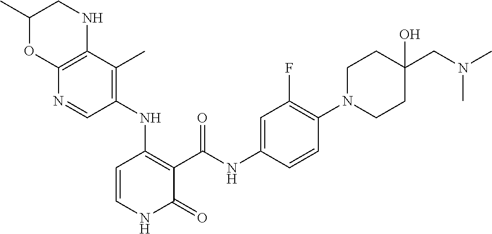 | 4-((3,8-dimethyl-2,3-dihydro- 1H-pyrido[2,3-b][1,4]oxazin- 7-yl)amino)-N-(4-(4- ((dimethylamino)methyl)-4- hydroxypiperidin-1-yl)-3- fluorophenyl)-2-oxo-1,2- dihydropyridine-3- carboxamide | 99.1 | 526.38 |
| 389 | 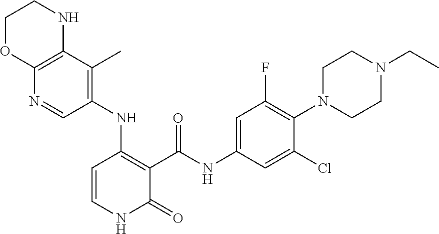 [SMILES: CCN1CCN(c2c(F)cc(NC(=O)c3c(Nc4cnc5c(c4C)NCCO5)cc[nH]c3=O)cc2Cl)CC1] | N-(3-chloro-4-(4- ethylpiperazin-1-yl)-5- fluorophenyl)-4-((8-methyl- 2,3-dihydro-1H-pyrido[2,3- b][1,4]oxazin-7-yl)amino)-2- oxo-1,2-dihydropyridine-3- carboxamide | 99.8 | 542.33 |
| 390 | 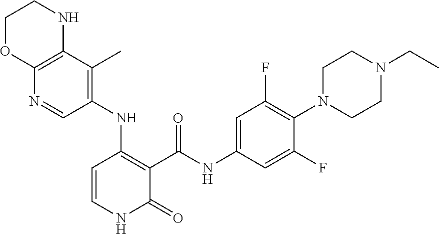 [SMILES: CCN1CCN(c2c(F)cc(NC(=O)c3c(Nc4cnc5c(c4C)NCCO5)cc[nH]c3=O)cc2F)CC1] | N-(4-(4-ethylpiperazin-1-yl)- 3,5-difluorophenyl)-4-((8- methyl-2,3-dihydro-1H- pyrido[2,3-b][1,4]oxazin-7- yl)amino)-2-oxo-1,2- dihydropyridine-3- carboxamide | 98.6 | 526.36 |
| 391 | 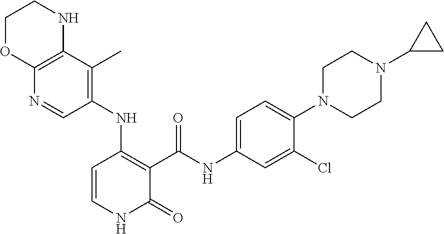 [SMILES: Cc1c(Nc2cc[nH]c(=O)c2C(=O)Nc2ccc(N3CCN(C4CC4)CC3)c(Cl)c2)cnc2c1NCCO2] | N-(3-chloro-4-(4- cyclopropylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.9 | 536.35 |
| 392 | 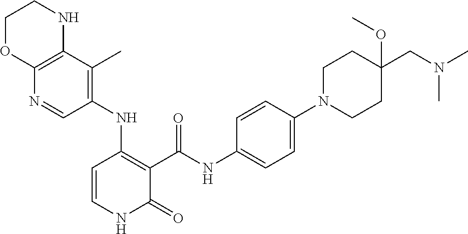 [SMILES: COC1(CN(C)C)CCN(c2ccc(NC(=O)c3c(Nc4cnc5c(c4C)NCCO5)cc[nH]c3=O)cc2)CC1] | N-(4-(4- ((dimethylamino)methyl)-4- methoxypiperidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.3 | 548.43 |
| 393 | 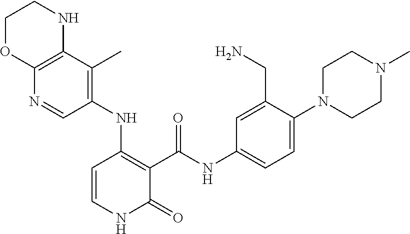 [SMILES: Cc1c(Nc2cc[nH]c(=O)c2C(=O)Nc2ccc(N3CCN(C)CC3)c(CN)c2)cnc2c1NCCO2] | N-(3-(aminomethyl)-4-(4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 95.4 | 502.91 |
| 394 | 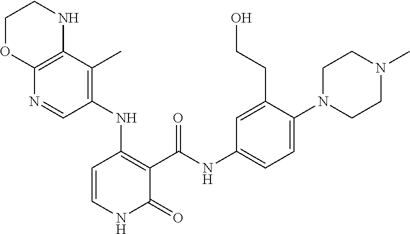 [SMILES: Cc1c(Nc2cc[nH]c(=O)c2C(=O)Nc2ccc(N3CCN(C)CC3)c(CCO)c2)cnc2c1NCCO2] | N-(3-(2-hydroxyethyl)-4-(4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 99.2 | 520.36 |
| 395 | 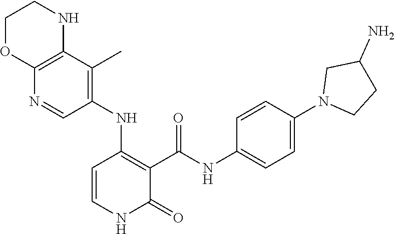 [SMILES: Cc1c(Nc2cc[nH]c(=O)c2C(=O)Nc2ccc(N3CCC(N)C3)cc2)cnc2c1NCCO2] | N-(4-(3-aminopyrrolidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 98.1 | 461.96 |
| 396 | 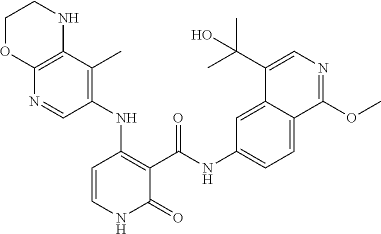 [SMILES: COc1ncc(C(C)(C)O)c2cc(NC(=O)c3c(Nc4cnc5c(c4C)NCCO5)cc[nH]c3=O)ccc12] | N-(4-(2-hydroxypropan-2-yl)- 1-methoxyisoquinolin-6-yl)- 4-((8-methyl-2,3-dihydro-1H- pyrido[2,3-b][1,4]oxazin-7- yl)amino)-2-oxo-1,2- dihydropyridine-3- carboxamide | 99.79 | 517.36 |
| 397 | 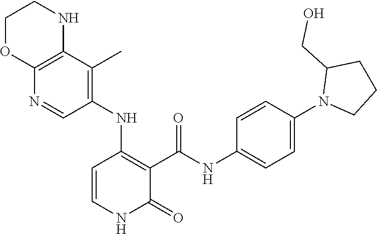 [SMILES: Cc1c(Nc2cc[nH]c(=O)c2C(=O)Nc2ccc(N3CCCC3CO)cc2)cnc2c1NCCO2] | N-(4-(2- (hydroxymethyl)pyrrolidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.7 | 476.96 |
| 398 | 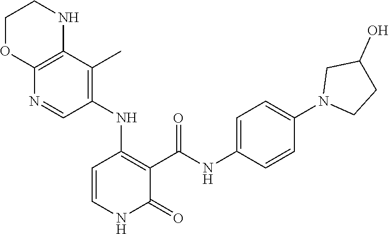 [SMILES: Cc1c(Nc2cc[nH]c(=O)c2C(=O)Nc2ccc(N3CCC(O)C3)cc2)cnc2c1NCCO2] | N-(4-(3-hydroxypyrrolidin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.0 | 463.38 |
| 399 | 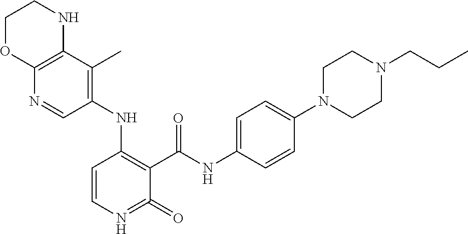 | 4-((8-methyl-2,3-dihydro-1H- pyrido[2,3-b][1,4]oxazin-7- yl)amino)-2-oxo-N-(4-(4- propylpiperazin-1-yl)phenyl)- 1,2-dihydropyridine-3- carboxamide | 98.4 | 504.34 |
| 400 | 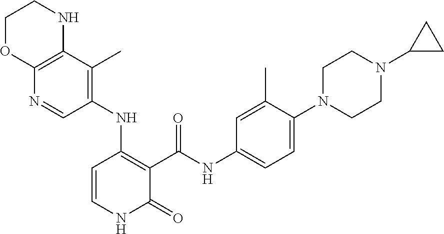 [SMILES: Cc1cc(NC(=O)c2c(Nc3cnc4c(c3C)NCCO4)cc[nH]c2=O)ccc1N1CCN(C2CC2)CC1] | N-(4-(4- cyclopropylpiperazin-1-yl)-3- methylphenyl)-4-((8-methyl- 2,3-dihydro-1H-pyrido[2,3- b][1,4]oxazin-7-yl)amino)-2- oxo-1,2-dihydropyridine-3- carboxamide | 99.9 | 516.35 |
| 401 | 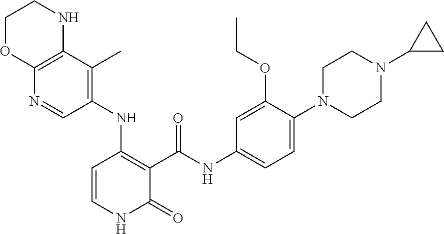 [SMILES: CCOc1cc(NC(=O)c2c(Nc3cnc4c(c3C)NCCO4)cc[nH]c2=O)ccc1N1CCN(C2CC2)CC1] | N-(4-(4- cyclopropylpiperazin-1-yl)-3- ethoxyphenyl)-4-((8-methyl- 2,3-dihydro-1H-pyrido[2,3- b][1,4]oxazin-7-yl)amino)-2- oxo-1,2-dihydropyridine-3- carboxamide | 99.7 | 924.22 |
| 402 | 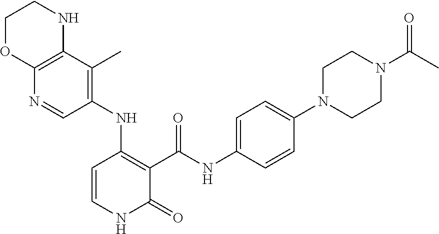 [SMILES: CC(=O)N1CCN(c2ccc(NC(=O)c3c(Nc4cnc5c(c4C)NCCO5)cc[nH]c3=O)cc2)CC1] | N-(4-(4-acetylpiperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.9 | 504.43 |
| 403 | 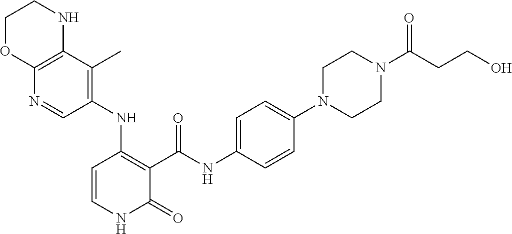 [SMILES: Cc1c(Nc2cc[nH]c(=O)c2C(=O)Nc2ccc(N3CCN(C(=O)CCO)CC3)cc2)cnc2c1NCCO2] | N-(4-(4-(3- hydroxypropanoyl)piperazin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 98.6 | 534.21 |
| 404 | 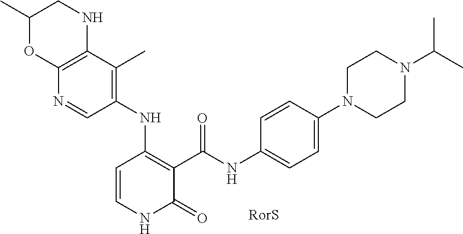 [SMILES: Cc1c(Nc2cc[nH]c(=O)c2C(=O)Nc2ccc(N3CCN(C(C)C)CC3)cc2)cnc2c1NCC(C)O2] | 4-((3,8-dimethyl-2,3-dihydro- 1H-pyrido[2,3-b][1,4]oxazin- 7-yl)amino)-N-(4-(4- isopropylpiperazin-1- yl)phenyl)-2-oxo-1,2- dihydropyridine-3- carboxamide | 99.66 (chiral purity 99.92) | 518.2 |
| 405 | 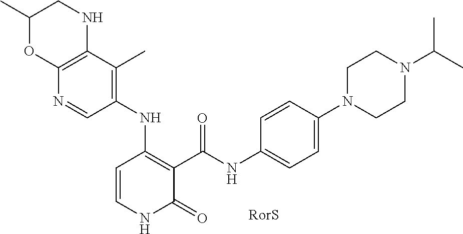 [SMILES: Cc1c(Nc2cc[nH]c(=O)c2C(=O)Nc2ccc(N3CCN(C(C)C)CC3)cc2)cnc2c1NCC(C)O2] | 4-((3,8-dimethyl-2,3-dihydro- 1H-pyrido[2,3-b][1,4]oxazin- 7-yl)amino)-N-(4-(4- isopropylpiperazin-1- yl)phenyl)-2-oxo-1,2- dihydropyridine-3- carboxamide | 99.51 (chiral purity 99.11) | 518.4 |
| 406 | 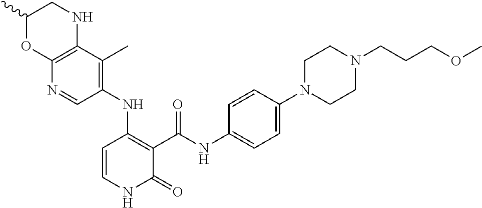 | 4-((3,8-dimethyl-2,3-dihydro- 1H-pyrido[2,3-b][1,4]oxazin- 7-yl)amino)-N-(4-(4-(3- methoxypropyl)piperazin-1- yl)phenyl)-2-oxo-1,2- dihydropyridine-3- carboxamide | 99.92 | 548.34 |
| 407 | 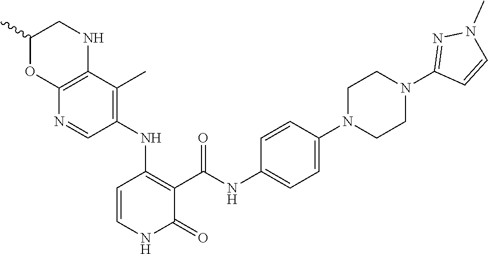 [SMILES: Cc1c(Nc2cc[nH]c(=O)c2C(=O)Nc2ccc(N3CCN(c4ccn(C)n4)CC3)cc2)cnc2c1NCC(C)O2] | 4-((3,8-dimethyl-2,3-dihydro- 1H-pyrido[2,3-b][1,4]oxazin- 7-yl)amino)-N-(4-(4-(1- methyl-1H-pyrazol-3- yl)piperazin-1-yl)phenyl)-2- oxo-1,2-dihydropyridine-3- carboxamide | 99.26 | 556.33 |
| 408 | 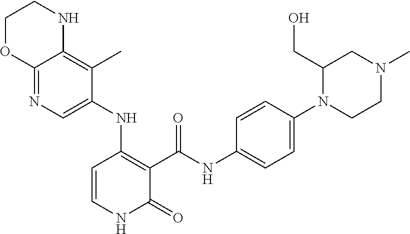 [SMILES: Cc1c(Nc2cc[nH]c(=O)c2C(=O)Nc2ccc(N3CCN(C)CC3CO)cc2)cnc2c1NCCO2] | N-(4-(2-(hydroxymethyl)-4- methylpiperazin-1-yl)phenyl)- 4-((8-methyl-2,3-dihydro-1H- pyrido[2,3-b][1,4]oxazin-7- yl)amino)-2-oxo-1,2- dihydropyridine-3- carboxamide | 98.83 | 506.25 |
| 409 | 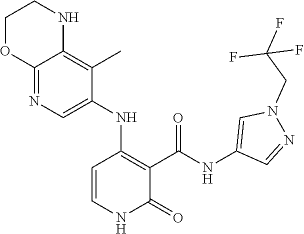 [SMILES: Cc1c(Nc2cc[nH]c(=O)c2C(=O)Nc2cnn(CC(F)(F)F)c2)cnc2c1NCCO2] | 4-((8-methyl-2,3-dihydro-1H- pyrido[2,3-b][1,4]oxazin-7- yl)amino)-2-oxo-N-(1-(2,2,2- trifluoroethyl)-1H-pyrazol-4- yl)-1,2-dihydropyridine-3- carboxamide | 99.75 | 450.13 |
| 410 | 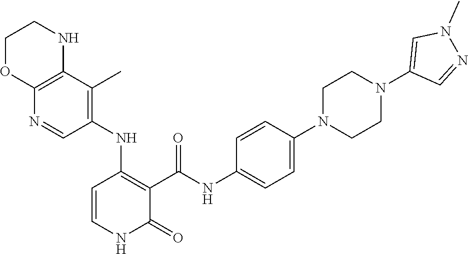 [SMILES: Cc1c(Nc2cc[nH]c(=O)c2C(=O)Nc2ccc(N3CCN(c4cnn(C)c4)CC3)cc2)cnc2c1NCCO2] | N-(4-(4-(1-methyl-1H- pyrazol-4-yl)piperazin-1- yl)phenyl)-4-((8-methyl-2,3- dihydro-1H-pyrido[2,3- b][1,4]oxazin-7-yl)amino)-2- oxo-1,2-dihydropyridine-3- carboxamide | 99.76 | 542.26 |
| 411 | 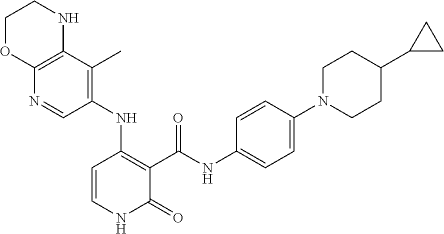 [SMILES: Cc1c(Nc2cc[nH]c(=O)c2C(=O)Nc2ccc(N3CCC(C4CC4)CC3)cc2)cnc2c1NCCO2] | N-(4-(4-cyclopropylpiperidin- 1-yl)phenyl)-4-((8-methyl- 2,3-dihydro-1H-pyrido[2,3- b][1,4]oxazin-7-yl)amino)-2- oxo-1,2-dihydropyridine-3- carboxamide | 99.87 | 501.27 |

### Example 98:4-((8-Methyl-2,3-dihydro-1H-pyrido[2,3-b][1,4]oxazin-7-yl)amino)-N-(4-(4-methylpiperazin-1-yl)phenyl)-6-oxo-1,6-dihydropyrimidine-5-carboxamide

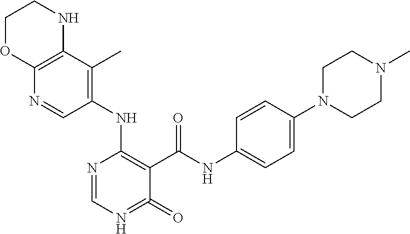

Example 98 was prepared according to the methods described in General Procedures 1-3, 8 and the methods described below.

### Preparation 6:4-Methoxy-6-((8-methyl-2,3-dihydro-1H-pyrido[2,3-b][1,4]oxazin-7-yl)amino)pyrimidine-5-carboxylic acid

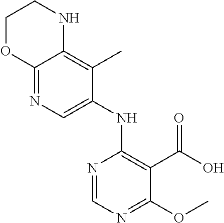 [SMILES: COc1ncnc(Nc2cnc3c(c2C)NCCO3)c1C(=O)O]

### Step 1: Methyl 4-chloro-6-((8-methyl-2,3-dihydro-1H-pyrido[2,3-b][1,4]oxazin-7-yl)amino)pyrimidine-5-carboxylate

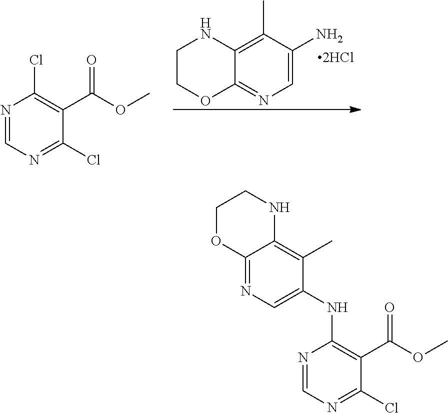

To a stirred solution of commercially available methyl 4,6-dichloropyrimidine-5-carboxylate (300 mg, 1.449 mmol) in t-BuOH (10 mL) was added 8-methyl-2,3-dihydro-1H-pyrido[2,3-b][1,4]oxazin-7-amine. 2HCl (Preparation 9, Step 4) (239.41 mg, 1.449 mmol) followed by DIPEA (1.262 mL, 7.246 mmol) at RT. The resulting reaction mixture was heated at 80° C. for 18 h. Progress of the reaction was monitored by LCMS and after completion the solvent was evaporated to dryness. Then it was diluted with water and extracted with ethyl acetate. The combined organic layers were distilled off to obtain the crude compound which was purified over a silica gel bed using ethyl acetate and hexane as eluent to afford the title compound (90 mg, 18% yield) as a brown solid. LCMS m/z: 336.18 [M+H].

### Step 2:4-Methoxy-6-((8-methyl-2,3-dihydro-1H-pyrido[2,3-b][1,4]oxazin-7-yl)amino)pyrimidine-5-carboxylic acid

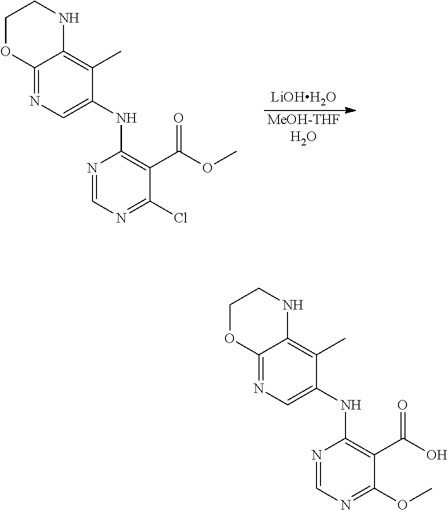 [SMILES: COC(=O)c1c(Cl)ncnc1Nc1cnc2c(c1C)NCCO2 ; "MeOH-THFH2O" ; COc1ncnc(Nc2cnc3c(c2C)NCCO3)c1C(=O)O]

To a stirred solution of methyl 4-chloro-6-((8-methyl-2,3-dihydro-1H-pyrido[2,3-b][1,4]oxazin-7-yl)amino)pyrimidine-5-carboxylate (Preparation 6, Step 1) (90 mg, 0.268 mmol) in THF (3 mL) and methanol (1.5 mL) was added an aqueous solution of LiOH.H₂O (44.99 mg, 1.072 mmol, 1.5 mL water) at RT. The whole was stirred at RT overnight. Progress of the reaction was checked by LCMS and after completion the solvent was evaporated in vacuo to give a residue which was diluted with water and pH adjusted with citric acid to make the solution acidic to obtain a solid precipitate. The precipitate was filtered, washed and dried to afford the title compound (65 mg, 76% yield) as an off-white solid. LCMS m/z: 318.21 [M+H].

### Preparation 7:4-Methoxy-6-((8-methyl-2,3-dihydro-1H-pyrido[2,3-b][1,4]oxazin-7-yl)amino)-N-(4-(4-methylpiperazin-1-yl)phenyl)pyrimidine-5-carboxamide

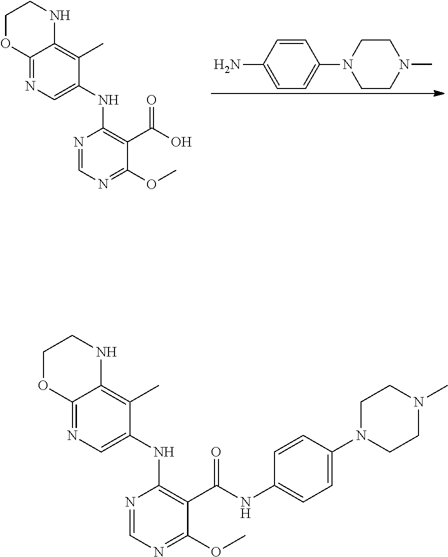 [SMILES: COc1ncnc(Nc2cnc3c(c2C)NCCO3)c1C(=O)O ; CN1CCN(c2ccc(N)cc2)CC1 ; COc1ncnc(Nc2cnc3c(c2C)NCCO3)c1C(=O)Nc1ccc(N2CCN(C)CC2)cc1]

To a stirred solution of 4-methoxy-6-((8-methyl-2,3-dihydro-1H-pyrido[2,3-b][1,4]oxazin-7-yl)amino)pyrimidine-5-carboxylic acid (Preparation 6, Step 2) (55 mg, 0.173 mmol) in THF (3 mL) was added HATU (79.08 mg, 0.207 mmol) and after 15 minutes, DIPEA (0.090 mL, 0.519 mmol) and 4-(4-methylpiperazin-1-yl) aniline (39.70 mg, 0.207 mmol) were added into the reaction vessel. The resulting reaction mixture was stirred at RT for 3 h. Progress of the reaction was monitored by TLC/LC-MS and after completion the reaction mass was diluted with water and extracted with 10% methanol in DCM. The combined organic layers were distilled off to obtain the crude product which was purified over a silica gel bed using methanol and DCM as eluent to afford the title compound (85 mg, 100% yield) as an off-white sticky solid. LCMS m/z: 491.35 [M+H].

### Preparation 8:4-((8-Methyl-2,3-dihydro-1H-pyrido[2,3-b][1,4]oxazin-7-yl)amino)-N-(4-(4-methylpiperazin-1-yl)phenyl)-6-oxo-1,6-dihydropyrimidine-5-carboxamide (Example 98)

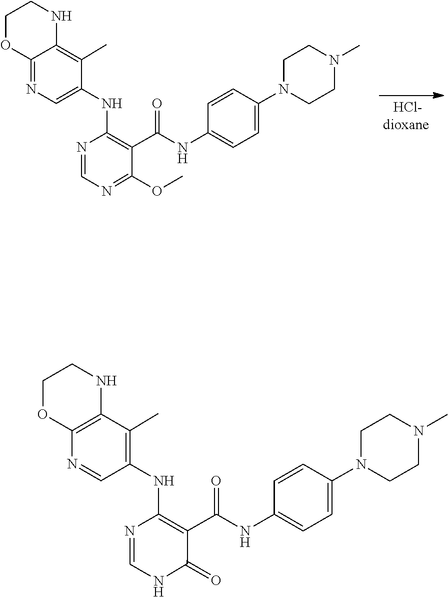 [SMILES: COc1ncnc(Nc2cnc3c(c2C)NCCO3)c1C(=O)Nc1ccc(N2CCN(C)CC2)cc1 ; Cc1c(Nc2nc[nH]c(=O)c2C(=O)Nc2ccc(N3CCN(C)CC3)cc2)cnc2c1NCCO2]

A stirred suspension of 4-methoxy-6-((8-methyl-2,3-dihydro-1H-pyrido[2,3-b][1,4]oxazin-7-yl)amino)-N-(4-(4-methylpiperazin-1-yl)phenyl)pyrimidine-5-carboxamide (Preparation 7) (75 mg, 0.394 mmol) in 4M HCl in dioxane (3 mL) was allowed to stir at 80° C. for 3 h. Progress of the reaction was monitored by LCMS and after completion the solvent was evaporated in vacuo to give a residue which was triturated with diethyl ether and finally purified by prep-HPLC to afford the title compound (18 mg, 25% yield) as a yellow solid. HPLC purity: 99.53%; ¹H NMR (500 MHz; DMSO-d₆): δ 1.86 (s, 3H), 2.15 (s, 3H), 2.38 (s, 4H, merged with DMSO), 3.02 (s, 4H), 3.25 (s, 2H, merged with DMSO water)4.16 (d, J=3.65 Hz, 2H), 5.60 (s, 1H), 6.85 (d, J=9.0 Hz, 2H), 7.36 (s, 1H), 7.40 (d, J=8.9 Hz, 2H), 7.96 (s, 1H), 11.79 (s, 1H), 12.07 (s, 1H), 12.42 (bh, 1H); LCMS m/z: 477.43 [M+H].

The examples in the table below were prepared according to the above methods used to make Example 98 as described in General Procedures 1-8 using the appropriate amines. Purification was as stated in the aforementioned methods.

| | | | Purity | LCMS |
|---|---|---|---|---|
| Ex | Structure | IUPAC Name | (%) | [M + H] |
| 98 | 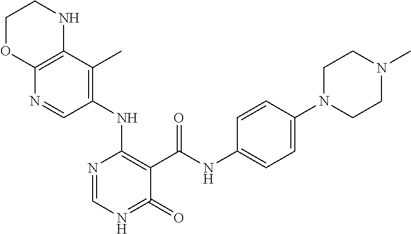 | 4-((8-methyl-2,3-dihydro- 1H-pyrido[2,3-b][1,4] oxazin-7-yl)amino)-N- (4-(4-methylpiperazin-1- yl)phenyl)-6-oxo-1,6- dihydropyrimidine-5- carboxamide | 99.53 | 477.43 |
| 412 | 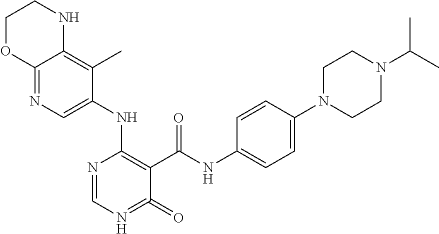 | N-(4-(4-isopropylpiper- azin-1-yl)phenyl)-4-((8- methyl-2,3-dihydro-1H- pyrido[2,3-b][1,4]oxazin- 7-yl)amino)-6-oxo-1,6- dihydropyrimidine-5- carboxamide | 99.25 | 505.34 |
| 413 | 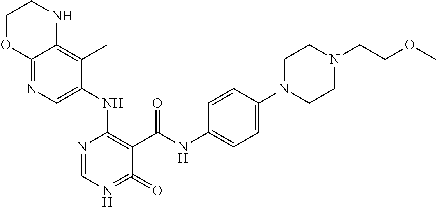 | N-(4-(4-(2-methoxyethyl)- piperazin-1-yl)phenyl)-4- ((8-methyl-2,3-dihydro- 1H-pyrido[2,3-b][1,4] oxazin-7-yl)amino)-6-oxo- 1,6-dihydropyrimidine-5- carboxamide | 99.31 | 521.34 |

### Biological Testing

HPK-1 biochemical enzyme assay

Compound inhibitory potency was measured in an HPK-1 kinase inhibition assay. Briefly, recombinant full length HPK-1 enzyme (6.8 nM) was incubated with 10 μM ATP and 12.5 μM swine myelin basic protein (MBP) for 30 mins. at 25° C. in the presence of various concentrations of test compound or vehicle in 40 mM Tris.Cl pH7.4 buffer containing 20 mM MgCl₂, 50 μM DTT and 0.1 mg/mL BSA. Reactions were quenched and reaction mixtures were then analyzed by an ADP-Glo kit which measures the formed ADP. The percent inhibition was calculated from the substrate conversion considering no enzyme control reactions for 100% inhibition and vehicle only reactions for 0% inhibition. The compounds were dissolved in DMSO and evaluated at 10 concentrations to determine an IC₅₀ value.

In the below table A denotes an HPK-1 IC₅₀≤10 nM, B denotes an HPK-1 IC₅₀>10 nM but≤100 nM and C denotes an HPK-1 IC₅₀>100 nM but≤1000 nM.

| | HPK-1 | | HPK-1 | | HPK-1 | | HPK-1 |
|---|---|---|---|---|---|---|---|
| Ex. | IC₅₀ | Ex. | IC₅₀ | Ex. | IC₅₀ | Ex. | IC₅₀ |
| 168 | A | 207 | B | 244 | B | 276 | A |
| 169 | A | 208 | C | 245 | C | 277 | A |
| 170 | A | 45 | A | 246 | B | 278 | A |
| 171 | A | 209 | B | 247 | A | 130 | B |
| 172 | B | 210 | B | 248 | B | 279 | A |
| 173 | B | 211 | B | 249 | B | 132 | B |
| 174 | A | 212 | A | 250 | B | 133 | B |
| 175 | A | 213 | B | 251 | A | 134 | A |
| 176 | A | 161 | B | 93 | B | 135 | A |
| 177 | A | 214 | A | 252 | B | 136 | A |
| 178 | A | 215 | C | 253 | C | 137 | A |
| 179 | A | 216 | B | 96 | A | 138 | A |
| 180 | B | 217 | C | 97 | A | 139 | A |
| 181 | B | 218 | C | 254 | B | 140 | A |
| 182 | A | 219 | A | 255 | B | 280 | B |
| 183 | A | 220 | A | 256 | A | 281 | B |
| 184 | A | 221 | C | 257 | B | 143 | A |
| 185 | A | 222 | C | 258 | B | 144 | B |
| 186 | A | 162 | A | 259 | A | 145 | B |
| 187 | A | 223 | B | 260 | C | 146 | B |
| 188 | B | 224 | B | 105 | A | 147 | A |
| 189 | B | 225 | A | 106 | A | 282 | A |
| 190 | B | 163 | B | 261 | A | 283 | A |
| 191 | B | 226 | A | 262 | B | 284 | A |
| 192 | B | 227 | A | 263 | A | 151 | B |
| 193 | A | 228 | B | 264 | B | 152 | B |
| 194 | A | 229 | C | 265 | B | 153 | A |
| 195 | C | 230 | B | 266 | A | 154 | A |
| 196 | B | 231 | B | 267 | B | 285 | A |
| 197 | B | 232 | B | 268 | B | 156 | B |
| 164 | B | 233 | B | 115 | B | 286 | A |
| 198 | B | 234 | B | 269 | B | 287 | B |
| 199 | B | 235 | A | 270 | A | 288 | A |
| 200 | A | 236 | B | 118 | B | 289 | A |
| 201 | B | 237 | B | 271 | B | 290 | A |
| 202 | B | 238 | A | 165 | A | 291 | B |
| 203 | B | 239 | C | 98 | B | 292 | A |
| 204 | B | 240 | B | 272 | B | 293 | A |
| 205 | B | 241 | A | 273 | C | 125 | B |
| 206 | B | 242 | B | 274 | A | 126 | B |
| 167 | A | 243 | C | 275 | A | 294 | A |
| 295 | B | 296 | C | 297 | C | 298 | A |
| 299 | B | 300 | A | 301 | B | 302 | A |
| 303 | A | 304 | A | 305 | A | 306 | A |
| 307 | A | 308 | A | 309 | A | 310 | A |
| 311 | A | 312 | A | 313 | B | 314 | A |
| 315 | A | 316 | B | 317 | A | 318 | A |
| 319 | A | 320 | A | 321 | B | 322 | B |
| 323 | B | 324 | A | 325 | A | 326 | A |
| 327 | A | 328 | A | 329 | A | 330 | C |
| 33 | B | 332 | A | 333 | A | 334 | A |
| 335 | B | 336 | A | 337 | A | 338 | B |
| 339 | A | 340 | A | 341 | A | 342 | A |
| 343 | A | 344 | A | 345 | A | 346 | A |
| 347 | A | 348 | A | 349 | A | 350 | A |
| 351 | B | 352 | B | 353 | B | 354 | B |
| 355 | A | 356 | B | 357 | A | 358 | B |
| 359 | B | 360 | B | 361 | B | 362 | B |
| 363 | B | 364 | B | 365 | B | 366 | A |
| 367 | B | 368 | B | 369 | B | 370 | A |
| 371 | B | 372 | A | 373 | B | 374 | B |
| 375 | A | 376 | A | 377 | A | 378 | A |
| 379 | A | 380 | B | 381 | B | 382 | B |
| 383 | A | 384 | B | 385 | B | 386 | A |
| 387 | B | 388 | A | 389 | A | 390 | B |
| 391 | A | 392 | A | 393 | A | 394 | B |
| 395 | B | 396 | C | 397 | B | 398 | B |
| 399 | A | 400 | B | 401 | B | 402 | B |
| 403 | A | 404 | A | 405 | B | 406 | B |
| 407 | B | 408 | B | 409 | B | 410 | B |
| 411 | B | 412 | B | 413 | B | | |

1. A compound of formula (I): 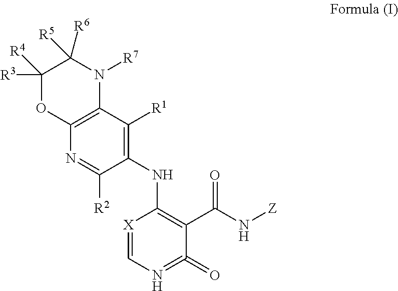
wherein:
X is CH or N;
Z is a phenyl or 5 or 6 membered heteroaryl, wherein the phenyl or heteroaryl is substituted with one or more substituents selected from the group consisting of a halogen, an optionally substituted C₁-C₆ alkyl, an optionally substituted
C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 10 membered heterocyclyl, an optionally substituted 5 to 10 membered heteroaryl, an optionally substituted C₆₋₁₀ aryl or an optionally substituted C₃₋₉ cycloalkyl; and/or where adjacent substituents of the phenyl or heteroaryl, together with the atoms to which they are attached, may combine to form an optionally substituted 3 to 6 membered heterocycle or an optionally substituted 5 or 6 membered heteroaryl;
R¹ to R⁷ are independently hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹, NR¹⁰R¹¹, an optionally substituted
C₃-C₆ cycloalkyl, an optionally substituted 3 to 8 membered heterocyclyl, an optionally substituted 5 to 10 membered heteroaryl or an optionally substituted phenyl; and/or R³ and R⁴ and/or R⁵ and R⁶, together with the C atom they are bound to, form a C═O group, an optionally substituted C₃-C₆ cycloalkyl or an optionally substituted 3 to 8 membered heterocyclyl; and/or R³ and R⁵, together with the C atoms they are bound to, form an optionally substituted C₃-C₆ cycloalkyl or an optionally substituted 3 to 8 membered heterocyclyl;
R⁸ and R⁹ are independently hydrogen, an optionally substituted C₁-C₁₂ alkyl, an optionally substituted C₂-C₁₂ alkenyl, an optionally substituted C₂-C₁₂ alkynyl, optionally substituted C₆₋₁₂ aryl, optionally substituted C₃-C₆ cycloalkyl, optionally substituted 3 to 8 membered heterocyclyl, and an optionally substituted 5 to 10 membered heteroaryl; and
R¹⁰ and R¹¹ are independently hydrogen, an optionally substituted C₁-C₆ alkyl, an optionally substituted C₂-C₆ alkenyl, an optionally substituted C₂-C₆ alkynyl, optionally substituted C₆₋₁₂ aryl, optionally substituted C₃-C₆ cycloalkyl, optionally substituted 3 to 8 membered heterocyclyl, and an optionally substituted 5 to 10 membered heteroaryl;
or a pharmaceutically acceptable complex, salt, solvate, tautomeric form or polymorphic form thereof.

2. The compound according to claim 1, wherein R¹ is hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹ or NR¹⁰R¹¹.

3. The compound according to claim 1, wherein R² is hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹ or NR¹⁰R¹¹.

4. The compound according to claim 1, wherein R³ and R⁴ are independently hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl or R³ and R⁴ together with the C atom they are bound to, form a C═O group, an optionally substituted C₃-C₆ cycloalkyl or an optionally substituted 3 to 8 membered heterocyclyl.

5. The compound according to claim 1, wherein R⁵ and R⁶ are independently hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹, NR¹⁰R¹¹, an optionally substituted C₃-C₆ cycloalkyl, an optionally substituted 3 to 8 membered heterocyclyl, an optionally substituted 5 to 10 membered heteroaryl or an optionally substituted phenyl or R⁵ and R⁶ together with the C atom they are bound to, form a C═O group, an optionally substituted C₃-C₆ cycloalkyl or an optionally substituted 3 to 8 membered heterocyclyl.

6. The compound according to claim 1, wherein R⁷ is hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹ or NR¹⁰R¹¹.

7. The compound according to claim 1, wherein:
R¹ is hydrogen, methyl, CN or OCH₃;
R² is hydrogen;
R³ and R⁴ are independently hydrogen, methyl, ethyl, i-propyl or fluorine, or R³ and R⁴ together with the C atom they are bound to form cyclopropyl;
R⁵ and R⁶ are hydrogen, or R⁵ and R⁶ together with the C atom they are bound to form a C═O group; and
R⁷ is hydrogen or methyl.

8. The compound according to claim 1, wherein Z is a substituted phenyl, a substituted pyrazole or a substituted pyridinyl group.

9. The compound according to claim 1, wherein adjacent substituents of the phenyl or 5 or 6 membered heteroaryl group Z are linked to form, with the C atoms of the phenyl or heteroaryl group they are bound to, a 5- or 6-membered heterocyclic or heteroaromatic group, optionally wherein adjacent substituents of the heterocyclic or heteroaryl group, together with the atoms to which they are attached, combine to form a further optionally substituted 3 to 6 membered heterocycle or a further optionally substituted 5 or 6 membered heteroaryl, such that the group Z is an optionally substituted bicyclic fused group or an optionally substituted tricyclic fused group.

10. The compound according to claim 9, wherein the optionally substituted bicyclic or tricyclic fused group is an optionally substituted 8- to 14-membered heterocyclic or heteroaromatic group, and is unsubstituted or substituted with one or more of optionally substituted C₁-C₆ alkyl, optionally substituted C₂-C₆ alkenyl, optionally substituted C₂-C₆ alkynyl, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl, and R¹⁶ and R¹⁷ are independently hydrogen, an optionally substituted C₁-C₆ alkyl, an optionally substituted C₂-C₆ alkenyl, an optionally substituted C₂-C₆ alkynyl, optionally substituted C₆₋₁₂ aryl, optionally substituted C₃-C₆ cycloalkyl, optionally substituted 3 to 8 membered heterocyclyl or an optionally substituted 5 to 10 membered heteroaryl.

11. The compound according to claim 1, wherein Z has formula: 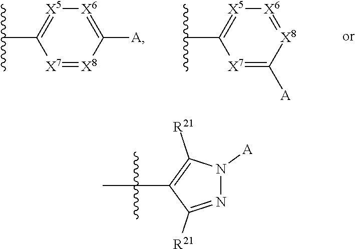
wherein X⁵, X⁶, X⁷ and X⁸ are N or CR²¹ with the proviso that no more than one of X⁵, X⁶, X⁷ and X⁸ is N;
R²¹ in each occurrence is independently H, a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 6 membered heterocyclyl, an optionally substituted 5 or 6 membered heteroaryl, an optionally substituted C₆₋₁₀ aryl or an optionally substituted C₃₋₉ cycloalkyl; and
A is selected from an optionally substituted C₁-C₆ alkyl, OR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 10 membered heterocyclyl and an optionally substituted 5 to 10 membered heteroaryl.

12. The compound according to claim 1, wherein the phenyl or 5 or 6 membered heteroaryl group Z is substituted with an optionally substituted 3 to 10 membered heterocyclyl, an optionally substituted 5 to 10 membered heteroaryl, an optionally substituted C₆₋₁₀ aryl or an optionally substituted C₃₋₉ cycloalkyl.

13. The compound according to claim 12, wherein the phenyl or 5 or 6 membered heteroaryl group Z is substituted a heterocyclyl group of formula (i) or (j): 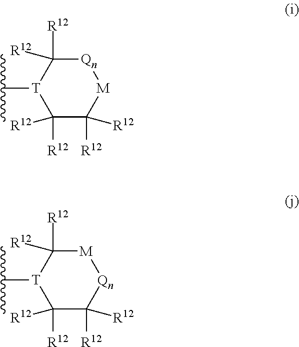
wherein:
T is N and M is NR¹³, CR¹⁴R¹⁵, O, S or SO₂; or T is CR¹⁸ and M is NR¹³, O, S or SO₂;
Q is C(R¹²)₂ and n is 0, 1 or 2;
R¹² in each occurrence is independently H, optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl; and/or two R¹² groups bonded to the same carbon may define an oxo group, two R¹² groups bonded to adjacent carbon atoms may be linked to form a fused group or two R¹² groups bonded to non-adjacent carbon atoms may be linked to form a bicyclic bridged group;
R¹³ is H, optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl;
R¹⁴ and R¹⁵ are each independently selected from H, optionally substituted C₁-C₆ alkyl, optionally substituted C₂-C₆ alkenyl, optionally substituted C₂-C₆ alkynyl, optionally substituted C₁-C₆ alkoxy, halogen, oxo, CN, OR¹⁶, SR¹⁶, SOR¹⁶, SO₂R¹⁶, COR¹⁶, COOR¹⁶, CONR¹⁶R¹⁷, NR¹⁶COR¹⁷, NR¹⁶R¹⁷, optionally substituted C₆₋₁₂ aryl, optionally substituted C₃₋₆ cycloalkyl, optionally substituted 3 to 6 membered heterocycle or optionally substituted 5 to 10 membered heteroaryl.

14. The compound according to claim 1, wherein the phenyl or 5 or 6 membered heteroaryl group Z is substituted with one or more substituents selected from the group consisting of a halogen, an optionally substituted C₁-C₆ alkyl, an optionally substituted C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹ and NR⁸R⁹.

15. The compound according to claim 1, wherein the compound is:
4-((8-methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((1,8-dimethyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
N-(3-fluoro-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(1-methylpiperidin-4-yl)phenyl)-2-oxo-1,2-dihydropyridine-3-carboxamide;
N-(4-(4-(2-hydroxyethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-isopropylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((1-methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((8-methyl-2-oxo-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(pyridin-2-yl)phenyl)-1,2-dihydropyridine-3-carboxamide;
4-((8-methoxy-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(tetrahydro-2H-pyran-4-yl)phenyl)-1,2-dihydropyridine-3-carboxamide;
N-(3-fluoro-4-(4-isopropylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-ethylpiperazin-1-yl)-3-fluorophenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-ethylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N,N-dimethyl-4-(4-(4-((8-methyl-2,3-dihydro-1H-pyrido[2,3-b][1,4]oxazin-7-yl)amino)-2-oxo-1,2-dihydropyridine-3-carboxamido)phenyl) piperazine-1-carboxamide;
N-(4-(1,1-dioxidothiomorpholino)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(piperazin-1-yl)phenyl)-1,2-dihydropyridine-3-carboxamide;
N-(4-(4-(2-methoxyethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3,3-difluoropyrrolidine-1-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(1H-pyrazol-4-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(1H-pyrazol-3-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(oxazol-5-yl)phenyl)-2-oxo-1,2-dihydropyridine-3-carboxamide;
N-(4-(4-cyclopentylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(pyridin-3-yl)phenyl)-1,2-dihydropyridine-3-carboxamide;
4-((8-methyl-2,3-dihydro-1H-pyrido[2,3-b][1,4]oxazin-7-yl)amino)-2-oxo-N-(4-(pyridin-4-yl)phenyl)-1,2-dihydropyridine-3-carboxamide;
N-(4-(3-aminopiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-aminopiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-(2-hydroxyethyl) piperazin-1-yl)phenyl)-2-oxo-1,2-dihydropyridine-3-carboxamide;
N-(4-(4,4-difluoropiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(8-methyl-3,8-diazabicyclo[3.2.1]octan-3-yl)phenyl)-2-oxo-1,2-dihydropyridine-3-carboxamide;
N-(2-methyl-1,2,3,4-tetrahydroisoquinolin-7-yl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
N-(4-(4-cyclopropylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(3-methyl-1,2,3,4,4a,5-hexahydrobenzo[b]pyrazino[1,2-d][1,4]oxazin-8-yl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-isopropylpiperazin-1-yl)phenyl)-2-oxo-1,2-dihydropyridine-3-carboxamide;
N-(4-(4-(2-hydroxypropyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-(2-methoxyethyl) piperazin-1-yl)phenyl)-2-oxo-1,2-dihydropyridine-3-carboxamide;
N-(4-(4-methylpiperazin-1-yl)phenyl)-2-oxo-4-((3,3,8-trimethyl-2,3-dihydro-1H-pyrido[2,3-b][1,4]oxazin-7-yl)amino)-1,2-dihydropyridine-3-carboxamide;
N-(4-(4-(2-methoxyethyl) piperazin-1-yl)phenyl)-2-oxo-4-((3,3,8-trimethyl-2,3-dihydro-1H-pyrido[2,3-b][1,4]oxazin-7-yl)amino)-1,2-dihydropyridine-3-carboxamide;
N-(4-(4-((1H-pyrazol-5-yl)methyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3-hydroxypropyl) pipcrazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-((5-oxopyrrolidin-2-yl)methyl) piperazin-1-yl)phenyl)-1,2-dihydropyridine-3-carboxamide;
N-(4-(4-isopropylpiperazin-1-yl)phenyl)-2-oxo-4-((3,3,8-trimethyl-2,3-dihydro-1H-pyrido[2,3-b][1,4]oxazin-7-yl)amino)-1,2-dihydropyridine-3-carboxamide;
N-(4-(4-(2-cyanoethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-cyclobutylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(2-methyl-3-oxo-1,2,3,4-tetrahydroisoquinolin-7-yl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(8-methyl-3,8-diazabicyclo[3.2.1]octan-3-yl)phenyl)-2-oxo-1,2-dihydropyridine-3-carboxamide;
4-(4-(4-((3,8-dimethyl-2,3-dihydro-1H-pyrido[2,3-b][1,4]oxazin-7-yl)amino)-2-oxo-1,2-dihydropyridine-3-carboxamido)phenyl)-N,N-dimethylpiperazine-1-carboxamide;
4-((3,8-dimethyl-2,3-dihydro-1H-pyrido[2,3-b][1,4]oxazin-7-yl)amino)-N-(3-fluoro-4-(4-isopropylpiperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(1-methyl-5-oxopyrrolidin-3-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(3-fluoro-4-(4-(2-methoxyethyl) piperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(3-fluoro-4-(4-(2-hydroxyethyl) piperazin-1-yl)phenyl)-2-oxo-1,2-dihydropyridine-3-carboxamide;
N-(4-(4-cyclopentylpiperazin-1-yl)phenyl)-4-((3,8-dimethyl-2,3-dihydro-1H-pyrido[2,3-b][1,4]oxazin-7-yl)amino)-2-oxo-1,2-dihydropyridine-3-carboxamide;
4-((3-ethyl-8-methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((3-isopropyl-8-methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((8′-methyl-1′,2′-dihydrospiro[cyclopropane-1,3′-pyrido[2,3-b][1,4]oxazin]-7′-yl)amino)-N-(4-(4-methylpiperazin-1-yl)phenyl)-2-oxo-1,2-dihydropyridine-3-carboxamide;
N-(3-cyano-4-(4-methylpiperazin-1-yl)phenyl)-4-((3,8-dimethyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(1-(1-methylpiperidin-4-yl)-1H-pyrazol-4-yl)-2-oxo-1,2-dihydropyridine-3-carboxamide;
N-(4-(4-((1H-pyrazol-5-yl)methyl) piperazin-1-yl)phenyl)-4-((3,8-dimethyl-2,3-dihydro-1H-pyrido[2,3-b][1,4]oxazin-7-yl)amino)-2-oxo-1,2-dihydropyridine-3-carboxamide;
N-(3-fluoro-4-(4-(2-methoxyethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(2-methyl-4-(4-methylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-methylpiperazin-1-yl)-3-(trifluoromethyl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(2-(methoxymethyl)-4-methylpiperazin-1-yl)phenyl)-2-oxo-1,2-dihydropyridine-3-carboxamide;
4-((3-fluoro-8-methyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(3-methyl-4-(4-methylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(hexahydropyrrolo[1,2-a]pyrazin-2 (1H)-yl)phenyl)-2-oxo-1,2-dihydropyridine-3-carboxamide;
N-(4-(1-(2-methoxyethyl) piperidin-4-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(3-methoxy-4-(4-methylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(3-(hydroxymethyl)-4-(4-methylpiperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-morpholinophenyl)-2-oxo-1,2-dihydropyridine-3-carboxamide;
4-((8-methyl-2,3-dihydro-1H-pyrido[2,3-b][1,4]oxazin-7-yl)amino)-N-(4-((4-methylpiperazin-1-yl)methyl)phenyl)-2-oxo-1,2-dihydropyridine-3-carboxamide;
N-(2-fluoro-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(3-(dimethylcarbamoyl)-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-((1-methylpiperidin-4-yl)amino)phenyl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-sulfamoylphenyl)-1,2-dihydropyridine-3-carboxamide;
4-((8-methyl-2,3-dihydro-1H-pyrido[2,3-b][1,4]oxazin-7-yl)amino)-N-(4-(4-methylpiperazin-1-yl)-3-(4H-1,2,4-triazol-4-yl)phenyl)-2-oxo-1,2-dihydropyridine-3-carboxamide;
N-(4-(3-(2-hydroxypropan-2-yl) piperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-((4-methylpiperazin-1-yl)methyl)-3-(trifluoromethyl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(1-methylpiperidin-3-yl)phenyl)-2-oxo-1,2-dihydropyridine-3-carboxamide;
5-(4-((8-methyl-2,3-dihydro-1H-pyrido[2,3-b][1,4]oxazin-7-yl)amino)-2-oxo-1,2-dihydropyridine-3-carboxamido)-2-(4-methylpiperazin-1-yl)benzoic acid;
1-(4-(4-((8-methyl-2,3-dihydro-1H-pyrido[2,3-b][1,4]oxazin-7-yl)amino)-2-oxo-1,2-dihydropyridine-3-carboxamido)phenyl) piperidine-4-carboxylic acid;
4-((8-methyl-2,3-dihydro-1H-pyrido[2,3-b][1,4]oxazin-7-yl)amino)-N-(2-methyl-4-(methylsulfonamido)phenyl)-2-oxo-1,2-dihydropyridine-3-carboxamide;
N-(1-ethyl-3,3-dimethyl-2-oxoindolin-5-yl)-4-((8-methyl-2,3-dihydro-1H-pyrido[2,3-b][1,4]oxazin-7-yl)amino)-2-oxo-1,2-dihydropyridine-3-carboxamide;
(S)—N-(4-(3,4-dimethylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(R)—N-(4-(3,4-dimethylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(3-oxo-3,4-dihydro-2H-benzo[b][1,4]oxazin-6-yl)-1,2-dihydropyridine-3-carboxamide;
4-((8-methyl-2,3-dihydro-1H-pyrido[2,3-b][1,4]oxazin-7-yl)amino)-N-(3-(4-methylpiperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(methylsulfonyl)phenyl)-2-oxo-1,2-dihydropyridine-3-carboxamide;
N-(2-methyl-1H-benzo[d]imidazol-5-yl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(3-(methylsulfonyl) propyl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(4-(2-morpholinocthyl) piperazin-1-yl)phenyl)-2-oxo-1,2-dihydropyridine-3-carboxamide;
N-(3-((dimethylamino)methyl)-4-(4-methylpipcrazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(S)-4-((8-methyl-2,3-dihydro-1H-pyrido[2,3-b][1,4]oxazin-7-yl)amino)-2-oxo-N-(4-((2-oxooxazolidin-4-yl)methyl)phenyl)-1,2-dihydropyridine-3-carboxamide;
N-(5-chloro-6-(2H-1,2,3-triazol-2-yl) pyridin-3-yl)-4-((8-methyl-2,3-dihydro-1H-pyrido[2,3-b][1,4]oxazin-7-yl)amino)-2-oxo-1,2-dihydropyridine-3-carboxamide;
N-(2,3-dihydrobenzo[b][1,4]dioxin-6-yl)-4-((8-methyl-2,3-dihydro-1H-pyrido[2,3-b][1,4]oxazin-7-yl)amino)-2-oxo-1,2-dihydropyridine-3-carboxamide;
N-(4-(N-cyclopropylsulfamoyl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-((1,1-dioxidothiomorpholino)methyl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(pyridin-4-ylmethyl)phenyl)-1,2-dihydropyridine-3-carboxamide;
N-(1H-indazol-5-yl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(5-methyl-2,5-diazabicyclo[2.2.2]octan-2-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-((1S,4S)-5-methyl-2,5-diazabicyclo[2.2.1]heptan-2-yl)phenyl)-2-oxo-1,2-dihydropyridine-3-carboxamide;
N-(1H-indazol-6-yl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-(azetidin-3-yl)ethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-methyl-3-oxo-3,4-dihydro-2H-benzo[b][1,4]oxazin-6-yl)-2-oxo-1,2-dihydropyridine-3-carboxamide;
N-(3,4-dihydro-2H-benzo[b][1,4]oxazin-6-yl)-4-((8-methyl-2,3-dihydro-1H-pyrido[2,3-b][1,4]oxazin-7-yl)amino)-2-oxo-1,2-dihydropyridine-3-carboxamide;
(R)—N-(4-(2,4-dimethylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-carbamoylphenyl)-4-((8-methyl-2,3-dihydro-1H-pyrido[2,3-b][1,4]oxazin-7-yl)amino)-2-oxo-1,2-dihydropyridine-3-carboxamide;
N-(6-acctamidopyridin-3-yl)-4-((8-methyl-2,3-dihydro-1H-pyrido[2,3-b][1,4]oxazin-7-yl)amino)-2-oxo-1,2-dihydropyridine-3-carboxamide;
N-(benzo[d][1,3]dioxol-5-yl)-4-((8-methyl-2,3-dihydro-1H-pyrido[2,3-b][1,4]oxazin-7-yl)amino)-2-oxo-1,2-dihydropyridine-3-carboxamide;
N-(3-((2-hydroxyethyl) sulfonyl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(1,2,3,4-tetrahydroquinolin-7-yl)-1,2-dihydropyridine-3-carboxamide;
N-(4-(4-carbamoylpiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(benzo[d]thiazol-6-yl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(2-methylbenzo[d]thiazol-6-yl)-2-oxo-1,2-dihydropyridine-3-carboxamide;
4-((8-methyl-2,3-dihydro-1H-pyrido[2,3-b][1,4]oxazin-7-yl)amino)-N-(1-methyl-2-oxo-1,2,3,4-tetrahydroquinolin-6-yl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-propionylpiperazin-1-yl)phenyl)-1,2-dihydropyridine-3-carboxamide;
4-((8-methyl-2,3-dihydro-1H-pyrido[2,3-b][1,4]oxazin-7-yl)amino)-2-oxo-N-(2-oxo-1,2,3,4-tetrahydroquinolin-6-yl)-1,2-dihydropyridine-3-carboxamide;
N-(4-(4-methoxyphenoxy)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(1,2,3,4-tetrahydroquinolin-6-yl)-1,2-dihydropyridine-3-carboxamide;
N-(4-(1H-imidazol-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(oxetan-3-yl) piperazin-1-yl)phenyl)-2-oxo-1,2-dihydropyridine-3-carboxamide;
N-(1H-benzo[d]imidazol-6-yl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(2-oxo-1,2,3,4-tetrahydroquinolin-7-yl)-1,2-dihydropyridine-3-carboxamide;
N-(4-(4-cyclopentylpiperazin-1-yl)-3-fluorophenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(1-oxo-1,2,3,4-tetrahydroisoquinolin-7-yl)-1,2-dihydropyridine-3-carboxamide;
N-(3-chloro-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(methylsulfonyl) piperazin-1-yl)phenyl)-2-oxo-1,2-dihydropyridine-3-carboxamide;
N-(3-amino-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(1-ethyl-1H-pyrazol-5-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(1-ethyl-3-(trifluoromethyl)-1H-pyrazol-5-yl)phenyl)-4-((8-methyl-2,3-dihydro-1H-pyrido[2,3-b][1,4]oxazin-7-yl)amino)-2-oxo-1,2-dihydropyridine-3-carboxamide;
(S)—N-(4-(2,4-dimethylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-methyl-2-oxopiperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(3-oxopiperazin-1-yl)phenyl)-1,2-dihydropyridine-3-carboxamide;
4-((8-methyl-2,3-dihydro-1H-pyrido[2,3-b][1,4]oxazin-7-yl)amino)-2-oxo-N-(3-oxo-3,4-dihydro-2H-benzo[b][1,4]oxazin-7-yl)-1,2-dihydropyridine-3-carboxamide;
N-(3-bromo-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(1-isopropyl-1H-pyrazol-5-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-methyl-3-oxopiperazin-1-yl)phenyl)-2-oxo-1,2-dihydropyridine-3-carboxamide;
N-(3-hydroxyquinoxalin-6-yl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(4-methylpiperazin-1-yl) piperidin-1-yl)phenyl)-2-oxo-1,2-dihydropyridine-3-carboxamide;
N-(4-(4-(2-methoxyethyl) piperazin-1-yl)-3-methylphenyl)-4-((8-methyl-2,3-dihydro-1H-pyrido[2,3-b][1,4]oxazin-7-yl)amino)-2-oxo-1,2-dihydropyridine-3-carboxamide;
N-(3-fluoro-4-(1-(2-methoxyethyl) piperidin-4-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3-methoxypropyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-hydroxypiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(1-isopropylpiperidin-4-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-methylpiperidin-1-yl)phenyl)-2-oxo-1,2-dihydropyridine-3-carboxamide;
N-(4-((2-ethylhexyl) carbamoyl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-hydroxy-2-methylpropyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(cyclopentanccarbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-methoxyacetyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3-methoxypropanoyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-(pyridin-3-yl) piperazin-1-yl)phenyl)-1,2-dihydropyridine-3-carboxamide;
N-methyl-4-(4-(4-((8-methyl-2,3-dihydro-1H-pyrido[2,3-b][1,4]oxazin-7-yl)amino)-2-oxo-1,2-dihydropyridine-3-carboxamido)phenyl) piperazine-1-carboxamide;
4-((8-methyl-2,3-dihydro-1H-pyrido[2,3-b][1,4]oxazin-7-yl)amino)-2-oxo-N-(4-(4-(pyridin-4-yl) piperazin-1-yl)phenyl)-1,2-dihydropyridine-3-carboxamide;
4-((8-methyl-2,3-dihydro-1H-pyrido[2,3-b][1,4]oxazin-7-yl)amino)-2-oxo-N-(4-(4-(pyridin-2-yl) piperazin-1-yl)phenyl)-1,2-dihydropyridine-3-carboxamide;
N-(2-methoxyethyl)-N-methyl-4-(4-(4-((8-methyl-2,3-dihydro-1H-pyrido[2,3-b][1,4]oxazin-7-yl)amino)-2-oxo-1,2-dihydropyridine-3-carboxamido)phenyl) piperazine-1-carboxamide;
N-(3-(dimethylamino)-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(1-methylpiperidin-4-yl) piperazin-1-yl)phenyl)-2-oxo-1,2-dihydropyridine-3-carboxamide;
N-(4-(4-((1S,2S)-2-fluorocyclopropane-1-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(3-amino-4-(4-methylpiperazin-1-yl)phenyl)-4-((3,8-dimethyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(3-(methylamino)-4-(4-methylpiperazin-1-yl)phenyl)-2-oxo-1,2-dihydropyridine-3-carboxamide;
N-(2-((1S,2S)-2-fluorocyclopropane-1-carboxamido)benzo[d]thiazol-6-yl)-4-((8-methyl-2,3-dihydro-1H-pyrido[2,3-b][1,4]oxazin-7-yl)amino)-2-oxo-1,2-dihydropyridine-3-carboxamide;
N-(2-((1S,2S)-2-fluorocyclopropane-1-carboxamido)benzo[d]thiazol-5-yl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-(dimethylamino)-2-oxoethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((1H-indazol-3-yl)amino)-N-(4-(4-methylpiperazin-1-yl)phenyl)-2-oxo-1,2-dihydropyridine-3-carboxamide;
4-((8-cyano-2,3-dihydro-1H-pyrido[2,3-b][1,4]oxazin-7-yl)amino)-N-(4-(4-methylpiperazin-1-yl)phenyl)-2-oxo-1,2-dihydropyridine-3-carboxamide;
N-(4-(4-(2-hydroxypropan-2-yl) piperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(hydroxymethyl)-4-methylpiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(1-hydroxyethyl) piperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(1H-pyrazole-5-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3,3-dimethylcyclobutane-1-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(2-methylcyclopropane-1-carbonyl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(4-(1-methylcyclobutane-1-carbonyl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(4-(3-methylcyclobutane-1-carbonyl) piperazin-1-yl)phenyl)-2-oxo-1,2-dihydropyridine-3-carboxamide;
N-(4-(4-((1-methyl-1H-pyrazol-5-yl)methyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2,2-dimethylcyclopropane-1-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-methoxy-2-methylpropyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(3-amino-4-(4-cyclopentylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-cyclopropylacetyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(3-chloro-4-(4-propionylpiperazin-1-yl)phenyl)-4-((3,8-dimethyl-2,3-dihydro-1H-pyrido[2,3-b][1,4]oxazin-7-yl)amino)-2-oxo-1,2-dihydropyridine-3-carboxamide;
N-(4-(4-(cyclobutanecarbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-isobutyrylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(1-methyl-1H-pyrazole-5-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3,3-dimethylazetidine-1-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3,3-difluorocyclobutane-1-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3,3-difluoroazetidine-1-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-azaspiro[3.3]heptane-2-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-oxa-6-azaspiro[3.3]heptane-6-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(1-methyl-1H-pyrazole-3-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(R)—N-(4-(hexahydropyrazino[2,1-c][1,4]oxazin-8 (1H)-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-(pyrrolidine-1-carbonyl) piperazin-1-yl)phenyl)-1,2-dihydropyridine-3-carboxamide;
N-(4-(4-(azetidine-1-carbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(3-fluoro-4-(4-(4-isopropylpiperazin-1-yl) piperidin-1-yl)phenyl)-2-oxo-1,2-dihydropyridine-3-carboxamide;
(S)—N-(4-(hexahydropyrazino[2,1-c][1,4]oxazin-8 (1H)-yl)phenyl)-4-((8-methyl-2,3-dihydro-1H-pyrido[2,3-b][1,4]oxazin-7-yl)amino)-2-oxo-1,2-dihydropyridine-3-carboxamide;
(S)—N-(4-(hexahydropyrrolo[1,2-a]pyrazin-2 (1H)-yl)phenyl)-4-((8-methyl-2,3-dihydro-1H-pyrido[2,3-b][1,4]oxazin-7-yl)amino)-2-oxo-1,2-dihydropyridine-3-carboxamide;
(R)—N-(4-(hexahydropyrrolo[1,2-a]pyrazin-2 (1H)-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-cyclohexylphenyl)-4-((8-methyl-2,3-dihydro-1H-pyrido[2,3-b][1,4]oxazin-7-yl)amino)-2-oxo-1,2-dihydropyridine-3-carboxamide;
N-([1,l′-biphenyl]-4-yl)-4-((8-methyl-2,3-dihydro-1H-pyrido[2,3-b][1,4]oxazin-7-yl)amino)-2-oxo-1,2-dihydropyridine-3-carboxamide;
N-(4-(4-(cyclopropanecarbonyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(2-methylbutanoyl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-(2,2,3,3-tetramethylcyclopropane-1-carbonyl) piperazin-1-yl)phenyl)-1,2-dihydropyridine-3-carboxamide;
4-((3,8-dimethyl-2,3-dihydro-1H-pyrido[2,3-b][1,4]oxazin-7-yl)amino)-N-(4-(4-isobutyrylpiperazin-1-yl)phenyl)-2-oxo-1,2-dihydropyridine-3-carboxamide;
N-(4-(4-(1-methyl-1H-pyrazol-3-yl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(2-methylpyridin-3-yl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-(pyrimidin-5-yl) piperazin-1-yl)phenyl)-1,2-dihydropyridine-3-carboxamide;
N-(4-(4-(1-methyl-1H-1,2,4-triazol-3-yl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(2-methylpyridin-4-yl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-pivaloylpiperazin-1-yl)phenyl)-1,2-dihydropyridine-3-carboxamide;
N-(4-(4-(2-fluoro-2-methylpropanoyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide; (S)—N-(4-(4-isobutyryl-3-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-methoxy-2-methylpropanoyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(S)-4-((8-methyl-2,3-dihydro-1H-pyrido[2,3-b][1,4]oxazin-7-yl)amino)-N-(4-(3-methyl-4-propionylpiperazin-1-yl)phenyl)-2-oxo-1,2-dihydropyridine-3-carboxamide;
(S)—N-(4-(4-(2-methoxyethyl)-3-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(R)-4-((8-methyl-2,3-dihydro-1H-pyrido[2,3-b][1,4]oxazin-7-yl)amino)-N-(4-(3-methyl-4-propionylpiperazin-1-yl)phenyl)-2-oxo-1,2-dihydropyridine-3-carboxamide;
(R)—N-(4-(4-(2-methoxyethyl)-3-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(R)—N-(4-(4-isobutyryl-3-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(S)—N-(4-(4-(2-methoxypropanoyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(1-methoxypropan-2-yl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-cyanopiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(R)—N-(4-(4-(2-methoxypropanoyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(methylalanyl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(4-(1-methylcyclopropane-1-carbonyl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(4-(1-methylpyrrolidine-3-carbonyl) piperazin-1-yl)phenyl)-2-oxo-1,2-dihydropyridine-3-carboxamide;
N-(4-(4-(2-hydroxy-2-methylpropanoyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(dimethylalanyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
(S)-4-((8-methyl-2,3-dihydro-1H-pyrido[2,3-b][1,4]oxazin-7-yl)amino)-2-oxo-N-(4-(6-oxohexahydropyrrolo[1,2-a]pyrazin-2 (1H)-yl)phenyl)-1,2-dihydropyridine-3-carboxamide;
4-((3,8-dimethyl-2,3-dihydro-1H-pyrido[2,3-b][1,4]oxazin-7-yl)amino)-N-(3-fluoro-4-(4-methylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-ethylpiperazin-1-yl)-3-fluorophenyl)-2-oxo-1,2-dihydropyridine-3-carboxamide;
4-((3,8-dimethyl-2,3-dihydro-1H-pyrido[2,3-b][1,4]oxazin-7-yl)amino)-2-oxo-N-(4-(4-propionylpiperazin-1-yl)phenyl)-1,2-dihydropyridine-3-carboxamide;
4-((8-methyl-2,3-dihydro-1H-pyrido[2,3-b][1,4]oxazin-7-yl)amino)-N-(4-(4-(6-methylpyridin-3-yl) piperazin-1-yl)phenyl)-2-oxo-1,2-dihydropyridine-3-carboxamide;
N-(4-(4-(2-(methoxymethyl)pyridin-4-yl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N,N-dimethyl-4-(4-(4-(4-((8-methyl-2,3-dihydro-1H-pyrido[2,3-b][1,4]oxazin-7-yl)amino)-2-oxo-1,2-dihydropyridine-3-carboxamido)phenyl) piperazin-1-yl) picolinamide;
N-(4-(4-(1-methyl-1H-imidazol-2-yl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-(thiazol-4-yl) piperazin-1-yl)phenyl)-1,2-dihydropyridine-3-carboxamide;
N-(4-(4-(2-ethoxyethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(2-methoxypropyl) piperazin-1-yl)phenyl)-4-((8-methyl-2,3-dihydro-1H-pyrido[2,3-b] [1,4]oxazin-7-yl)amino)-2-oxo-1,2-dihydropyridine-3-carboxamide;
N-(4-(4-isobutylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-(thiazol-2-yl) piperazin-1-yl)phenyl)-1,2-dihydropyridine-3-carboxamide;
N-(4-(4-(cyclopropanecarbonyl) piperazin-1-yl)phenyl)-4-((3,8-dimethyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(1-methylazetidine-3-carbonyl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(6-oxooctahydro-2H-pyrido[1,2-a]pyrazin-2-yl)phenyl)-1,2-dihydropyridine-3-carboxamide;
N-(4-(4-((dimethylamino)methyl)-4-hydroxypiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-cyclopropylpiperazin-1-yl)-3-fluorophenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(2-methyl-3-oxohexahydroimidazo[1,5-a]pyrazin-7 (1H)-yl)phenyl)-2-oxo-1,2-dihydropyridine-3-carboxamide;
N-(4-(4-(1-(1H-pyrazol-5-yl)ethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(3-fluoro-4-(4-(oxetan-3-yl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-(3-methyloxetan-3-yl) piperazin-1-yl)phenyl)-2-oxo-1,2-dihydropyridine-3-carboxamide;
4-((8-methyl-2,3-dihydro-1H-pyrido[2,3-b][1,4]oxazin-7-yl)amino)-N-(4-(3-(N-methylisobutyramido) pyrrolidin-1-yl)phenyl)-2-oxo-1,2-dihydropyridine-3-carboxamide;
N-(4-(6-azaspiro[2.5]octan-6-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(tert-butyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(4-(1-isopropylpiperidin-4-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-ethylpiperazin-1-yl)-3-fluorophenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-ethylpiperazin-1-yl)-3-fluorophenyl)-2-oxo-1,2-dihydropyridine-3-carboxamide;
N-(3-fluoro-4-(4-(2-fluoroethyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-((dimethylamino)methyl)-4-hydroxypiperidin-1-yl)-3-fluorophenyl)-2-oxo-1,2-dihydropyridine-3-carboxamide;
N-(3-chloro-4-(4-ethylpiperazin-1-yl)-5-fluorophenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-ethylpiperazin-1-yl)-3,5-difluorophenyl)-4-((8-methyl-2,3-dihydro-1H-pyrido[2,3-b][1,4]oxazin-7-yl)amino)-2-oxo-1,2-dihydropyridine-3-carboxamide;
N-(3-chloro-4-(4-cyclopropylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-((dimethylamino)methyl)-4-methoxypiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(3-(aminomethyl)-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(3-(2-hydroxyethyl)-4-(4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(3-aminopyrrolidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(2-hydroxypropan-2-yl)-1-methoxyisoquinolin-6-yl)-4-((8-methyl-2,3-dihydro-1H-pyrido[2,3-b][1,4]oxazin-7-yl)amino)-2-oxo-1,2-dihydropyridine-3-carboxamide;
N-(4-(2-(hydroxymethyl) pyrrolidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(3-hydroxypyrrolidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(4-(4-propylpiperazin-1-yl)phenyl)-1,2-dihydropyridine-3-carboxamide;
N-(4-(4-cyclopropylpiperazin-1-yl)-3-methylphenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-cyclopropylpiperazin-1-yl)-3-ethoxyphenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-acetylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-(3-hydroxypropanoyl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-isopropylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-isopropylpiperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-(3-methoxypropyl) piperazin-1-yl)phenyl)-2-oxo-1,2-dihydropyridine-3-carboxamide;
4-((3,8-dimethyl-2,3-dihydro-1H-pyrido[2,3-b][1,4]oxazin-7-yl)amino)-N-(4-(4-(1-methyl-1H-pyrazol-3-yl) piperazin-1-yl)phenyl)-2-oxo-1,2-dihydropyridine-3-carboxamide;
N-(4-(2-(hydroxymethyl)-4-methylpiperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-2-oxo-N-(1-(2,2,2-trifluoroethyl)-1H-pyrazol-4-yl)-1,2-dihydropyridine-3-carboxamide;
N-(4-(4-(1-methyl-1H-pyrazol-4-yl) piperazin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
N-(4-(4-cyclopropylpiperidin-1-yl)phenyl)-4-((8-methyl-2,3-dihydro-1H-pyrido[2,3-b][1,4]oxazin-7-yl)amino)-2-oxo-1,2-dihydropyridine-3-carboxamide;
4-((8-methyl-2,3-dihydro-1H-pyrido[2,3-b][1,4]oxazin-7-yl)amino)-N-(4-(4-methylpiperazin-1-yl)phenyl)-6-oxo-1,6-dihydropyrimidine-5-carboxamide;
N-(4-(4-isopropylpiperazin-1-yl)phenyl)-4-((8-methyl-2,3-dihydro-1H-pyrido[2,3-b][1,4]oxazin-7-yl)amino)-6-oxo-1,6-dihydropyrimidine-5-carboxamide; or
N-(4-(4-(2-methoxyethyl) piperazin-1-yl)phenyl)-4-((8-methyl-2,3-dihydro-1H-pyrido[2,3-b][1,4]oxazin-7-yl)amino)-6-oxo-1,6-dihydropyrimidine-5-carboxamide.

16. A PROTAC of formula (II):
PTM-L-ULM (II),
wherein PTM is a protein targeting moiety, and is a compound of formula (I), as defined claim 1;
L is a linker; and
ULM is an E3 ubiquintinylating ligase complex.

17. A pharmaceutical composition comprising a compound according to claim 1, or a pharmaceutically acceptable salt, solvate, tautomeric form or polymorphic form thereof, and a pharmaceutically acceptable vehicle.

18. (canceled)

19. A method of modulating activity of the HPK-1 protein comprising administering a therapeutically effective amount of a compound of formula (I), as defined by claim 1, or a pharmaceutically acceptable complex, salt, solvate, tautomeric form or polymorphic form thereof, to a subject in need thereof.

20. A method of treating, ameliorating or preventing a disease selected from cancer, viral infection and immune-mediated disorder, the method comprising administering, to a subject in need of such treatment, a therapeutically effective amount of a compound of formula (I), as defined by claim 1, or a pharmaceutically acceptable complex, salt, solvate, tautomeric form or polymorphic form thereof.

21. A method of modulating activity of the HPK-1 protein comprising administering a therapeutically effective amount of a compound of formula (I): 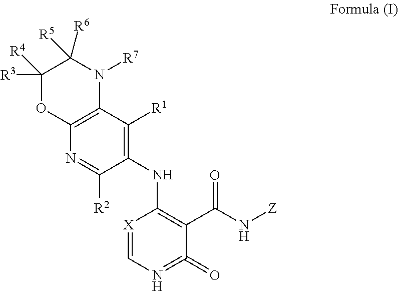
wherein X is CH or N;
Z is a phenyl or 5 or 6 membered heteroaryl, wherein the phenyl or heteroaryl is substituted with one or more substituents selected from the group consisting of a halogen, an optionally substituted C₁-C₆ alkyl, an optionally substituted
C₂-C₆ alkenyl, an optionally substituted C₂-C₆ alkynyl, CN, OR⁸, SR⁸, SOR⁸, SO₂R⁸, SO₂NR⁸R⁹, COR⁸, COOR⁸, CONR⁸R⁹, NR⁸COR⁹, NR⁸SO₂R⁹, NR⁸R⁹, an optionally substituted 3 to 10 membered heterocyclyl, an optionally substituted 5 to 10 membered heteroaryl, an optionally substituted C₆₋₁₀ aryl or an optionally substituted C₃₋₉ cycloalkyl; and/or where adjacent substituents of the phenyl or heteroaryl, together with the atoms to which they are attached, may combine to form an optionally substituted 3 to 6 membered heterocycle or an optionally substituted 5 or 6 membered heteroaryl;
R¹ to R⁷ are independently hydrogen, an optionally substituted C₁-C₆ alkyl, an optionally substituted C₂-C₆ alkenyl, an optionally substituted C₂-C₆ alkynyl, a halogen, CN, OR¹⁰, SR⁸, SOR⁸, SO₂R⁸, SO₂NR⁸R⁹, COR¹⁰, COOR⁸, CONR¹⁰R¹¹, NR¹⁰COR¹¹, NR¹⁰SO₂R¹¹, NR¹⁰R¹¹, an optionally substituted
C₃-C₆ cycloalkyl, an optionally substituted 3 to 8 membered heterocyclyl, an optionally substituted 5 to 10 membered heteroaryl or an optionally substituted phenyl; and/or R³ and R⁴ and/or R⁵ and R⁶, together with the C atom they are bound to, form a C═O group, an optionally substituted C₃-C₆ cycloalkyl or an optionally substituted 3 to 8 membered heterocyclyl; and/or R³ and R⁵, together with the C atoms they are bound to, form an optionally substituted C₃-C₆ cycloalkyl or an optionally substituted 3 to 8 membered heterocyclyl;
R⁸ and R⁹ are independently hydrogen, an optionally substituted C₁-C₁₂ alkyl, an optionally substituted C₂-C₁₂ alkenyl, an optionally substituted C₂-C₁₂ alkynyl, optionally substituted C₆₋₁₂ aryl, optionally substituted C₃-C₆ cycloalkyl, optionally substituted 3 to 8 membered heterocyclyl, and an optionally substituted 5 to 10 membered heteroaryl; and
R¹⁰ and R¹¹ are independently hydrogen, an optionally substituted C₁-C₆ alkyl, an optionally substituted C₂-C₆ alkenyl, an optionally substituted C₂-C₆ alkynyl, optionally substituted C₆₋₁₂ aryl, optionally substituted C₃-C₆ cycloalkyl, optionally substituted 3 to 8 membered heterocyclyl, and an optionally substituted 5 to 10 membered heteroaryl;
or a pharmaceutically acceptable complex, salt, solvate, tautomeric form or polymorphic form thereof, to a subject in need thereof.
